SEQUENCE LISTING (354 sequences)

SEQ ID NO: 1 - DNA, 25 bp, Bacteroides thetaiotaomicron
  source 1..25: mol_type = genomic DNA
ggtagtccacacagtaaacgatgaa

SEQ ID NO: 2 - DNA, 23 bp, Bacteroides thetaiotaomicron
  source 1..23: mol_type = genomic DNA
cccgtcaaattcctttgagtttc

SEQ ID NO: 3 - DNA, 22 bp, Bacteroides thetaiotaomicron
  source 1..22: mol_type = genomic DNA
tggttgtcggctatcaggaagt

SEQ ID NO: 4 - DNA, 21 bp, Bacteroides thetaiotaomicron
  source 1..21: mol_type = genomic DNA
acatctgccatgttggctttc

SEQ ID NO: 5 - DNA, 20 bp, Bacteroides thetaiotaomicron
  source 1..20: mol_type = genomic DNA
agcgtaaagccgacctgaca

SEQ ID NO: 6 - DNA, 21 bp, Bacteroides thetaiotaomicron
  source 1..21: mol_type = genomic DNA
tcaccttgcttctggatttcg

SEQ ID NO: 7 - DNA, 45 bp, Pseudorhabdus luminescens
  source 1..45: mol_type = genomic DNA
gctctagaactagtggatcctgatctggaagaagcaatgaaagct

SEQ ID NO: 8 - DNA, 41 bp, Pseudorhabdus luminescens
  source 1..41: mol_type = genomic DNA
catattcgtttaattaaataaataatttattttattttaaa

SEQ ID NO: 9 - DNA, 52 bp, Pseudorhabdus luminescens
  source 1..52: mol_type = genomic DNA
atttatttaattaaacgaatatgactaaaaaaatttcattcattattaacgg

SEQ ID NO: 10 - DNA, 50 bp, Pseudorhabdus luminescens
  source 1..50: mol_type = genomic DNA
aagataggcaattagtcgactcaactattaaatgcttggtttaagcttaa

SEQ ID NO: 11 - DNA, 47 bp, Pseudorhabdus luminescens
  source 1..47: mol_type = genomic DNA
tttacaatttgccatgcggattacgggacaaatacaaggaacttatc

SEQ ID NO: 12 - DNA, 78 bp, Pseudorhabdus luminescens
  source 1..78: mol_type = genomic DNA
tccgcatggcaaattgtaaattgtaaatcgtaaaatagtaatatattaatggaaaataaatccaaatataaaaccatc

SEQ ID NO: 13 - DNA, 42 bp, Pseudorhabdus luminescens
  source 1..42: mol_type = genomic DNA
attctttatcctcctccttattaagacagcgaaatcgcttga

SEQ ID NO: 14 - DNA, 50 bp, Pseudorhabdus luminescens
  source 1..50: mol_type = genomic DNA
taaggaggaggataaagaatatgacttcatatgttgataaacaagagatc

SEQ ID NO: 15 - DNA, 68 bp, Pseudorhabdus luminescens
  source 1..68: mol_type = genomic DNA
agtgttaccttcattcatccttcttcacccttcatttatatcaactattaaatgcttggtttaagctt

SEQ ID NO: 16 - DNA, 89 bp, Pseudorhabdus luminescens
  source 1..89: mol_type = genomic DNA
ggatgaatgaaggtaacactcataaactcgaaattcttcattcttaatttttaattaaaatatatgaaatttggaaactttttgcttac

SEQ ID NO: 17 - DNA, 50 bp, Pseudorhabdus luminescens
  source 1..50: mol_type = genomic DNA
ccattgtcttattcctttctttataatagcgaacgttgtttttctttaag

SEQ ID NO: 18 - DNA, 49 bp, Pseudorhabdus luminescens
  source 1..49: mol_type = genomic DNA
agaaaggaataagacaatggatatgaaatttggattgttcttccttaac

SEQ ID NO: 19 - DNA, 55 bp, Pseudorhabdus luminescens
  source 1..55: mol_type = genomic DNA
aagataggcaattagtcgacttacatgtggtactttttaatattatcatcaacaa

SEQ ID NO: 20 - DNA, 71 bp, Pseudorhabdus luminescens
  source 1..71: mol_type = genomic DNA
gctcggtacccggggatccactagtcactcccgcattttaaaataaaataaattatttatttaattaaacg

SEQ ID NO: 21 - DNA, 59 bp, Pseudorhabdus luminescens
  source 1..59: mol_type = genomic DNA
tgcatgcctgcaggtcgacttacatgtggtactttttaatattatcatcaacaatattg

SEQ ID NO: 22 - DNA, 44 bp, Pseudorhabdus luminescens
  source 1..44: mol_type = genomic DNA
gctcggtacccggggatcctgatctggaagaagcaatgaaagct

SEQ ID NO: 23 - DNA, 60 bp, Pseudorhabdus luminescens
  source 1..60: mol_type = genomic DNA
aaatgcgggagtgactagtcaaagttacgacaaataatttgttaacatacatatttaggc

SEQ ID NO: 24 - DNA, 44 bp, Pseudorhabdus luminescens
  source 1..44: mol_type = genomic DNA
gctcggtacccggggatccatctggaagaagtaatgaaagctgc

SEQ ID NO: 25 - DNA, 54 bp, Pseudorhabdus luminescens
  source 1..54: mol_type = genomic DNA
aaatgcgggagtgactagtcaaagttacgacaaataatttgttaacatacaaaa

SEQ ID NO: 26 - DNA, 53 bp, Pseudorhabdus luminescens
  source 1..53: mol_type = genomic DNA
gctcggtacccggggatcctatcattcagttttctgttggttactttgagtga

SEQ ID NO: 27 - DNA, 66 bp, Pseudorhabdus luminescens
  source 1..66: mol_type = genomic DNA
aaatgcgggagtgactagttagtttaatgttattaatttaaaagtacgaattttctcttttcgatg

SEQ ID NO: 28 - DNA, 43 bp, Pseudorhabdus luminescens
  source 1..43: mol_type = genomic DNA
gctcggtacccggggatccaaaatggaactgggcaatgacagg

SEQ ID NO: 29 - DNA, 52 bp, Pseudorhabdus luminescens
  source 1..52: mol_type = genomic DNA
aaatgcgggagtgactagtcctttttcttgtctggttggatagatgtttttt

SEQ ID NO: 30 - DNA, 50 bp, Pseudorhabdus luminescens
  source 1..50: mol_type = genomic DNA
gctcggtacccggggatccttgtttgttggagattgttttcatatcgttg

SEQ ID NO: 31 - DNA, 56 bp, Pseudorhabdus luminescens
  source 1..56: mol_type = genomic DNA
aaatgcgggagtgactagttagtttgatgttattaaattaaaagtacgaattttac

SEQ ID NO: 32 - DNA, 39 bp, Bacteroides thetaiotaomicron
  source 1..39: mol_type = genomic DNA
gctctagaactagtggatccgacagcctccagctgacgg

SEQ ID NO: 33 - DNA, 25 bp, Bacteroides thetaiotaomicron
  source 1..25: mol_type = genomic DNA
catcctttttcttgtctggttggat

SEQ ID NO: 34 - DNA, 56 bp, Bacteroides thetaiotaomicron
  source 1..56: mol_type = genomic DNA
accagacaagaaaaaggatgaaagcattaaaaataacaatcatagctctattggca

SEQ ID NO: 35 - DNA, 47 bp, Bacteroides thetaiotaomicron
  source 1..47: mol_type = genomic DNA
aagataacattcgagtcgacatagaagctggctctttcgaaatagtc

SEQ ID NO: 36 - DNA, 33 bp, Bacteroides thetaiotaomicron
  source 1..33: mol_type = genomic DNA
cgggatccgtggactacttttgctgaaagcgga

SEQ ID NO: 37 - DNA, 40 bp, Bacteroides thetaiotaomicron
  source 1..40: mol_type = genomic DNA
tccccccgggttcatagttctttctgtaatccaattaaga

SEQ ID NO: 38 - DNA, 35 bp, Bacteroides thetaiotaomicron
  source 1..35: mol_type = genomic DNA
tccccccgggtttcattgatatcgtaaagagggat

SEQ ID NO: 39 - DNA, 30 bp, Bacteroides thetaiotaomicron
  source 1..30: mol_type = genomic DNA
acgcgtcgactgccacacttccgtgcactt

SEQ ID NO: 40 - DNA, 42 bp, Bacteroides thetaiotaomicron
  source 1..42: mol_type = genomic DNA
gctctagaactagtggatcccagtaatgaagagacattacgg

SEQ ID NO: 41 - DNA, 47 bp, Bacteroides thetaiotaomicron
  source 1..47: mol_type = genomic DNA
tagtttaatgttattaatttaaaagtacgaattttctcttttcgatg

SEQ ID NO: 42 - DNA, 59 bp, Bacteroides thetaiotaomicron
  source 1..59: mol_type = genomic DNA
aaattaataacattaaactaatgaaaaagataatatatatagcaacaatcggaattacc

SEQ ID NO: 43 - DNA, 47 bp, Bacteroides thetaiotaomicron
  source 1..47: mol_type = genomic DNA
aagataacattcgagtcgacctgtttcaggtcttcttcgttgattcc

SEQ ID NO: 44 - DNA, 40 bp, Bacteroides thetaiotaomicron
  source 1..40: mol_type = genomic DNA
gctctagaactagtggatccgcttcttccgtcagtcttct

SEQ ID NO: 45 - DNA, 32 bp, Bacteroides thetaiotaomicron
  source 1..32: mol_type = genomic DNA
ttatttacacaagtagttgattgcattgagag

SEQ ID NO: 46 - DNA, 46 bp, Bacteroides thetaiotaomicron
  source 1..46: mol_type = genomic DNA
tcaactacttgtgtaaataatgaaaactacaccggcaagtaacatc

SEQ ID NO: 47 - DNA, 39 bp, Bacteroides thetaiotaomicron
  source 1..39: mol_type = genomic DNA
aagataacattcgagtcgactatcgcaacggggcggtgt

SEQ ID NO: 48 - DNA, 43 bp, Bacteroides thetaiotaomicron
  source 1..43: mol_type = genomic DNA
gctctagaactagtggatccctggaagatttgaaagcaactac

SEQ ID NO: 49 - DNA, 26 bp, Bacteroides thetaiotaomicron
  source 1..26: mol_type = genomic DNA
tcaataagtgcttacctgaacgtctg

SEQ ID NO: 50 - DNA, 42 bp, Bacteroides thetaiotaomicron
  source 1..42: mol_type = genomic DNA
ttcaggtaagcacttattgaaaaacgactttcttctccctgc

SEQ ID NO: 51 - DNA, 40 bp, Bacteroides thetaiotaomicron
  source 1..40: mol_type = genomic DNA
aagataacattcgagtcgactgctccccacatggcaatgt

SEQ ID NO: 52 - DNA, 42 bp, Bacteroides thetaiotaomicron
  source 1..42: mol_type = genomic DNA
tcaactacttgtgtaaataaaaaacgactttcttctccctgc

SEQ ID NO: 53 - DNA, 45 bp, Bacteroides thetaiotaomicron
  source 1..45: mol_type = genomic DNA
gctctagaactagtggatccccctctcaattggcgaaagaaaatc

SEQ ID NO: 54 - DNA, 34 bp, Bacteroides thetaiotaomicron
  source 1..34: mol_type = genomic DNA
agctattttatttattagtttgtaaaatcggagt

SEQ ID NO: 55 - DNA, 60 bp, Bacteroides thetaiotaomicron
  source 1..60: mol_type = genomic DNA
cgattttacaaactaataaataaaatagctacggaaatcaaaagctatctttgttttcag

SEQ ID NO: 56 - DNA, 40 bp, Bacteroides thetaiotaomicron
  source 1..40: mol_type = genomic DNA
aagataacattcgagtcgacttccactggtaggctcgatg

SEQ ID NO: 57 - DNA, 40 bp, Bacteroides thetaiotaomicron
  source 1..40: mol_type = genomic DNA
gctctagaactagtggatcctgggaacatttggctcctgc

SEQ ID NO: 58 - DNA, 45 bp, Bacteroides thetaiotaomicron
  source 1..45: mol_type = genomic DNA
ggccccccctcgaggtcgacatcattgtcctgtgtatagagtccc

SEQ ID NO: 59 - DNA, 48 bp, Bacteroides thetaiotaomicron
  source 1..48: mol_type = genomic DNA
cgctctagaactagtggatccagaaaaccgtgttactcagtttgatcg

SEQ ID NO: 60 - DNA, 49 bp, Bacteroides thetaiotaomicron
  source 1..49: mol_type = genomic DNA
gggccccccctcgaggtcgacaaataacagagaacacattcgagttacc

SEQ ID NO: 61 - DNA, 40 bp, Bacteroides thetaiotaomicron
  source 1..40: mol_type = genomic DNA
gattagcattatgaggatcctttggctatcccggcatcga

SEQ ID NO: 62 - DNA, 51 bp, Bacteroides thetaiotaomicron
  source 1..51: mol_type = genomic DNA
tgacgtgaatagttttgatttctcatttttttcgtatttctttctatgacc

SEQ ID NO: 63 - DNA, 34 bp, Bacteroides thetaiotaomicron
  source 1..34: mol_type = genomic DNA
aatcaaaactattcacgtcagaatacaataaatc

SEQ ID NO: 64 - DNA, 47 bp, Bacteroides thetaiotaomicron
  source 1..47: mol_type = genomic DNA
tccaccgcggtggcggccgcgcagtatatacaaatagggttacgtct

SEQ ID NO: 65 - DNA, 51 bp, Bacteroides thetaiotaomicron
  source 1..51: mol_type = genomic DNA
gctctagaactagtggatcctatcattcagttttctgttggttactttgag

SEQ ID NO: 66 - DNA, 60 bp, Bacteroides thetaiotaomicron
  source 1..60: mol_type = genomic DNA
cgtacttttaaattaataacattaaactaatgatgaaaaatatgatcttacctatagcat

SEQ ID NO: 67 - DNA, 44 bp, Bacteroides thetaiotaomicron
  source 1..44: mol_type = genomic DNA
aagataggcaattagtcgactcaataagtgcttacctgaacgtc

SEQ ID NO: 68 - DNA, 59 bp, Bacteroides thetaiotaomicron
  source 1..59: mol_type = genomic DNA
agaaggagatatacatatgcatcaccatcaccatcacagtgatgacttcaaatccggcc

SEQ ID NO: 69 - DNA, 51 bp, Bacteroides thetaiotaomicron
  source 1..51: mol_type = genomic DNA
gcttatcatcgataagcttttatttacacaagtagttgattgcattgagag

SEQ ID NO: 70 - DNA, 73 bp, Bacteroides thetaiotaomicron
  source 1..73: mol_type = genomic DNA
agaaggagatatacatatgcatcaccatcaccatcacggaggtggaggtgacgggctggacgaagcggtaggt

SEQ ID NO: 71 - DNA, 42 bp, Bacteroides thetaiotaomicron
  source 1..42: mol_type = genomic DNA
agcttatcatcgataagcttattccactacgtttaccaccat

SEQ ID NO: 72 - DNA, 50 bp, Bacteroides thetaiotaomicron; identical to SEQ ID NO: 30
  source 1..50: mol_type = genomic DNA

SEQ ID NO: 73 - DNA, 56 bp, Bacteroides thetaiotaomicron; identical to SEQ ID NO: 31
  source 1..56: mol_type = genomic DNA

SEQ ID NO: 74 - DNA, 300 bp, Bacteroides thetaiotaomicron
  source 1..300: mol_type = genomic DNA
tatcattcagttttctgttggttactttgagtgattatatgcttgttatatgaataatatccctttttacaaggacttaaattactatacaaaggtagtattttctatgattgagagagaatataatcccgactttcttatgatggagaattattcttataaaaccctataaaatagcgttctatgcaacatatcttacagcatttgaaacatttttttcagtcggttgacttaaatcaaccgtaatttgcatcatcgaaaagagaaaattcgtacttttaaattaataacattaaacta

SEQ ID NO: 75 - DNA, 300 bp, Bacteroides thetaiotaomicron
  source 1..300: mol_type = genomic DNA
aaaatggaactgggcaatgacaggcatccccagctgtacgatttatccgccgatccatcggagaaaaacaatgtagcgaaacagcatccggaagttgtcagggagctgtccgaactgcttgaatcggtaaagacaagataaccaatgagaatagcagatttgtattattttgtatcaatcttgaataattctatccatatgagaacaatcaatgcgtatatctttgccatcgtaaattcaacaaaatcacttaatttttaatatatgaaaaaacatctatccaaccagacaagaaaaagg

SEQ ID NO: 76 - DNA, 300 bp, Bacteroides thetaiotaomicron
  source 1..300: mol_type = genomic DNA
ttgtttgttggagattgttttcatatcgttgattttttcagttgactacaaccagtagttaactctatttttaagttcttatcacctgcaaaggtaatattttccatacttactggagaatagatattctataattcttctatttcgaagaataatgtataaaaccctataaaatagcgttctatgcaacaaatgttatagcctttgaaacatttttttcagtcgtttgatataaatcaaccgtaatttgcatcatcgatatagtaaaattcgtacttttaatttaataacatcaaacta

SEQ ID NO: 77 - DNA, 43 bp, Bacteroides thetaiotaomicron
  source 1..43: mol_type = genomic DNA
agctcggtacccggggatccaggacgaaaatgtaactttgcca

SEQ ID NO: 78 - DNA, 48 bp, Bacteroides thetaiotaomicron
  source 1..48: mol_type = genomic DNA
atgcgggagtgactagtacttactattttttctcgctgcaaaattcca

SEQ ID NO: 79 - DNA, 52 bp, Bacteroides thetaiotaomicron
  source 1..52: mol_type = genomic DNA
gctcggtacccggggatcccttccaatgatactgaagagaaaatcattgctg

SEQ ID NO: 80 - DNA, 54 bp, Bacteroides thetaiotaomicron
  source 1..54: mol_type = genomic DNA
atgcgggagtgactagtaacattaaatttgaggtttaaaaatagtattaaattc

SEQ ID NO: 81 - DNA, 46 bp, Bacteroides thetaiotaomicron
  source 1..46: mol_type = genomic DNA
gctcggtacccggggatcctctggagtccgtacttaaatccatcga

SEQ ID NO: 82 - DNA, 63 bp, Bacteroides thetaiotaomicron
  source 1..63: mol_type = genomic DNA
aaatgcgggagtgactagtaaatttgtgcgttttagtaattgactaaagttattaaaaacaag

SEQ ID NO: 83 - DNA, 40 bp, Bacteroides thetaiotaomicron
  source 1..40: mol_type = genomic DNA
agctcggtacccggggatcccgtgtggcacggctgcagct

SEQ ID NO: 84 - DNA, 74 bp, Bacteroides thetaiotaomicron
  source 1..74: mol_type = genomic DNA
aaatgcgggagtgactagtctttgatacttgaatttaaattaatgaattgatttatttttacagatttatctac

SEQ ID NO: 85 - DNA, 55 bp, Bacteroides thetaiotaomicron
  source 1..55: mol_type = genomic DNA
gctcggtacccggggatccgtctaagtcttctctttataggaaaatgaaattagc

SEQ ID NO: 86 - DNA, 67 bp, Bacteroides thetaiotaomicron
  source 1..67: mol_type = genomic DNA
aaatgcgggagtgactagtactattagatttataaagttatttagaccaaataagtattagttgtgc

SEQ ID NO: 87 - DNA, 57 bp, Bacteroides thetaiotaomicron
  source 1..57: mol_type = genomic DNA
gctcggtacccggggatccttcaatttcaataataacttttgtgtgttagagtccct

SEQ ID NO: 88 - DNA, 58 bp, Bacteroides thetaiotaomicron
  source 1..58: mol_type = genomic DNA
aaatgcgggagtgactagtaatactgctcttttaaggttaataaaaattgtctgtgtg

SEQ ID NO: 89 - DNA, 42 bp, Bacteroides thetaiotaomicron
  source 1..42: mol_type = genomic DNA
gctcggtacccggggatcctcaatgtgacaccaagcgcactg

SEQ ID NO: 90 - DNA, 47 bp, Bacteroides thetaiotaomicron
  source 1..47: mol_type = genomic DNA
aaatgcgggagtgactagtaagtagtaacggccatttccttgtcttc

SEQ ID NO: 91 - DNA, 40 bp, Bacteroides thetaiotaomicron
  source 1..40: mol_type = genomic DNA
agctcggtacccggggatccaagtggattgacgatgcacg

SEQ ID NO: 92 - DNA, 47 bp, Bacteroides thetaiotaomicron
  source 1..47: mol_type = genomic DNA
atgcgggagtgactagtgtttactagtgataataaacagaatgtcgt

SEQ ID NO: 93 - DNA, 52 bp, Bacteroides thetaiotaomicron
  source 1..52: mol_type = genomic DNA
gctcggtacccggggatccaaaataggaatttgcgtctgatgtacaaaaacg

SEQ ID NO: 94 - DNA, 42 bp, Bacteroides thetaiotaomicron
  source 1..42: mol_type = genomic DNA
aaatgcgggagtgactagtccccgaaactccgtaatagacca

SEQ ID NO: 95 - DNA, 43 bp, Bacteroides thetaiotaomicron
  source 1..43: mol_type = genomic DNA
gctcggtacccggggatccgctatggctacggatacggttacg

SEQ ID NO: 96 - DNA, 66 bp, Bacteroides thetaiotaomicron
  source 1..66: mol_type = genomic DNA
aaatgcgggagtgactagtaattatcattttttaagggataatacaaatataacaaaaaaaacggg

SEQ ID NO: 97 - DNA, 41 bp, Bacteroides thetaiotaomicron
  source 1..41: mol_type = genomic DNA
gctcggtacccggggatcctattgcccatcgtctggaacgt

SEQ ID NO: 98 - DNA, 65 bp, Bacteroides thetaiotaomicron
  source 1..65: mol_type = genomic DNA
aaatgcgggagtgactagtttttatcactattaaaggttaacatatagttatttatcgtagctgc

SEQ ID NO: 99 - DNA, 57 bp, Bacteroides thetaiotaomicron
  source 1..57: mol_type = genomic DNA
agctcggtacccggggatccgaatattgcaaaggtaatttttaaaattgtttttccc

SEQ ID NO: 100 - DNA, 63 bp, Bacteroides thetaiotaomicron
  source 1..63: mol_type = genomic DNA
aaatgcgggagtgactagtaattttgattttaaaataataactagtttaccggttttgtttgt

SEQ ID NO: 101 - DNA, 50 bp, Bacteroides thetaiotaomicron
  source 1..50: mol_type = genomic DNA
gctcggtacccggggatccggtggattacaatgatcttatccattcgtcc

SEQ ID NO: 102 - DNA, 77 bp, Bacteroides thetaiotaomicron
  source 1..77: mol_type = genomic DNA
aaatgcgggagtgactagtagattaaattttaatttatataaaatgatatattacatcatcagacatctattcacat

SEQ ID NO: 103 - DNA, 46 bp, Bacteroides thetaiotaomicron
  source 1..46: mol_type = genomic DNA
gctcggtacccggggatcctggtgtttcctttaaaaccaaaatgcc

SEQ ID NO: 104 - DNA, 40 bp, Bacteroides thetaiotaomicron
  source 1..40: mol_type = genomic DNA
aaatgcgggagtgactagttgtgacttttaggggttgggc

SEQ ID NO: 105 - DNA, 64 bp, Bacteroides thetaiotaomicron
  source 1..64: mol_type = genomic DNA
gctcggtacccggggatcctagtttttcagaactacttaatgctctatttatcaaatgattatg

SEQ ID NO: 106 - DNA, 68 bp, Bacteroides thetaiotaomicron
  source 1..68: mol_type = genomic DNA
aaatgcgggagtgactagtcttattctttttaattagaaatagtttttaggtagttaacaaacatgtg

SEQ ID NO: 107 - DNA, 40 bp, Bacteroides thetaiotaomicron
  source 1..40: mol_type = genomic DNA
agctcggtacccggggatccattaagggggggctatgctg

SEQ ID NO: 108 - DNA, 51 bp, Bacteroides thetaiotaomicron
  source 1..51: mol_type = genomic DNA
aaatgcgggagtgactagtcttgattcgttaaatctctgtagattccccag

SEQ ID NO: 109 - DNA, 41 bp, Bacteroides thetaiotaomicron
  source 1..41: mol_type = genomic DNA
gctcggtacccggggatccagaccaggcaccactatctgag

SEQ ID NO: 110 - DNA, 68 bp, Bacteroides thetaiotaomicron
  source 1..68: mol_type = genomic DNA
aaatgcgggagtgactagttttcttagtattaggttattaactaaattatttcacggtaattgaatag

SEQ ID NO: 111 - DNA, 42 bp, Bacteroides thetaiotaomicron
  source 1..42: mol_type = genomic DNA
gctcggtacccggggatcctcttaaaagcgcttggtttggcc

SEQ ID NO: 112 - DNA, 55 bp, Bacteroides thetaiotaomicron
  source 1..55: mol_type = genomic DNA
aaatgcgggagtgactagtaattttgtggcttaggatttatttgcttgttctaac

SEQ ID NO: 113 - DNA, 40 bp, Bacteroides thetaiotaomicron
  source 1..40: mol_type = genomic DNA
gctcggtacccggggatccaaggggaaagtcgaaaggtgg

SEQ ID NO: 114 - DNA, 59 bp, Bacteroides thetaiotaomicron
  source 1..59: mol_type = genomic DNA
aaatgcgggagtgactagttaagtccttttacttttaattattattaaaaacagccgca

SEQ ID NO: 115 - DNA, 42 bp, Bacteroides thetaiotaomicron
  source 1..42: mol_type = genomic DNA
gctcggtacccggggatcccatacacatcatcgtccctccgc

SEQ ID NO: 116 - DNA, 63 bp, Bacteroides thetaiotaomicron
  source 1..63: mol_type = genomic DNA
aaatgcgggagtgactagtacttttgtgagttaatcattaatactaatttaagtgtacacaac

SEQ ID NO: 117 - DNA, 49 bp, Bacteroides thetaiotaomicron
  source 1..49: mol_type = genomic DNA
gctcggtacccggggatccgggcggaagagtttttaagagagaatatag

SEQ ID NO: 118 - DNA, 67 bp, Bacteroides thetaiotaomicron
  source 1..67: mol_type = genomic DNA
aaatgcgggagtgactagtaagtttattaattaagattaccttttatataagtagcgaagtctttcc

SEQ ID NO: 119 - DNA, 40 bp, Bacteroides thetaiotaomicron
  source 1..40: mol_type = genomic DNA
agctcggtacccggggatccgtactatgcagagagttacg

SEQ ID NO: 120 - DNA, 72 bp, Bacteroides thetaiotaomicron
  source 1..72: mol_type = genomic DNA
aaatgcgggagtgactagttcgttttaatttaaagttaatattaattttcctttgtttagtaaaatacaatt

SEQ ID NO: 121 - DNA, 48 bp, Bacteroides thetaiotaomicron
  source 1..48: mol_type = genomic DNA
agctcggtacccggggatccgcttaattttcgcttaattgattattaa

SEQ ID NO: 122 - DNA, 64 bp, Bacteroides thetaiotaomicron
  source 1..64: mol_type = genomic DNA
aaatgcgggagtgactagtcgtgattttcaaataatttttcacggtctcttttttatattatac

SEQ ID NO: 123 - DNA, 40 bp, Bacteroides thetaiotaomicron
  source 1..40: mol_type = genomic DNA
agctcggtacccggggatcctggaacgtgaacttgaaaac

SEQ ID NO: 124 - DNA, 41 bp, Bacteroides thetaiotaomicron
  source 1..41: mol_type = genomic DNA
atgcgggagtgactagtatttatgctttctataaaggagac

SEQ ID NO: 125 - DNA, 67 bp, Bacteroides thetaiotaomicron
  source 1..67: mol_type = genomic DNA
gctcggtacccggggatcctgtaatctatctaattttatgttcgtttcataaatttaaaaccataca

SEQ ID NO: 126 - DNA, 63 bp, Bacteroides thetaiotaomicron
  source 1..63: mol_type = genomic DNA
aaatgcgggagtgactagtactctgtgattttaaaaagtaagtcataagggttttatattaga

SEQ ID NO: 127 - DNA, 40 bp, Bacteroides thetaiotaomicron
  source 1..40: mol_type = genomic DNA
agctcggtacccggggatcccgaccggcttccggaacgtt

SEQ ID NO: 128 - DNA, 46 bp, Bacteroides thetaiotaomicron
  source 1..46: mol_type = genomic DNA
atgcgggagtgactagtcattcaatcttttttagtgattaatcggt

SEQ ID NO: 129 - DNA, 47 bp, Bacteroides thetaiotaomicron
  source 1..47: mol_type = genomic DNA
agctcggtacccggggatccttgcttataaatctattcgtctgacag

SEQ ID NO: 130 - DNA, 43 bp, Bacteroides thetaiotaomicron
  source 1..43: mol_type = genomic DNA
atgcgggagtgactagtattgcgcgcttttaataagaagacac

SEQ ID NO: 131 - DNA, 40 bp, Bacteroides thetaiotaomicron
  source 1..40: mol_type = genomic DNA
agctcggtacccggggatccgctggcactggcctatggta

SEQ ID NO: 132 - DNA, 55 bp, Bacteroides thetaiotaomicron
  source 1..55: mol_type = genomic DNA
atgcgggagtgactagtacgtattaggtttttaaataaaacaataagtaaataac

SEQ ID NO: 133 - DNA, 57 bp, Bacteroides thetaiotaomicron
  source 1..57: mol_type = genomic DNA
gctcggtacccggggatccaataaaagcgcctcttatagcatgatactattttttgt

SEQ ID NO: 134 - DNA, 49 bp, Bacteroides thetaiotaomicron
  source 1..49: mol_type = genomic DNA
aaatgcgggagtgactagtaccaatatacgaaatctgcaaagtctttgc

SEQ ID NO: 135 - DNA, 58 bp, Bacteroides thetaiotaomicron
  source 1..58: mol_type = genomic DNA
gctcggtacccggggatccggtggtgagaaatattcgggaaatataaaaatagtatcc

SEQ ID NO: 136 - DNA, 48 bp, Bacteroides thetaiotaomicron
  source 1..48: mol_type = genomic DNA
aaatgcgggagtgactagtaccgatgtacgaaatttgcaaagtctttg

SEQ ID NO: 137 - DNA, 54 bp, Bacteroides thetaiotaomicron
  source 1..54: mol_type = genomic DNA
gctcggtacccggggatccaaagatagtaaaaatcaacccattccgattcaaag

SEQ ID NO: 138 - DNA, 63 bp, Bacteroides thetaiotaomicron
  source 1..63: mol_type = genomic DNA
aaatgcgggagtgactagtgttttttgtttaattgtatgttcactaattggtttttgaatagt

SEQ ID NO: 139 - DNA, 48 bp, Bacteroides thetaiotaomicron
  source 1..48: mol_type = genomic DNA
gctcggtacccggggatccctacgacctttaccgctaccattatcaac

SEQ ID NO: 140 - DNA, 65 bp, Bacteroides thetaiotaomicron
  source 1..65: mol_type = genomic DNA
aaatgcgggagtgactagtacttttatatttaaaaatacatttgctataaagtcgatacaaagga

SEQ ID NO: 141 - DNA, 62 bp, Bacteroides thetaiotaomicron
  source 1..62: mol_type = genomic DNA
gctcggtacccggggatccgcaaatgataaaaaaagcaattttgaatgaaaagatatacatg

SEQ ID NO: 142 - DNA, 64 bp, Bacteroides thetaiotaomicron
  source 1..64: mol_type = genomic DNA
aaatgcgggagtgactagtgtttttctataagattttattagtttgaaaaaaggtcttttaggc

SEQ ID NO: 143 - DNA, 45 bp, Bacteroides thetaiotaomicron
  source 1..45: mol_type = genomic DNA
agctcggtacccggggatccttgcaatcattacctgaacaaacct

SEQ ID NO: 144 - DNA, 48 bp, Bacteroides thetaiotaomicron
  source 1..48: mol_type = genomic DNA
atgcgggagtgactagtctttttatcgttttcatttatataacgattc

SEQ ID NO: 145 - DNA, 40 bp, Bacteroides thetaiotaomicron
  source 1..40: mol_type = genomic DNA
agctcggtacccggggatccgaaacgccagacgaatcagg

SEQ ID NO: 146 - DNA, 47 bp, Bacteroides thetaiotaomicron
  source 1..47: mol_type = genomic DNA
atgcgggagtgactagtcaatacttttttaattttcatttcttttgc

SEQ ID NO: 147 - DNA, 38 bp, Bacteroides thetaiotaomicron
  source 1..38: mol_type = genomic DNA
gctcggtacccggggatccaagtgcaggagcatgagct

SEQ ID NO: 148 - DNA, 54 bp, Bacteroides thetaiotaomicron
  source 1..54: mol_type = genomic DNA
aaatgcgggagtgactagtagtattaaaaattaagactatgcaaatgaaagcaa

SEQ ID NO: 149 - DNA, 54 bp, Bacteroides thetaiotaomicron
  source 1..54: mol_type = genomic DNA
gctcggtacccggggatccagatagatattgggagtgcatatgtttgtttttac

SEQ ID NO: 150 - DNA, 74 bp, Bacteroides thetaiotaomicron
  source 1..74: mol_type = genomic DNA
aaatgcgggagtgactagtttctatattctttatatttgattagtttctattatatgattaaacagcggatatc

SEQ ID NO: 151 - DNA, 47 bp, Bacteroides thetaiotaomicron
  source 1..47: mol_type = genomic DNA
gctcggtacccggggatcctatgaaagtcagcttttacgcctctttg

SEQ ID NO: 152 - DNA, 75 bp, Bacteroides thetaiotaomicron
  source 1..75: mol_type = genomic DNA
aaatgcgggagtgactagtgcatagttttctacattaataattggtttatataatctcaaaaatgatatcttata

SEQ ID NO: 153 - DNA, 45 bp, Bacteroides thetaiotaomicron
  source 1..45: mol_type = genomic DNA
gctcggtacccggggatcctgttccttacaaagcctccttttcca

SEQ ID NO: 154 - DNA, 70 bp, Bacteroides thetaiotaomicron
  source 1..70: mol_type = genomic DNA
aaatgcgggagtgactagtactgtttaatttgataggttaataatatatttagtttatagttaggtcaac

SEQ ID NO: 155 - DNA, 40 bp, Bacteroides thetaiotaomicron
  source 1..40: mol_type = genomic DNA
agctcggtacccggggatccactacctcaaaacgcagatc

SEQ ID NO: 156 - DNA, 68 bp, Bacteroides thetaiotaomicron
  source 1..68: mol_type = genomic DNA
aaatgcgggagtgactagtgtatcagaattttaaattagtattagagttcacatttaaggataaatca

SEQ ID NO: 157 - DNA, 41 bp, Bacteroides thetaiotaomicron
  source 1..41: mol_type = genomic DNA
agctcggtacccggggatccgttctaacttttaccgcaaac

SEQ ID NO: 158 - DNA, 68 bp, Bacteroides thetaiotaomicron
  source 1..68: mol_type = genomic DNA
aaatgcgggagtgactagttttctttattattttggttaaacatttatatcatgatccttataaaagg

SEQ ID NO: 159 - DNA, 39 bp, Bacteroides thetaiotaomicron
  source 1..39: mol_type = genomic DNA
gctcggtacccggggatcctgtggacattggtacggacg

SEQ ID NO: 160 - DNA, 79 bp, Bacteroides thetaiotaomicron
  source 1..79: mol_type = genomic DNA
aaatgcgggagtgactagtaatattatagtatttagttatcacttaaaaatttagttattatcttattgttgattattc

SEQ ID NO: 161 - DNA, 40 bp, Bacteroides thetaiotaomicron
  source 1..40: mol_type = genomic DNA
agctcggtacccggggatccccgggcgaatggcacaccta

SEQ ID NO: 162 - DNA, 45 bp, Bacteroides thetaiotaomicron
  source 1..45: mol_type = genomic DNA
atgcgggagtgactagtagcattattcattttttaattatgatac

SEQ ID NO: 163 - DNA, 43 bp, Bacteroides thetaiotaomicron
  source 1..43: mol_type = genomic DNA
gctcggtacccggggatccgtacatgcaggatctatatccccg

SEQ ID NO: 164 - DNA, 56 bp, Bacteroides thetaiotaomicron
  source 1..56: mol_type = genomic DNA
aaatgcgggagtgactagtatataattagattattgtttcgactgattgacgcaga

SEQ ID NO: 165 - DNA, 42 bp, Bacteroides thetaiotaomicron
  source 1..42: mol_type = genomic DNA
gctcggtacccggggatcccttcggaattgctaacacctacg

SEQ ID NO: 166 - DNA, 76 bp, Bacteroides thetaiotaomicron
  source 1..76: mol_type = genomic DNA
aaatgcgggagtgactagtaagttagactttttgaataacataataattaagtcaatataaatagttaagtatagt

SEQ ID NO: 167 - DNA, 46 bp, Bacteroides thetaiotaomicron
  source 1..46: mol_type = genomic DNA
agctcggtacccggggatccaatgtaaggacaagtcctgagaagag

SEQ ID NO: 168 - DNA, 47 bp, Bacteroides thetaiotaomicron
  source 1..47: mol_type = genomic DNA
atgcgggagtgactagtgatatgattcttttagtattagctgatggg

SEQ ID NO: 169 - DNA, 44 bp, Bacteroides thetaiotaomicron
  source 1..44: mol_type = genomic DNA
gctcggtacccggggatccgatgtgcactatgtcttggctgatg

SEQ ID NO: 170 - DNA, 72 bp, Bacteroides thetaiotaomicron
  source 1..72: mol_type = genomic DNA
aaatgcgggagtgactagtaaattattagattagttaatgataaatacttgttttccatttaaaagagattc

SEQ ID NO: 171 - DNA, 46 bp, Bacteroides thetaiotaomicron
  source 1..46: mol_type = genomic DNA
gctcggtacccggggatccacaatcggctaaagtcataaacctgac

SEQ ID NO: 172 - DNA, 58 bp, Bacteroides thetaiotaomicron
  source 1..58: mol_type = genomic DNA
aaatgcgggagtgactagtcttttttataatattaatgagttctattgaattcacgtc

SEQ ID NO: 173 - DNA, 44 bp, Bacteroides thetaiotaomicron
  source 1..44: mol_type = genomic DNA
agctcggtacccggggatccaaaaacaatgcaagaaatggaaac

SEQ ID NO: 174 - DNA, 67 bp, Bacteroides thetaiotaomicron
  source 1..67: mol_type = genomic DNA
aaatgcgggagtgactagtaatttattgtattagattataaactctattcaattgttgattgatcca

SEQ ID NO: 175 - DNA, 41 bp, Bacteroides thetaiotaomicron
  source 1..41: mol_type = genomic DNA
gctcggtacccggggatccagttgtgtttcttgcagccaac

SEQ ID NO: 176 - DNA, 65 bp, Bacteroides thetaiotaomicron
  source 1..65: mol_type = genomic DNA
aaatgcgggagtgactagtaacgtttttctttttacatattaaattattggttcactaaaaattc

SEQ ID NO: 177 - DNA, 51 bp, Bacteroides thetaiotaomicron
  source 1..51: mol_type = genomic DNA
gctcggtacccggggatccgctactctgtgaccactatttataattaccgg

SEQ ID NO: 178 - DNA, 66 bp, Bacteroides thetaiotaomicron
  source 1..66: mol_type = genomic DNA
aaatgcgggagtgactagtgtacatcaatttaaagttaatattaggattactttttgttttcactg

SEQ ID NO: 179 - DNA, 41 bp, Bacteroides thetaiotaomicron
  source 1..41: mol_type = genomic DNA
gctcggtacccggggatcctaatgtagctggcagacatccg

SEQ ID NO: 180 - DNA, 63 bp, Bacteroides thetaiotaomicron
  source 1..63: mol_type = genomic DNA
aaatgcgggagtgactagtatgattctaaaaagttaaacgttatttatgtatgattgtgatgc

SEQ ID NO: 181 - DNA, 58 bp, Bacteroides thetaiotaomicron
  source 1..58: mol_type = genomic DNA
gctcggtacccggggatccacaaaaaaacattcattcctaaaataaaggatgatggac

SEQ ID NO: 182 - DNA, 68 bp, Bacteroides thetaiotaomicron
  source 1..68: mol_type = genomic DNA
aaatgcgggagtgactagtggataatttaaaattaatattaggttaatacatttcaggcaactagatc

SEQ ID NO: 183 - DNA, 47 bp, Bacteroides thetaiotaomicron
  source 1..47: mol_type = genomic DNA
gctcggtacccggggatccctgcaaaacgtcctgtttctaaaaaatg

SEQ ID NO: 184 - DNA, 68 bp, Bacteroides thetaiotaomicron
  source 1..68: mol_type = genomic DNA
aaatgcgggagtgactagtctgtttcctttttttcataatactacatttaataataaagattcatact

SEQ ID NO: 185 - DNA, 57 bp, Bacteroides thetaiotaomicron
  source 1..57: mol_type = genomic DNA
gctcggtacccggggatccaaggaagtgtttagatgacataatgattatttgaacag

SEQ ID NO: 186 - DNA, 46 bp, Bacteroides thetaiotaomicron
  source 1..46: mol_type = genomic DNA
aaatgcgggagtgactagtcgttgtacctttcactaatacggatgc

SEQ ID NO: 187 - DNA, 43 bp, Bacteroides thetaiotaomicron
  source 1..43: mol_type = genomic DNA
gctcggtacccggggatcccctgtcacctttttaggtgtttgg

SEQ ID NO: 188 - DNA, 64 bp, Bacteroides thetaiotaomicron
  source 1..64: mol_type = genomic DNA
aaatgcgggagtgactagtattctcctataacctattttcatactaattatttatatctaatat

SEQ ID NO: 189 - DNA, 39 bp, Bacteroides thetaiotaomicron
  source 1..39: mol_type = genomic DNA
gctcggtacccggggatcctccttgcttttgtgggggtg

SEQ ID NO: 190 - DNA, 71 bp, Bacteroides thetaiotaomicron
  source 1..71: mol_type = genomic DNA
aaatgcgggagtgactagtaattttatctatttctaatgaggattttatctttgctttactataattatac

SEQ ID NO: 191 - DNA, 49 bp, Bacteroides thetaiotaomicron
  source 1..49: mol_type = genomic DNA
gctcggtacccggggatcctctcttgaaactgtgaagactcaaaagaag

SEQ ID NO: 192 - DNA, 57 bp, Bacteroides thetaiotaomicron
  source 1..57: mol_type = genomic DNA
aaatgcgggagtgactagtattatttcaggttatttatagcaaagacgactaagaag

SEQ ID NO: 193 - DNA, 44 bp, Bacteroides thetaiotaomicron
  source 1..44: mol_type = genomic DNA
agctcggtacccggggatccatattcctgaaagccggagaatcc

SEQ ID NO: 194 - DNA, 46 bp, Bacteroides thetaiotaomicron
  source 1..46: mol_type = genomic DNA
atgcgggagtgactagtgacgctatgtgtaatttttaccttgtgtg

SEQ ID NO: 195 - DNA, 61 bp, Bacteroides thetaiotaomicron
  source 1..61: mol_type = genomic DNA
gctcggtacccggggatccgaatacaatttataattatcgggcgaaagtaaaaaacaaagc

SEQ ID NO: 196 - DNA, 63 bp, Bacteroides thetaiotaomicron
  source 1..63: mol_type = genomic DNA
aaatgcgggagtgactagtgcgtattaattttaaagtttataattaaggtatgtgcttgacag

SEQ ID NO: 197 - DNA, 39 bp, Bacteroides thetaiotaomicron
  source 1..39: mol_type = genomic DNA
gctcggtacccggggatccacatttctcccttgaagggc

SEQ ID NO: 198 - DNA, 71 bp, Bacteroides thetaiotaomicron
  source 1..71: mol_type = genomic DNA
aaatgcgggagtgactagtaaatataaataataaaatggttaaagtgcatccgaacaataaatattattgc

SEQ ID NO: 199 - DNA, 57 bp, Bacteroides thetaiotaomicron
  source 1..57: mol_type = genomic DNA
gctcggtacccggggatccaaagttagaagtcatcaattaatagaccttcattttgg

SEQ ID NO: 200 - DNA, 56 bp, Bacteroides thetaiotaomicron
  source 1..56: mol_type = genomic DNA
gctcggtacccggggatccgtttgtagatcctctttataaatacatcaatgaaact

SEQ ID NO: 201 - DNA, 77 bp, Bacteroides thetaiotaomicron
  source 1..77: mol_type = genomic DNA
aaatgcgggagtgactagtgattttgttttttttcatacgtaaaaaattaatgattaataaaatattatattgttgg

SEQ ID NO: 202 - DNA, 39 bp, Bacteroides thetaiotaomicron
  source 1..39: mol_type = genomic DNA
gctcggtacccggggatccccattggactcccgcaaaga

SEQ ID NO: 203 - DNA, 73 bp, Bacteroides thetaiotaomicron
  source 1..73: mol_type = genomic DNA
aaatgcgggagtgactagttttacatttcatgtttttcattctagttaataataaatgtatagttaattaaac

SEQ ID NO: 204 - DNA, 50 bp, Bacteroides thetaiotaomicron
  source 1..50: mol_type = genomic DNA
gctcggtacccggggatccgaataaatgtcgaatttgactcagcgctaag

SEQ ID NO: 205 - DNA, 64 bp, Bacteroides thetaiotaomicron
  source 1..64: mol_type = genomic DNA
aaatgcgggagtgactagtaatattattattttataacttaatacttacaggcatatgagcccc

SEQ ID NO: 206 - DNA, 47 bp, Bacteroides thetaiotaomicron
  source 1..47: mol_type = genomic DNA
gctcggtacccggggatccgtctgcctgatgtaaaagagtagttgca

SEQ ID NO: 207 - DNA, 52 bp, Bacteroides thetaiotaomicron
  source 1..52: mol_type = genomic DNA
aaatgcgggagtgactagttcgttactatttaggttgaagtttattctgccg

SEQ ID NO: 208 - DNA, 39 bp, Bacteroides thetaiotaomicron
  source 1..39: mol_type = genomic DNA
gctcggtacccggggatccaatgtcgcgatgagcaacag

SEQ ID NO: 209 - DNA, 69 bp, Bacteroides thetaiotaomicron
  source 1..69: mol_type = genomic DNA
aaatgcgggagtgactagtagtttactttttttagtttctactctttttagtatatatttaacctattg

SEQ ID NO: 210 - DNA, 46 bp, Bacteroides thetaiotaomicron
  source 1..46: mol_type = genomic DNA
gctcggtacccggggatcccgtaaagggaactatagtgcatcttgc

SEQ ID NO: 211 - DNA, 72 bp, Bacteroides thetaiotaomicron
  source 1..72: mol_type = genomic DNA
aaatgcgggagtgactagtagcaaaaatatttaagatattagtaaataaaaaattaaccgttcattaattga

SEQ ID NO: 212 - DNA, 46 bp, Bacteroides thetaiotaomicron
  source 1..46: mol_type = genomic DNA
gctcggtacccggggatccatttttccagttccaatcggcattatg

SEQ ID NO: 213 - DNA, 58 bp, Bacteroides thetaiotaomicron
  source 1..58: mol_type = genomic DNA
aaatgcgggagtgactagtaaatattcctattagattatataccgcaaatgtaacaac

SEQ ID NO: 214 - DNA, 43 bp, Bacteroides thetaiotaomicron
  source 1..43: mol_type = genomic DNA
agctcggtacccggggatcctacttcctgcctcatctgctttc

SEQ ID NO: 215 - DNA, 47 bp, Bacteroides thetaiotaomicron
  source 1..47: mol_type = genomic DNA
atgcgggagtgactagttctatttatggtattaaattataagctaac

SEQ ID NO: 216 - DNA, 47 bp, Bacteroides thetaiotaomicron
  source 1..47: mol_type = genomic DNA
agctcggtacccggggatccatcgtctggatggaaaatctcataaag

SEQ ID NO: 217 - DNA, 52 bp, Bacteroides thetaiotaomicron
  source 1..52: mol_type = genomic DNA
atgcgggagtgactagtattataagttgagttactttataagtaataaacag

SEQ ID NO: 218 - DNA, 39 bp, Bacteroides thetaiotaomicron
  source 1..39: mol_type = genomic DNA
gctcggtacccggggatccatcggtggcggagcctgtcc

SEQ ID NO: 219 - DNA, 46 bp, Bacteroides thetaiotaomicron
  source 1..46: mol_type = genomic DNA
aaatgcgggagtgactagtaacagaatccaaagaaaggatgctccc

SEQ ID NO: 220 - DNA, 41 bp, Bacteroides thetaiotaomicron
  source 1..41: mol_type = genomic DNA
agctcggtacccggggatccttatatggaagacctggaagg

SEQ ID NO: 221 - DNA, 70 bp, Bacteroides thetaiotaomicron
  source 1..70: mol_type = genomic DNA
aaatgcgggagtgactagtactacttgtttttaattaataataataaggttaccaaatacgtttaatacg

SEQ ID NO: 222 - DNA, 43 bp, Bacteroides thetaiotaomicron
  source 1..43: mol_type = genomic DNA
gctcggtacccggggatccagaggaactatagtgcttcttgcg

SEQ ID NO: 223 - DNA, 63 bp, Bacteroides thetaiotaomicron
  source 1..63: mol_type = genomic DNA
aaatgcgggagtgactagtgttactttgttttttttaattaatactaaggttatcaatcgcct

SEQ ID NO: 224 - DNA, 48 bp, Bacteroides thetaiotaomicron
  source 1..48: mol_type = genomic DNA
gctcggtacccggggatccatatttgactccggattattttcgctggt

SEQ ID NO: 225 - DNA, 73 bp, Bacteroides thetaiotaomicron
  source 1..73: mol_type = genomic DNA
aaatgcgggagtgactagttagtttttatataaatattagaatcgatattccatttattattttcgattcaca

SEQ ID NO: 226 - DNA, 43 bp, Bacteroides thetaiotaomicron
  source 1..43: mol_type = genomic DNA
agctcggtacccggggatccagaatataacaacctttatcacg

SEQ ID NO: 227 - DNA, 69 bp, Bacteroides thetaiotaomicron
  source 1..69: mol_type = genomic DNA
aaatgcgggagtgactagtaaattaatattagttttattaaaggtaaaaagggtaaaaacaagttgaag

SEQ ID NO: 228 - DNA, 42 bp, Bacteroides thetaiotaomicron
  source 1..42: mol_type = genomic DNA
agctcggtacccggggatccctattatcagaagatggagtga

SEQ ID NO: 229 - DNA, 75 bp, Bacteroides thetaiotaomicron
  source 1..75: mol_type = genomic DNA
aaatgcgggagtgactagtagaaaataccgtttttaagaatttatataataataatgatcataattttactttcg

SEQ ID NO: 230 - DNA, 42 bp, Bacteroides thetaiotaomicron
  source 1..42: mol_type = genomic DNA
gctcggtacccggggatccattttccgggtagtgctgacttt

SEQ ID NO: 231 - DNA, 75 bp, Bacteroides thetaiotaomicron
  source 1..75: mol_type = genomic DNA
aaatgcgggagtgactagttattattagttctttttaatatgtgtactattttattgttatttatctaatgacga

SEQ ID NO: 232 - DNA, 54 bp, Bacteroides thetaiotaomicron
  source 1..54: mol_type = genomic DNA
gctcggtacccggggatccttgagtaaagaatacaccaatctggaggtatatct

SEQ ID NO: 233 - DNA, 64 bp, Bacteroides thetaiotaomicron
  source 1..64: mol_type = genomic DNA
aaatgcgggagtgactagtcttttgttcttttatattagattattaatattcgagtgtgtccac

SEQ ID NO: 234 - DNA, 39 bp, Bacteroides thetaiotaomicron
  source 1..39: mol_type = genomic DNA
gctcggtacccggggatccaattgagtgtccatcaggcg

SEQ ID NO: 235 - DNA, 75 bp, Bacteroides thetaiotaomicron
  source 1..75: mol_type = genomic DNA
aaatgcgggagtgactagtcatttcaatttaaagttaatacatattactatctaatcaatacttatgttacatgc

SEQ ID NO: 236 - DNA, 44 bp, Bacteroides thetaiotaomicron
  source 1..44: mol_type = genomic DNA
gctcggtacccggggatccagtacctaccaatggagaattcagc

SEQ ID NO: 237 - DNA, 60 bp, Bacteroides thetaiotaomicron
  source 1..60: mol_type = genomic DNA
aaatgcgggagtgactagtaccatacaaacaaaacaggattaatatttgtagtattcttc

SEQ ID NO: 238 - DNA, 62 bp, Bacteroides thetaiotaomicron
  source 1..62: mol_type = genomic DNA
gctcggtacccggggatccacaattatatttgaaaagtttgccatattgcttttatatatgg

SEQ ID NO: 239 - DNA, 46 bp, Bacteroides thetaiotaomicron
  source 1..46: mol_type = genomic DNA
aaatgcgggagtgactagtcttgtcctcacaagagagaaaaaaagg

SEQ ID NO: 240 - DNA, 50 bp, Bacteroides thetaiotaomicron
  source 1..50: mol_type = genomic DNA
gctcggtacccggggatccgaatgttatggatatcaattggaaggacgga

SEQ ID NO: 241 - DNA, 62 bp, Bacteroides thetaiotaomicron
  source 1..62: mol_type = genomic DNA
aaatgcgggagtgactagtgatttaacttgttgtgaggttaaatattgggttaattattaga

SEQ ID NO: 242 - DNA, 63 bp, Bacteroides thetaiotaomicron
  source 1..63: mol_type = genomic DNA
gctcggtacccggggatccctggaattttttttatgtaatataccctttacttcttattacgg

SEQ ID NO: 243 - DNA, 63 bp, Bacteroides thetaiotaomicron
  source 1..63: mol_type = genomic DNA
aaatgcgggagtgactagttgtttttctgtgattaaaggttaataattagttgggttaatatg

SEQ ID NO: 244 - DNA, 43 bp, Bacteroides thetaiotaomicron
  source 1..43: mol_type = genomic DNA
gctcggtacccggggatcctggagagcaatagagaccttatgc

SEQ ID NO: 245 - DNA, 68 bp, Bacteroides thetaiotaomicron
  source 1..68: mol_type = genomic DNA
aaatgcgggagtgactagttgtttttctgtgataaaaggttaataattagattggttaaaaaaaagtg

SEQ ID NO: 246 - DNA, 43 bp, Bacteroides thetaiotaomicron
  source 1..43: mol_type = genomic DNA
agctcggtacccggggatccagaagggagtcttgcactatttc

SEQ ID NO: 247 - DNA, 48 bp, Bacteroides thetaiotaomicron
  source 1..48: mol_type = genomic DNA
atgcgggagtgactagtcccttattccacctttttattatatgacaag

SEQ ID NO: 248 - DNA, 41 bp, Bacteroides thetaiotaomicron
  source 1..41: mol_type = genomic DNA
gctcggtacccggggatccggaagttgatgctgaagatgtt

SEQ ID NO: 249 - DNA, 49 bp, Bacteroides thetaiotaomicron
  source 1..49: mol_type = genomic DNA
aaatgcgggagtgactagtcattaatttcttcctttatacactaaatac

SEQ ID NO: 250 - DNA, 46 bp, Bacteroides thetaiotaomicron
  source 1..46: mol_type = genomic DNA
gctcggtacccggggatcctctttgtctccgaaaaacaagagtccg

SEQ ID NO: 251 - DNA, 50 bp, Bacteroides thetaiotaomicron
  source 1..50: mol_type = genomic DNA
aaatgcgggagtgactagtttcaataagttgtttttcaccatctccgtgc

SEQ ID NO: 252 - DNA, 48 bp, Bacteroides thetaiotaomicron
  source 1..48: mol_type = genomic DNA
gctcggtacccggggatcctaaagccaaaaagaaacttactgtacctc

SEQ ID NO: 253 - DNA, 52 bp, Bacteroides thetaiotaomicron
  source 1..52: mol_type = genomic DNA
aaatgcgggagtgactagttttcaatgttgatttatcttaggacaatctcaa

SEQ ID NO: 254 - DNA, 63 bp, Bacteroides thetaiotaomicron
  source 1..63: mol_type = genomic DNA
gctcggtacccggggatcctttttttcttcttttattgaatgatgacggattttaaaatcatc

SEQ ID NO: 255 - DNA, 65 bp, Bacteroides thetaiotaomicron
  source 1..65: mol_type = genomic DNA
aaatgcgggagtgactagtactttattaggttttataattattaaaagtgttattttactgttgc

SEQ ID NO: 256 - DNA, 45 bp, Bacteroides thetaiotaomicron
  source 1..45: mol_type = genomic DNA
gctcggtacccggggatcctgtctcccgtggaatatatcaagtcc

SEQ ID NO: 257 - DNA, 49 bp, Bacteroides thetaiotaomicron
  source 1..49: mol_type = genomic DNA
aaatgcgggagtgactagtgctatcaaatttagtggtttgttgatagcg

SEQ ID NO: 258 - DNA, 51 bp, Bacteroides thetaiotaomicron
  source 1..51: mol_type = genomic DNA
gctcggtacccggggatcccaggcagatgatacattcacaaagaaaaagat

SEQ ID NO: 259 - DNA, 65 bp, Bacteroides thetaiotaomicron
  source 1..65: mol_type = genomic DNA
aaatgcgggagtgactagtaactaaaggtagttttagatttaaagcatatttaggatataatgca

SEQ ID NO: 260 - DNA, 51 bp, Bacteroides thetaiotaomicron
  source 1..51: mol_type = genomic DNA
gctcggtacccggggatccacttcctaaagaatactttacgggaacgattc

SEQ ID NO: 261 - DNA, 56 bp, Bacteroides thetaiotaomicron
  source 1..56: mol_type = genomic DNA
aaatgcgggagtgactagtatatcaaaacttttaagttgtttgacaggtataccga

SEQ ID NO: 262 - DNA, 700 bp, Bacteroides thetaiotaomicron
  source 1..700: mol_type = genomic DNA
agaatataacaacctttatcacgctgtgtactatccaaatagcaattaaatagtttctcccttacacccgtaattaaacgactgccaaaatgctttatatttaagaagtgagtttttcgcccggtctgaccgaaacaatcaaccaattaaaaaggaatagaggaataattaaactaagttcttgtagaacttagcacaaaaaacagtctgttaatccatttgtatctaaactaacgcatctaactaagcaatagatttaccatcttttttgctaaaaatcaacgattcgtgcgcaaatatactaattagtttgaaaaagtcatactatttttcaataatatttcatatttaaattacaaataagtttttcaattattctcatttaatcaatcaacgggtagaaacagagtgagtatttcactattacacgtttacagcccccaatacttcccattgattatcaacaacatgcattctattccatcttatcatctactatattatagttctacaagaactaagacatagaacctattatttttctgactttaaatcagcatagttcagcacacaaataaaagttttaacttctaattttaatgcctatgagaaagaaataagtattccaaatccaagagtacccacaagtacttcaacttgtttttaccctttttacctttaataaaactaatattaattt

SEQ ID NO: 263 - DNA, 700 bp, Bacteroides thetaiotaomicron
  source 1..700: mol_type = genomic DNA
ctattatcagaagatggagtgataaaagggcaatatagtactatgggagatacttatgttaattggacacgtgatgacaccataacgggagatgtatatcaaaagagttattttgatcgtatgattgaaagaaatcaagtaaatgcttatctgaaagaaaaggaagaagctgttcgtatttggaaggaaatagaactgatatctcaaaaactacattttccctcagaagaattgaatcattttattcgtatttcctgttcgtatggacgcattaaatatgagctttttgctgtttcgtggcagattatgctatgcggttatgtggcagatacaacaaaaaagtcttttaaccggatagagatggataaatatattactgcatttgatgatttgtggaaagaatggaacgacttatctctagaaaacgataattgcccttcaatgtataagatttcttctaacttttttggttttcctgtaggtatacaagagaccattgataaatacaggaaatagttttcattttttcttcttatatgttatagaatataaaaaaataaatgaatattacttagtggttagtattagtctagtcgtcttactattaatagtagcaaaaagtatgagaaagaaacataaaataacgaaagtaaaattatgatcattattattatataaattcttaaaaacggtattttct

SEQ ID NO: 264 - DNA, 300 bp, Bacteroides thetaiotaomicron
  source 1..300: mol_type = genomic DNA
attttccgggtagtgctgacttttgtcgtccttatttttatcgtattttcaattgggaaagcatatatgatgaagatgattgatagctactgacggattcatcttggtaaattgatttagaaagtcatttcttcataaaagggagaaatgactttcttttttaatatgttctgagctttcctgcatacaagtaaatttattaataaaaaacatgtaattgttttagtggtatatcatttttaaatcgtcattagataaataacaataaaatagtacacatattaaaaagaactaataata

SEQ ID NO: 265 - DNA, 300 bp, Bacteroides thetaiotaomicron
  source 1..300: mol_type = genomic DNA
ttgagtaaagaatacaccaatctggaggtatatctgaatgagctagtgaatcatctgtatcctgcggattctaaaaaataaaactaaaagcccgaataaacagttataaaagattaagacgagagtacatttccccataaatcctcgtctttattcattttatgccggaaaaacaaaaaatactatcgtaaaaacaaatattacacatcccacaagccccaaaaacataattttgcaccagtgagaactctaagagtggacacactcgaatattaataatctaatataaaagaacaaaag

SEQ ID NO: 266 - DNA, 400 bp, Bacteroides thetaiotaomicron
  source 1..400: mol_type = genomic DNA
aattgagtgtccatcaggcggctagcatttagagtatgatgaatgctagccgcctgatggacacttttattctaatccaagcaaatactgaaaatgactagtagaaattactatgtaaagtaaaatatggaatttttggtattttaatcccaaaatctcgtatctgatattttagtgataaaatagtgcacctaaacgctcaaaaagtcccctctctttatgttttatataacttcttttgcacatatgaaacaaaaaataagaactataaatcaggaatattaatgaataaacatgagatgattcctgtttcaattttccccatgttttcaacgattgatttgcatgtaacataagtattgattagatagtaatatgtattaactttaaattgaaatga

SEQ ID NO: 267 - DNA, 500 bp, Bacteroides thetaiotaomicron
  source 1..500: mol_type = genomic DNA
agtacctaccaatggagaattcagcaacattatgcacaacccattgcctcaaacggtctatttcaatcaaaaagttcaggcacgctttatcaaactggaagccaccactcccactgatacagccgcaaaagttgaaccaaatgaaatcggagtaacaatagccccttgattttacattcatcacaagtactaactaaaagcaaaaatcagaatagaaaagacgccatctattttgatttttacactatataaatcttcttattatcaaattaactccaatgaaggcaaagaaagagaaattagtcttaatgaagataacagatctttaatatataaggactgtctatccaaacttatcgaaaaatttaattaaccacaagattcatgcttattgaaaacacaagaatgaattgttaacacttttaatttctatgattaatgaaaaacaaaatagtggtggaagaatactacaaatattaatcctgttttgtttgtatggt

SEQ ID NO: 268 - DNA, 400 bp, Bacteroides thetaiotaomicron
  source 1..400: mol_type = genomic DNA
acaattatatttgaaaagtttgccatattgcttttatatatggttattcttgtttcaagaaaaaccggaataaattgtattatctacactctttcttatttaaaaaacgcgttcagagcatgtattttcgttcaaaaggcggtattttccatgtttatgtacgattcttcattgctatttcttatataagtcgttaagtttgcaacagtttttcacaggtattatgataaagtgcccgaagaaaaacagaatctaacttttgtttaattaaaaaatagataaaatagatttatattgttacttaacagattttaatattatgagaaatgtgatgaatcgtaagcggcattggctgctattattgctcctctcacctttttttctctcttgtgaggacaag

SEQ ID NO: 269 - DNA, 300 bp, Bacteroides thetaiotaomicron
  source 1..300: mol_type = genomic DNA
gaatgttatggatatcaattggaaggacggaagatacaacttcaaagaaaccccattatcccagctcatttcgattataaatcaaatataccaatcaaatataattctagaaggaaagtttactaaacagccttcattcacaggcagcatccgttatgatgaaacattggatgacgtgatagataaactctgtttcagccttaaccttacatacaaaaaacagaatagaaaaatagtaatatataattaaccatatttctaataattaacccaatatttaacctcacaacaagttaaatc

SEQ ID NO: 270 - DNA, 900 bp, Bacteroides thetaiotaomicron
  source 1..900: mol_type = genomic DNA
ctggaattttttttatgtaatataccctttacttcttattacggcctattcgccagagattgttggtgaaggagaaaatattattatatatggtagaaattaaggtgtgcattttatttcttatgcatttattcttttggggatctactaccttttttatttagtatatttgaaaataaatagttgtgcttggctgttatgattcatttttatatttaacagattgggataagttgctctagaacttggtagttttataccaataagtgtattttttcctgttttcataggctattcaaaggttattaagtttgtgtgctacttaagacggtgtaaagttattaataaaattggtgaacgcaatgttttttttattttaattatcggcaaacgatgttttttttaatttttcattctactactcagatcgtaataagatagatataatcttttaattattgtatattgttgattattagtattttatcgctttggataccaagttctagagcgacttattccaatcggtcaattacgactatatttcttaataattatttatgataatgcgaaattataggccgcctccgggctgtgattaaatttttattatcgtttcttcagcatagatcagaacagtgctgaagagagattgaattcattaaaaatttcaatacccccaagaaaaacaaatgaatgataacagtcaaacacaactatatatattccaaaacttactaaatattaagataaagcatacacacccaaaaacaaacgtattagagtaaaaatgcacatcttatttctaccacattatcataatgctagatatagaatagtaagttttctacttatccatattaacccaactaattattaacctttaatcacagaaaaaca

SEQ ID NO: 271 - DNA, 700 bp, Bacteroides thetaiotaomicron
  source 1..700: mol_type = genomic DNA
tggagagcaatagagaccttatgccaaattgaataattttgaagaagtcgtaataggacttggtatgtatgtgtataagtgtcttatatataattgattaattggttgatggatgcttgggatgacaatttatgaactatatagtagaattgagattctaaaaaaatggtgtttgttgataattaaaataaaaaagatattgtatttttcgattttattaataactttacaccgtcttagaatagtacatgcacttaataacctttgaatagcctatggaaacagtagaaaatatactttggggtataaaaataccaagtcctattacgacttagccccaattaatcaatatgattataaccttaaattaacatttatgaaaactcttagcaacagccctccgcctttcaaaatcggagaattcgatctcatttttttacttttatttcagcacagatcagaataggtatgatatgtattctgagttcattatcaacttcaatacccccaatttaagaagatgaataatagcagtcaaacacacccatatattttcaaacttactcatacatacaaaaacaaacgtataccccttgtatgcataccctagttctacccacatagtagacacacaagagtaggtttccctatcacttttttttaaccaatctaattattaaccttttatcacagaaaaaca

SEQ ID NO: 272 - DNA, 300 bp, Bacteroides thetaiotaomicron
  source 1..300: mol_type = genomic DNA
cgttcacgggaacctttacaaatcaacgtttggaaaagattctggaatattttaaagtatcctccaaaatacgctggaaacatatcaatgatgacaaggatggaagtgacagaaaaaaagagataatagagatttattgattaacctttaacaatttagtagtatgaaagcaaaacccacttagaaaaaagtatgggaagattttggcagatctccccatactaaaatgaatcaattgaaaatgcttgtttggcagacaagcatagttaactcattttcaacttaaatcaatacaaaatt

SEQ ID NO: 273 - DNA, 497 bp, Bacteroides thetaiotaomicron
  source 1..497: mol_type = genomic DNA
ggaagttgatgctgaagatgttgctcaagatattttctgtaagctttggttacaaccggaaatatggcttgacaatgaaaatgaattagataactacctctttataatgaccagaaacattattctgaatatcttcaagcatcagcaaatcgaacgggaatatcaggaaggataccttgagaaaacagtacttcatgaattgatagaaggggaagatgcccttaataatatatattatgaagaaatgttgctggttgttcgcctgactttggaaaaaatgcctgaacggcgaaggttaatattcgagctgagtcgttttaagggattaagttataaagaaatcgcagaaaagcttaatgtttccatccgtacggtagagcaccaggtgtatttagcactgatagaactaaaaaaagtgcttctatttttattttttctattttcctttaagtagtgtttttcttttagttgtatttagtgtataaaggaagaaatta

SEQ ID NO: 274 - DNA, 300 bp, Bacteroides thetaiotaomicron
  source 1..300: mol_type = genomic DNA
tctttgtctccgaaaaacaagagtccgacaatgcgccacatatcattgttcaggttcggataataaaaatccatcgaccaccgttttctttgtggcggaaaacttcccagcatcagcaactttgcttttggtgggagaaagggttttaaaggatgtttttcgatttcaatctccatattgatgtaatttcctacaaaaataacgaatctattcttttccttcaaaatagttgcggaaaaaaacaaatttaaaaatatttggctatttttgcacggagatggtgaaaaacaacttattgaa

SEQ ID NO: 275 - DNA, 500 bp, Bacteroides thetaiotaomicron
  source 1..500: mol_type = genomic DNA
ctcccaataacttattaagttataatgaacaggaaggcatccatatccaatcggtagacgtacaaaaatatatcgcctggaaagatggtttttacctcttccaaacagaaaaactaaaagacattgctacaaaactcagcgattactatggcaaaaagatcatgatagacagtccattgaagaccattacctgcagtggtaaactagatttgaaagaagatcttgacgaagttcttcagacactaatacggactgtcccggcaagaatagaaaaatcagatggaattatccatatttatgtaaaacattaattaaaatcgaaagcctatgaataaacattatccttaaaaaaataaggccgaataatacccttaatatcatccggcctttgattaaattacttacacgttaatattaattaatctaatcattacaaatgtatgaaaaagcaccgattatttagtcaccaaaggactaaagataaacaactattactaatc

SEQ ID NO: 276 - DNA, 300 bp, Bacteroides thetaiotaomicron
  source 1..300: mol_type = genomic DNA
tttttttcttcttttattgaatgatgacggattttaaaatcatccatccaaacaacctaaattgtacagaacctattccatatcatattacaatgtcataacattatgacacataaaaaactaggatacaggagataaacagcttttttaaacaaaaacaacatatgtctcaacaaatttgatatcaaaaaaatcatttttgaacaaagtatgaacaacctaaattaaagaatctaattaatgaggttacatttgcaacagtaaaataacacttttaataattataaaacctaataaagt

SEQ ID NO: 277 - DNA, 300 bp, Bacteroides thetaiotaomicron
  source 1..300: mol_type = genomic DNA
tgtctcccgtggaatatatcaagtccatccgcatgaagaaagctgccatgttattgcaacaaaagaaattcaccgttgccgaagtgatgtatatggtaggattttccaatcattcttatttctccaaatgctttcaggcagagtttggaaaaacaccgcgccaatacttgaatgacgggctgtagaggcattttagggcttttcgtcctatttttatccttatatgtccaatctgtgcttttatgaaaatgccaggcagtctatctttgccgctatcaacaaaccactaaatttgatagc

SEQ ID NO: 278 - DNA, 300 bp, Bacteroides thetaiotaomicron
  source 1..300: mol_type = genomic DNA
caggcagatgatacattcacaaagaaaaagataatagtagatttatattagtaaccataaaaatgagaatagcctatgagataacacaaagatcgtaaaaaaaggaagaaagctgctaccaacagctgtcttcccccaatttttcagattcgcggtaaggagtcttgcaaacgctaaccggaatcaaaccacagttcattatagtaataaactttagttaaccgcaaatgtatgaaaaaaaatcattcatttattgcattatatcctaaatatgctttaaatctaaaactacctttagtt

SEQ ID NO: 279 - DNA, 300 bp, Bacteroides thetaiotaomicron
  source 1..300: mol_type = genomic DNA
acttcctaaagaatactttacgggaacgattcctagtggcggcatacagaacgccttgaatattctcatgcttacttcgcctttttattatgaaatggatggctcggtcattgtcttgaaagagaagtagataagcatctttatcatgaaattagatatagccattcctgagttttcccccggaatggctttttaatttatgaagaaccgttagttaacaagagttaaagatagatagccgattggacgaaaggggagagcagtcggtatacctgtcaaacaacttaaaagttttgatat

SEQ ID NO: 280 - DNA, 500 bp, Bacteroides thetaiotaomicron
  source 1..500: mol_type = genomic DNA
aggacgaaaatgtaactttgccataacaaaaaactttatgtctaaagatatagaaactttggataataacaaagcccgggcttgcgaagtctgggctttgcgcataaaaaagggtgtgccaacgttgaagtgcacctcaaaagttagataaaaaacttttggggtcatttcacatttgacacaatctctttcttttcaactttcttatacttttatgaccaaagagaaagaaaaagattatgtgcgtttgaatatgttaaaatatgacccgatttgtcacgtttaggtggtatttcttttttgtcgaactaggaattttgtttttgcgacctacagattttccatttcctgttttcagactgcaaatttacgaataatccatatttttatttctatatatgtaataataacaaattaaagaatttaataattccttaatattcccgaaagaaatgtgaaataaaatatatggaattttgcagcgagaaaaaatagtaagt

SEQ ID NO: 281 - DNA, 363 bp, Bacteroides thetaiotaomicron
  source 1..363: mol_type = genomic DNA
cttccaatgatactgaagagaaaatcattgctgatgatttgatggcgatggaacggacacggttggcagccatgcccgaacagcgacggctgatctatacgttaaatcgttttgaggataaatcttctccggagatagcctccgaattagaattgagttgccgtacagtggaaaaccatctttttctcggacgccgtgatatgcgtgacttcttccgaaactgtatataaaattattcatataataaacaacctttggtgcttatggaaaaggaatcaatactgagtgagaggtatctttatgcctcaacaaattttagagaacaagaatttaatactatttttaaacctcaaatttaatgtt

SEQ ID NO: 282 - DNA, 300 bp, Bacteroides thetaiotaomicron
  source 1..300: mol_type = genomic DNA
tctggagtccgtacttaaatccatcgaatttatcagtgatatcaattataagaaagaaggcaggaatatacttctttataaaaaatgagaattaaaaaatagatcaatgtttaacccgttaataaggacagaatgaaagcatcaccctcatagaaactccctaaaaagatagaagcactcctaaggaatgcttctatccgcaacccggacggatattcattcgcttcgacccaaatgaataacgtatcgcacgttgcttgtttttaataactttagtcaattactaaaacgcacaaattt

SEQ ID NO: 283 - DNA, 700 bp, Bacteroides thetaiotaomicron
  source 1..700: mol_type = genomic DNA
cgtgtggcacggctgcagctacttttacgaatccgtagttcatcttgtacctttttatatatagtgggtacaaaaataggaaatagttttggatttttgcccttttgactctgaatcctcaaaatgaggtgtcttatgtcgttttaatgactattgcataagcaactaataagaatgtcaaatggaaagtaatatggtcttatttttgacggtttatatgggatatatctatttgtttattaatattttgttaattatgcaataaaaagacatttgaggaaatttggtataaaaaatgagactttttgttttttctatttaactattataattatatttgtagccaaattaaagaattttataggcaatttacacctaaacaaacgataagcacttattaatatctataattttagagagatggacatacactttattagtattgcagcgacacaagacatttatttagacatatggcgtaatattaaaaatcagccaagctaatttccctattctatatattgtatagactatttgatagtctgtcctggaacgatgataaaagtatacttttcaaagtcgctccaaggggagaagtatattactgtttcatgcgtgaagcgtaatatgctaagttttcgttttgtagataaatctgtaaaaataaatcaattcattaatttaaattcaagtatcaaag

SEQ ID NO: 284 - DNA, 500 bp, Bacteroides thetaiotaomicron
  source 1..500: mol_type = genomic DNA
gtctaagtcttctctttataggaaaatgaaattagcaacaggactgtcacccattgagtttattcgaaatataagattaaaacatggaagtcaattattgaaggataaatcaatatccgtagcagaggtagcttatgaatgcggattttctaatcctaaatattttgcaacctgttttaaagaagaatttggcgtaactccaaaggaatatcaaaaatcttgttaggataaaaataatatgaacttaaaatacacaagtacgcgtcttaatggaagatgcgtacttttccatatatcaatgatctatcccatttgaatgattcctgaacttatattgaacgatttttagacctgttatagttaatagcgattatggtccaattttggaagtttttgaatgattagagaacttctttctactggataactcgcacttttgtgacgcatttgatgcacaactaatacttatttggtctaaataactttataaatctaatagt

SEQ ID NO: 285 - DNA, 300 bp, Bacteroides thetaiotaomicron
  source 1..300: mol_type = genomic DNA
ttcaatttcaataataacttttgtgtgttagagtccctggaatgctgagtgtctacagcagttccagggatttctttattatcttttcatttaatgccaaagaaggttcattctgttttgaatattgtaattttgttagccgatataattattctttttgtttaaagaaaaaaaactacttattttcttgttatttaaaataaattcatatttttgtaatcgtagtcagaaccaaactatgaaatattgacgtcctctctacacacagacaatttttattaaccttaaaagagcagtatt

SEQ ID NO: 286 - DNA, 300 bp, Bacteroides thetaiotaomicron
  source 1..300: mol_type = genomic DNA
tcaatgtgacaccaagcgcactgctgaataacgaagaataactgatataagggttgctattatgaaacaatgatagcagcccttcgtcttaaaatgagctcattttaagagacatccgtccaagattatactcaaagtgtaaaaaagacacttatataaccgtttatccacctcgaaaattcatttgtccacctttaatatactcatcggttgtatttttgcgacactacaacgagtagaattattgttgaaccattaatatcaatatctatgaagacaaggaaatggccgttactactt

SEQ ID NO: 287 - DNA, 300 bp, Bacteroides thetaiotaomicron
  source 1..300: mol_type = genomic DNA
ttcattatccggccagaagtttgatgaaaaacaattttatcaatcaattactgacttcgaatatgagtggacattatccaaagagcaccatcctataatttctggagagaacccgatattgttggccaagacattatctgagaaatatatgcaatatttttattaagtacatacctcaaaataattataaattataaaacaaaacagcctatgaatataataccgaacttataattaaggaaattatcaaatctaaagaagaaaaaaatacagaattacttaactttaaattattaatgt

SEQ ID NO: 288 - DNA, 300 bp, Bacteroides thetaiotaomicron
  source 1..300: mol_type = genomic DNA
aaaataggaatttgcgtctgatgtacaaaaacggtgtctgaagctttcacaaagatagagcaccctaacaagagaataagggtaaataaataatgatttcttttcataaatgggggtattagttaatttaacgaaggcaaagataagattttctctgcatatatttaacagtcaaagcgacaaacttgcgttttttaccaagttctcgtaggactagtcaacactctgaccaccgttcttgctcttgtgatcattgacagggtcggccgcaagcaactggtctattacggagtttcgggg

SEQ ID NO: 289 - DNA, 300 bp, Bacteroides thetaiotaomicron
  source 1..300: mol_type = genomic DNA
gctatggctacggatacggttacggacaggaaaaaggtgctaaatcgtaatccggttctaagaataaacctgagaataaacgatagcccgtttcaatctgattgaccacagtatatcccccaagatatagtcagtcagatatagccgaccgcttgattttccccaatcaagcggtttgttgtattcatctacgttgacaatataatttttatatcccatatcaaagaaaagcgggtatccggacaaccagatgcccgttttttttgttatatttgtattatcccttaaaaaatgataatt

SEQ ID NO: 290 - DNA, 300 bp, Bacteroides thetaiotaomicron
  source 1..300: mol_type = genomic DNA
tattgcccatcgtctggaacgtaaattccaggtgaagatcagcatagaaagcgaacgtctgaaggccgaacacttctccggttctttcgacagcaatcagaatatttatgatatattacacgaaatcaacgttgagaaacaatacacgtggaaagtcagtggtgacaccatctttattaccgataaaagaaagggggtaagataaaaggaaggagaaagaaaatggacacagccgatagatataccggcagtccgcagctacgataaataactatatgttaacctttaatagtgataaaa

SEQ ID NO: 291 - DNA, 300 bp, Bacteroides thetaiotaomicron
  source 1..300: mol_type = genomic DNA
ttgatgctttttttaaaagttatttcttcgtttaccctatcttttccttttataattctgataaagtaatatttcaggaggtatttgaaattaatatgtactcataatactcatcccaccaagttctccatgaacttataccgccttagagtttactaaattctatatttcacaacctaatttttaatcaaaaatcaacacacaatcaatgcctatggaaatattacagtattatgatttaccatccctatcgtttacaaacaaaaccggtaaactagttattattttaaaatcaaaatt

SEQ ID NO: 292 - DNA, 300 bp, Bacteroides thetaiotaomicron
  source 1..300: mol_type = genomic DNA
ggtggattacaatgatcttatccattcgtcctttatatgaatcggttaaagtggaagttaaaacgatgtgaagatgtggagaagaacgatgattggaatataaaatatttaaaagaaaattttcaacaataatggggaggggaattatgatatagtttaataaaactaagagaaaaaacttcagattctattttttattctttttaagagattacgattcttttagttgtatctttttatttatgtgaatagatgtctgatgatgtaatatatcattttatataaattaaaatttaatct

SEQ ID NO: 293 - DNA, 300 bp, Bacteroides thetaiotaomicron
  source 1..300: mol_type = genomic DNA
tggtgtttcctttaaaaccaaaatgccgttccttttaaaccaacgactgctccgttcgtatattttaaagctctttcatagaattgaagatggcatttttatgcactaagcaattgttttatgcaaacacatcgcataatcttgcaataaattatacatttttttgctatttattagatttcatagactttgactacctggctattcttaaaaatagaacataaagaagtatgaaaacagctataaattcattactcccaatcaatcacgcttgccaacgcccaacccctaaaagtcaca

SEQ ID NO: 294 - DNA, 300 bp, Bacteroides thetaiotaomicron
  source 1..300: mol_type = genomic DNA
tagtttttcagaactacttaatgctctatttatcaaatgattatgtatttatttttcaaaaaaatgcttagtctaaccattttttacgaaaaaaaatcttattttcgtttagtatggttgcagtatcaaatgtattactgatgaataagcgcttatgaaaaatcatttttagtgtaacctttaaatttataaatatgagaaacagagatacatagatcctttgtatgagtggcggttatatttattacccgcacatgtttgttaactacctaaaaactatttctaattaaaaagaataag

SEQ ID NO: 295 - DNA, 500 bp, Bacteroides thetaiotaomicron
  source 1..500: mol_type = genomic DNA
acaaaaggagataaatgaccagatatataacagtagtattacattaaggtaaagaacaaccatggtatagtattattgcaatttaactatttaaagtttgtttataatcaaatcatttaaagatttttgtccttctatgattttttgtgtttgataaatgtcgtaattaaataaaaaatatcacgcataatgactttttcttttttctaaatttcttcaaatgacaaattattgttacctttgtaatgtaatgtagttaatggttgcgcttataaagtccttggaaaggatttatgtataaaagaggtagatgtttactctgttcttgtttggattatcagcatcactgatatgaaataacaaaaaaaatatacaatggctaagcggacggacaggcagggaaatagtacagattcccaatccccctctttataatacattgcgtataagatttattaactgtctggtctggggaatctacagagatttaacgaatcaag

SEQ ID NO: 296 - DNA, 300 bp, Bacteroides thetaiotaomicron
  source 1..300: mol_type = genomic DNA
agaccaggcaccactatctgaggtaatggatgtgatagtaaatgtagtaggtcaaatgaactataagataaaaggagaccgctgctacttaatcccaaagtaatgggtatcatcccaaaagaaaaacagtaaaaaaacaacaatataataacctttttaaccactaaagattcatgcaaaaaagtatccggaacggagccctaactctatcccggatatgaaatcaagattctcaatattagagttttactctattcaattaccgtgaaataatttagttaataacctaatactaagaaa

SEQ ID NO: 297 - DNA, 300 bp, Bacteroides thetaiotaomicron
  source 1..300: mol_type = genomic DNA
tcttaaaagcgcttggtttggccggtatcacagaatcagtttgcagactatcgctcataagggtatctctcctttcagcctgggcctgtaaggagagtgacagaaacagaaaaagagatagaacaatttgcttcatcctgtgatttttcgttgttatgattgatgctgttcgtttcacgaatgtactacctttcgttgatttatactaataatcgtctaaaaaaatgtttcgtttcctaaacaaatgagatatgtattctgttagttagaacaagcaaataaatcctaagccacaaaatt

SEQ ID NO: 298 - DNA, 300 bp, Bacteroides thetaiotaomicron
  source 1..300: mol_type = genomic DNA
aaggggaaagtcgaaaggtggcacaaaggcagcctgctttttttcctgtgcatacccctccacgcaacaggccaataataaagcagcaatatatcttttcattatcttcttcacttttttactcctgcaaatgtacattattccaactagattacgaaatatccgggaagaatgatagcagatgaacctagttaataattccttttattattatattcgcaactaaactgtaatcattatattattaatattattacttttgcggctgtttttaataataattaaaagtaaaaggactta

SEQ ID NO: 299 - DNA, 300 bp, Bacteroides thetaiotaomicron
  source 1..300: mol_type = genomic DNA
catacacatcatcgtccctccgcatctggctaccaccgatgtatataccatcaagttcaaacaaaaagcccctatcaaggagatcatgaatatcgtgaccaaaaccataggaaacatagactataaagtcgaagacgagaacattctcttaatttattcacccctaaataagaaaggaggccgatagacagaaaaaagggagtcgatatttcgcggataccaactcccccaaatcaaaattacttcatttaaattagttgtgtacacttaaattagtattaatgattaactcacaaaagt

SEQ ID NO: 300 - DNA, 300 bp, Bacteroides thetaiotaomicron
  source 1..300: mol_type = genomic DNA
gggcggaagagtttttaagagagaatatagtatcatgagtatattcgggtaattagagttaatgaaaggcggagatattcggaaacggatattgccgcctttctttttttgtctcgaaagttgtctgataatgtctgttgacctcgaaaaatgcgataaaatggactattttagacatttttggaagaatcatagccaaaatcgaacatcttaaatcttagatataacctaaatttgtggcagtgaatgtcaggaaagacttcgctacttatataaaaggtaatcttaattaataaactt

SEQ ID NO: 301 - DNA, 700 bp, Bacteroides thetaiotaomicron
  source 1..700: mol_type = genomic DNA
gtactatgcagagagttacggacacgcagccagttgaccggaaaaaagtcgttctttcggaagaaacggtaaccgaacatggaattatccagattgcggaattcaataagaatgcaggtgcgtgatgcttcttgtgtgagatgctccagcatttcaccgaaaatttcttcctccttctctttacttgccggtagagaagtgtccagaaattcacctgcaccatataccacacattgtttgacaagagaggagggaagccactttttggtcttgcgtatggcggcaagcaggcgtgctacaggtctgccttcttctgttgccactatcagtagcggggtatatcccggagtcgattcatagatctgaaacaactccttggaatgaaaagtattggttcccggtaaatcaggaatgtcttttccgcgataatatgttgttagtttaagtggcatccaaatacaaatttacactttttattcttcattcaacaataaaaaagagaatttgttggcttagcctaaaatttgtctttttatcctttgcatatatttgaggaaaaaaaggcttggaaaagcgaatttttgtattattgtcgtcctttgtgagtacagaagtaaaatggaaacgtctatataaaagattatgctaattgtattttactaaacaaaggaaaattaatattaactttaaattaaaacga

SEQ ID NO: 302 - DNA, 700 bp, Bacteroides thetaiotaomicron
  source 1..700: mol_type = genomic DNA
gcttaattttcgcttaattgattattaaatatcccttaaatgtagaatttatgcaaataagtatgctgaagaggggataaaagttagctgtagttataaatattcctgttattccttattatataggaaacttatattatatgagaaatagggaatagtacaacttaaagcttatatcatggggagcgctttgaaaatctatcaggaataacgccattgtaatactaaaaatccacatcttatataatcggataagtcactaattggatatctcctaatttgtgttatgtttgtatagtatttgccttatttacctttttagcctttctacttgaaatattcttctttttgcaggaagtgaatactcatttagataaagaaatagggtgtggacagcatactacaggatataaaacttcccccttttcctgaaattaatacctaagattgcgaaaaattataagaagatagtattagttttaaaattaattagaaattaatcagagtttaatcgtattttataaaaaatcttaagcaggctcttgttactttgcatcagaactcaaaaataaccttaaatattaagtttgttcagtcgacgctttcttaaataatagataaaaagggtgttttcgaggtgtaacaaatagcaaatgtataatataaaaaagagaccgtgaaaaattatttgaaaatcacg

SEQ ID NO: 303 - DNA, 300 bp, Bacteroides thetaiotaomicron
  source 1..300: mol_type = genomic DNA
tggaacgtgaacttgaaaacgaaatatataaggctatcgataaattgccggacgaatgccgtcgggtttttgctaaaagtcgctttgaaggaaaatcatacgaagaaatttccggtgaactgggaatatctattaatacagttaagtatcatataaagaatgctttagcttctttacacgctcatttgagtaagtacctgatttctctattactattttttttcaggtaaagcgaaaaaaaatcattttctgactacccttaattaaaaaacaactgtctcctttatagaaagcataaat

SEQ ID NO: 304 - DNA, 300 bp, Bacteroides thetaiotaomicron
  source 1..300: mol_type = genomic DNA
tgtaatctatctaattttatgttcgtttcataaatttaaaaccatacacacaaagccgcattaacatttattctcaaaacaaccattcatatataaatttatattaaataaataaatctttctagttttcctgctttaaatatatatatttgcaatatgtatatacatatagacattatataatacatatatcataagcaattaatactatgtagattaaaatatacagcctttatatacaagaaatcacattaaatctaatataaaacccttatgacttactttttaaaatcacagagt

SEQ ID NO: 305 - DNA, 300 bp, Bacteroides thetaiotaomicron
  source 1..300: mol_type = genomic DNA
cgaccggcttccggaacgttgccgggaaatattcatccgtatccgtgaagaaaaacaaagttatgcccaagtagcagaagaattggatatcagcataaagacagttgatgcccagctacagaaagctgtctcccgactgaaagagatgatttcgtccagcggataaccggagatcacccgaaccgttcagcacttcttttttatattaacttttatttgcacaaaaggcgtagggagtttctgtcaatctccggtctttatcagcaaataaaccgattaatcactaaaaaagattgaatg

SEQ ID NO: 306 - DNA, 300 bp, Bacteroides thetaiotaomicron
  source 1..300: mol_type = genomic DNA
ttgcttataaatctattcgtctgacagttgttgaactttcgtctgacagttgttgaactttcgtctaacaactgtcagacgaaagttcaacaactgtcagacgattaaatttaagtataatgaatactaaatctgtgtacatcaagaattaaatcttagagtaaatccgaacagattagttcgtttcaaaacagttcttagcttaaacttattggattttacttcccttccttgattttttttcattttcctttggtggtaaatgtaagttgctgtgtcttcttattaaaagcgcgcaat

SEQ ID NO: 307 - DNA, 300 bp, Bacteroides thetaiotaomicron
  source 1..300: mol_type = genomic DNA
gctggcactggcctatggtatgcatgcccttaaccatacaggttcgtcacaagaagcaattctgcaatattccctgtttgtgcgtgagtacaagcaaatgatgaatgaagaatattctacttcttatgcagagctgatgagcaaaaatcctccttttcatcgataaaaacatatcggttaatgatatttattcgcttattaatccgttattaacccatcactaataagttgtttaaccttgctacattatgtttgcagcgatgttatttacttattgttttatttaaaaacctaatacgt

SEQ ID NO: 308 - DNA, 300 bp, Bacteroides thetaiotaomicron
  source 1..300: mol_type = genomic DNA
aataaaagcgcctcttatagcatgatactattttttgtttataatagctccttgttttatagatataatattgattataagataaatagttttcatttcggtagagaagtggtattacataaaagagagtaaaacattagctgaattagggtaattaactgtcaaatagatatataagctatttctgcttgataaaatatcgactggtgttactgatttatcccctatatacctaactgattatcagttatatataaatccattagttctgcaaagactttgcagatttcgtatattggt

SEQ ID NO: 309 - DNA, 300 bp, Bacteroides thetaiotaomicron
  source 1..300: mol_type = genomic DNA
ggtggtgagaaatattcgggaaatataaaaatagtatccaataataatacctcatataaataaaataaagacataactttcttataatgaatgaactaaatgattgaaatggatagaaacagggtatcattcgttaaagaaaagggtagttataatgctgataaataattgattatcacgtttatatatggatacaataacatatatgacgattaggtttatatttctcattattataataatcaacactttatataaagaattagttctgcaaagactttgcaaatttcgtacatcggt

SEQ ID NO: 310 - DNA, 300 bp, Bacteroides thetaiotaomicron
  source 1..300: mol_type = genomic DNA
aaagatagtaaaaatcaacccattccgattcaaagtcaactttattttttttcaatccacaacgctctatgcctatttttgtccaatcagtctaaataaactgatattgatagtggcgccacatcaaatacatacactcagagattgaccgtcaattcacggtacaatcgcagttttaaccaataaaatctaatagcctatggagtaaatcaaatcggtataatcggaacatagatttaaatcattcagagatttaactattcaaaaaccaattagtgaacatacaattaaacaaaaaac

SEQ ID NO: 311 - DNA, 300 bp, Bacteroides thetaiotaomicron
  source 1..300: mol_type = genomic DNA
ctacgacctttaccgctaccattatcaacggcagtgagtatcagaatccgggatattaaattgtaagacctatatacccaatcattatcggtacgataatccatatagccacgctatttatcgtaccaatgattggggatattttccacacctttccacatttttccacacctccccaataatatcgtttttttcaactctgctgttaatctgtatcttatcctttcagcatttctttatatcctattggtatatcctttgtatcgactttatagcaaatgtatttttaaatataaaagt

SEQ ID NO: 312 - DNA, 300 bp, Bacteroides thetaiotaomicron
  source 1..300: mol_type = genomic DNA
gcaaatgataaaaaaagcaattttgaatgaaaagatatacatggatgaataaaagcatatgctcctgattccttgaaataccctaagtttgtatcctgaaaaccgaacggaaagtgtgcttcacaattatatatagccatttacacctttgaatctataatatttaaaacaattaatcacatgagagaaaaagccaaaagcccttaagtcaaattgaaatcaattagaatctattaaaccttatttaaatagaaagcctaaaagaccttttttcaaactaataaaatcttatagaaaaac

SEQ ID NO: 313 - DNA, 300 bp, Bacteroides thetaiotaomicron
  source 1..300: mol_type = genomic DNA
ttgcaatcattacctgaacaaacctcccgtatatttactctcagccgatttggaaataaaacaaatcgggaaattgcctgcgaattaaatatttcaatcaaagatgtggaatatcatatatccaaatcacttaaggctcttagaaaaacgctaaaagattacctgcctttattttatttctttttttaccatatgtaacaccgattactacgcacgttcctttatcttttgaaaaaaatattctttttcgtttagggtaaaccgaagatgaatcgttatataaatgaaaacgataaaaag

SEQ ID NO: 314 - DNA, 700 bp, Bacteroides thetaiotaomicron
  source 1..700: mol_type = genomic DNA
gaaacgccagacgaatcaggactttaccgaaaagcaatacgagcacatccacttcatgatgaagctgaccgatgatgccctcgcacaaatgattgtggtggtagagaaaccggaacaccaaagcattgatatcaacaagtctttcaacatcgaaaacgaaatcaataactaccgtaaccaactgaaaaatcagaatattctggatgtcaacaacaaggaatatgattatcagatgggagtctattatatggatattatcgccgaatgtgagaaactgggcgactatgtagtgaatgtggtagaagccagcagtgatgtaaaagagaagaaagcctcttaaataaagaggaactcaatcaaagaaatggatagccaatttatgcgttgcagcataaaattgactatccatttcttttttatttccacacagacaaaacaactcatgtccgtttacaaggacactcacactttacaaagacgttacattttttacagacgttacgattacatttcttttccgtgaagttcccgttaagcttttactgcgcctattgcaggtcttttttttgcatatacatttgcgccaaaatacacaggggcctatgtttagtaccattttccaatgaccggacaaaggcattccaaaagaaaaccaaatctctaatttaaagcaaaagaaatgaaaattaaaaaagtattg

SEQ ID NO: 315 - DNA, 542 bp, Bacteroides thetaiotaomicron
  source 1..542: mol_type = genomic DNA
aagtgcaggagcatgagctaatccagtatgaatcccgttccatcacccgaagtctatcttcgcccaaagagcagacttttaaaacagaacgtatcattttatggtttctgaataaaagaaatattcccatcctgaaaaaagacagagaagcattttgggaaaagttgtctcctgagattcatgaattatataataataaatatgaaagtcagcttttacgcctctttgactttacggcgtggatggaatccaaaattcggaaagaaaaactgtctgaagtcctcagagcacgtgctagcgcaaaagaatgttaaagagtaaggtgtttgattcgtatttcagacgataaaaacactcttttcaaatctccattttctctttgtaattatataaattcgcttcacattacctctatattctctcctctatacaaggaaaatccaatataagatatcatttttgagattatataaaccaattattaatgtagaaaactatgcatgattattgctttcatttgcatagtcttaatttttaatact

SEQ ID NO: 316 - DNA, 300 bp, Bacteroides thetaiotaomicron
  source 1..300: mol_type = genomic DNA
agatagatattgggagtgcatatgtttgtttttacaagatgataaagtgtattatagcgacaatatagtaaatatttgtgccaagagaaagagactgattaaatattctgagggaatggaagtttgaaaacttacatttgcatataattgacttgtgatttttattattagaatagttattatttgttatacttttaaattaaaatgcttatgaatattatagaaagacatccaccatgattaatgatatccgctgtttaatcatataatagaaactaatcaaatataaagaatatagaa

SEQ ID NO: 317 - DNA, 300 bp, Bacteroides thetaiotaomicron
  source 1..300: mol_type = genomic DNA
tatgaaagtcagcttttacgcctctttgactttacggcgtggatggaatccaaaattcggaaagaaaaactgtctgaagtcctcagagcacgtgctagcgcaaaagaatgttaaagagtaaggtgtttgattcgtatttcagacgataaaaacactcttttcaaatctccattttctctttgtaattatataaattcgcttcacattacctctatattctctcctctatacaaggaaaatccaatataagatatcatttttgagattatataaaccaattattaatgtagaaaactatgc

SEQ ID NO: 318 - DNA, 300 bp, Bacteroides thetaiotaomicron
  source 1..300: mol_type = genomic DNA
tgttccttacaaagcctccttttccattccgtctataattttgtatcaaggtctacccatttgttacagatagtgtttctagctatttgtgcacctaatttggtgaatattgcacgtccatttttcacatcacccctaattttgcatcatcattaaaagataaatgaagttaaccttattgcacataagcaaaggatatatttagaaactgaagagagaataaagagaataaaagtcacccccaacccggttgacctaactataaactaaatatattattaacctatcaaattaaacagt

SEQ ID NO: 319 - DNA, 700 bp, Bacteroides thetaiotaomicron
  source 1..700: mol_type = genomic DNA
actacctcaaaacgcagatcgtatccttgctgtataaccgtcagaaagaacgcgaagcattctccaagcgtccgttcttcccgacgaataagatgcagatgaataaggtggatgaagagtttatgaataaagtgattcgtaccatcgaggaaaatatcatagataccaatctgaacgtagagcatcttgccgagattctgggcatgagtcgttccagtctgctgcggaaaataaagatgctttccaatctgtctcctgtggacttcatccgtttaatccgattgaggaaggctgccgaactgattcatgaaggtaaatacctgattggagatatctgctttatggtaggtatcaactcaccgtcctatttcagcaagcttttcctgaaacagtttggcatgacacctaaagatttcgagaaacaaagtcaggcagacaaagaaaagatagacatgccttcgtgaccgtccgggtgcactataacaaatgatgcacaaaaatgaaacatctgcggaaaatatgcttcagatgtagtctggtgctgcaaaaatggtattttttgcagtgatttttattataccgcctatatatatactcctacttttgccatcgttccggataaggaaagataacccctttcggtgaacggattgatttatccttaaatgtgaactctaatactaatttaaaattctgatac

SEQ ID NO: 320 - DNA, 700 bp, Bacteroides thetaiotaomicron
  source 1..700: mol_type = genomic DNA
gttctaacttttaccgcaaactgaaaggagtattggatatgagtccgaatgagtatcttcgtttgatacgtttaaagaaagctgcccagcttctgaaagatggaagatatgggattgtagaaatttcttatatggtaggattcaattcaccttcttatttctcaaactgttttaagaaacaatttggagtgttaccaaaagatttcatgcaatgaacaaattcttataaaattgctctaaaactgattctgactatctctgcactgttttttggatagaatttaataatatcaggttaatttcttatatccttttcaggtaggttccgataactttgcgaatgtctcgaaggtaggaacttattttctgaccagtctatttggttgggaaaaaactttcttctagatcaattaatatagaagtaacggataaataccagattagaaactggctttataagctattaattattattaatcaatatctaatttttcgcgcgaatatgattttgtatgtattgtttggtagatagggagaagagttgatacataaataaggaattggcaggtattagtttttttataaaactcttcttccacttttcttcgctgaagtatataatccttaaataatacgatttatgaacaaattccttttataaggatcatgatataaatgtttaaccaaaataataaagaaa

SEQ ID NO: 321 - DNA, 300 bp, Bacteroides thetaiotaomicron
  source 1..300: mol_type = genomic DNA
tgtggacattggtacggacgagcgttatccagttatggcttacttcaaggattgacaggagtccgatatgatgctgtagataaaacactgtatattaattcaaaaataggagactttattagctttatcagtacggagtctggatttggtaatatagaacttcgttccggaaaaccttttgtaaaagtagtatccggtcatatagaggttgacagatttgtcgtatcggggaaagtagttgaataatcaacaataagataataactaaatttttaagtgataactaaatactataatatt

SEQ ID NO: 322 - DNA, 700 bp, Bacteroides thetaiotaomicron
  source 1..700: mol_type = genomic DNA
ccgggcgaatggcacacctattatcctaattatcaaacaggttggagtcattattggatcggcttttgcggtccggaagttgatagctggatgacgaatgagtattgttcgaaagaaagtcctgtatttaaggtcggtatcaatgatgagatcgtttctttattcagaaaagctattgatgttgctaacgaagaacctactttatatcaacgcgtattgagtggattggtgacctatctggtagcactaatgtgtagtatcgataagaaccttcaggtagagaatgatgatttctcttctaaaatagattatgcttgtgtgctcatgaaggaattgatagatcaaccagtttctatgcaagagattgccaaaaaagcaggtatgggatattctctttttcgaaagttgtttaaggaacagaagaattatgcacctgtgcaatatttccagaatctgaaaatacagaaagccatagagttgttgactactactactattccggtaaaagagatagcatataggctcgattttgaatccccggcttatttctctgcccggttcaagaaacaaacaggaaaatcacctatagaatatcgagaagaatttcgtatcaaataatgatagtgttctggcaattatcgaaagtctttctcttttaagatgactaagtttgtatcataattaaaaaatgaataatgct

SEQ ID NO: 323 - DNA, 300 bp, Bacteroides thetaiotaomicron
  source 1..300: mol_type = genomic DNA
gtacatgcaggatctatatccccgttgcattgcgcatatatacacaatgtacagggcatatagatcctgcatattgcatatatatagcttatctgcctcttttttattgcaatattttatgtgcttttatgtggtatatgaaacaattatctcattcctgtttcatttggtatagatttgctattctttcttctatttgagacattattgcgatttattgggatatttatattacccaaaaactatctcatccgctaattttgtctgcgtcaatcagtcgaaacaataatctaattatat

SEQ ID NO: 324 - DNA, 700 bp, Bacteroides thetaiotaomicron
  source 1..700: mol_type = genomic DNA
cttcggaattgctaacacctacgggactaaagttacttacaggtacagtaattattcttgccggaatcattttctttgcgatagcagggcacaacagagatagcaaaagccgccagacgcactctgaaacacctattaagaaaaatttcaggaaagggttaattatctgtttgcttgcaggaatattcggacctatgattaactttgctttcgtttatggggcacccttacaagaaaaggctgtagcaacgggagcatcttctctttatgcagccaacgtgatatggagtatcgctttgagtgccggatttattattaatctccttgaatgtatccgtttgttcggcaagaatcaatcttggaaaacttaccgacatcgtacaaccggaggcctgataatggcttctctggcaggggtattatggtatctgagcattatgttctatggtatgggaggcagttttatgggagttttaggagcttctgtgggctgggctaccatgcaatcgacagctattattgcaggtaatgtggccggacttgcttccggcgagtggaaaggagctacccgctatgccatccggatgatggtgataggattggtatgtctcatcggaggagtggtggtgattgccctctaacgactatacttaactatttatattgacttaattattatgttattcaaaaagtctaactt

SEQ ID NO: 325 - DNA, 300 bp, Bacteroides thetaiotaomicron
  source 1..300: mol_type = genomic DNA
aatgtaaggacaagtcctgagaagaggttatattcactcttgcaatatgaaacttttcatttgcactcagaaaataggatagcttaaaaacggaatctctcatattcttttttgatttgattgcaatattaagagttagccttcacaaacacaacccgtgaagactcttttttatatgtcaaagcataaaaagtcggaatagcgtaaaaaaactaccgctatgaataatttctatcggaaaaacgtatatggacaacctctacctttgtccccatcagctaatactaaaagaatcatatc

SEQ ID NO: 326 - DNA, 300 bp, Bacteroides thetaiotaomicron
  source 1..300: mol_type = genomic DNA
gatgtgcactatgtcttggctgatgtaaccttggaacaaggtaagaatacaattaaaaccgttgcaacatataatggaaaagaatacactgacgagattgaatggaactataccggcgaaaagaagcgaagtgccgattggagtgaaaacaaagaagagcacgcaggttggtaatcggagacgtttctgtatgtaatctgaaaaatgttgaatctaaaatataaaagcctatgaatgaaatgagaaagaatctcttttaaatggaaaacaagtatttatcattaactaatctaataattt

SEQ ID NO: 327 - DNA, 300 bp, Bacteroides thetaiotaomicron
  source 1..300: mol_type = genomic DNA
atcaacgagtatgactggcgctcaagtaaatatcgctttaccaacaagccattacaggaattcatcgattttatcaaccgttcataccatgtaaacatcataattaaagaagaaaagctcaaagagctgaagtttaacggaactatacgtaaagacgagccgcttaccaatatcatcgaaaagatatgtatcagcctcgatttaaaagaaaaacaagaaaataacaacatcatattatattaatacataagaatcaattatcaatatattcactattaaccttttagaaacatgaaaaac

SEQ ID NO: 328 - DNA, 700 bp, Bacteroides thetaiotaomicron
  source 1..700: mol_type = genomic DNA
aaaaacaatgcaagaaatggaaactgctttcatcatatccggcaagaatattaagaaaggactccggtttgacgatgtaagcatgatgcaatacgatgtggcgtccactatcgcccgcatctttcatttagaacaaccacaagtttggataggtagacccatggagattgttttcaaataatcattcaggatgattaataggatgggaaaaggagaagctaaaataactggcttctccttttttatttatctatccacgctcataagataggagattataaatactatttcttataagtaacaaaatgagcagtcataatagttttatttcctataagtaatagaaatcacacctccacagctgttaattaccgaataatacttgcatcaacatacatatccttatacctttgtcccccggtaacacaacataatataacttcaatcataaaatgtactcttttgacattcttaataagcaatccaactgattaaagcagcattcaaacattctgataggacatcttatcttttgtttattcaaattaaaatacaatacgatagaattgtattagtaaatgataaaatactacttgtctgacggagcattatcgcctacttttgcatcatcagttattggttgaagtttccatggatcaatcaacaattgaatagagtttataatctaatacaataaatt

SEQ ID NO: 329 - DNA, 300 bp, Bacteroides thetaiotaomicron
  source 1..300: mol_type = genomic DNA
agttgtgtttcttgcagccaacgtgtgtaaatcgtttatgaatgattctctacgttttttgaatgatttttataatgtaaaatctacactcattcgtatctttgctgtagataacaacgaaataaactaatattatgaaaatgaaaactaatataatctttaaaaaactaaagtcattcgtcaatcgatgtctttttcctgattatatgtgctgttttatcttcctgcgtttataacgctgatatgccaataaagaatttttagtgaaccaataatttaatatgtaaaaagaaaaacgtt

SEQ ID NO: 330 - DNA, 300 bp, Bacteroides thetaiotaomicron
  source 1..300: mol_type = genomic DNA
gctactctgtgaccactatttataattaccggacacgtgtccgcaacaaggcactcggcgaacgggatgagtttgaggctaaagtaatgaaaatagggaaggtggaagagtaagaaatccactacttttttagcacggtcgctattctataatattcttattattagtgttttatgctgttttgtactgtataaaccactatatttttgctatctggcaaaagaagcgcatttaattgtggatacttttgtttcagtgaaaacaaaaagtaatcctaatattaactttaaattgatgtac

SEQ ID NO: 331 - DNA, 300 bp, Bacteroides thetaiotaomicron
  source 1..300: mol_type = genomic DNA
taatgtagctggcagacatccggaactgcttgagcggttaaagcaggaattttttgtgcaaacagatggattttatcgtagtgaggtggaagaagaaccattaaaataagcccgataaccccttctggaaagcatttgattgattaggtattagattgttttgcaggaattggtgtagaaggatagtttctgaacatgtgtgctcataataaaacatattttctatcgtctacaccttttttcttttacttttgctgcatcacaatcatacataaataacgtttaactttttagaatcat

SEQ ID NO: 332 - DNA, 300 bp, Bacteroides thetaiotaomicron
  source 1..300: mol_type = genomic DNA
acaaaaaaacattcattcctaaaataaaggatgatggactaatttatatatccatgaataaaggagataatcttaccataatcatcccttaataacaattttgcagtaaatgcgttattttttgctgcgtttatacactattaaaataacacatttacctaattttgactcgttgaatttagaaaattatcacattaaatctaataatatatgaaacgaattcgagatttccccaccactgacaagacaacgatctagttgcctgaaatgtattaacctaatattaattttaaattatcc

SEQ ID NO: 333 - DNA, 300 bp, Bacteroides thetaiotaomicron
  source 1..300: mol_type = genomic DNA
ctgcaaaacgtcctgtttctaaaaaatgcgggtttttgcctaaaaaacagtttgtttgtcactgcacgggaagtgaatcaccatctattggcatttgtttttgatcacttctctatcaataatgatcatttagcggattgcacagggttcttttgatcattattgaattttaattgatgatttttgaatgtttccttttatgctgtggttgtatttttgtcgaagattgaaacttattgttagctagtgaaagtatgaatctttattattaaatgtagtattatgaaaaaaaggaaacag

SEQ ID NO: 334 - DNA, 400 bp, Bacteroides thetaiotaomicron
  source 1..400: mol_type = genomic DNA
aaggaagtgtttagatgacataatgattatttgaacagggctaaatttgcacatactgacataaatgtgacattatgatagtttgatgatatatatttatcttcataactatcataatgtatttacaatagttttcaaatttattttgaataattgttttgtatttataaatatgtgcttatatttgcaaaatagcactaagcgaaaataagcgacttactaattcttataataataaagagtgtttatgaaaagaaaattaatgctgttattggcctgcttgctggcaagcataggtctagtgattgcccaaacacctaaaaaggtgacaggtgtcgtcatttctgaggaagatgatcagcctgttgtgggagcatccgtattagtgaaaggtacaacg

SEQ ID NO: 335 - DNA, 414 bp, Bacteroides thetaiotaomicron
  source 1..414: mol_type = genomic DNA
cctgtcacctttttaggtgtttgggcaatcactagacctatgcttgccagcaagcaggccaataacagcattaattttcttttcataaacactctttattattataagaattagtaagtcgcttattttcgcttagtgctattttgcaaatataagcacatatttataaatacaaaacaattattcaaaataaatttgaaaactattgtaaatacattatgatagttatgaagataaatatatatcatcaaactatcataatgtcacatttatgtcagtatgtgcaaatttagccctgttcaaataatcattatgtcatctaaacacttcctttttatctttaactaagccttgaataatataaaaattatattagatataaataattagtatgaaaataggttataggagaat

SEQ ID NO: 336 - DNA, 500 bp, Bacteroides thetaiotaomicron
  source 1..500: mol_type = genomic DNA
tccttgcttttgtgggggtgaagtgactgtgatgtggcttatttaagtggatgagccgttatgtttaacaggtaaacaaagcttggttagcatttttagtaagctatatgtgttcgttccatgtctgtgtcttattgcattgtatatcaattagctaagtccgaaagtgacggatgatgataaaatatgagtttcaaactccttagtaaaagctaaaaaatacatcattttaatagcggcaacgaaaaaaaacaaatattttttaagatgcctaattgtcatttattgatggctgaattttgattctgaaaagggaatgatttgttaaatatgtcatttttgttgctgaataatgtcataatgatagtttttcattcttttccgttttcgtacggagagtatctcataaaaaataatcatctttttgtcacccatttatggacgttatgtataattatagtaaagcaaagataaaatcctcattagaaatagataaaatt

SEQ ID NO: 337 - DNA, 300 bp, Bacteroides thetaiotaomicron
  source 1..300: mol_type = genomic DNA
tctcttgaaactgtgaagactcaaaagaagagagcaatgtccttccttagaaaaaaattaggctcctatcattttctattattacaaatgttacttccctaaaaagaagcataacactcaatccgctttacttgttaacacctgtttgcaatgaacaaaatgttcgttgtttttcttgattattgtacaaaaagaacggtcttctaaataaataaagtatcttttgaataaaaaaacttcagaatcctgtccctcattttttcttcttagtcgtctttgctataaataacctgaaataat

SEQ ID NO: 338 - DNA, 300 bp, Bacteroides thetaiotaomicron
  source 1..300: mol_type = genomic DNA
atattcctgaaagccggagaatccacaactgtcaagatgacattacctaaagaatcatttatgtattttgatgtcaacagaactcagttcgtcactgatccgggtatttacaatataatgcttggcttctcctctagagatattaaagcacaaaaaaatattcaatatacactatagattgcataatcaataagtaacatgaaatatatttgtatcaaaactgtagcaatttacttgctcgcaaccttacctttattggcgaacgcctcaacacacaaggtaaaaattacacatagcgtc

SEQ ID NO: 339 - DNA, 300 bp, Bacteroides thetaiotaomicron
  source 1..300: mol_type = genomic DNA
gaatacaatttataattatcgggcgaaagtaaaaaacaaagctcgtggttccagagaagatttcgaagatttagttcggaaaatacgttgatcgatgttgtaacctatccactttttaggcctctttggttgatattaacttgctgtttattagtggtttgtatgtttgtataatccacttattataccctcattgtcaaatcacttcagtttctttatacttttgacctgtgcttctgaaatagtggcgaaagtcctgtcaagcacataccttaattataaactttaaaattaatacgc

SEQ ID NO: 340 - DNA, 500 bp, Bacteroides thetaiotaomicron
  source 1..500: mol_type = genomic DNA
acatttctcccttgaagggcatatttgcacagatattccccaaaagcctgcttaattatttaatgtaattgaaaatgtaattataataatctgactaatatataatatattctggtcatttcaaaaaaaaggcattctaactcttctacatttttattatatttatttgtaattaaccgattgattgttaattataattatcatcatcacacctacataaagttatgcaaactaaataagaaattacttatttagttagctttgccatgtcttttctcagtcaaactcttcgttgatgagaatcagacatatatagatatttgtacttaaatctacaacatattatgagcacaacatgccaacatcaacaaggtgaaggatacctaaaactcctaaagatttagatacatattatgcttataataagaaagataggcaaacgttgtaagcaataatatttattgttcggatgcactttaaccattttattatttatattt

SEQ ID NO: 341 - DNA, 500 bp, Bacteroides thetaiotaomicron
  source 1..500: mol_type = genomic DNA
aaagttagaagtcatcaattaatagaccttcattttgggttggtaaggataaaatctgctcaaaaaaggataaatactgctcaaacaacgtcagagcccctattccgtatgttaaaagaacgtttgaaatccaaaaaagaatagattttgtcctttcttgaacacagattttccttgtttcttttattaattccattattttgtgtcatcaagatgagatactatggaaaataaagagtttgttaaagctcgatatttgctagaaatacacgatttgttagccgcttctccataacatttcatcctaaatatgacagtactgatattatatattaaaggtataggaaaacgggtataaagattgcaattattcaaaggtgagatagtattaaggtaaaaagtggatttaaaagaatacttagggcttgtttttttatgaggtaaataagattgatggatggataaccttggatttgaagattaatttaaagcaaatattt

SEQ ID NO: 342 - DNA, 300 bp, Bacteroides thetaiotaomicron
  source 1..300: mol_type = genomic DNA
gtttgtagatcctctttataaatacatcaatgaaactacttcacgtgtgcctatcagtgactggcatgataccaaaactggcagaatgaccggatttaaggcacgttccgtaattggaggatattggatacaagtgttaatggataagatgaatcattaaaaatatggacaataagttgaaagaatactttttcgcatggatgaatcatttcagcataatacaacaagagataagtaatgaaccaacaatataatattttattaatcattaattttttacgtatgaaaaaaaacaaaatc

SEQ ID NO: 343 - DNA, 300 bp, Bacteroides thetaiotaomicron
  source 1..300: mol_type = genomic DNA
ccattggactcccgcaaagagtatacaaagtctgactggattatgtggacagctgctatgtcttcagatttagaaactttcaagaaattcatagatccactctataaatatatcaatgaaactacttcgcgggtacctatcagtgattggcatcacacagacagtggtgaatgggttgggtttaaagcgcgttcggttattggaggatactggatgcaagtgttaatggacaaaactcgatgaaatgtttaattaactatacatttattattaactagaatgaaaaacatgaaatgtaaa

SEQ ID NO: 344 - DNA, 300 bp, Bacteroides thetaiotaomicron
  source 1..300: mol_type = genomic DNA
gaataaatgtcgaatttgactcagcgctaagtaagataaaatgttccggaaaaattgatttgaaagacaactttgagacagtaattaatggattaacctttgtagcaccaatatcttatgcctatgatggacagtataaaacttatcgggtcgtgaaaaaatagacacagaaaaatatagtaattaataaaataaatctcaagaatttatggcgatttttataaagagtggataatactgctgtattatccagatggggctcatatgcctgtaagtattaagttataaaataataatatt

SEQ ID NO: 345 - DNA, 300 bp, Bacteroides thetaiotaomicron
  source 1..300: mol_type = genomic DNA
gtctgcctgatgtaaaagagtagttgcattacctccatcatgacaagcagagagagacaaaatcaggaacaaccctataatataagaaagaagtagcttcatagttcgtttcgttttacaaatgtaaatagaataaaacaaacaagcaacaaaaagacaaataattttttcacacatcataattacaatgttaaaattgccctttctctctctttccctaatcttttttgcctttcatttgttcactttgttactttcgccggaagacggcagaataaacttcaacctaaatagtaacga

SEQ ID NO: 346 - DNA, 500 bp, Bacteroides thetaiotaomicron
  source 1..500: mol_type = genomic DNA
aatgtcgcgatgagcaacagagtcaatcatgacccaacccctgaatcaaacgaaattatatatgatttttttaaatggtacttgcattcagcaaataaatctaccaaagagtagaaagatgcgttttttgaatacactttttcgtttttttgcgcaaaaaatcaactctggatgcctaactttgttctatcaattcatccaagtaacctaaattacttgactaaaatgattagcctatgaaaaatatacactagaaaacataaaatgccccatctgcgaatggggcaatattaatgccatattaataaaactaatcataaattatcgcccaattaataaataacacaataataaattcataattaataaattaaaagtgctagctcaaaggaattaataaagctagcagatgattccgagttagtaataaatattaaactattaaaaattcaataggttaaatatatactaaaaagagtagaaactaaaaaaagtaaact

SEQ ID NO: 347 - DNA, 400 bp, Bacteroides thetaiotaomicron
  source 1..400: mol_type = genomic DNA
cgtaaagggaactatagtgcatcttgcgtaccactatagtggcacgaactaatatgactaacaaaaaacttaattgttcgtagctaaggacttaagaatgtgagcgataacctgcaaggagtgaactgatttcattaacggtttaccatataacaaattattgttttattgatgttatgttatatgtggtttttattttacagatcagattatccttattccttggtcgaattatagtttaaaaaggaggaatatctataattcgacctcagataactcatattggacctcatctggagtgacttaacttacatttgcacttgttaattggtcgttgaaatcataaatcaattaatgaacggttaattttttatttactaatatcttaaatatttttgct

SEQ ID NO: 348 - DNA, 300 bp, Bacteroides thetaiotaomicron
  source 1..300: mol_type = genomic DNA
atttttccagttccaatcggcattatgaatagccaaacctatgttcaaaacaagcgggtgcaatttatcaacaaatgaagccatagcatatagggtttattaatgtgaagataaagatatataaaaaataaatataactcttttgatgtcgataattttaaataatatatcgatattgttttataataaatcctgctatagtcgttcatgtcgatattgtattactctaaatcgtattagtcctacaatgtggactgccgagttgttacatttgcggtatataatctaataggaatattt

SEQ ID NO: 349 - DNA, 300 bp, Bacteroides thetaiotaomicron
  source 1..300: mol_type = genomic DNA
tacttcctgcctcatctgctttcgatttattgacagagaaaaaggttgaattaagaaatgaattaacacttccaagtcgtgaaatataccttttatccttttaataagttcacttcaacttatatttatcagaatacagaaaagaaataaactactgtaaaacaaaacattaacattaatacaaacttgtatttttattctagacaacccaaagtctcaagatggttgctatatttgcaatatccggaacagagcggagtaaaaatattagttagcttataatttaataccataaataga

SEQ ID NO: 350 - DNA, 300 bp, Bacteroides thetaiotaomicron
  source 1..300: mol_type = genomic DNA
atcgtctggatggaaaatctcataaagaaatagctgaagaattgggtatcagtgtcaaaggagtggagtatcatataagcaaagctgtgaagctgctgcgtgataatctgaaagagtatgctcctttcctgatattttttatttgatgatcttgaaaatatctttttctcctctttttttaataacagcaaaaaaagttgtatttctcactagggattatttttagtgagtggtattctatatgaaagggggagcaaatatactcctgtttattacttataaagtaactcaacttataat

SEQ ID NO: 351 - DNA, 500 bp, Bacteroides thetaiotaomicron
  source 1..500: mol_type = genomic DNA
atcggtggcggagcctgtcctcatcggtgccggaaataggtctcatcggtacccgaaataaatccttgcggagtaaggcactcattataccatcaaataataattgtttttttttatcatgaaaacattattgataactagatacttatgcgaaaagaatgaaattctttatcgctattaatctaaaattaatatggcattaaggactggaattctatatttgcttcagaaacggacgggatatcctccactatgtttctgatgacttgagcggatcgaactataaccttataaaatgatggaaatatgaaaaacgacacttcttaactactgcttggacttacttgtcagagaaagttttcttgagatgaaaacggtttgaatcaattaatgttaatctataaattatgaaaacaggaaagagcaaagatcatttgcaaagagtttttttaaggttcctttatttgataatggggagcatcctttctttggattctgtt

SEQ ID NO: 352 - DNA, 702 bp, Bacteroides thetaiotaomicron
  source 1..702: mol_type = genomic DNA
ttatatggaagacctggaaggctgggcagattctattgtttgtaccactacggaacaggcggattttgtcaggaagttcttgcaggtcgtggagcagaatctggatcgtgaagatttgggctccacgtttgtggcagaacaaatgttgatgagttcccgtcagttctaccggaggtttaaggagatttcgggtatgtcacccagcgatttgattaaagattaccggatggagaaggccgcccgtttgttgcagaacgaggaactgtctattcaggatgtaatttccgatgtaggtatatccagtcgtgcttacttctacaaagagtttacccgtaagtttggcgtaactcctaaggtatatagagagaagcttttagggaatgacactcataaaaatcaggagtgaacagccaggaagcggtctggtattagttgccaagaaataaattcccatgctgatctgtgctgctttttaaggttggtctcctttgtactctcttttgttttccattatttattattctgatatatagggtattatccagtactctattttaccatctgtactttaaacagacattttttctcttctctgttttttggcgcacaggagtatctcatttctttggctattcctttaatttgcgtcaacgtattaaacgtatttggtaaccttattattattaattaaaaacaagtagt

SEQ ID NO: 353 - DNA, 500 bp, Bacteroides thetaiotaomicron
  source 1..500: mol_type = genomic DNA
agaggaactatagtgcttcttgcgtccaactatagtggaacgaaagggaaactacagtagaacgaatgaatttggactgataagaaattagtttttcaaagtctttcggttaatgattcgggattccttcattccttctgttctttggtcgtttggggtgacagatatatttgaattcatgtctgtatagattttatttattactgcttacgaataaactttttaataaaagtggcgaaaaggcggctattataacttgatacaatgtgcgcaaacttgtgctctgtttgtacgtgaatgaatgctatgtgttgatatatagatgtttataatgtgctttatttctcctttctgttctataaatgagcattttcattttgacataattttgtcacttctgcggaatattcttaatattaaaagtttttaatttgcttccgcttataagaattatataggcgattgataaccttagtattaattaaaaaaaacaaagtaac

SEQ ID NO: 354 - DNA, 300 bp, Bacteroides thetaiotaomicron
  source 1..300: mol_type = genomic DNA
atatttgactccggattattttcgctggttgtatacggcatttactcgcgccacgaagacattgtatctggtgaattatcccaaagaacaaattttataaaagatttcttttttttcttcgtaaataattgattcccagtctagatgaatggggagatactcgttgtgaaaaaataaatatgagattatatttattttttttcaaaaacctctaagtgtttttcaaggtatgggttgttatataaatgtgaatcgaaaataataaatggaatatcgattctaatatttatataaaaacta

(19) United States
(12) Patent Application Publication
(10) Pub. No.: US 2025/0257382 A1
(43) Pub. Date: Aug. 14, 2025

(54) HARNESSING GUT MICROBES FOR GLYCAN DETECTION AND QUANTIFICATION

(71) Applicant: THE PENN STATE RESEARCH FOUNDATION, University Park, PA (US)

(72) Inventors: Guy Edmund TOWNSEND, University Park, PA (US); Jennifer L. MODESTO, University Park, PA (US)

(21) Appl. No.: 18/854,812

(22) PCT Filed: Apr. 7, 2023
(86) PCT No.: PCT/US2023/065548

§ 371 Date: Oct. 7, 2024

Related U.S. Application Data
(60) Provisional application No. 63/328,555, filed on Apr. 7, 2022.

(51) Int. Cl. C12Q 1/02; C12N 9/02; C12N 15/74; C12Q 1/66

(52) U.S. Cl. CPC C12Q 1/02; C12N 9/0071; C12N 15/74; C12Q 1/66; C12Y 114/14003; G01N 2400/10

(57) ABSTRACT

The present disclosure relates compositions and methods of engineering microbial strains to detect and quantify glycan molecules.

### RELATED APPLICATION

This PCT application claims priority to, and the benefit of, U.S. Provisional Patent Application No. 63/328,555, filed Apr. 7, 2022, entitled “HARNESSING GUT MICROBES FOR GLYCAN DETECTION AND QUANTIFICATION,” which is incorporated by reference herein in its entirety.

### STATEMENT REGARDING FEDERALLY SPONSORED RESEARCH

This invention was made with government support under Grant No. AI149319 awarded by the National Institutes of Health/NIAID. The Government has certain rights in the invention.

### REFERENCE TO SEQUENCE LISTING

The sequence listing submitted on Apr. 7, 2023, as an .XML file entitled “11196-101WO1_Sequence_Listing.xml” created on Apr. 7, 2023, and having a file size of 360,902 bytes is hereby incorporated by reference pursuant to 37 C.F.R. § 1.52 (e) (5).

### FIELD

The present disclosure relates compositions and methods of engineering microbial strains to detect and quantify glycan molecules.

### BACKGROUND

Characterization of glycan molecules present in heterogenous mixtures has generally been performed using lectins, nuclear magnetic resonance, or liquid or gas chromatography (LC or GC, respectively) followed by mass spectrometry methods. Using these methods are proven to be expensive and requires tremendous investments in expertise, instrumentation, and consumable products. Furthermore, these approaches require glycan separation and derivatization prior to implementation, which can result in non-uniform detection and often necessitate tandem parallel approaches to achieve high-confidence results. Detection of glycan molecules in heterogenous mixtures allows for profiling microbes present in a patient, specifically in the gastrointestinal tract of the patient. Further, glycan detection can allow for early detection, treatment, and/or prevention of pathogenic microbes within a patient.

Given the limitations described above, there is a need to effectively and efficiently detect and quantify glycans in heterogenous mixtures using improved and optimized methods.

The compositions and methods disclosed herein address these needs.

### SUMMARY

The present disclosure provides a system for sensing and detecting glycan molecules using a bacterial reporter plasmid.

In one aspect, one aspect, disclosed herein are reporter plasmids comprising a luciferase reporter cassette, a first polysaccharide utilization locus (PUL), and a first glycan-specific promoter.

Also disclosed herein are reporter plasmids of any preceding aspect, wherein the first PUL encodes any combination of proteins comprising a surface glycan binding protein (SGBP), an outer membrane channel, a digestive enzyme, a glycan importer, or a glycan sensor.

In one aspect disclosed herein are reporter plasmids of any preceding aspect, wherein the first PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof.

Also disclosed herein are reporter plasmids of any preceding aspect, wherein the first glycan specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof including, but not limited to glycan specific promoters comprising the nucleic acids sequence as set forth in SEQ ID NOs 74-76, or SEQ ID NOs: 262-354.

In one aspect disclosed herein are reporter plasmids of any preceding aspect, wherein the reporter plasmid further comprises a second PUL. In some embodiments, the second PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof. In some embodiments, the first PUL is interchangeable with the second PUL.

Also disclosed herein are reporter plasmids of any preceding aspect, wherein the reporter plasmid further comprises a second glycan-specific promoter. In some embodiments, the first glycan-specific promoter is interchangeable with the second glycan-specific promoter. In some embodiments, the second glycan-specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof.

In one aspect, disclosed herein are reporter plasmids of any preceding aspect, wherein the second glycan-specific promoter comprises the nucleic acids sequence as set forth in SEQ ID NOs 74-76, or SEQ ID NOs: 262-354.

Also disclosed herein are reporter plasmids of any preceding aspect, wherein the reporter plasmid comprises a Bacteroides-optimized lux (Bolux) plasmid.

Also disclosed herein are engineered bacteria comprising the reporter plasmid of any preceding aspect. For example, disclosed herein are engineered bacteria harboring a reporter plasmid wherein the reporter plasmid comprises a luciferase reporter cassette, a first polysaccharide utilization locus (PUL), and a first glycan-specific promoter. In some aspects, the first PUL encodes any combination of proteins comprising a surface glycan binding protein (SGBP), an outer membrane channel, a digestive enzyme, a glycan importer, or a glycan sensor. In some embodiments, the first PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof. In some aspects, the first glycan specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof.

In one aspect, disclosed herein are engineered bacteria of any preceding aspect, wherein the first glycan-specific promoter comprises the nucleic acids sequence as set forth in SEQ ID NOs 74-76, or SEQ ID NOs: 262-354.

Also disclosed herein are engineered bacteria of any preceding aspect, further comprising a second PUL. In some aspects, the second PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof. In some aspects, the first PUL is interchangeable with the second PUL.

In one aspect, disclosed herein are engineered bacteria of any preceding aspect, further comprising a second glycan-specific promoter. In some embodiments, the first glycan-specific promoter is interchangeable with the second glycan-specific promoter. In some aspects, the second glycan-specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof.

Also disclosed herein are engineered bacteria of any preceding aspect, wherein the second glycan-specific promoter comprises the nucleic acids sequence as set forth in SEQ ID NOs 74-76, or SEQ ID NOs: 262-354.

In some aspects, disclosed herein are engineered bacteria of any preceding aspect, wherein the engineered bacteria originates from a gastrointestinal bacterium. In some aspects, the engineered bacteria originates from a Bacteroides bacterium including, but not limited to Bacteroides thetaiotaomicron (Bt), Bacteroides ovatus (Bo), Bacteroides fragilis (Bf), Bacteroides caccae (Bc) or a variant strain thereof.

Also disclosed herein are engineered bacteria of any preceding aspect, wherein the engineered bacteria overexpresses the chondroitin sulfate (CS)-specific PUL when exposed to a CS glycan, or derivatives thereof.

In some aspects, disclosed herein are engineered bacteria of any preceding aspect, wherein the engineered bacteria overexpresses the levan-specific PUL when exposed to a levan glycan, or derivatives thereof.

Also disclosed herein are engineered bacteria of any preceding aspect, wherein the engineered bacteria overexpresses the inulin-specific PUL when exposed to an inulin glycan, or derivatives thereof.

In some aspects, disclosed herein are engineered bacteria of any preceding aspect, wherein the engineered bacteria overexpresses the arabinogalactan-specific PUL when exposed to an arabinogalactan, or derivatives thereof.

Also disclosed herein are engineered bacteria of any preceding aspect, wherein the engineered bacteria overexpresses the dextran-specific PUL when exposed to a dextran glycan, or derivatives thereof.

In some aspects, disclosed herein are engineered bacteria of any preceding aspect, wherein the engineered bacteria overexpresses the hyaluronan (HA)-specific PUL when the engineered bacteria is exposed to a HA glycan, or derivatives thereof.

Also disclosed herein are engineered bacteria of any preceding aspect, wherein the engineered bacteria overexpresses the heparan sulfate (HS)-specific PUL when exposed to a HS glycan, or derivatives thereof.

In some aspects, disclosed herein are engineered bacteria of any preceding aspect, wherein the engineered bacteria overexpresses the O-glycan(OG)-specific PUL when exposed to an OG glycan mixture, or derivatives thereof.

Also disclosed herein are engineered bacteria of any preceding aspect, wherein the engineered bacteria emits a light signal when overexpressing any PUL of any preceding aspect. In some aspects, the light signal is decreased when the glycan molecule is broken down into a monosaccharide molecule.

In one aspect, disclosed herein are glycan-sensing systems comprising the engineered bacteria or reporter plasmid of any preceding aspect. For example, disclosed herein is a glycan-sensing system comprising an engineered bacteria harboring a reporter plasmid, wherein the reporter plasmid comprises a luciferase reporter cassette, a first polysaccharide utilization locus (PUL), and a first glycan-specific promoter, wherein the glycan-sensing system detects and quantifies a glycan molecule.

In some embodiments, the glycan molecule comprises chondroitin sulfate, arabinan, heparan sulfate, hyaluronan, fructan, levan, mannan, or derivatives thereof. In some embodiments, the glycan molecule is located in the gastrointestinal tract of a mammal.

In some embodiments, the first PUL encodes any combination of proteins comprising a surface glycan binding protein (SGBP), an outer membrane channel, a digestive enzyme, a glycan importer, or a glycan sensor.

In some embodiments, the first PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof.

In some embodiments, the first glycan specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof.

In some embodiments, the system further comprises a second PUL. In some embodiments, the second PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof. In some embodiments, the first PUL is interchangeable with the second PUL.

In some embodiments, the system further comprises a second glycan-specific promoter. In some embodiments, the first glycan-specific promoter is interchangeable with the second glycan-specific promoter. In some embodiments, the second glycan-specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan (OG)-specific promoter, or variants thereof.

In some embodiments, the first or second glycan-specific promoter comprises the nucleic acids sequence as set forth in SEQ ID NOs 74-76, or SEQ ID NOs: 262-354.

In some embodiments, the reporter plasmid comprises a Bacteroides-optimized lux (Bolux) plasmid.

In some embodiments, the engineered bacteria originates from a gastrointestinal bacterium. In some embodiments, the engineered bacteria originates from a Bacteroides bacterium. In some embodiments, the engineered bacteria originates from a Bacteroides thetaiotaomicron (Bt), Bacteroides ovatus (Bo), Bacteroides fragilis (Bf), Bacteroides caccae (Bc) or a variant strain thereof.

In some embodiments, the engineered bacteria are grown in an anaerobic environment. In some embodiments, the engineered bacteria are grown in a 96-well, a 384-well, or a 1536-well microplate.

In some embodiments, the engineered bacteria overexpresses the chondroitin sulfate (CS)-specific PUL when exposed to a CS glycan, or derivatives thereof.

In some embodiments, the engineered bacteria overexpresses the levan-specific PUL when exposed to a levan glycan, or derivatives thereof.

In some embodiments, the engineered bacteria overexpresses the inulin-specific PUL when exposed to an inulin glycan, or derivatives thereof.

In some embodiments, the engineered bacteria overexpresses the arabinogalactan-specific PUL when exposed to an arabinogalactan, or derivatives thereof.

In some embodiments, the engineered bacteria overexpresses the dextran-specific PUL when exposed to a dextran glycan, or derivatives thereof.

In some embodiments, the engineered bacteria overexpresses the hyaluronan (HA)-specific PUL when the engineered bacteria is exposed to a HA glycan, or derivatives thereof.

In some embodiments, the engineered bacteria overexpresses the heparan sulfate (HS)-specific PUL when exposed to a HS glycan, or derivatives thereof.

In some embodiments, the engineered bacteria overexpresses the O-glycan(OG)-specific PUL when exposed to an OG glycan mixture, or derivatives thereof.

In some embodiments, the engineered bacteria emits a light signal when overexpressing any PUL of any preceding aspect.

In some embodiments, the light signal is decreased when the glycan molecule is broken down into a monosaccharide molecule.

In one aspect, disclosed herein is a method of detecting a glycan, the method comprising isolating and preserving a tissue sample from a subject, preparing, and culturing the glycan-sensing system of any preceding aspect in a microplate in an anaerobic environment, adding the tissue sample into the microplate, and detecting and quantifying a light signal emitted from the system of any preceding aspect.

In some embodiments, the tissue sample comprises at least one glycan. In some embodiments, the light signal is detected and quantified using a microplate reader, a spectrophotometer, or an instrument capable of measuring the light signal. In some embodiments, the tissue sample comprises a tissue biopsy, a blood sample, or a saliva sample.

In some embodiments, the method prevents, treats, or reduces a gastrointestinal disease or disorder in the subject.

In one aspect, disclosed herein is a method of treating or preventing a gastrointestinal disease or disorder in a subject in need thereof, the method comprising isolating and preserving a tissue sample from a subject, preparing and culturing the glycan-sensing system of any preceding aspect in a microplate in an anaerobic environment, adding the tissue sample into the microplate, detecting and quantifying a light signal emitted from the system, and performing or administering a therapeutic procedure to the subject comprising pathological amounts of a glycan relative to a control tissue comprising normal amounts of the glycan.

In some embodiments, the tissue sample comprises at least one glycan. In some embodiments, the light signal is detected and quantified using a microplate reader, a spectrophotometer, or an instrument capable of measuring the light signal. In some embodiments, the tissue sample comprises a tissue biopsy, a blood sample, or a saliva sample.

In some embodiments, the therapeutic procedure comprises a surgical procedure, a therapeutic agent, a lifestyle change, or a combination thereof.

In some embodiments, the surgical procedure comprises bariatric surgery, esophageal surgery, foregut surgery, hernia surgery, gastroesophageal reflux disease surgery, surgical nutrition, colorectal surgery, gallbladder surgery, liver surgery, bile duct surgery, pancreatic surgery, tumor excision, or combinations thereof. In some embodiments, the therapeutic agent comprises a laxative, an antacid, a proton pump inhibitor, a histamine blocker, an anti-inflammatory agent, a steroid, a vitamin, an intravenous fluid, an antibiotic, a probiotic, or combinations thereof. In some embodiments, the lifestyle change comprises a dietary alteration, an exercise program, a sedentary lifestyle, or combinations thereof.

In some embodiments, the gastrointestinal disease or disorder comprises heartburn, irritable bowel syndrome, lactose intolerance, gallstones, cholecystitis, cholangitis, anal fissure, hemorrhoids, proctitis, colon polyps, infective colitis, ulcerative colitis, ischemic colitis, Crohn's disease, radiation colitis, celiac disease, diarrhea (chronic or acute), constipation (chronic or acute), diverticulosis, diverticulitis, acid reflux (gastroesophageal reflux (GER) or gastroesophageal reflux disease (GERD)), Hirschsprung disease, abdominal adhesions, achalasia, acute hepatic porphyria (AHP), anal fistulas, bowel incontinence, centrally mediated abdominal pain syndrome (CAPS), clostridioides difficile infection, cyclic vomiting syndrome (CVS), dyspepsia, eosinophilic gastroenteritis, globus, inflammatory bowel disease, malabsorption, scleroderma, or volvulus.

In some embodiments, the subject is a human.

In one aspect, disclosed herein is a kit for detecting or purifying a glycan molecule, the kit comprising an apparatus comprising a binding medium and a surface glycan binding protein (SGBP), wherein the binding medium comprises a matrix of biomolecules, the SGBP comprises a peptide label, and the SGBP is attached to the matrix; a container comprising an equilibration buffer, a container comprising a releasing buffer, and a container comprising a washing buffer.

In some embodiments, the glycan molecule comprises chondroitin sulfate, arabinan, heparan sulfate, hyaluronan, fructan, levan, mannan, or derivatives thereof.

In some embodiments, the SGBP comprises a chondroitin sulfate (CS)-specific SGBP, a levan-specific SGBP, an inulin-specific SGBP, an arabinogalactan-specific SGBP, a dextran-specific SGBP, a hyaluronan (HA)-specific SGBP, a heparan sulfate (HS)-specific SGBP, an O-glycan(OG)-specific SGBP, or variants thereof.

In some embodiments, the matrix comprises a nickel-conjugated agarose polymer. In some embodiments, the peptide label comprises a hexa-histidine label. In some embodiments, the releasing buffer comprises a histidine solution. In some embodiments, the washing buffer comprises a buffered solution comprising water, phosphate buffered saline solution, a tris-buffered saline solution, or variants thereof.

In some embodiments, the kit is combined with the glycan-sensing system of any preceding aspect to detect the glycan molecule.

### BRIEF DESCRIPTION OF FIGURES

The accompanying figures, which are incorporated in and constitute a part of this specification, illustrate several aspects described below.

FIGS. 1A, 1B, 1C, 1D, 1E, 1F, 1G, 1H, and 1I show the (FIG. 1A) PUL-encoded surface glycan binding proteins (SGBPs) sequester target glycans prior to their translocation across the outer membrane by the corresponding SusC channel. Once in the periplasm, the glycan is depolymerized into mono, di-, or oligo-saccharide signatures that bind their cognate sensor, which subsequently activates PUL transcription in the cytoplasm. The sensor ligand is subsequently further degraded into monosaccharide components and transported across the inner membrane, where they enter central metabolism, thereby deactivating the sensor. FIG. 1B shows a cartoon depicting the strategy employed in this proposal to harness the PUL-encoded glycan detection machinery to report the presence of distinct glycans. FIG. 1C shows the growth of wild-type (wt, black) or mutant Bt strains deficient for the PUL-encoded susC transporter (ΔBT3332, blue), lyases (ΔBT3324 ΔBT3350 ΩBT4410, purple), sensor (ΔBT3334, red) or glycosyl hydrolase (gh, ΔBT3348, orange) supplied CS as the sole carbon source. n=8, error bars are SEM. FIG. 1D shows the fold increase in susC transcript levels were measured by qPCR 120 minutes following the introduction of 0.2% CS to cultures of wild-type Bt or a strain deficient for the corresponding PUL sensor, BT3334. FIG. 1E shows the fold change in susC transcript levels were measured by qPCR 120 minutes following the introduction of 0.2, 0.02, or 0.002% CS prepared with galactose to a final carbohydrate content of 0.5%. FIG. 1F shows the fold increase in susC transcript levels were measured by qPCR 120 minutes following the introduction of 0.2% levan to cultures of wild-type Bt or a strain deficient for the corresponding PUL sensor, BT1754. FIG. 1G shows the growth of wild-type (wt, black) or mutant Bt strains deficient for the PUL-encoded susC transporter (ΔBT1763, blue), levanases (ΔBT1760-59 ΔBT3082 ΩBT1765, purple), sensor (ΔBT1754, orange) or fructose importer (ΩBT1758, purple) supplied levan as the sole carbon source. n=8, error bars are SEM. FIGS. 1H and 1I show the fold change in susC transcript levels were measured by qPCR after (FIG. 1H) 120 or (FIG. 1I) 60 minutes following the introduction of 0.2, 0.02, or 0.002% levan prepared with galactose to a final carbohydrate content of 0.5%. For FIGS. 1D-1G, n=6, error bars are SEM, p-values were calculated by paired two-tailed Student's t-test, and R2 values were computed by Microsoft Excel power trendline functions. ns indicates values <0.05, *<0.05, **<0.01, and ***<0.001.

FIGS. 2A, 2B, and 2C show the construction of a Bacteroides-optimized bioluminescent reporter. FIG. 2A shows the schematic depicting the construction of a bioluminescent reporter that encodes the entire Pl lux cassette under control of the Bt rpoD promoter and rpiL* RBS (top); a Bacteroides optimized lux cassette with rearranged luxA-E (indicated by the shaded regions) and Bt intergenic regions from a constitutively expressed Bt operon (BT1160-1155; middle); or pBolux which has BamHI and SpeI sites positioned upstream of the Bacteroides optimized lux cassette (bottom) in the multi-copy plasmid pLYL01. FIG. 2B shows the relative luminescence (solid lines) or growth (dashed lines) from Bt strains harboring an empty vector (black) or plasmids containing either the lux operon from Pl (blue) or the Bacteroides-optimized lux cassette (red) expressed from the Bt rpoD promoter and rpiL* RBS were measured during growth in minimal media containing 0.5% galactose as the sole carbon source. FIG. 2C shows the relative luminescence (solid lines) or growth (dashed lines) of Bt strains harboring empty pBolux (black) or a plasmid with the Bt rpoD promoter cloned into the BamHI and SpeI sites (red) during growth in galactose as the sole carbon source. All values in FIGS. 2B and 2C are the mean of 8 biological replicates and error bars are SEM in color-matched shading. Source data are provided as a Source Data file.

FIGS. 3A, 3B, 3C, 3D, 3E, and 3F show the construction of a glycan-responsive reporter in Bt. FIG. 3A shows the relative luminescence from wild-type Bt strains harboring pBolux (black) or a plasmid including the promoter region preceding the CS-inducible susC gene (P-BT3332, pink lines) following the introduction of CS as the sole carbon source normalized by the relative luminescence of identical cultures supplied galactose. Values are the mean of 12 biological replicates and error is SEM in color-matched shading. FIG. 3B shows the relative luminescence from wild-type Bt (black) or strains lacking a CS-inducible susC (ΔBT3332, blue), 3 CS-specific lyases (ΔBT3324 ΔBT3350 ΩBT4410, purple), CS-sensor (ΔBT3334, red), or a glucuronyl hydrolase (ΔBT3348, orange) harboring P-BT3332 following the introduction of an equal mixture of CS and galactose normalized to measurements from identical strains supplied galactose alone. FIG. 3C shows the relative luminescence from wild-type Bt (black) or strains lacking 3 CS-specific lyases (ΔBT3324 ΔBT3350 ΩBT4410, purple) or the CS-sensor (ΔBT3334, red) harboring P-BT3332 following the introduction of a mixture of unsulfated CS disaccharide (di0S) and galactose normalized to measurements from identical strains supplied galactose alone. Values are the mean of 6 biological replicates and error is SEM in color-matched shading. FIGS. 3D-3F shows the relative luminescence from wild-type Bt (solid lines) or strains lacking the CS-sensor (ΔBT3334, dashed lines) harboring P-BT3332 following the introduction of a mixture of galactose and either hyaluronic acid (HA, green) or heparin (blue) and galactose normalized to measurements from identical strains supplied galactose alone. For FIGS. 3B and 3D, values are the mean of 8 biological replicates and error is SEM in color-matched shading. Source data are provided as a Source Data file.

FIGS. 4A, 4B, 4C, 4D, 4E, 4F, 4G, and 4H show the levan-responsive reporter reveals multiple levanases coordinate fructan utilization in Bt. FIG. 4A shows the relative luminescence from wild-type Bt harboring pBolux (black) or a plasmid including the promoter region preceding the levan inducible susC gene (P-BT1763, pink) following the introduction of levan as the sole carbon source and normalized by the relative luminescence of identical cultures supplied galactose. Values are the mean of 12 biological replicates and error is SEM in color-matched shading. FIGS. 4B and 4C show the relative luminescence from wild-type Bt (black) or strains lacking the levan-inducible susC (ΔBT1763, blue), 4 levan-specific hydrolases (ΔBT1760-1759 ΔBT3082 ΩBT1765, purple), fructan sensor (ΔBT1754, red), or a putative inner membrane fructose transporter (ΩBT1758, orange) harboring P-BT1763 were measured following the introduction of an equal mixture of galactose and (FIG. 4B) levan or (FIG. 4C) fructose and normalized by the relative luminescence of identical cultures supplied galactose alone. FIG. 4D shows the growth of wild-type Bt (black) or strains lacking the levan-inducible susC (ΔBT1763, blue), 4 levan-specific hydrolases (ΔBT1760-1759 ΔBT3082 ΩBT1765, purple), fructan sensor (ΔBT1754, red), or a putative inner membrane fructose transporter (ΩBT1758, orange) were measured during anaerobic culture in minimal media containing fructose as a sole carbon source. FIG. 4E shows the relative luminescence of wild-type Bt or strains lacking all other levanases except BT1760 (ΔBT1759 ΔBT3082 ΩBT1765, pink), BT1759 (ΔBT1760 ΔBT3082 ΩBT1765, teal), BT3082 (ΔBT1760-59 ΩBT1765, lavender), or BT1765 (ΔBT1760-59 ΔBT3082, purple) harboring the levan-responsive reporter following the introduction of a mixture of levan and galactose normalized with measurements from identical cultures supplied galactose alone. FIG. 4F-4H show the relative luminescence of wild-type Bt or strains lacking BT1760 (pink), BT1759 (teal), BT3082 (lavender), or BT1765 (purple) harboring P-BT1763 following the introduction of a mixture of levan and galactose normalized with measurements from identical cultures supplied galactose alone. For FIGS. 4B-4F, values are the mean of 8 biological replicates and error is SEM in color-matched shading. Source data are provided as a Source Data file.

FIGS. 5A, 5B, 5C, 5D, 5E, and 5F show the species-specific responses that enable PUL reporters to distinguish between compositionally identical yet structurally distinct glycans. FIG. 5A shows the growth of wild-type Bt (gray) and Bo (black) or a strain lacking the Bo inulin sensor (ΔBACOVA_04496, red) were measured during anaerobic culture in minimal media containing inulin as a sole carbon source. FIG. 5B shows the relative luminescence from wild-type Bo harboring pBolux (black) or a plasmid including the promoter region preceding the inulin-inducible susC gene (P-BACOVA_04505, pink) were measured following the introduction of inulin as the sole carbon source and normalized by the relative luminescence from identical cultures supplied galactose. Values are the mean of 12 biological replicates and error is SEM in color-matched shading. FIGS. 4C-4F show the relative luminescence from wild-type Bo harboring P-BACOVA_04505 (black) or an isogenic strain lacking the Bo inulin sensor (ΔBACOVA_04496, red) were measured following the introduction of an equal mixture of galactose and (FIG. 4C) inulin or (FIG. 4D) fructose and normalized by the relative luminescence of identical cultures supplied galactose alone. For FIGS. 4A, 4C, and 4D, values are the mean of 8 biological replicates and error is SEM in color-matched shading. Source data are provided as a Source Data file.

FIGS. 6A, 6B, 6C, 6D, 6E, and 6F show the PUL-reporters reflect dose-dependent transcription. FIG. 6A shows the relative luminescence from a wild-type Bt strain harboring P-BT3332 following the introduction of 2-fold serial dilutions of 0.4% CS containing galactose to a total carbohydrate content of 0.5% and normalized to identical cultures supplied galactose alone. FIG. 6B shows the relative luminescence from a wild-type Bt strain harboring P-BT1763 following the introduction of 2-fold serial dilutions of 0.4% levan containing galactose to a total carbohydrate content of 0.5% and normalized to identical cultures supplied galactose alone. For FIGS. 6A-6B, values are the mean of 12 biological replicates and error is SEM in color-matched shading. FIG. 6C shows the AUC of response curves measured from wild-type Bt (black, see panel a) or a mutant lacking the glucuronyl hydrolase (Δgh, orange, see panel f) harboring P-BT3332 supplied 2-fold dilutions of CS to each strain and normalized by identical cultures supplied galactose alone. FIG. 6D shows the AUC of response curves measured from wild-type Bt (black, see panel b) or a mutant lacking a putative inner membrane transporter harboring P-BT1763 supplied 2-fold dilutions of levan to each strain and normalized by identical cultures supplied galactose alone. For FIGS. 6C-6D, values are the mean of 12 biological replicates, error bars are standard deviation, and P-values were computed by 2-way ANOVA with Dunnett correction and *** indicates values <0.001, **<0.01, *<0.05, and ns >0.05. FIG. 6E The AUC of response curves measured from wild-type Bt strains harboring either P-BT3332 (open blue squares) supplied mixtures containing 2-fold serial dilutions of levan or P-BT1763 (open red circles) supplied 2-fold serial dilutions of CS with galactose to a total carbohydrate content of 0.5% and normalized by identical cultures supplied galactose alone. Values are the mean of 6 biological replicates, error bars represent standard deviation, and P-values were computed using 2-way ANOVA with Tukey's honest significance test and ns indicates values >0.05.

FIG. 6F shows the relative luminescence from a gh-deficient Bt strain harboring P-BT3332 following the introduction of 2-fold serial dilutions of 0.4% CS containing galactose to a final carbohydrate content of 0.5% and normalized to identical cultures supplied galactose alone. Values are the mean of 12 biological replicates and error is SEM in color-matched shading. Source data are provided as a Source Data file.

FIGS. 7A, 7B, 7C, 7D, 7E, 7F, 7G, 7H, 7I, 7J, 7K, and 7L show the development of a glycan affinity isolation strategy using PUL-reporters. FIG. 7A shows a cartoon depicting an affinity isolation strategy whereby recombinant, PUL-encoded SGBP is immobilized using metal affinity chromatography and binds its target when incubated with heterogenous glycan mixtures, which subsequently co-elutes following the addition of excess histidine. FIGS. 7B-7D show the migration of purified recombinant his-tagged BT3330 and BT1761 proteins were examined by affinity-PAGE using acrylamide gels containing (FIG. 7B) CS, (FIG. 7C) no glycan, or (FIG. 7D) levan. FIGS. 7E-7F show the fold increases in reporter activity (AUC) from Bt strains harboring either a levan-specific reporter (P-BT1763, black bars) or a CS-specific reporter (P-BT3332, black bars) supplied 8 step-wise eluates from (FIG. 7E) BT3330 or (FIG. 7F) BT1761 pre-incubated with an equal mixture of CS and levan normalized by reporter strains supplied eluates from identical reactions containing E. coli lysates harboring an empty vector. Inset: SDS-PAGE analysis of E1-E8 demonstrating the amount of (FIG. 7E) BT3330 or (FIG. 7F) BT1761 present in each fraction. FIGS. 7G-7J show the pooled, concentrated eluates from (FIG. 7G & FIG. 7I) BT3330 or (FIG. 7H & FIG. 7J) BT1761 pre-incubated with a CS and levan mixture were supplied Bt strains harboring (FIG. 7G) P-BT3332 or (FIG. 7H) P-BT1763 and compared to standard curves of identical strains supplied 2-fold diluted CS or levan, respectively. Alternatively, the material was analyzed for total glycosaminoglycans (FIG. 7I) or fructans (FIG. 7J) using commercially available colorimetric assays according to the manufacturer's instructions. n=4, error bars are standard deviation; p-values were computed using a paired, two-tailed, Student's t-test; R2 values were computed using simple linear regression analysis in Prism and the 95% confidence interval is displayed as a dashed line. FIGS. 7K-7L show the pooled, concentrated BT3330 eluates pre-incubated with either (FIG. 7K) a CS and levan mixture or (FIG. 7L) a 0.1% PMOG solution were supplied to reporter strains differentially activated by CS or PMOGs (see FIGS. 8F&G, respectively). For all panels n=4, error bars are standard deviation, p-values were computed using a two-tailed student's t-test and ns indicates p-values >0.05, *<0.05, **, 0.01, ***<0.001.

FIGS. 8A, 8B, 8C, 8D, and 8E show the PULs are required for glycan-specific growth conditions and transcriptional responses. FIG. 8A shows growth of wild-type Bt (GT23; black) or strains lacking a CS-inducible susC (ΔBT3332; GT2926; blue), 3 CS-specific lyases (ΔBT3324 ΔBT3350 ΩBY4410; GT3086; purple), CS-sensor (ΔBT3334; GT150; red), or a glucuronyl hydrolyase (ΔBT3348; VR69; orange were measured during anaerobic culture in minimal media containing galactose as a sole carbon source. FIG. 8B shows the growth of wild-type Bt (GT231 black) or strains lacking a levan-inducible susC (ΔBT1763; GT3196; blue), 4 levanases (ΔBT1760-1759 ΔBT3082 ΩBT1765; GT3348; purple), fructan-sensor (ΔBT1754; GT16S; red), or a putative inner membrane fructose importer (ΩBT1758; GT3379; orange) were measured during anaerobic culture in minimal media containing galactose as a sole carbon source. For FIGS. 8A and 8B, values are the mean of 8 biological replicates and error bars are SEM in color-matched shading. FIGS. 8C-8E show the fold increase of (FIGS. 8C and 8D) BT3332 or (FIG. 8E) BT1763 mRNA levels in wildtype Bt following the introduction of mixtures containing either 0.2%, 0.02%, or 0.02% CS or levan supplemented with galactose to 0.5% total carbohydrate. The fold increase was calculated as the change in transcript levels between cultures before and after 2 hours or 1 hour following induction of glycan mixtures. Values are average of 6 independent measurements, error bars represent SEM, and P-values were calculated by 2-way ANOVA with Tukey's honest significance test and *** represents values <0.001, *<0.05, and ns indicates values >0.05.

FIGS. 9A, 9B, 9C, and 9D show the bioluminescence during anaerobic growth across Bacteroides species and growth conditions. FIG. 9A shows the relative luminescence (solid lines) or growth (dashed lines) from Bt strains harboring an empty vector (GT1866; black) or plasmids containing either the lux operon from P. luminescens (GT3137; blue) or the Bacteroides-optimized lux cassette (GT1541; red) expressed from the Bt rpoD promoter and rpiL* RBS was measured during growth in minimal media containing 0.5% glucose. FIG. 9B shows the relative luminescence of Bt strains harboring empty pBolux (GT1867; dashed lines) or a plasmid with the corresponding rpoD promoter cloned into the BamHI and SpeI sites (GT1868; solid lines) during growth in glucose (black), fructose (red), arabinose (purple), or xylose (green) as the sole carbon source. FIG. 9C shows the relative luminescence (solid lines) or growth (dashed lines) of Bo strains harboring empty pBolux (GT3489; black) or a plasmid with the Bo rpoD promoter cloned into the BamHI and SpeI sites (GT3490; red) during growth in galactose as the sole carbon source. FIG. 9D shows the relative luminescence of Bo strains harboring empty pBolux (GT3489; dashed lines) or a plasmid with the corresponding rpoD promoter cloned into BamHI and SpeI sites (GT3490; solid lines) during growth in glucose (black), fructose (red), arabinose (purple), or xylose (green) as the sole carbon source. For all figures, values are the mean of 8 biological replicates and error is SEM in color-matched shading.

FIGS. 10A, 10B, 10C, 10D, 10E, and 10F show that the BT3332 promoter confers CS and HA-inducible bioluminescence in pBolux. FIGS. 10A and 10B show the relative luminescence from wild-type Bt (GT1934, black lines) or strains lacking the CS-inducible susC (ΔBT3334; GT2939; blue), 3 CS-specific lyases (ΔBT3324 ΔBT3350 ΩBT4410; GT3117; purple), CS-sensor (ΔBT3334; BT2618; red), or a glucuronyl hydrolyase (ΔBT3348; GT3102; orange) harboring pBolux including the promoter region preceding BT3332 (P-BT3332) following the introduction of an equal mixture of CS and galactose or galactose alone. FIG. 10C shows the relative luminescence from wild-type Bt (GT1934) harboring P-BT3332 following the introduction of galactose alone (black lines) or an equal mixture of galactose and HA (green lines) or HS (blue lines). FIGS. 10D AND 10E show the growth of wild-type Bt (GT23; black) or a strain lacking the CS-sensor (ΔBT3334; GT150; red) were measured during anaerobic culture in minimal media containing HS or HA as a sole carbon source. FIG. 10F the relative luminescence from a CS-sensor deficient strain (ΔBT3334; GT2618) harboring P-BT3332 following the introduction of galactose alone (black lines) or an equal mixture of galactose and HA (green lines) or HS (blue lines). For all figures, values are the mean of 8 biological replicates, error bars are SEM in color-matched shading.

FIGS. 11A, 11B, 11C, 11D, 11E, 11F, 11G, and 11H show a fructan-responsive reporter reveal new insights into Bt levan utilization. FIG. 11A shows the relative luminescence from wild-type Bt harboring empty pBolux (black) or a plasmid including the region upstream of the levan-inducible susC (P-BT1763, pink) following the introduction of 0.5% levan (solid lines) or 0.5% galactose (dashed lines). Values are the mean of 12 biological replicates, error is SEM in color-matched shading. FIGS. 11B and 11C show the growth of wild-type Bt harboring empty pBolux (black) or P-BT1763 (pink) following the introduction of 0.5% galactose or 0.5% levan. For FIGS. 11A, 11B, and 11C, values are the mean of 12 biological replicates, error is SEM in color-matched shading. FIGS. 11D and 11F show the growth of wild-type Bt or strains lacking all other levanases except BT1760 (ΔBT1759 ΔBT3082 ΩBT1765; GT3347; pink), BT1759 (ΔBT1760 ΔBT3082 Ω1765; GT3346; teal), BT3082 (ΔBT1760-59 ΩBT1765; gt3401; lavender), or BT1765 (ΔBT1760-59 Δ3082; GT3308; purple) in 0.1% fructose or galactose as a sole carbon source. FIGS. 11E and 11G show the growth wild-type Bt (GT23, black) or strains lacking either BT1760 (GT3181; pink), BT1759 (GT3226; teal), BT3082 (GT3303; lavender), or BT1765 (GT; purple) 0.1% fructose or galactose as a sole carbon source. FIG. 11H shows the growth of wild-type Bt (GT2111. Black) or BT1760-deficient strains (GT3215; pink) harboring empty pNBU2 or a plasmid encoding BT1760 (GT3216, green) in 0.1% levan as the sole carbon source.

FIGS. 12A, 12B, 12C, and 12D show a fructan-responsive PUL in Bo exhibits inulin-inducible activity. FIGS. 12A, 12B, and 12C show the growth of wild-type Bo (ATCC 8483; black) or a strain lacking the Bo inulin sensor (ΔBACOVA_04496; GT3183; red) were measured during anerobic culture in minimal media containing levan, fructose, or galactose as the sole carbon source. Values are the mean of 8 biological replicates and errors bars are SEM in color-matched shading.

FIG. 12D shows the relative luminescence from wild-type Bo harboring empty pBolux (black) or a plasmid that includes the region preceding an inulin-inducible susC (P-BACOVA_04505; pink) were measured following the introduction of 0.5% inulin (solid lines) or 0.5% galactose (dashed lines). Values are the mean of 12 biological replicates and error is SEM in color-matched shading.

FIGS. 13A, 13B, 13C, 13D, 13E, and 13F show that PUL reporters display concentration dependent responses to target glycans. FIG. 13A shows the relative luminescence from a wild-type Bt strain harboring a P-BT1763 (GT1893) following the introduction of mixtures containing 2-fold serial dilutions of 0.4% levan balanced with galactose to a final carbohydrate content of 0.5% and normalized to identical cultures supplied galactose alone. Values are the mean of 12 biological replicates and error bars are SEM in color-matched shading. FIGS. 13B and 13C show the log 10 AUC responses within the linear range from wild-type Bt (black; FIG. 13B: GT1934; FIG. 13C: GT1893) or mutants defective for PUL-sensor deactivation (orange; FIG. 13B: GT3102; FIG. 13C: GT3393) harboring P-BT3332 or P-BT1763 and supplied 2× serial dilutions of 0.4% CS or levan balanced with galactose to 0.5% total carbohydrate content and normalized to response from identical cultures supplied galactose alone. Values are the mean of 12 biological replicates and error is standard deviation. Solid lines represent the simple linear regression models corresponding to responses from each strain and color-matched dashed lines represents the 95% confidence intervals computed in Prism. FIGS. 13D and 13E show the fold difference between the AUC of response from wild-type Bt strains harboring either P-BT3332 (blue squares) or P-BT1763 (red circles) supplied glycan mixtures containing 2-fold serial dilutions of 0.2% CS or levan, respectively, in the presence or absence of constant 0.2% levan or CS, respectively, and balanced with galactose to 0.5% total carbohydrate normalized by the AUC of responses from identical cultures supplied alone. FIG. 13E shows the AUC of responses from wild-type Bt strains harboring either P-BT3332 (GT1934; blue squares) or P-BT1763 (GT1893; red circles) supplied glycan mixtures containing 2-fold serial dilutions of 0.2% CS or levan, respectively, in the presence of constant 0.2% levan or CS, respectively, and balanced with galactose to 0.5% total carbohydrate normalized by the AUC of responses from identical cultures supplied galactose alone. Values represent the average of 6 biological replicates and error bars are standard deviation. P-values were calculated with 2-way ANOVA with Tukey's honest significance test and *** represents values <0.001, **<0.01, *<0.05, and ns >0.05. FIG. 13F shows the relative luminescence from a BT1758-deficient Bt strain harboring P-BT1763 (GT3393) following the introduction of 2-fold serial dilutions of 0.4% levan containing galactose to a final carbohydrate content of 0.5% and normalized to identical cultures supplied galactose alone. Values are the mean of 12 biological replicates and error bars are SEM in color-matched shading.

FIGS. 14A, 14B, 14C, 14D, 14E, 14F, 14G, 14H, and 14I show the glycan-responsive reporter strains can indicate target glycan abundance. FIGS. 14A and 14B show the AUC of responses from wild-type Bt strains harboring either a P-BT3332 (GT1934; open bars) or P-BT1763 (GT1893, filled bars) supplied elution fractions from nickel-NTA agarose incubated with E. Coli whole cell lysates from strains containing empty pT7-7 vector, or plasmids engineered to overexpress BT1761 or BT3330 and pre0incubated with a mixture of 0.1% of both levan and CS. All elution fractions were supplemented with 0.4% galactose. Values represent an average of 4 total replicates from two independent experiments and error bars are SEM. FIG. 14C shows the Coomassie stained SDS-PAGE gels showing the corresponding protein levels for BT3330 (top 2 gels) or BT1761 (bottom 2 gels) in each elution fraction. FIGS. 14D and 14E show the AUC responses from wild-type Bt strains harboring either P-BT3332 (GT1934; open bars) or P-BT1763 (GT1893, filled bars) supplied galactose alone or concentrated material co-purifying with BT3330 or BT1761 supplemented with 0.4% galactose. Values are the average of 4 measurements from 2 independent experiments, error is standard deviation and P-values were computed using 1-way ANOVA with Tukey's honest significance test and *** represents values <0.001 and ns indicates values >0.05. FIG. 14F shows the AUC of responses from a BT3348-deficient Bt strain harboring P-BT3332 supplied galactose alone (none) or concentrated material co-purifying BT1761 supplemented with 0.4% galactose. FIG. 14G shows identical cultures to those described in FIG. 14F were supplied 2-fold serial dilutions of CS containing galactose to 0.5% total carbohydrate content and all measurements were normalized to responses from cultures supplied galactose alone. FIG. 14H shows the AUC of responses from a BT1758-deficient Bt strain harboring P-BT1763 supplied galactose alone or concentrated material co-purifying with BT3330 and supplemented with 0.4% galactose. FIG. 14I shows identical cultures to those described in FIG. 14H were supplied 2-fold dilutions of levan containing galactose to 0.5% total carbohydrate content and all measurements were normalized to responses from cultures supplied galactose alone. For FIGS. 14F-14I, values represent the average of 2 measurements from a single experiment and error bars are standard deviation. For FIGS. 14G-14H, solid lines represent simple linear regression models and color-matched dashed lines are the 95% confidence intervals computed in Prism.

FIGS. 15A, 15B, 15C, and 15D show a Bacteroides transcriptional reporter functions across many species. Relative luminescence (left axis) and growth (right axis) detected from 4 different wild-type Bacteroides species, (FIG. 15A) Bt, (FIG. 15B) Bo, (FIG. 15C) Bf, or (FIG. 15D) Bc harboring a promoter-less Bacteroides-optimized lux cassette control plasmid (pBolux, black lines) or one encoding the constitutively active rpoD promoter (red lines) cultured in minimal media containing 0.5% galactose as the sole carbon source. n=4, error bars are SEM.

FIGS. 16A, 16B, 16C, and 16D show how PUL-reporters respond to target glycans. FIG. 16A shows the reporter activity over 18 hours in wild-type Bt harboring a promoter-less reporter plasmid (pBolux, gray) or one with the promoter preceding the CS-specific susC gene (P-BT3332, black) in 0.5% CS normalized to identical strains supplied 0.5% galactose. n=6. FIG. 16B shows the normalized reporter activity over time harboring the CS-responsive PUL reporter in wild-type Bt (wt, black) or mutants lacking either the CS-sensor (Δsen, red), CS-specific susC gene (ΔsusC, blue), 3 CS-specific lyases (Δlyase, purple), or a CS-disaccharide specific glucuronyl hydrolase (Δgh, orange) supplied a mixture of 0.2% CS and 0.2% galactose and normalized to identical strains supplied 0.4% galactose. FIG. 16C shows the AUC for all strains in (B) were computed for 18 hours following the introduction of CS. n=8. FIG. 16D shows the AUC computed from normalized reporter activity from wild-type Bt (wt, black) or strains lacking 3 CS-specific lyases (Δlyase, purple) or the CS-specific sensor (Δsen, red) harboring the P-BT3332 reporter plasmid and supplied CS-disaccharide. n=6. For all panels, error bars are SEM. For (C-D) p-values were calculated using paired, two-tailed student's t-tests. ns indicates values >0.05, *<0.05, **<0.01, and ***<0.001.

FIGS. 17A, 17B, 17C, 17D, 17E, and 17F show the species-specific PUL-reporters differentially detect compositionally identical but structurally distinct glycans. FIGS. 17A-17C show the growth of wild-type Bt (black) or Bo (gray) strains or mutants lacking the corresponding fructan PUL-sensor genes, BT1754 (BtΔsen, red) or BACOVA_04496 (BoΔsen, yellow), respectively, cultured in (FIG. 17A) levan, (FIG. 17B) inulin, or (FIG. 17C) fructose as the sole carbon source. FIGS. 17D-17F show the fructan-responsive reporter activity of the strains described for (FIGS. 17A-17C) following the introduction of galactose supplemented with equal amounts and (FIG. 17D) fructose, (FIG. 17E) levan, or (FIG. 17F) inulin and normalized to the non-inducing condition (galactose alone). The area under each curve was computed for 18 hours following the introduction of each glycan mixture. For all panels: n=8, error bars are SEM, P-values were calculated by paired, two-tailed Student's t-test. ns indicates values <0.05, *<0.05, **<0.01, and ***<0.001.

FIGS. 18A, 18B, 18C, 18D, and 18E show that PUL-reporters elicit specific dose-dependent responses to target glycans. FIG. 18A shows the reporter activity from wild-type Bt harboring the levan-reporters supplied 2× dilutions of the corresponding glycan in galactose to a final carbohydrate content of 0.5% and normalized to identical strains supplied galactose alone. FIG. 18B shows the quantified CS-reporter activity normalized to a non-inducing condition (0.5% galactose) following the introduction of 2× serial dilutions of CS (filled circles) or levan (open circles) in galactose to a final glycan concentration of 0.5%. FIG. 18C shows the quantified levan-dependent reporter activity normalized to a non-inducing condition (0.5% galactose) following the introduction of 2× serial dilutions of levan (open circles) or CS (filled circles) in galactose to a final glycan concentration of 0.5%. FIG. 18D shows the quantified CS-reporter (P-BT3332; filled circles) or levan-reporter (P-BT1763; open circles) activity from strains supplied mixtures containing 2× serial dilutions of CS in constant 0.2% levan and galactose to final carbohydrate content of 0.5%. FIG. 18E shows the quantified CS-reporter (P-BT3332; filled circles) or levan-reporter (P-BT1763; open circles) activity from strains supplied mixtures containing 2× serial dilutions of CS in constant 0.2% levan and galactose to final carbohydrate content of 0.5%. For all panels: n=4, error bars are SEM, p-values were calculated using a paired, two-tailed Student's t-test. * indicates p-values below 0.05 compared to strains supplied galactose. R2 values were computed by MS Excel power trendline function.

FIGS. 19A, 19B, 19C, 19D, and 19E show the mutations disrupting PUL-encoding activities can narrow target specificity or increase reporter sensitivity. FIG. 19A shows the growth of wild-type Bt (black) or a strain lacking 3 CS-specific lyases (purple) in 0.1% HA as a sole carbon source. n=4, error is standard deviation. FIGS. 19B-19C shows the AUC from wild-type Bt (black) or a lyase-deficient mutant (purple) harboring the CS/HA-reporter (P-BT3332) following the introduction of 2-fold titrations of (FIG. 19B) CS or (FIG. 19C) HA and normalized to a non-inducing condition (0.5% galactose). n=4, error bars are standard deviation. FIG. 19D shows the reporter activity (AUC) was measured from wild-type Bt (black line) or the gh-deficient strain (orange) harboring the CS-reporter (P-BT3332) following the introduction of 2× serial dilutions of CS in galactose to a final glycan concentration of 0.5% and normalized to an identical strain supplied galactose alone. FIG. 19E shows the reporter activity (AUC) was measured from wild-type Bt (black line) or a fructose importer-deficient strain (orange) harboring the levan-reporter (P-BT1763) following the introduction of 2× serial dilutions of levan in galactose to a final glycan concentration of 0.5% and normalized to an identical strain supplied galactose alone. n=6 and error bars are standard deviation.

FIGS. 20A, 20B, 20C, 20D, 20E, 20F, 20G, 20H, and 20I show the glycan-independent validation of reporter plasmids. FIG. 20A shows a schematic depicting our approach to validate PUL reporters in the absence of known glycan ligands and identify optimal promoters for sensor/regulator-dependent glycan-induced transcription activation. FIGS. 20B-20I show the fold increase (AUC) in reporter activity in strains over-expressing a constitutively activated form of the PUL regulators: (FIG. 20B) BT2391, (FIG. 20C) BT3269, (FIG. 20D) BT3097, (FIG. 20E) BT3172, (FIG. 20F) BT1278, (FIG. 20G) BT1617, (FIG. 20H) BT3517, or (FIG. 20I) BT4137 and harboring promoter-less pBolux or a plasmid containing putative promoter regions from the corresponding PUL. The fold increase in reporter activity was calculated as the AUC from strains harboring each reporter plasmids and over-expressing the indicated regulator normalized by a strain harboring identical reporter plasmids and an empty over-expression vector. n=4, error bars are standard deviation, p-values were calculated using paired, two-tailed student's t-tests, ns indicates p-values >0.05, *, <0.05, and **<0.01.

FIGS. 21A, 21B, 21C, 21D, 21E, 21F, and 21G show a high-throughput Bt PUL-reporter array specifically detects target glycans individually and in biologically derived mixtures. FIG. 21A-21G show the fold increase in reporter activity (AUC) of strains harboring 95-distinct PUL reporter plasmids (labeled on the x-axis by the corresponding PUL-encoded susC gene) or the promoter-less pBolux plasmid (1st bar) following the introduction of a mixture of 0.1% galactose and 0.4% (FIG. 21A) arabinogalactan, (FIG. 21B) levan, (FIG. 21C) dextran, (FIG. 21D) hyaluronan, (FIG. 21E) heparan sulfate, (FIG. 21F) chondroitin sulfate, or (FIG. 21G) PMOGs. FIGS. 21A-21E are from a single experiment, FIG. 21F and FIG. 21G are the average of 3 or 5 independent replicates, respectively. Error bars are standard deviation, p-values were calculated using a paired, two-tailed student's t-test and * indicates p-values <0.05, **<0.01, and ***<0.001.

FIGS. 22A, 22B, and 22C show that PUL-sensors specifically activate target PULs. FIGS. 22A-22B show the bioluminescence from wild-type or BT3334-deficient Bt strains harboring P-BT1632, P-BT2818, P-BT3332, or P-BT4662 were measured in response to a mixture of galactose and (FIG. 22A) CS or (FIG. 22B) CS-disaccharide (di0S) and normalized by identical strains supplied galactose. FIG. 22C shows the bioluminescence from Bt strains harboring the reporters described in panels A&B and over-expressing a constitutively active BT3334 protein (BT3334*). Measurements were normalized by values from a strain harboring identical reporters and an empty over-expression vector. n=4, error bars are standard deviation, and p-values were calculated by two-tailed student's t-test. ns indicates p-values <0.05, *<0.05, **<0.01, and ***<0.001.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “control” is an alternative subject or sample used in an experiment for comparison purposes. A control can be “positive” or “negative.”

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A “promoter,” as used herein, refers to a sequence in DNA that mediates the initiation of transcription by an RNA polymerase. Transcriptional promoters may comprise one or more of a number of different sequence elements as follows: 1) sequence elements present at the site of transcription initiation; 2) sequence elements present upstream of the transcription initiation site and; 3) sequence elements down-stream of the transcription initiation site. The individual sequence elements function as sites on the DNA, where RNA polymerases and transcription factors facilitate positioning of RNA polymerases on the DNA bind.

As used herein, the term “buffer” refers to a solution consisting of a mixture of acid and its conjugate base, or vice versa. The solution is used as a means of keeping the pH at a nearly constant range to be used in a wide variety of chemical and biological applications.

“Culture” or “cell culture” is the process by which cells, including bacterial cells, are grown under controlled conditions, generally outside their natural environment. These conditions vary for each cell type, but generally consist of a suitable vessel with a substrate or medium that supplies the essential nutrients (amino acids, carbohydrates, vitamins, minerals), growth factors, hormones, and gases (CO₂, O₂), and regulates the physio-chemical environment (pH buffer, osmotic pressure, temperature). Most cells require a surface or an artificial substrate to form an adherent culture as a monolayer (one single-cell thick), whereas others can be grown free floating in a medium as a suspension culture.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “detect” or “detecting” refers to an output signal released for the purpose of sensing of physical phenomenon. An event or change in environment is sensed and signal output released in the form of light.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

“Transformation” of a cellular organism with DNA means introducing DNA into an organism so that at least a portion of the DNA is replicable, either as an extrachromosomal element or by chromosomal integration. The cell is termed “host cell” and it may be either prokaryotic or eukaryotic. Typical prokaryotic host cells include various strains of E. coli or Bacteroides. The introduced DNA sequence may be from the same species as the host cell of a different species from the host cell, or it may be a hybrid DNA sequence, containing some foreign and some homologous DNA.

The terms “treat,” “treating,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset (e.g., before obvious signs of disease]), during early onset (e.g., upon initial signs and symptoms of disease), or after an established development of disease.

The term “antibiotics” refers to a type of antimicrobial substance active against bacteria. These are the most important type of antimicrobial agent for fighting bacterial infections, and antibiotics medications are widely used in the treatment and prevention of such infections. They may either kill or inhibit the growth of bacteria.

Used herein, the term “probiotics” refers to live microorganisms promoted with claims that they provide health benefits when consumed, generally by improving or restoring the gut flora.

As used herein, “normal” refers to a standard of health where no disease, disorder, abnormal condition, mutation, or dysfunction exists within a particular subject or patient. “Normal” can also refer to an average or typical state or condition.

As used herein, “wild-type” refers to the genetic and physical characteristics of the typical form of a species as it occurs in nature. A wild-type or wild type characteristic is conceptualized as a product of the standard “normal” allele at a gene locus, in contrast to that produced by a non-standard “mutant” allele.

As used herein, “preserve,” “preserved,” “preservation,” “preserving” and any grammatical variations thereof as used herein, refers to the act of keeping any object, composition, or compound intact, alive, or free from decomposition/decay.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

As used herein, “harbor”, “harboring”, and any grammatical variations thereof refers to a subject, including, but not limited to a cell, vector, bacteria, virus, fungi, tissue, or any other organism containing or being the host to another organism, molecule, compound, nucleic acid, protein, or any other composition.

A “cassette” or “gene cassette” refers to a mobile genetic element that generally contains a gene and a recombination site. Gene cassettes can move around within an organism's genome or can be transferred to another organism in the environment.

“Quantify”, “quantifying”, “quantification”, and any other grammatical variations thereof refer to the process of acquiring numerical values to determine, express, or measure an amount of a substance or signal.

“Anaerobic” refers to living, active, occurring, or existing in the absence of oxygen.

As used herein, the term “apparatus” refers to a technical equipment or machinery needed for a particular activity or purpose. Herein, the apparatus is used to separate biomolecules from one another for further purification and collection. The apparatus can comprise one or multiple components to form a complex structure needed to perform a particular function.

As used herein, a “container” refers to a receptacle or enclosure for holding a product, substance, or composition used in storage, packaging, and transportation, including shipping. The product, substance, or composition are kept inside of the container to protect and prevent contamination from the outside environment. A container is also meant to partially or completely enclose the product, substance, or composition.

### Reporter Plasmids

As used herein, a “reporter plasmid” refers to a type of expression vector containing a reporter gene, also called “reporter vector”, that can be used to identify and characterize promoter and/or enhancer element functions relative to gene expression. Expression of the reporter gene corresponds to the transcriptional activity of the reporter gene. In general, reporter genes are nucleic acid sequences whose products, or proteins, can be readily assayed, measured, or quantified following transformation or transfection into a host cell. Reporter genes include, but are not limited to luciferase reporter genes (lux or luc), beta-galactosidase reporter gene (lacZ), chloramphenicol acetyltransferase (cat), green fluorescent protein reporter gene (gfp), red fluorescent protein reporter gene (rfp), and derivatives thereof. It should be noted that luciferase reporter gene are the preferred reporter gene because luciferase reporter genes are readily expressed in an anaerobic environment.

The reporter plasmid can comprise any combination of gene elements to allow for transcription including, but not limited to promoter sequences, activator and/or enhanced elements (including, but not limited to transcription factor binding sites and polymerase binding sites), and operator and/or silenced elements (including, but not limited to repressor protein binding sites).

Thus in one aspect, disclosed herein are reporter plasmids comprising a luciferase reporter cassette, a first polysaccharide utilization locus (PUL), and a first glycan-specific promoter.

Also disclosed herein are reporter plasmids, wherein the first PUL encodes any combination of proteins comprising a surface glycan binding protein (SGBP), an outer membrane channel, a digestive enzyme, a glycan importer, or a glycan sensor.

In one aspect disclosed herein are reporter plasmids, wherein the first PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof.

Also disclosed herein are reporter plasmids, wherein the first glycan specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof including, but not limited to glycan specific promoters comprising the nucleic acids sequence as set forth in SEQ ID NO: 74, SEQ ID NO: 75, SEQ ID NO: 76, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, or SEQ ID NO: 354.

In one aspect disclosed herein are reporter plasmids, wherein the reporter plasmid further comprises a second PUL. In some embodiments, the second PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan (OG)-specific PUL, or variants thereof. In some embodiments, the first PUL is interchangeable with the second PUL.

Also disclosed herein are reporter plasmids, wherein the reporter plasmid further comprises a second glycan-specific promoter. In some embodiments, the first glycan-specific promoter is interchangeable with the second glycan-specific promoter. In some embodiments, the second glycan-specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof.

In one aspect, disclosed herein are reporter plasmids, wherein the second glycan-specific promoter comprises the nucleic acids sequence as set forth in SEQ ID NOs 74-76, or SEQ ID NOS: 262-354.

Also disclosed herein are reporter plasmids, wherein the reporter plasmid comprises a Bacteroides-optimized lux (Bolux) plasmid.

### Engineered Bacteria

An engineered bacteria, or a genetically engineered bacteria, are bacteria harboring additional genetic material, usually in the form of a plasmid vector, that allows said bacteria the ability to efficiently express native or foreign proteins for a specific purpose. It should be noted that more than one gene and other genetic material can be inserted into a plasmid, which can then be further integrated into bacteria.

Generating engineered bacteria in the gastrointestinal (GI) microbiome serves as an option for treating multiple diseases by genetically altering the bacteria residing in the GI tract. Genetically altering the GI microbiome allows for molecular profiling of the GI tract, altering gut bacteria to destroy pathogenic bacteria, or using genetically engineered bacteria to modify deficient enzymes or proteins. Specifically, molecular profiling of the GI tract includes detecting and quantifying the presence of glycans, oligosaccharides, monosaccharides, and other biomolecules in the GI tract. Such an approach also indicates the presence of pathogenic microorganisms or deficient enzymes and/or proteins.

Also disclosed herein are engineered bacteria comprising any of the reporter plasmid disclosed herein. For example, disclosed herein are engineered bacteria harboring a reporter plasmid wherein the reporter plasmid comprises a luciferase reporter cassette, a first polysaccharide utilization locus (PUL), and a first glycan-specific promoter. In some aspects, the first PUL encodes any combination of proteins comprising a surface glycan binding protein (SGBP), an outer membrane channel, a digestive enzyme, a glycan importer, or a glycan sensor. In some embodiments, the first PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof. In some aspects, the first glycan specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof.

In one aspect, disclosed herein are engineered bacteria, wherein the first glycan-specific promoter comprises the nucleic acids sequence as set forth in SEQ ID NO: 74, SEQ ID NO: 75, SEQ ID NO: 76, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, or SEQ ID NO: 354.

Also disclosed herein are engineered bacteria, further comprising a second PUL. In some aspects, the second PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof. In some aspects, the first PUL is interchangeable with the second PUL.

In one aspect, disclosed herein are engineered bacteria, further comprising a second glycan-specific promoter. In some embodiments, the first glycan-specific promoter is interchangeable with the second glycan-specific promoter. In some aspects, the second glycan-specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof.

Also disclosed herein are engineered bacteria, wherein the second glycan-specific promoter comprises the nucleic acids sequence as set forth in SEQ ID NOs 74-76, or SEQ ID NOs: 262-354.

In some aspects, disclosed herein are engineered bacteria, wherein the engineered bacteria originates from a gastrointestinal bacterium. In some aspects, the engineered bacteria originates from a Bacteroides bacterium including, but not limited to Bacteroides thetaiotaomicron (Bt), Bacteroides ovatus (Bo), Bacteroides fragilis (Bf), Bacteroides caccae (Bc) or a variant strain thereof.

Also disclosed herein are engineered bacteria, wherein the engineered bacteria overexpresses the chondroitin sulfate (CS)-specific PUL when exposed to a CS glycan, or derivatives thereof.

In some aspects, disclosed herein are engineered bacteria, wherein the engineered bacteria overexpresses the levan-specific PUL when exposed to a levan glycan, or derivatives thereof.

Also disclosed herein are engineered bacteria, wherein the engineered bacteria overexpresses the inulin-specific PUL when exposed to an inulin glycan, or derivatives thereof.

In some aspects, disclosed herein are engineered bacteria, wherein the engineered bacteria overexpresses the arabinogalactan-specific PUL when exposed to an arabinogalactan, or derivatives thereof.

Also disclosed herein are engineered bacteria, wherein the engineered bacteria overexpresses the dextran-specific PUL when exposed to a dextran glycan, or derivatives thereof.

In some aspects, disclosed herein are engineered bacteria, wherein the engineered bacteria overexpresses the hyaluronan (HA)-specific PUL when the engineered bacteria is exposed to a HA glycan, or derivatives thereof.

Also disclosed herein are engineered bacteria, wherein the engineered bacteria overexpresses the heparan sulfate (HS)-specific PUL when exposed to a HS glycan, or derivatives thereof.

In some aspects, disclosed herein are engineered bacteria, wherein the engineered bacteria overexpresses the O-glycan(OG)-specific PUL when exposed to an OG glycan mixture, or derivatives thereof.

Also disclosed herein are engineered bacteria, wherein the engineered bacteria emits a light signal when overexpressing any PUL. In some aspects, the light signal is decreased when the glycan molecule is broken down into a monosaccharide molecule.

### Glycan-Sensing Systems and Compositions

Glycans are polymer compounds comprising several smaller monosaccharide molecules usually linked together by glycosidic bonds. Glycan can also exist as homopolymers or heteropolymer, wherein the monosaccharide units can also be linked in a linear or branched formation. Common types of glycans include, but are not limited to O-linked glycans, N-linked glycans, and glycosaminoglycans. Glycans are substantive components of the gastrointestinal (GI) tract system of eukaryotic organisms. Microorganisms that reside in the GI tract are adept at consuming glycans and other polysaccharide molecules establishing a symbiotic relationship between gut microorganisms and the host. Fluctuations in the abundance of glycans create a diverse and heterogeneous environment in which the gut microorganisms can strive and proliferate. Thus, glycans shape and influence the composition and abundance of gut microbiome. Further, abnormalities in the gut microbiome glycan composition can also implicate presence of diseases and/or disorder. Given the influence of glycans in the GI tract, there is a need to develop compositions, systems, and methods of detecting homogenous or heterogenous mixtures of glycans for profiling, treating, preventing, reducing, and/or decreasing GI diseases and/or disorders in a subject.

In one aspect, disclosed herein is a glycan-sensing system comprising any of the reporter plasmids disclosed herein and/or any of the engineered bacteria disclosed herein.

In one aspect, disclosed herein is a glycan-sensing system comprising an engineered bacteria harboring a reporter plasmid, wherein the reporter plasmid comprises a luciferase reporter cassette, a first polysaccharide utilization locus (PUL), and a first glycan-specific promoter, wherein the glycan-sensing system detects and quantifies a glycan molecule.

As used herein, a “PUL” refers to a co-regulated group of genes that sense the presence of surrounding nutrients and enable digestion of glycans and other polysaccharides molecules. The GI microbiome comprises numerous PULs that enable bacterial species to recognize and consume glycan molecules. It should be noted that a PUL can be glycan specific.

In some embodiments, the glycan molecule comprises chondroitin sulfate, arabinan, heparan sulfate, hyaluronan, fructan, levan, mannan, or derivatives thereof. In some embodiments, the glycan is an N-linked glycan. In some embodiments, the glycan is an O-linked glycan. In some embodiments, the glycan is a branched glycan. In some embodiments, the glycan is a linear glycan. In some embodiments, the glycan molecule comprises pectic galactan, starch, glucan, galactomannan, glucomannan, homogalacturonan, xyloglucan, dermatan, xylan, rhamnogalacturonan, or derivative thereof. In some embodiments, the glycan molecule is located in the gastrointestinal tract of a mammal.

The genes within a PUL can encode a glycan utilization system, including, but not limited to a starch utilization system (Sus system). The gene products of the glycan utilization system generally localize to the outer membrane or periplasm of a bacterium, and function to sequentially bind to the glycan on the bacterial surface, degrade it into oligosaccharides, and transport the oligosaccharides into the periplasmic space for further degradation into monosaccharides. In some embodiments, the first PUL encodes any combination of proteins comprising a surface glycan binding protein (SGBP), an outer membrane channel, a digestive enzyme, a glycan importer, or a glycan sensor. In some embodiments, the SGBP comprises a SusD, SusG, SusE, or Sus F protein. In some embodiments, the outer membrane channel comprises a Sus C channel protein. In some embodiments, the digestive enzyme comprises a lyase, an amylase, or an enzyme capable or degrade glycans, carbohydrates, or polysaccharides. In some embodiments, the digestive enzyme comprises a Sus A or SusB enzyme. In some embodiments, glycan importer is located on the inner membrane of a bacterial cell. In some embodiments, glycan sensor comprises a SusR protein.

In some embodiments, the first PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof.

In some embodiments, the first glycan specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof.

In some embodiments, the system further comprises a second PUL. In some embodiments, the second PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof. In some embodiments, the first PUL is interchangeable with the second PUL.

In some embodiments, the system further comprises a second glycan-specific promoter. In some embodiments, the first glycan-specific promoter is interchangeable with the second glycan-specific promoter. In some embodiments, the second glycan-specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan (OG)-specific promoter, or variants thereof.

In some embodiments, the first or second glycan-specific promoter comprises SEQ ID NO: 74, SEQ ID NO: 75, SEQ ID NO: 76, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, or SEQ ID NO: 354.

In some embodiments, the luciferase reporter cassette comprises one or more genes selected from lux C, lux D, lux E, lux A, and lux B. In some embodiments, the luciferase reporter cassette originates from a Photorhabdus bacteria. In some embodiments, the luciferase reporter cassette originates from a Photorhabdus luminescens (PI) bacteria. In some embodiments, the gene products of the luciferase reporter cassette emit a light signal.

In some embodiments, the reporter plasmid comprises a Bacteroides-optimized lux (Bolux) plasmid.

In some embodiments, the engineered bacteria originates from a gastrointestinal bacterium. In some embodiments, the engineered bacteria originates from a Bacteroides bacterium. In some embodiments, the engineered bacteria originates from a Bacteroides thetaiotaomicron (Bt), Bacteroides ovatus (Bo), Bacteroides fragilis (Bf), Bacteroides caccae (Bc) or a variant strain thereof. In some embodiments, the engineered bacteria originates from a Bacteroides acidifaciens (Ba), Bacteroides barnesiaes (Bb), Bacteroides caecicola, Bacteroides caecigallinarum, Bacteroides cellulosilyticus, Bacteroides cellulosolvens, Bacteroides ciarus, Bacteroides coagulans, Bacteroides coprocola, Bacteroides coprophilus, Bacteroides coprosuis, Bacteroides dorei, Bacteroides eggerthii, Bacteroidesgracilis, Bacteroides faecichinchillae, Bacteroides faecis, Bacteroides finegoldii, Bacteroides fluxus, Bacteroides galacturonicus, Bacteroides gallinaceum, Bacteroides gallinarum, Bacteroides goldsteinii, Bacteroides graminisolvens, Bacteroides helcogene, Bacteroides intestinalis, Bacteroides luti, Bacteroides massiliensis, Bacteroides nordii, Bacteroides oris, Bacteroides paurosaccharolyticus, Bacteroides plebeius, Bacteroides polypragmatus, Bacteroides proprionicifaciens, Bacteroides putredinis, Bacteroides pyogenes, Bacteroides reticulotermitis, Bacteroides rodentium, Bacteroides salanitronis, Bacteroides salyersiae, Bacteroides sartorii, Bacteroides sedmenti, Bacteroides stercoris, Bacteroides suis, Bacteroides tectus, Bacteroides uniformis, Bacteroides vulgatus, or Bacteroides xylanisolvens.

In some embodiments, the engineered bacteria are grown in an anaerobic environment. In some embodiments, the engineered bacteria are grown in a 96-well, a 384-well, or a 1536-well microplate.

In some embodiments, the engineered bacteria overexpresses the chondroitin sulfate (CS)-specific PUL when exposed to a CS glycan, or derivatives thereof.

In some embodiments, the engineered bacteria overexpresses the levan-specific PUL when exposed to a levan glycan, or derivatives thereof.

In some embodiments, the engineered bacteria overexpresses the inulin-specific PUL when exposed to an inulin glycan, or derivatives thereof.

In some embodiments, the engineered bacteria overexpresses the arabinogalactan-specific PUL when exposed to an arabinogalactan, or derivatives thereof.

In some embodiments, the engineered bacteria overexpresses the dextran-specific PUL when exposed to a dextran glycan, or derivatives thereof.

In some embodiments, the engineered bacteria overexpresses the hyaluronan (HA)-specific PUL when the engineered bacteria is exposed to a HA glycan, or derivatives thereof.

In some embodiments, the engineered bacteria overexpresses the heparan sulfate (HS)-specific PUL when exposed to a HS glycan, or derivatives thereof.

In some embodiments, the engineered bacteria overexpresses the O-glycan(OG)-specific PUL when exposed to an OG glycan mixture, or derivatives thereof.

In some embodiments, the engineered bacteria emits a light signal when overexpressing any PUL disclosed herein.

In some embodiments, the light signal is decreased when the glycan molecule is broken down into a monosaccharide molecule.

### Methods of Detecting Glycan Molecules

In one aspect, disclosed herein is a method of detecting a glycan, the method comprising isolating and preserving a tissue sample from a subject, preparing, and culturing the glycan-sensing system disclosed herein in a microplate in an anaerobic environment, adding the tissue sample into the microplate, and detecting and quantifying a light signal emitted from the system.

In one aspect, disclosed herein is a method of detecting a glycan, the method comprising isolating and preserving a tissue sample from a subject, preparing, and culturing any of the engineered bacteria disclosed herein in a microplate in an anaerobic environment, adding the tissue sample into the microplate, and detecting and quantifying a light signal emitted from the engineered bacteria.

In one aspect, disclosed herein is a method of detecting a glycan, the method comprising isolating and preserving a tissue sample from a subject, preparing any of the reporter plasmids disclosed herein in a microplate in an anaerobic environment, adding the tissue sample into the microplate, and detecting and quantifying a light signal emitted from the reporter plasmid.

In some embodiments, the tissue sample comprises at least one glycan. In some embodiments, the light signal is detected and quantified using a microplate reader, a spectrophotometer, or an instrument capable of measuring the light signal. In some embodiments, the tissue sample comprises a tissue biopsy, a blood sample, or a saliva sample. In some embodiments, the tissue sample comprises a tissue biopsy from the GI tract, including, but not limited to a tongue scraping, buccal swab, an esophageal tissue, a stomach tissue, an intestinal tissue (including the small intestines and/or the large intestines), a liver tissue, a gallbladder tissue, a pancreatic tissue, a colorectal tissue,

In some embodiments, the method prevents, treats, or reduces a gastrointestinal disease or disorder in the subject. In some embodiments, the gastrointestinal disease or disorder comprises heartburn, irritable bowel syndrome, lactose intolerance, gallstones, cholecystitis, cholangitis, anal fissure, hemorrhoids, proctitis, colon polyps, infective colitis, ulcerative colitis, ischemic colitis, Crohn's disease, radiation colitis, celiac disease, diarrhea (chronic or acute), constipation (chronic or acute), diverticulosis, diverticulitis, acid reflux (gastroesophageal reflux (GER) or gastroesophageal reflux disease (GERD)), Hirschsprung disease, abdominal adhesions, achalasia, acute hepatic porphyria (AHP), anal fistulas, bowel incontinence, centrally mediated abdominal pain syndrome (CAPS), clostridioides difficile infection, cyclic vomiting syndrome (CVS), dyspepsia, eosinophilic gastroenteritis, globus, inflammatory bowel disease, malabsorption, scleroderma, or volvulus.

### Methods of Treating or Preventing Gastrointestinal Diseases or Disorders

In one aspect, disclosed herein is a method of treating or preventing a gastrointestinal disease or disorder in a subject in need thereof, the method comprising isolating and preserving a tissue sample from a subject, preparing and culturing any of the glycan-sensing systems disclosed herein in a microplate in an anaerobic environment, adding the tissue sample into the microplate, detecting and quantifying a light signal emitted from the system, and performing or administering a therapeutic procedure to the subject comprising pathological amounts of a glycan relative to a control tissue comprising normal amounts of the glycan.

In one aspect, disclosed herein is a method of treating or preventing a gastrointestinal disease or disorder in a subject in need thereof, the method comprising isolating and preserving a tissue sample from a subject, preparing and culturing any of the engineered bacteria disclosed herein in a microplate in an anaerobic environment, adding the tissue sample into the microplate, detecting and quantifying a light signal emitted from the engineered bacteria, and performing or administering a therapeutic procedure to the subject comprising pathological amounts of a glycan relative to a control tissue comprising normal amounts of the glycan.

In one aspect, disclosed herein is a method of treating or preventing a gastrointestinal disease or disorder in a subject in need thereof, the method comprising isolating and preserving a tissue sample from a subject, preparing any of the reporter plasmids disclosed herein in a microplate in an anaerobic environment, adding the tissue sample into the microplate, detecting and quantifying a light signal emitted from the reporter plasmid, and performing or administering a therapeutic procedure to the subject comprising pathological amounts of a glycan relative to a control tissue comprising normal amounts of the glycan.

In some embodiments, the tissue sample comprises at least one glycan. In some embodiments, the tissue sample comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, or more glycans. In some embodiments, the tissue sample comprises N-linked glycans, O-linked glycans, or combinations thereof. In some embodiments, the tissue sample comprises branched glycans or linear glycans, or combinations thereof. In some embodiments, the tissue sample comprises one or any combination of glycans comprising chondroitin sulfate, arabinan, heparan sulfate, hyaluronan, fructan, levan, mannan, pectic galactan, starch, glucan, galactomannan, glucomannan, homogalacturonan, xyloglucan, dermatan, xylan, rhamnogalacturonan, or derivatives thereof.

In some embodiments, the light signal is detected and quantified using a microplate reader, a spectrophotometer, or an instrument capable of measuring the light signal. In some embodiments, the tissue sample comprises a tissue biopsy, a blood sample, or a saliva sample. In some embodiments, the tissue sample comprises a tissue biopsy, a blood sample, or a saliva sample. In some embodiments, the tissue sample comprises a tissue biopsy from the GI tract, including, but not limited to a tongue scraping, buccal swab, an esophageal tissue, a stomach tissue, an intestinal tissue (including the small intestines and/or the large intestines), a liver tissue, a gallbladder tissue, a pancreatic tissue, a colorectal tissue,

In some embodiments, the therapeutic procedure comprises a surgical procedure, a therapeutic agent, a lifestyle change, or a combination thereof.

In some embodiments, the surgical procedure comprises bariatric surgery, esophageal surgery, foregut surgery, hernia surgery, gastroesophageal reflux disease surgery, surgical nutrition, colorectal surgery, gallbladder surgery, liver surgery, bile duct surgery, pancreatic surgery, tumor excision, or combinations thereof. In some embodiments, the therapeutic agent comprises a laxative, an antacid, a proton pump inhibitor, a histamine blocker, an anti-inflammatory agent, a steroid, a vitamin, an intravenous fluid, an antibiotic, a probiotic, or combinations thereof. In some embodiments, the lifestyle change comprises a dietary alteration, an exercise program, a sedentary lifestyle, or combinations thereof.

In some embodiments, the gastrointestinal disease or disorder comprises heartburn, irritable bowel syndrome, lactose intolerance, gallstones, cholecystitis, cholangitis, anal fissure, hemorrhoids, proctitis, colon polyps, infective colitis, ulcerative colitis, ischemic colitis, Crohn's disease, radiation colitis, celiac disease, diarrhea (chronic or acute), constipation (chronic or acute), diverticulosis, diverticulitis, acid reflux (gastroesophageal reflux (GER) or gastroesophageal reflux disease (GERD)), Hirschsprung disease, abdominal adhesions, achalasia, acute hepatic porphyria (AHP), anal fistulas, bowel incontinence, centrally mediated abdominal pain syndrome (CAPS), clostridioides difficile infection, cyclic vomiting syndrome (CVS), dyspepsia, eosinophilic gastroenteritis, globus, inflammatory bowel disease, malabsorption, scleroderma, or volvulus.

In some embodiments, the subject is a human.

### Kits of Detecting a Glycan Molecule

In one aspect, disclosed herein is a kit for detecting or purifying a glycan molecule, the kit comprising an apparatus comprising a binding medium and a surface glycan binding protein (SGBP), wherein the binding medium comprises a matrix of biomolecules, the SGBP comprises a peptide label, and the SGBP is attached to the matrix; a container comprising an equilibration buffer, a container comprising a releasing buffer, and a container comprising a washing buffer.

In some embodiments, the glycan molecule comprises chondroitin sulfate, arabinan, heparan sulfate, hyaluronan, fructan, levan, mannan, or derivatives thereof.

In some embodiments, the SGBP comprises a chondroitin sulfate (CS)-specific SGBP, a levan-specific SGBP, an inulin-specific SGBP, an arabinogalactan-specific SGBP, a dextran-specific SGBP, a hyaluronan (HA)-specific SGBP, a heparan sulfate (HS)-specific SGBP, an O-glycan(OG)-specific SGBP, or variants thereof.

In some embodiments, the matrix comprises a nickel-conjugated agarose polymer. In some embodiments, the matrix comprises a stationary or porous composition of biomolecules, including, but not limited to a metal ion (including, but not limited to cobalt, nickel, and copper), an antibody, a protein, a nucleic acid, a hormone, and metabolite capable of reversibly binding a target composition, compound, or molecule.

In some embodiments, the SGBP is directly conjugated to a peptide label or the SGBP is conjugated to the peptide label by a linker. It is contemplated that the location and placement of the peptide label on the SGBP is not limited. In some embodiments, the peptide label comprises a hexa-histidine label. In some embodiments, the releasing buffer comprises a histidine solution. In some embodiments, the releasing buffer comprises a buffer with a high salt concentration. In some embodiments, the releasing buffer comprises a buffer with a low salt concentration. In some embodiments the releasing buffer comprises a buffer with a low pH. In some embodiments, the releasing buffer comprises a buffer with a high pH. In some embodiments, the releasing buffer is a buffer comprising an ionic charge. In some embodiments, the releasing buffer comprises a buffer with a neutral ionic charge. In some embodiments, the releasing buffer comprises a buffer with a neutral pH. In some embodiments, the washing buffer comprises a buffered solution comprising water, phosphate buffered saline solution, a tris-buffered saline solution, or variants thereof.

In some embodiments, the kit is combined with any of the glycan-sensing systems disclosed herein to detect the glycan molecule.

In some embodiments, the kit is combined with any of the engineered bacteria disclosed herein the glycan molecule.

In some embodiments, the kit is combined with any of the reporter plasmids disclosed herein to detect the glycan molecule.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Harnessing Gut Microbes for Glycan Detection and Quantification

The mammalian gut microbiota is a critical human health factor and a premier target for therapeutic modulation. The gut microbiota composition is influenced by the host diet, intestinal mucosa, and co-resident microbes, which all contribute to the availability of complex oligo- and poly-saccharides, collectively referred to herein as glycans. Glycans are highly abundant macromolecules found in every domain of life, mediate critical cellular processes, and exhibit extraordinary structural diversity conferred by unique non-linear arrangements, glycosidic linkages, and monosaccharide components. Altering glycan availability in the intestine modulates the gut microbiota by favoring the expansion of microbial populations that can consume individual glycan structures, which typically requires differentially available enzymatic activities. The mammalian intestine can exhibit disease-associated alterations in mucosal glycosylation influencing the abundance of gut microbial populations. Thus, evaluating glycans associated with intestinal diseases reveal disease biomarkers, therapeutic targets, or prebiotic compounds. However, unlike nucleic acids and proteins, glycan structural complexity limits high-throughput examinations of heterogenous glycan mixtures using individual methodologies frequently requiring parallel tandem approaches to comprehensively examine complex heterogenous glycan mixtures. Therefore, new tools and approaches are necessary to efficiently survey mixtures for unique glycan structures that could modulate microbial abundance and activity in the gut.

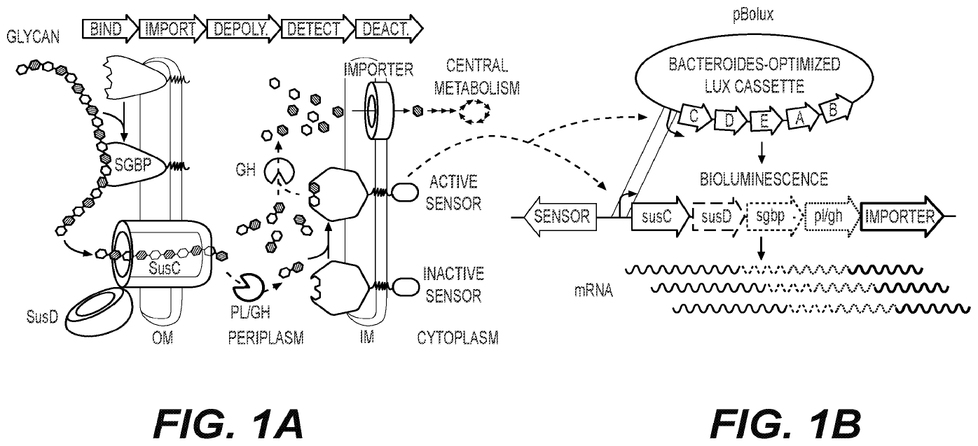

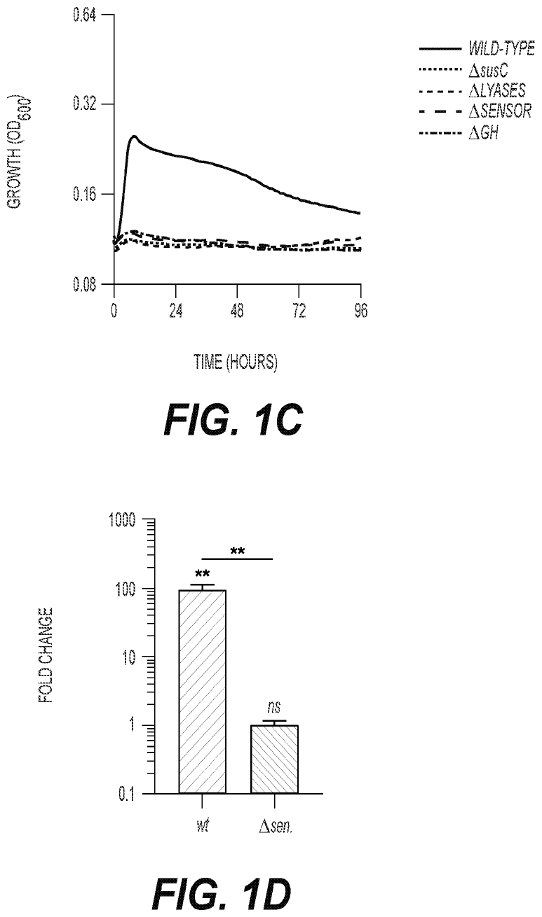

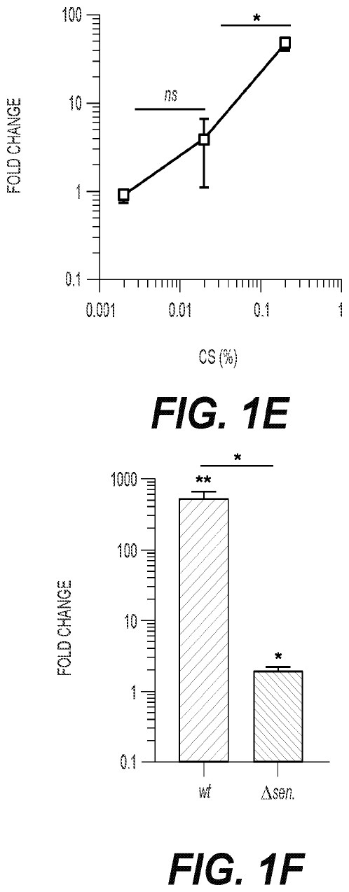

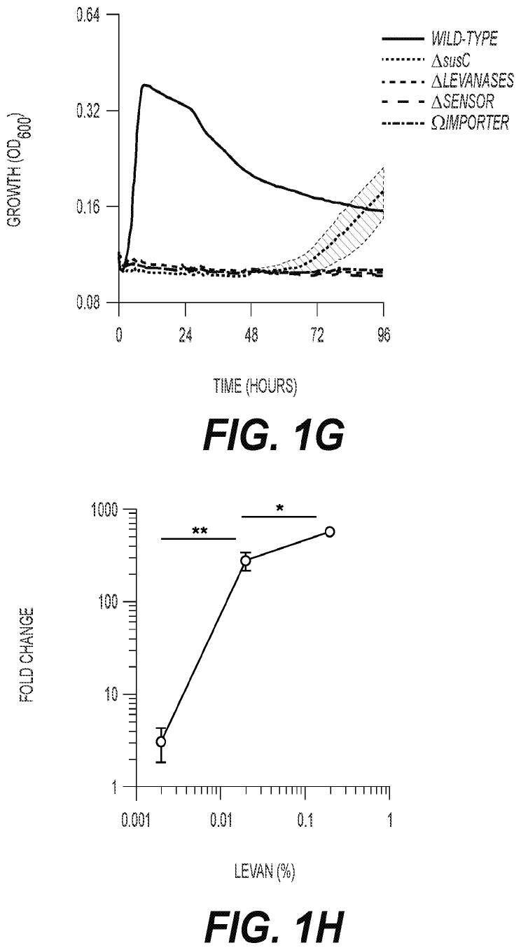

Microbes have evolved systems dedicated to glycan recognition and consumption from a myriad of biological sources. Members of the gut microbiota encode vast repertoires of glycan utilization machinery conferring unparalleled access to the diverse substrates present in the intestinal lumen and facilitate gut colonization. For example, a dominant and abundant bacterial group present in the mammalian gut, called the Bacteroides, encode discreet, tightly linked, coordinately regulated gene clusters called polysaccharide utilization loci (PULs) that mediate the consumption of structurally distinct glycans. Many Bacteroides species encode vast PUL libraries endowing these organisms with the ability to consume structurally diverse complex polysaccharides derived from the host diet, co-resident microbes, and the intestinal mucosa to successfully thrive in the mammalian gut. Most PULs are transcriptionally regulated such that individual genes are expressed at low constitutive levels until encountering their target glycans (FIG. 1A), resulting in rapid and dramatic increases in corresponding PUL transcripts (FIG. 1B). These responses are directed by sensor proteins that recognize mono-, di-, or oligo-saccharide signatures of their corresponding target glycan(s) and subsequently increase PUL transcript levels in a rapid and dramatic fashion (FIGS. 1A and 1B). The ability of PUL sensors to direct PUL transcription changes in response to glycan availability demonstrates their use as biosensors to be employed to surveille heterogenous mixtures.

Additionally, PULs encode proteins possessing glycan binding, transport, and depolymerization activities that exhibit substrate specificity and confer species-specific access to discreet glycan subsets (FIG. 1A). For example, B. thetaiotaomicron (Bt) and a closely related species, B. ovatus (Bo) both encode similar polyfructan-specific PULs that confer discreet target glycan specificities. The Bt fructan PUL facilitates the consumption of the β-2,6-linked poly-fructan, levan, which is inaccessible to Bo, while the Bo fructan PUL facilitates utilization of the β-2,1-linked polyfructan, inulin, which is inaccessible to Bt. Species specific poly-fructan utilization is mediated independently of the fructan PUL-sensor, which activates PUL transcription in both Bt and Bo following detection of monomeric fructose. Thus, species-specific differences in target glycan utilization can rely on PUL-encoded factors that distinguish between compositionally similar yet structurally distinct glycans. Moreover, the specificity exhibited by SGBPs (FIG. 1A) demonstrates their use as affinity purification reagents to isolate PUL-target glycans from complex heterogenous mixtures aiding in their structural characterization However, the paucity of specific, sensitive, and cost-effective glycan detection methods has hindered the development of such approaches.

Bacteroides PUL sensors are described to be harnessed as tools to detect their corresponding target glycans by examining changes in PUL transcription following the introduction of various biological samples. However, examining PUL transcription in response to glycan mixtures has relied on molecular approaches such a qPCR, microarrays, and RNAseq to detect transcriptional changes in Bacteroides species. Furthermore, the anaerobic growth requirements of Bacteroides species have previously limited the implementation of traditional transcriptional reporters such as GFP and luciferase to ex vivo measurements at discreet times. Herein, a Bacteroides-optimized luciferase reporter is generated to accurately reflect transcription during growth in anaerobic culture and is readily quantifiable. Reporter plasmids containing PUL promoters can sensitively and specifically indicate the presence of target glycans and these responses require PUL-encoded transport, degradation, and detection machinery in multiple Bacteroides species. Moreover, it was determined that target glycans elicit dose-dependent responses from their corresponding PUL reporters that can be used to quantify glycan abundance as accurately as commercially available kits. Finally, an affinity purification strategy was developed using PUL-encoded SGBPs to isolate individual target glycans from heterogenous mixtures by employing PUL-reporters as glycan detection and quantification tools. This microbial glycomics toolkit is readily scalable and will enable efficient characterization of unknown PUL ligands, identify previously concealed genetic determinants governing microbial glycan utilization, and ultimately reveal the glycomic interface between mammals and their gut microbiotas.

### Results

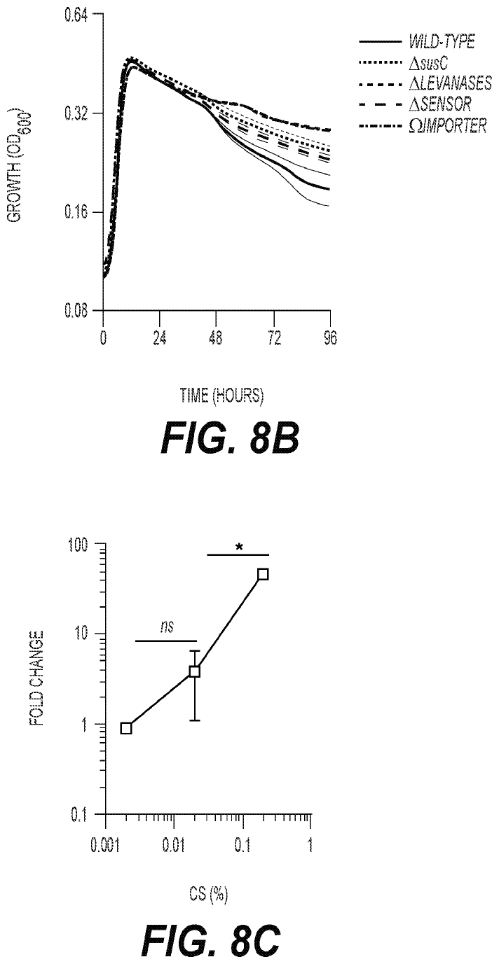

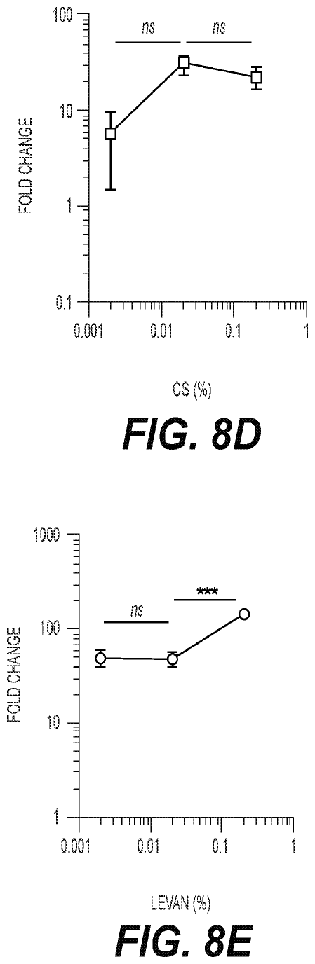

Bacteroides PUL sensors promote dose-dependent transcriptional responses to target glycans. Bacteroides species frequently encode many PULs (Bt and Bo encode 88 and 112, respectively, each putatively dedicated to the utilization of distinct glycan substrates. PULs include a susCD-encoded complex necessary for the translocation of a target glycans across the outer membrane (FIG. 1A) and the levels of susCD-containing transcripts increase rapidly and dramatically following exposure to their cognate target glycan (FIG. 1B). For example, transcripts corresponding to the susC gene, BT3332, located within a Bt PUL required for chondroitin sulfate (CS) utilization (FIG. 1C) increase 92-fold after 120 minutes following the addition of CS to the media (FIG. 1D). Similarly, transcripts corresponding to another susC gene, BT1763, located within the Bt PUL necessary for levan utilization (FIG. 1E), increase 530-fold following the addition of levan (FIG. 1F). These increases in susC transcript levels require sensor proteins that often bind target glycan-derived ligands in the periplasm and subsequently direct transcriptional responses in the cytoplasm (FIG. 1A). Accordingly, Bt mutants lacking either the CS or levan PUL sensors, are unable to grow on their corresponding target glycans as sole carbon sources (FIGS. 1C and 1E, respectively) or increase PUL transcription (FIGS. 1D and 1F, respectively), but exhibit no growth defects on galactose (FIGS. 8A and 8B, respectively).

Interestingly, BT1763 and BT3332 transcript levels exhibited corresponding decreases when Bt was supplied 10-fold dilutions of levan or CS (FIGS. 1G and 8C, respectively) indicating that Bacteroides PUL transcriptional responses are concentration-dependent. However, the relationship between susC-transcript and glycan concentrations detected at 120 minutes was not apparent after 60 minutes following glycan addition (FIGS. 8D and 8E), demonstrating the dynamic nature of PUL transcription. These results show that PUL sensors promote dose-dependent transcriptional responses over time, which are harnessed to report unknown target glycan abundances by measuring changes in corresponding susC-transcript levels.

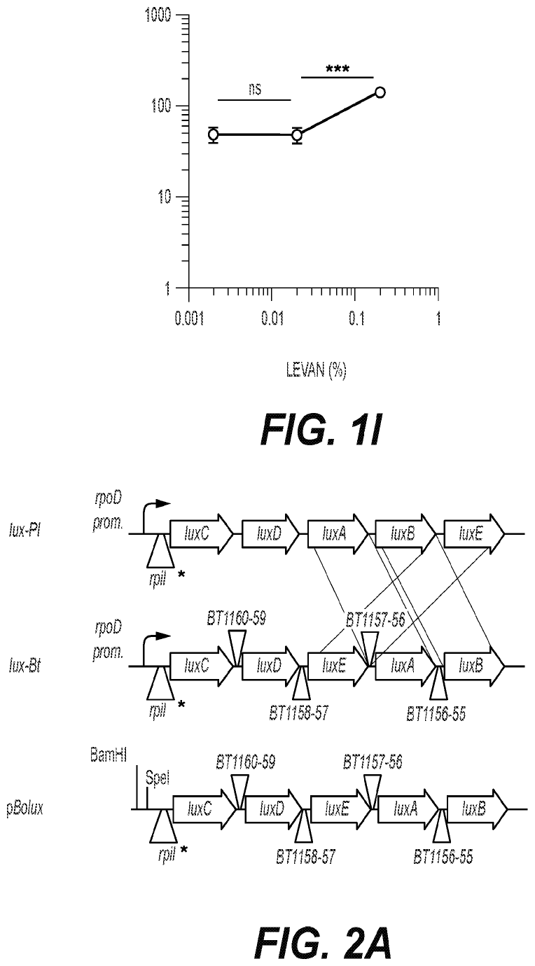

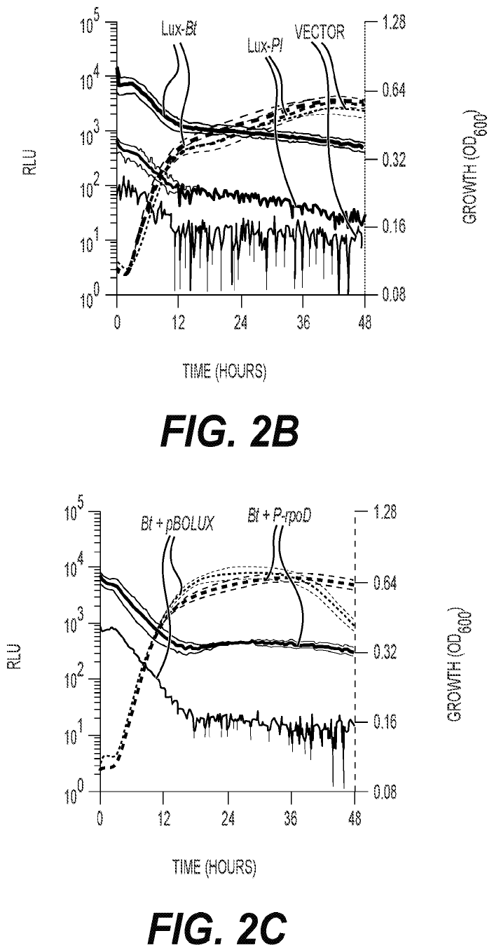

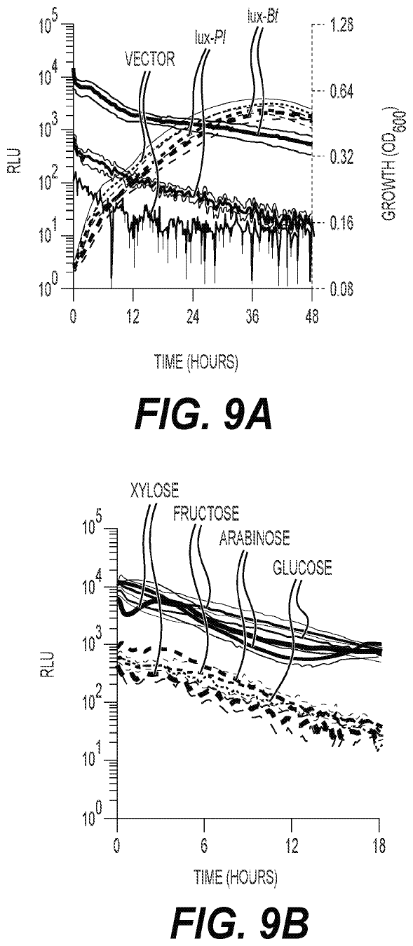

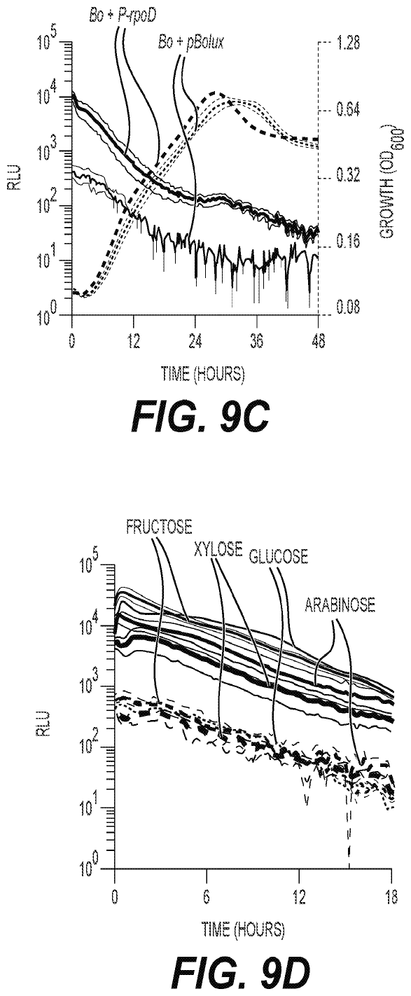

Construction of a Bacteroides-optimized bioluminescent transcriptional reporter. Quantifying susC transcription by qPCR or transcriptomics is expensive and inefficient, requiring kinetic sampling to accommodate variations in PUL transcriptional responses. Therefore, a transcriptional reporter was engineered that accurately reflects transcript levels in Bacteroides species over time during anaerobic growth without additional oxygenation. Previously constructed transcriptional reporters in Bt require terminal measurements following cell lysis limiting their applications for kinetic measurements and fluorophores such as GFP require exposure to oxygen for proper maturation or exhibit high background fluorescence. In contrast, the production of bacterial LuxCDABE proteins and biosynthesis of the luciferase long-chain aldehyde substrate can be achieved anaerobically although luciferase activity is oxygen dependent. It was contemplated that lux-mediated bioluminescence could function in Bacteroides species during growth under anaerobic conditions when expressed from Bacteroides promoters and ribosomal binding sites, which differ from those typically found in Firmicutes and Proteobacteria. Therefore, the Photorhabdus luminescens (Pl) luxCDABE cassette was engineered in the multi-copy plasmid, pLYL01, preceded by the Bt rpoD promoter (BT1311) and an optimized Bacteroides ribosome binding site, rpiL*, that facilitates high levels of gene expression (FIG. 2A). A wild-type Bt strain harboring the Pl lux cassette exhibited detectable luminescence during anaerobic growth in minimal media containing galactose (FIG. 2B) or glucose (FIG. 9A) and grew similarly to a strain harboring the empty vector under identical conditions (FIGS. 2B and 9A, respectively).

To further optimize luciferase activity, the lux operon was re-organized to luxCDEAB and exchanged the Pl intergenic regions for the Bt intergenic regions from BT1160-1155 (FIG. 2A), which increased luminescence output approximately 10-fold over the Pl cassette without altering growth kinetics (FIGS. 2B and 9A). A Bt strain harboring the Bacteroides-optimized lux cassette exhibited consistently higher relative luminescence than a strain expressing the Pl cassette during logarithmic growth (FIGS. 2B and 9A) and remained readily detectable even in late stationary phase over 48 hours (FIG. 2B). A plasmid containing tandem BamHI and SpeI restriction sites upstream of rpiL*-luxCDEAB (pBolux, FIG. 2A) were constructed for efficient cloning of promoters. A Bt strain harboring pBolux exhibited similar sustained luminescence over 18 hours in galactose (FIG. 2C), glucose, fructose, arabinose, or xylose as sole carbon sources (FIG. 9B). Introduction of the Bt rpoD promoter into the BamHI and SpeI sites increased activity 10-fold independently of carbon source (FIGS. 2C and 9B). Introduction of pBolux into Bo produced similar results across all conditions and a plasmid preceded by the region upstream of the Bo rpoD gene (BACOVA_00615) increased luminescence approximately 10-fold (FIGS. 9C and 9D). These data indicate that pBolux can reflect promoter-dependent transcription in multiple Bacteroides species during anaerobic culture with minimal cost to bacterial growth.

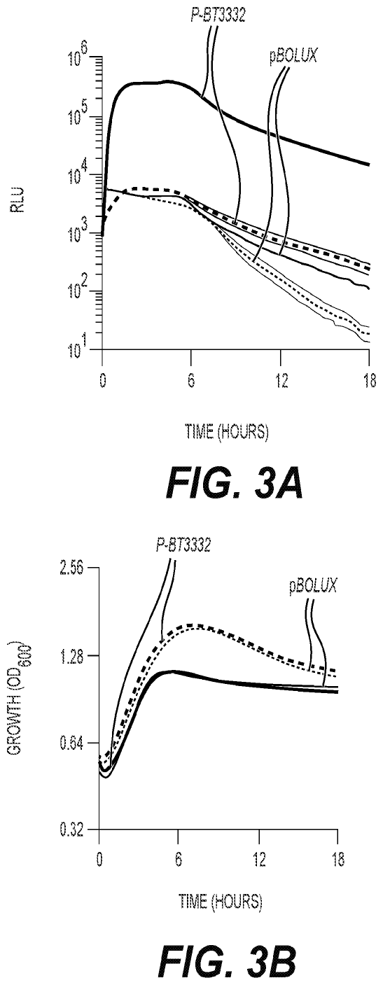

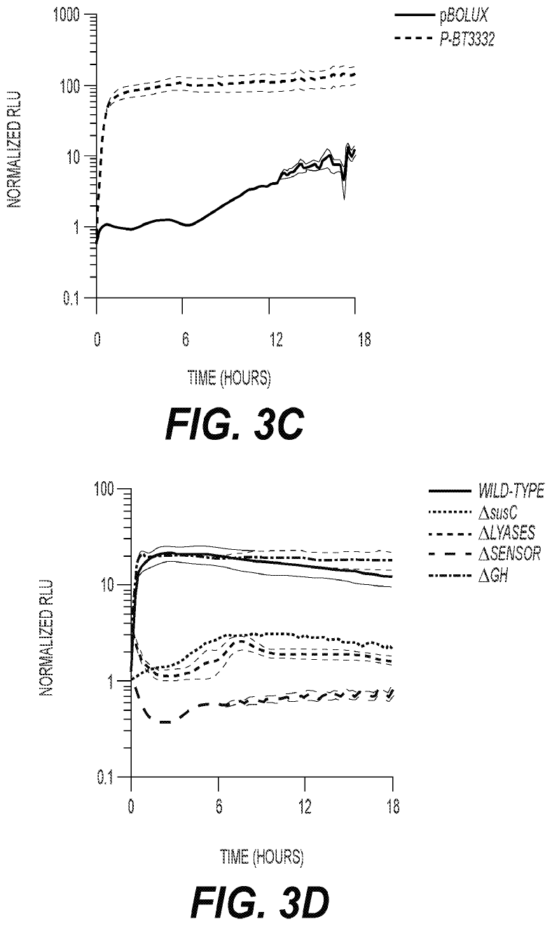

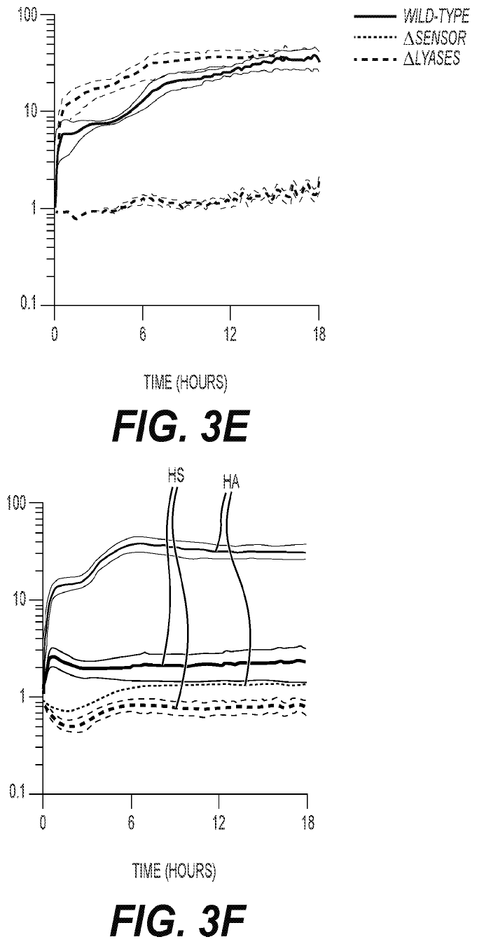

Engineering a glycan responsive PUL reporter. To determine if pBolux can accurately indicate changes in PUL transcription in response to target glycans, the 300 bp region was introduced immediately upstream of the susC gene from the CS PUL (P-BT3332). Luminescence (FIG. 3A) and growth (FIG. 3B) was measured in a wild-type Bt strain harboring P-BT3332 or promoter-less pBolux for 18 hours following the introduction of CS or galactose as the sole carbon source. After only 2 hours, the relative luminescence from a strain harboring P-BT3332 was 46-fold greater and reached a peak activity of 65-fold by 7 hours following the introduction of CS compared to cultures supplied galactose alone (FIGS. 3A and 3C). Conversely, a strain harboring pBolux exhibited identical luminescence in CS relative to galactose until 6 hours where it increased 7-fold over 18 hours (FIG. 3C). P-BT3332-driven reporter increases imposed no detectable growth alterations indicating that increased luminescence carries minimal fitness cost (FIG. 3B).

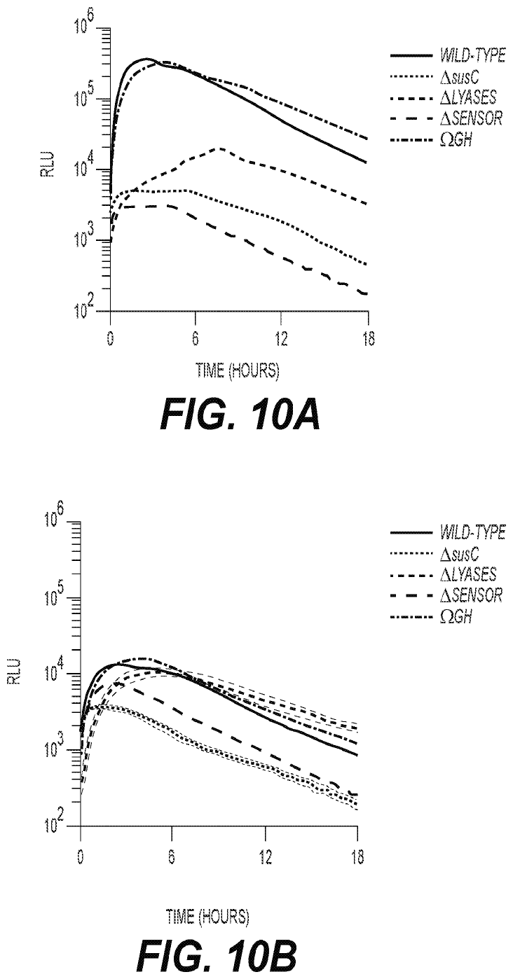

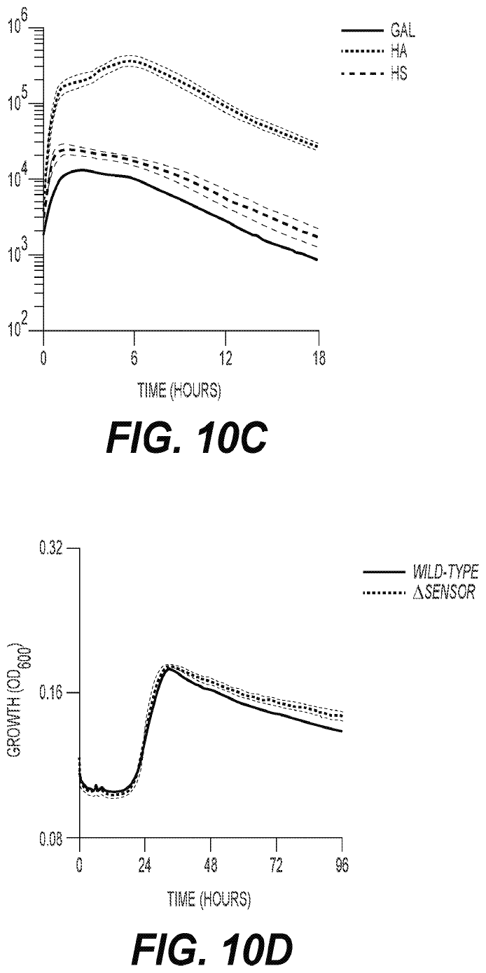

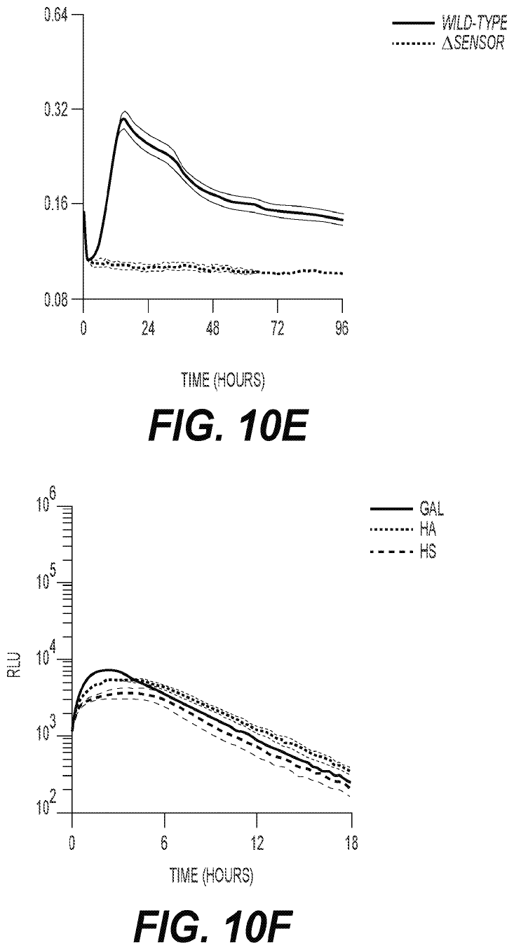

To examine whether genes encoded within the CS PUL (BT3328-BT3334 & BT4410-BT4411) are required for the observed P-BT3332-specific luminescence increases in CS, reporter activity in mutant Bt strains deficient for CS transport, depolymerization, and detection activities necessary for growth on CS (FIG. 1C) were compared following the introduction of a mixture of 0.2% CS and 0.2% galactose (FIG. 10A) (galactose was added as a supplemental carbon source to support growth and luciferase activity) or galactose alone (FIG. 10B). A mutant lacking the CS-sensor, BT3334, exhibited no increased luminescence over 18 hours following the introduction of CS (FIG. 3D). The reduced bioluminescence exhibited by this strain was not the consequence of disabling growth on CS because a mutant lacking a glucuronyl hydrolase, BT3348 (gh), is unable to utilize CS (FIG. 1C), but exhibits sensor-dependent PUL transcription and increased reporter activity similar to wild-type Bt (FIG. 3D). These data show that disabling genes necessary to generate the PUL-sensor ligand, the 6- or un-sulfated CS-derived disaccharide (di0S), in the periplasm prevent sensor-dependent reporter activity increases. Consistent with this notion, a mutant lacking the CS PUL-encoded susC transporter, BT3332, exhibited a dramatic reduction in reporter activity with a peak activity of 3.1-fold increase at 10 hours putatively by reducing CS importation from the extracellular environment (FIG. 3D). Furthermore, disabling 3 CS-specific polysaccharide lyases necessary for the conversion of CS-polymers into di0S (BT3324, BT3350, and BT4410) also dramatically reduced reporter activity in response to CS relative to wild-type Bt, peaking at 3.3-fold after 1 hour following the introduction of CS (FIG. 3D). However, supplying di0S increased luminescence 39.6-fold by 16 hours in the lyase-deficient but not the sensor-deficient mutant (FIG. 3E). Collectively, these data indicate that a strain harboring P-BT3332 can indicate the presence of CS in a manner dependent on PUL-encoded glycan importation, depolymerization, and detection activities.

To determine whether increased activity from a strain containing P-BT3332 were limited to CS-PUL substrates, luminescence was examined following the addition of a structurally similar but compositionally distinct glycosaminoglycan, heparin sulfate (HS, FIG. 10C), which is consumed by Bt independently of the CS-PUL (FIG. 10D). A strain harboring the CS-reporter exhibited a dramatically lower activity following the addition of HS compared to the addition of CS (FIG. 3F), peaking at 2.6-fold after an hour. Conversely, the addition of hyaluronic acid (HA), which also activates sensor-dependent CS-PUL gene expression required for HA utilization (FIG. 10E), increased reporter activity similarly to CS in wild-type Bt but not the sensor-deficient mutant (FIGS. 3F and 10F). Thus, the Bacteroides-optimized lux reporter can specifically indicate the presence of target glycans by reflecting PUL sensor-dependent responses to glycan-derived ligands.

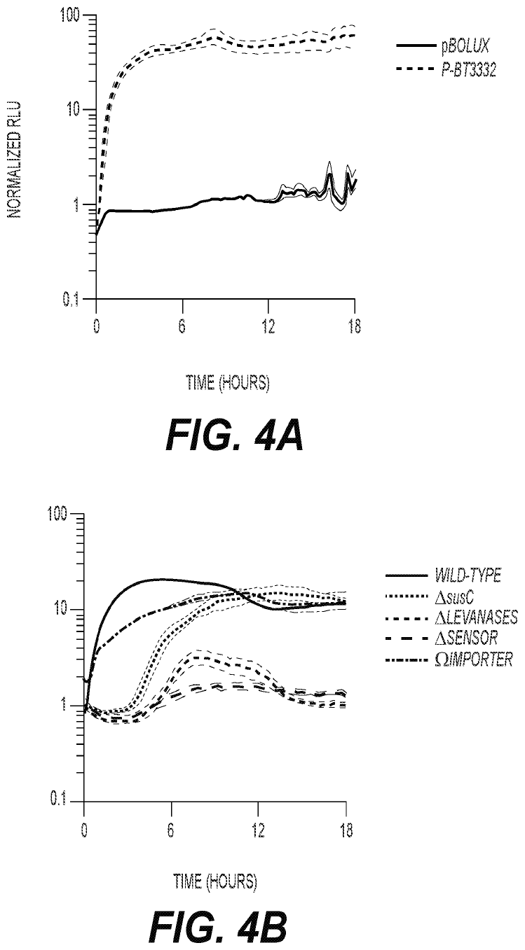

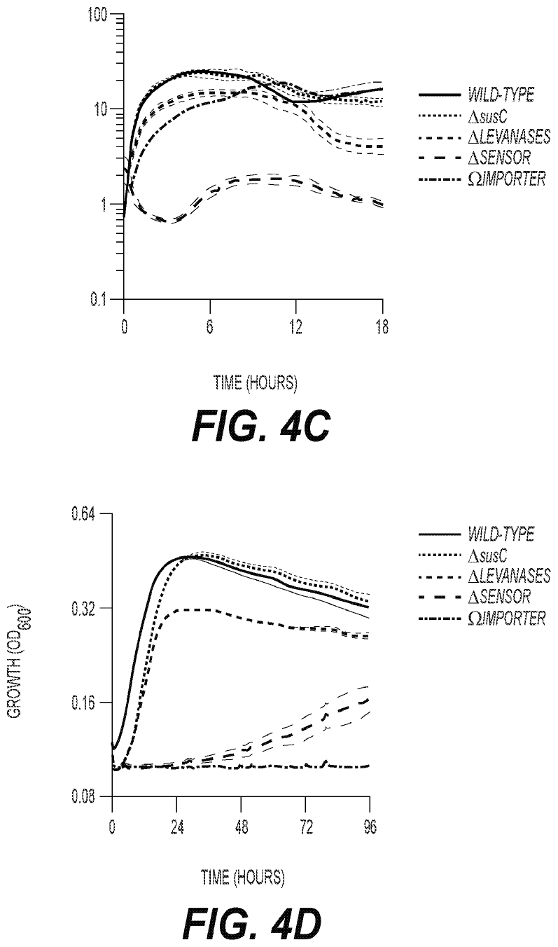

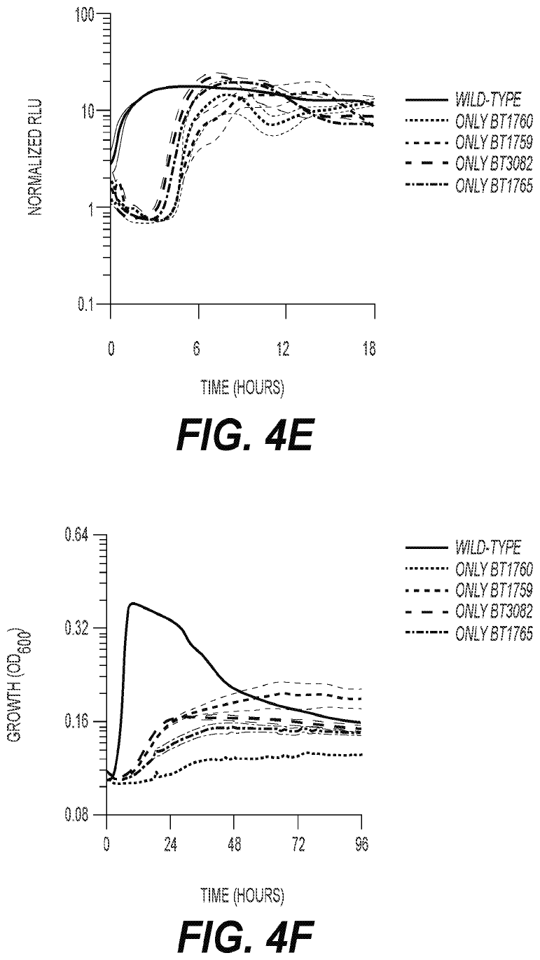

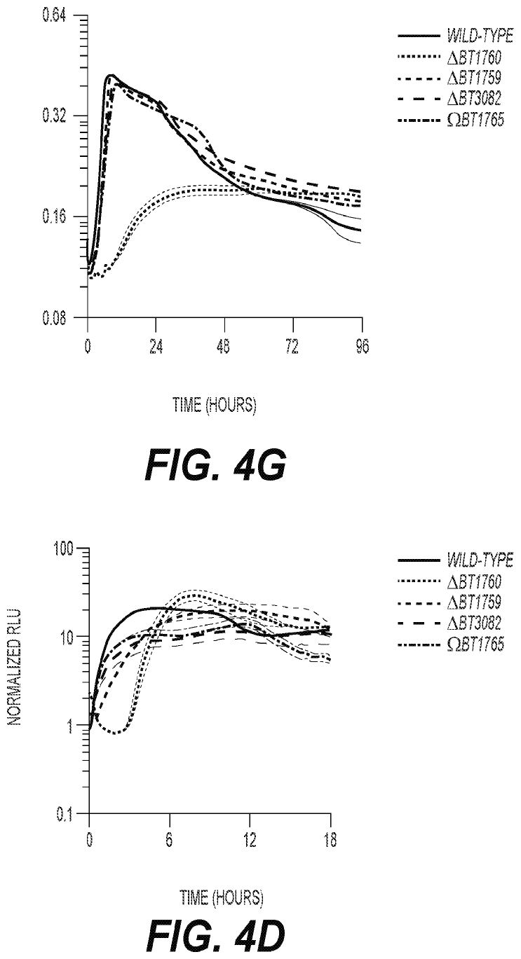

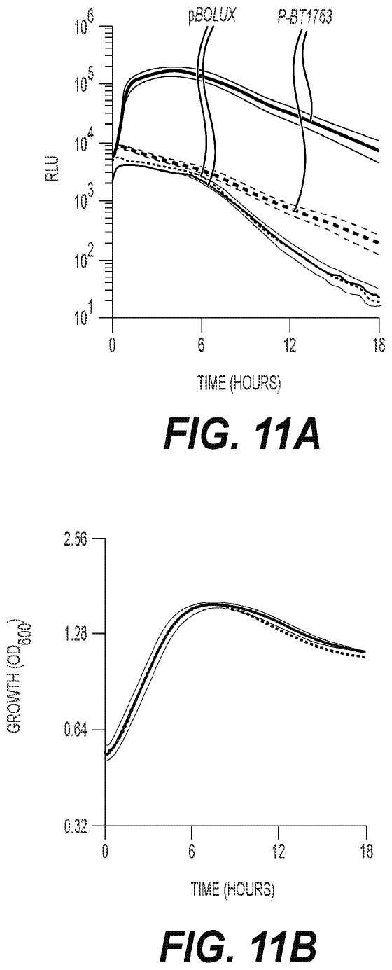

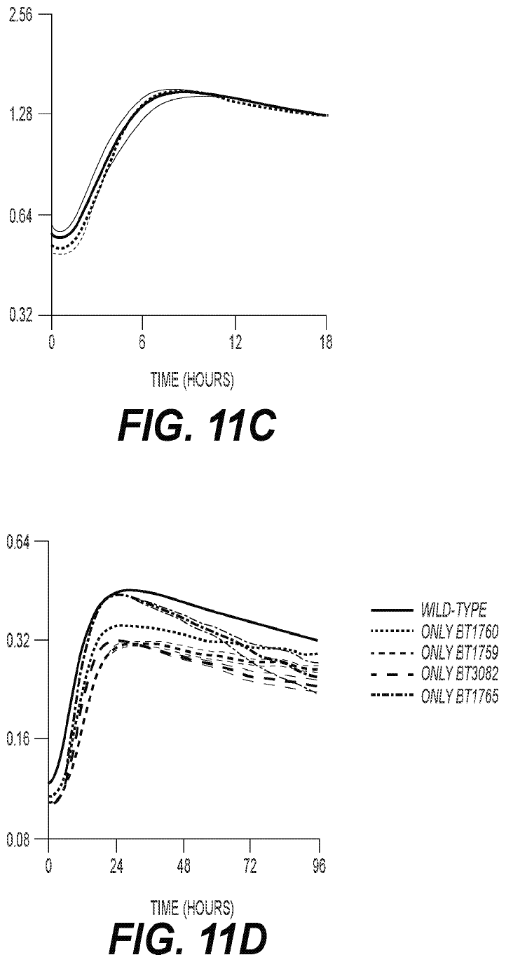

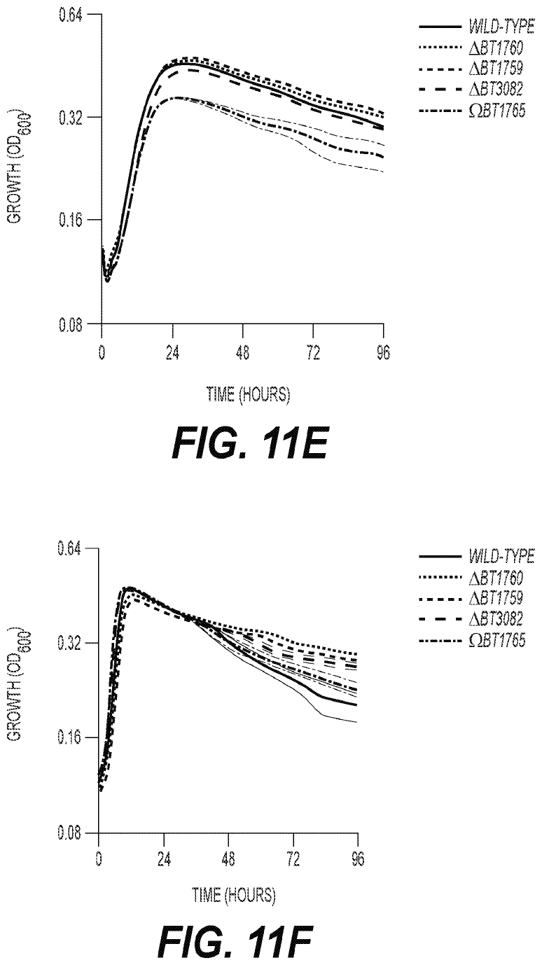

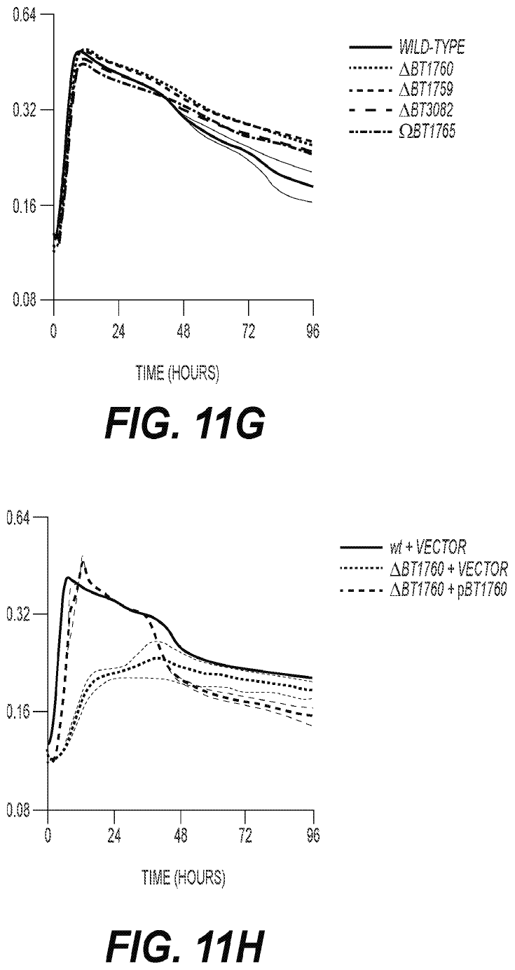

A fructan-responsive reporter reveals new insights into Bt levan utilization. Bt consumption of the polyfructan, levan, requires a distinct PUL encoded by BT1754-BT1765 and includes an unlinked but co-regulated exo-levanase, BT3082. A Bt strain harboring pBolux containing the region preceding the corresponding susC gene (P-BT1763) exhibits 35-fold increased activity 5.5 hours after the introduction of levan compared to galactose (FIGS. 4A and 11A). Levan-dependent reporter activity requires the BT1763 promoter region because a strain harboring the promoter-less control plasmid, pBolux, exhibits no change after 18 hours following the introduction of levan (FIGS. 4A and 11A). Differences in reporter-activity cannot be attributed to plasmid-dependent growth effects because both strains exhibit identical growth kinetics in either galactose (FIG. 11B) or levan (FIG. 11C). Reporter activation requires levan-derived fructose detection by the PUL sensor protein, BT1754, because a mutant lacking BT1754 displays only 1.4- or 1.7-fold increased reporter activity following the introduction of either levan (FIG. 4B) or fructose (FIG. 4C), respectively. Moreover, a strain lacking the corresponding susC gene exhibited a 3-hour delay before reporter activity increased following the addition of levan (FIG. 4B) and 60-hour lag before detectable growth on levan as the sole carbon source (FIG. 1E). Conversely, identical strains exhibit reporter activity (FIG. 3C) and growth kinetics (FIG. 3D) resembling wild-type Bt when supplied fructose, confirming that the fructan-specific SusC is dispensable for transport of monomeric fructose and internalizes levan-derived fructo-oligosaccharides. Consistent with this notion, it was determined that inactivation of 4 levanases (BT1760, BT1759, BT1765 and BT3083) reduced levan-dependent reporter activity to levels similar to the BT1754-deificient strain (FIG. 4B) and abolished utilization of levan as the sole carbon source (FIG. 1E). Strains harboring only a single levanase, generated by disabling various combinations of all other 3 levanases, exhibited reporter activity increases in levan after a 4-hour delay (FIG. 4E) and grow on levan as the sole carbon source (FIG. 4F) albeit at dramatically reduced rates and maximum cell densities compared to wild-type Bt. Unexpectedly, all strains lacking the exo-levanase, BT1765, exhibited reduced maximum cell densities during growth in fructose as the sole carbon source (FIGS. 11D and 11E), and this was specific because BT1765-deficient strains grew similarly to wild-type Bt in galactose (FIGS. 11F and 11G). Finally, reduced levan-dependent reporter activity in any mutant is not due to diminished growth because a mutant deficient for the inner membrane fructose importer, BT1758, cannot grow on levan (FIG. 1E) or fructose (FIG. 4D) as sole carbon sources but exhibit reporter activity resembling wild-type Bt in response to either carbohydrate (FIGS. 4B and 4C, respectively). Collectively, these data demonstrate that P-BT1763 containing strains indicate the presence of levan-derived fructose in a manner requiring PUL-encoded transport, depolymerization and detection proteins independently of its utilization.

These results reveal that Bt employs multiple levanases to consume levan and demonstrate that three distinct exo-levanases can function independently of the endo-levanase, BT1760, contrasting previous conflicting reports asserting this gene was either essential or dispensable for growth on levan. To examine the role of BT1760 in levan utilization, a BT1760-deficient strain was constructed, which exhibited reduced growth on levan as the sole carbon source (FIG. 4G) and delayed reporter activation in response to levan compared to wild-type Bt (FIG. 4H). This mutant exhibited growth and reporter activity resembling wild-type Bt when complemented in trans (FIG. 11H) indicating that this BT1760 mutation did not disrupt expression of downstream genes required for levan utilization (FIG. 1E). Furthermore, a mutant encoding only BT1760 and lacking all known exo-levanases (ΔBT1759, ΔBT3082 ΩBT1765), exhibited the greatest delay in reporter activity increases (FIG. 4E) and achieved the lowest maximum growth on levan as the sole carbon source (FIG. 4F) compared to any other combination of levanase mutations. Collectively, these data demonstrate that exo-levanases function independently of BT1760 to liberate fructose from levan highlighting that Bt levan utilization is not completely understood. Finally, these results illustrate how the pBolux reporter plasmid can be implemented to genetically dissect the contributions of individual PUL-encoded activities to target glycan consumption.

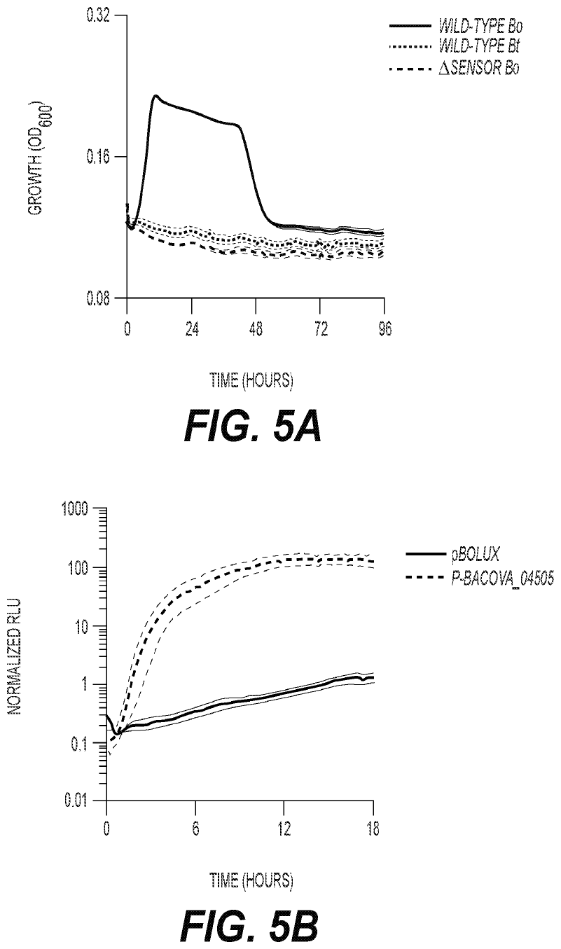

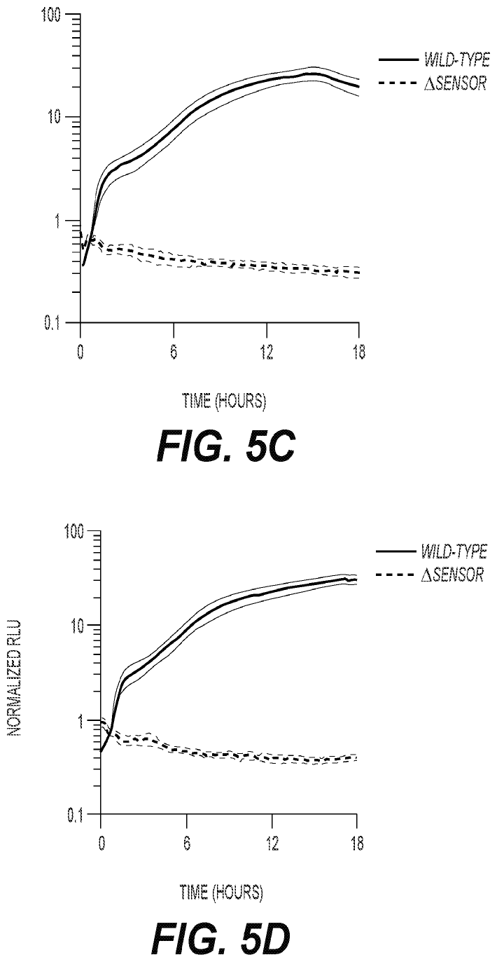

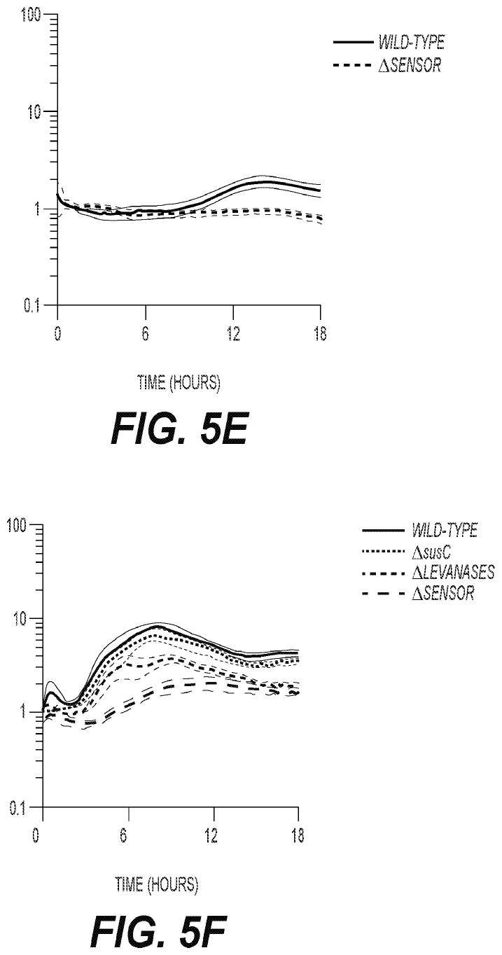

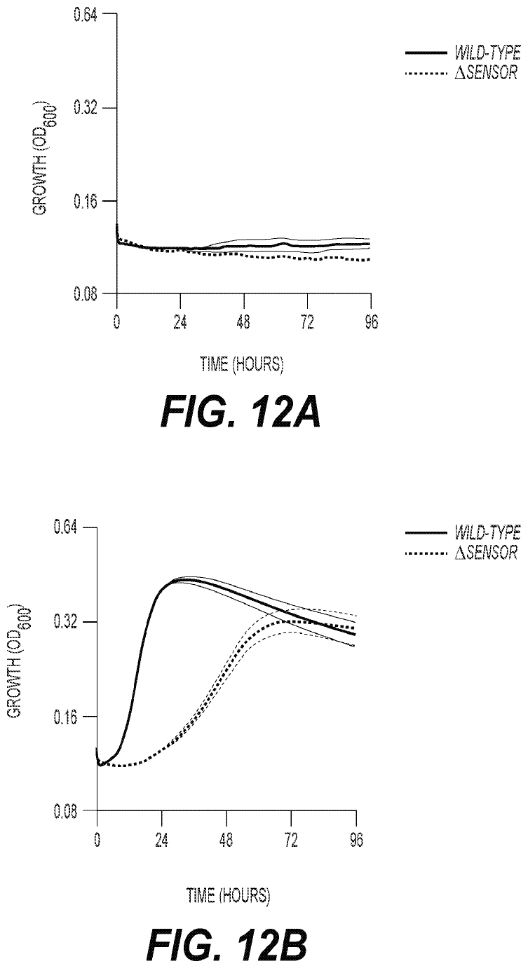

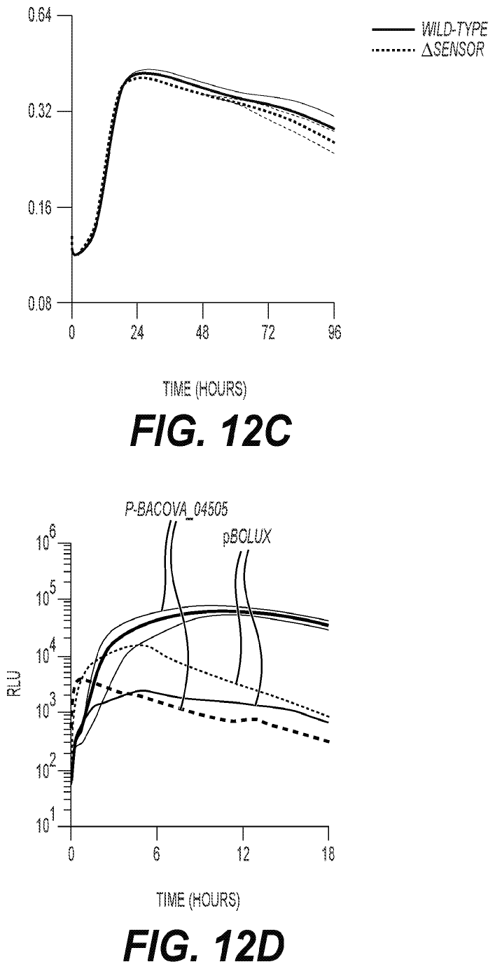

Species-specific reporter responses distinguish between compositionally identical glycans. Bacteroides species can differentially consume structurally distinct glycans comprised of identical monosaccharide components. This is exemplified by the fructan PUL, which is conserved across several species and confers Bt the ability to consume the β (2,6)-linked polyfructan, levan (FIG. 1E), but not the β (2,1)-linked polyfructan, inulin (FIG. 5A). Conversely, a similar PUL in Bo (BACOVA_04496-BACOVA_04507) confers inulin (FIG. 5A) but not levan utilization (FIG. 12A). Bo inulin utilization requires the PUL-sensor protein, BACOVA_04496, because a mutant lacking the corresponding gene is unable to grow on inulin as a sole carbon source (FIG. 5A) and exhibits reduced growth rates on fructose (FIG. 12B) but not galactose (FIG. 12C), showing that the Bo fructan PUL sensor detects monomeric fructose similarly to the Bt fructan PUL sensor. To examine how similar PULs encoded by closely related species can confer access to compositionally identical yet structurally distinct glycans, a Bo strain was generated harboring a reporter plasmid containing the region upstream of the corresponding susC gene (P-BACOVA_04505). This strain exhibits dramatically increased activity following the addition of inulin compared to galactose, in contrast to a promoter-less control plasmid (FIGS. 5B and 12D). Inulin-dependent reporter activity increases in Bo requiring the fructan PUL sensor because no change in reporter activity was observed when the sensor mutant was supplied inulin (FIG. 5C) or fructose (FIG. 5D). Collectively, these results indicate that the Bo fructan PUL sensor responds to monomeric fructose in a manner similar to the Bt sensor although each PUL confers access to structurally distinct glycans. Thus, identical PUL sensor specificities are employed to mediate utilization of distinct fructans showing that other PUL-encoded products facilitate species-specific growth on inulin and levan.

Interestingly, the addition of levan elicits detectable reporter activity increases in wild-type Bo harboring P-BACOVA_04505 compared to a strain lacking the corresponding PUL-sensor (FIG. 5E) although Bo is unable to grow on levan as a sole carbon source (FIG. 12A). Similarly, wild-type Bt harboring P-BT1763 exhibited detectable reporter activity increases when supplied inulin compared to a sensor-deficient strain (FIG. 5F), showing that Bo and Bt can derive fructose from levan and inulin, respectively, in quantities insufficient to support growth. It is contemplated that these responses reflect slow, non-specific, and extracellular liberation of fructose from non-target fructans because a Bt strain lacking the fructan-PUL encoded susC gene, BT1763, exhibits identical reporter activity relative to the wild-type strain following the addition of fructose (FIG. 4C) or inulin (FIG. 5F). Although inulin-dependent reporter activity was substantially reduced in a strain lacking 4 levanases, luminescence levels were greater than a sensor-deficient mutant suggesting Bt possesses unknown inulin-degrading activities that can likely liberate fructose monomers slowly and non-specifically compared to Bo (FIG. 5F). Collectively, these results demonstrate how PUL reporters exhibit highly sensitive responses in distinct gut Bacteroides species and can detect the presence of compositionally identical but structurally distinct glycans independently of their utilization.

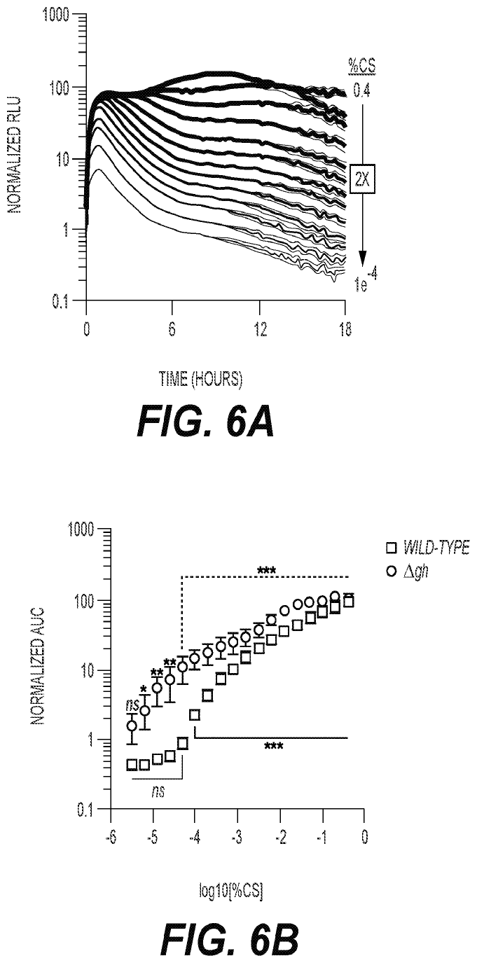

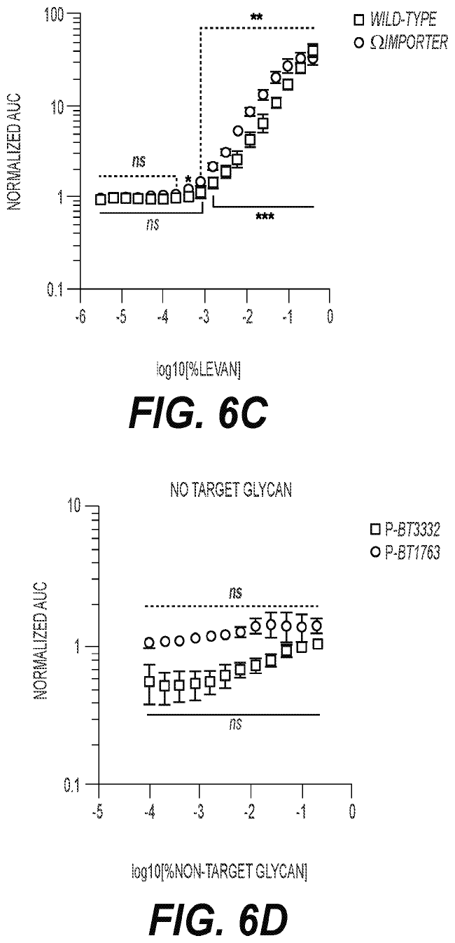

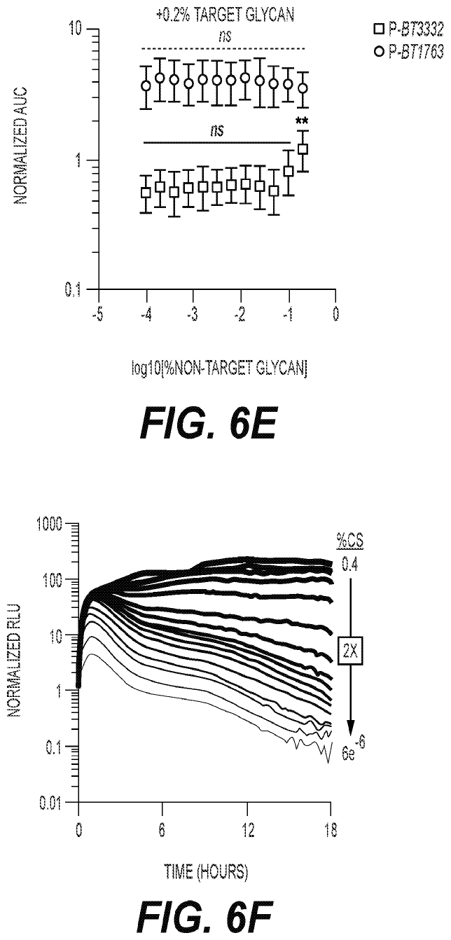

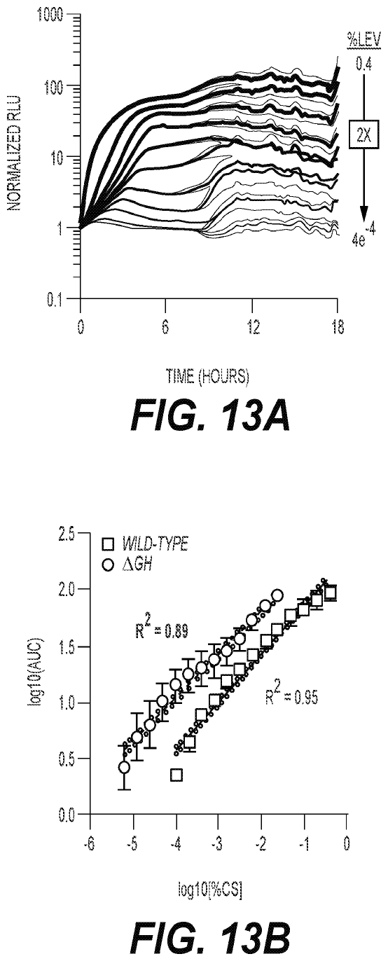

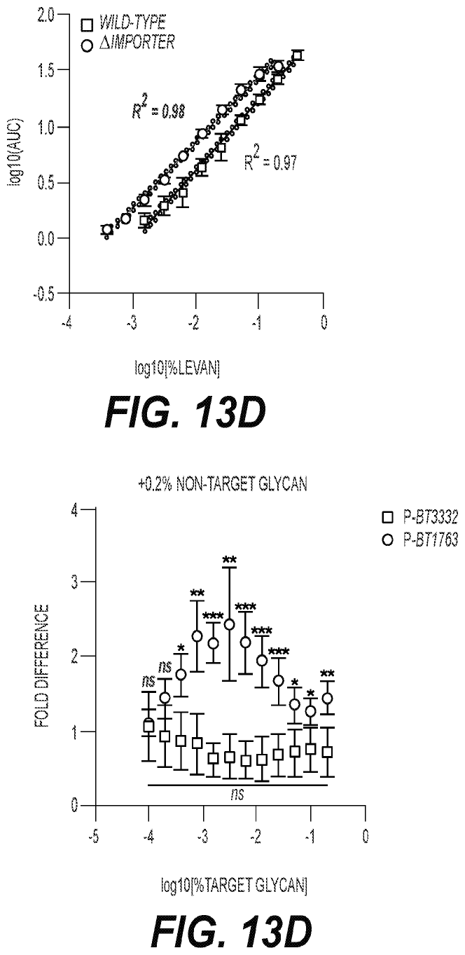

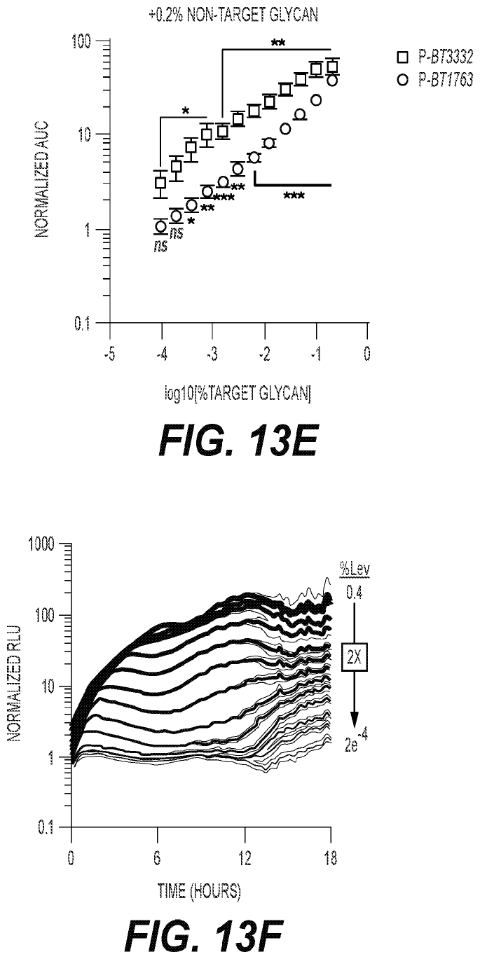

PUL reporters exhibit linear dose-dependent responses. To determine if dose-dependent PUL transcriptional responses elicited by target glycans (FIGS. 1G and 8C) are reflected by the corresponding PUL-reporter activity, 2-fold serial dilutions of either CS or levan were supplied to Bt strains harboring either the CS- or fructan-responsive reporter plasmids described above. A wild-type Bt strain containing P-BT3332 exhibited increased reporter activity when supplied between 0.0001% (1 μg/mL) and 0.4% (4 mg/mL) CS relative to identical cultures supplied only galactose (FIG. 6A). Similarly, a wild-type Bt strain containing P-BT1763 displayed concentration-dependent reporter activity increases when supplied between 0.0016% (16 μg/mL) and 0.4% levan (FIG. 13A). Quantifying the area under each curve (AUC) revealed that the CS- and levan-reporter responses increase linearly within these ranges (FIGS. 6B and 6C) and the logarithm of corresponding AUC values follows a linear regression model with an R2 of 0.95 and 0.97, respectively, when supplied their target ligands (FIGS. 13B and 13C). Reporter strains respond specifically to their corresponding target glycan because neither the CS- or levan-responsive strains exhibited significantly increased luminescence when supplied with any concentration of levan or CS, respectively (FIG. 6D). These data show that the linear, concentration-dependent reporter activity increases exhibited by engineered Bt strains in response to their target glycans could be used to estimate target glycan abundance in an unknown sample concentration. However, bacterial species including Bt exhibit hierarchical glycan utilization, facilitating the prioritized expression of genes necessary for the consumption of more-over less-preferred substrates, hindering target-glycan detection in PUL-reporter strains. However, the CS-reporter strain produced similar dose-dependent responses in the presence of levan (FIG. 13D) and exhibited an identical linear range of detection relative to those collected in the absence of levan (FIG. 13E). Interestingly, the levan-responsive strain exhibited greater reporter activity responses to levan in the presence of 0.2% CS (FIG. 13D) expanding the lower range of responsiveness down to 0.0004% (4 μg/mL) levan and suggesting that the presence of CS may enhance luciferase activity. Consistent with this notion, the addition of 0.2% CS significantly increased the levan-responsive reporter activity when supplied with 0.2% levan (FIGS. 6E and 13D) but did not significantly change fructan-reporter activity in the absence of levan (FIG. 6D). Taken together, these data show that PUL reporters can indicate the presence of target substrates in glycan mixtures.

Sensor-dependent PUL transcription by target glycans is typically resolved by negative feedback mechanisms whereby PUL-encoded activities remove the glycan-derived, PUL-sensor ligand from the periplasm, thereby reducing PUL sensor activation (FIG. 1A). For example, the CS-responsive PUL relies on a glucuronyl hydrolase (gh) to hydrolyze di0S into its monosaccharide components N-acetyl galactosamine and 5-keto 4-deoxyuronate, thereby removing the sensor ligand and reducing transcription activation. Therefore, a mutant lacking BT3348 exhibits increased PUL-transcription relative to wild-type Bt as the CS-sensor is deactivated by this activity. Accordingly, a gh-deficient strain harboring P-BT3332 exhibits detectable responses between 0.000006% (6 ng/ml) and 0.4% CS (FIG. 6F) but the linearity of these responses reached a maximum at 0.0125% (125 μg/mL) (FIGS. 6B and 13B). Similarly, a strain lacking the inner-membrane fructose importer, BT1758, harboring P-BT1763 exhibited increased luminescence at levan concentrations as low as 0.0004% and this response achieved its maximum at 0.2% levan (FIG. 6C) limiting the upper range of its linear responsiveness (FIG. 13C). Thus, PUL-reporter activity can be genetically tuned to extend target glycan sensitivity by preventing PUL-sensor deactivation, which horizontally shifts the linear ranges of glycan detection. Collectively, this data show PUL-reporters offer new specific and highly sensitive glycan detection reagents that estimate glycan abundances across wide linear ranges spanning more than 3 orders of magnitude.

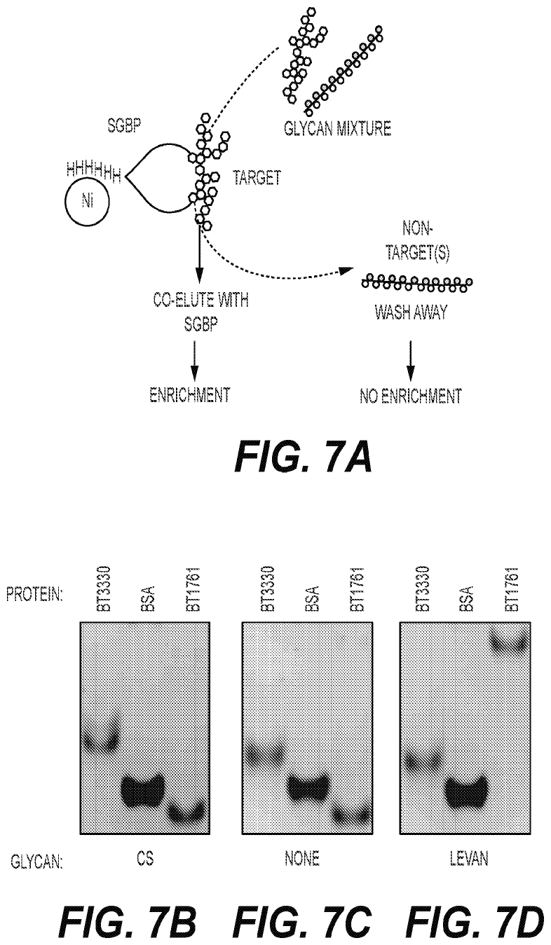

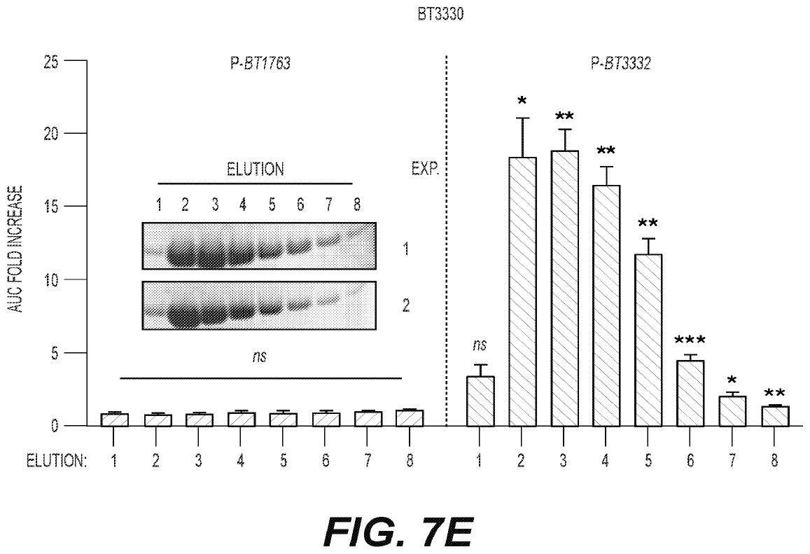

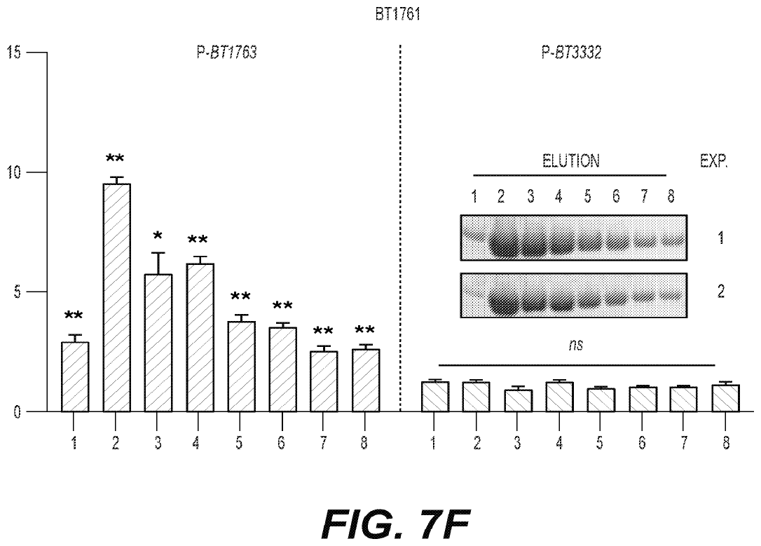

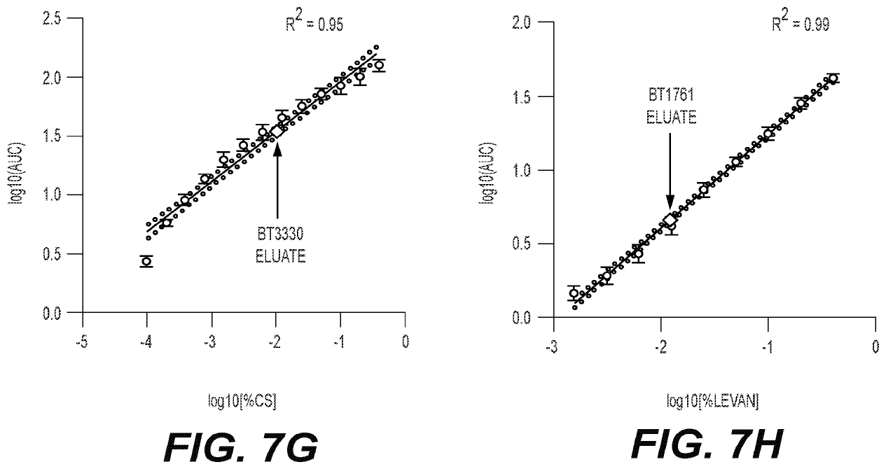

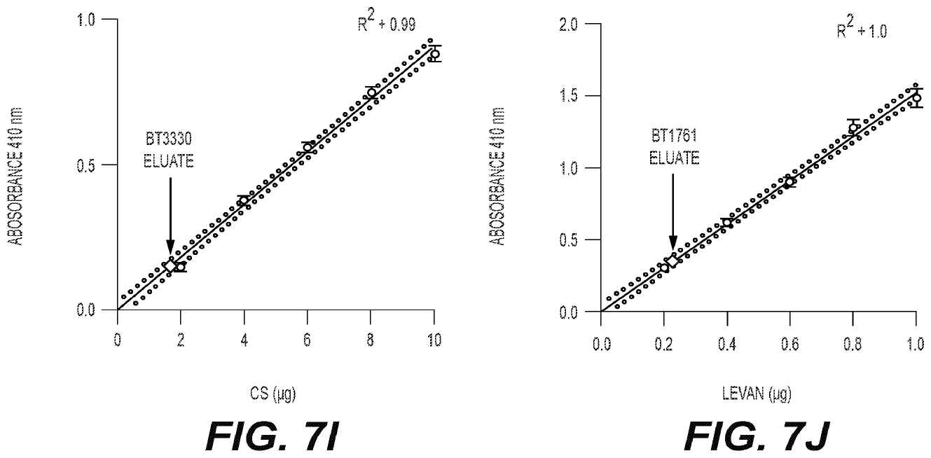

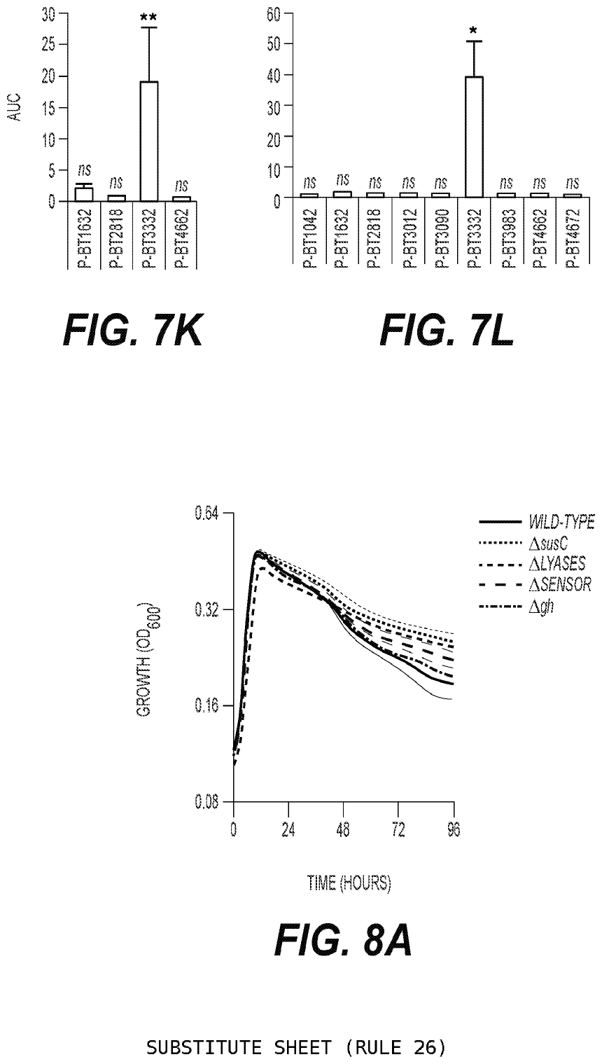

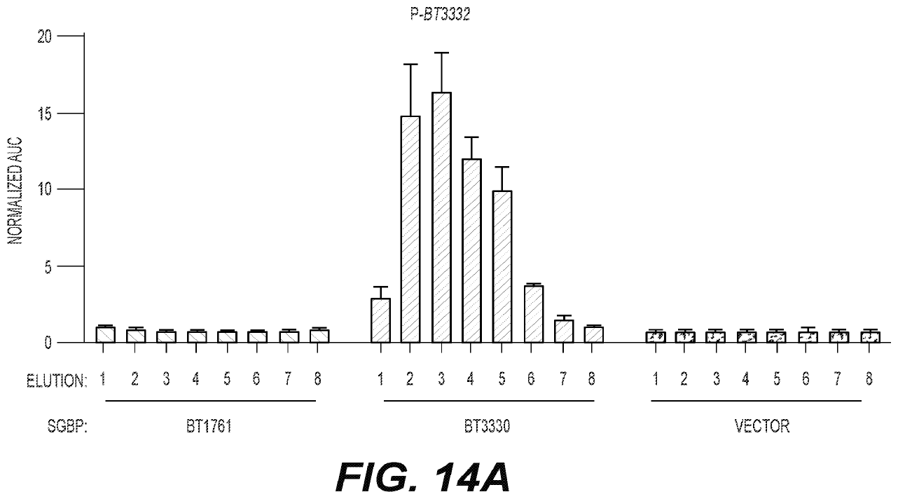

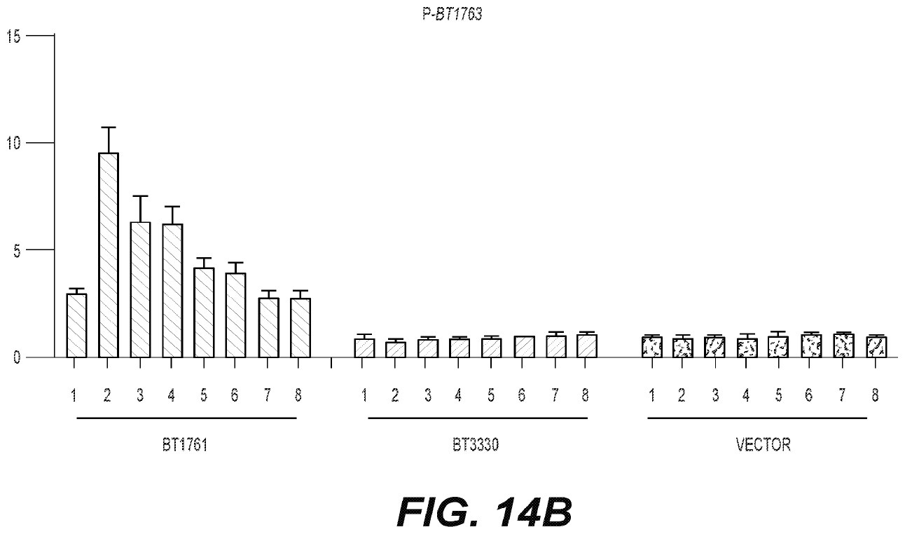

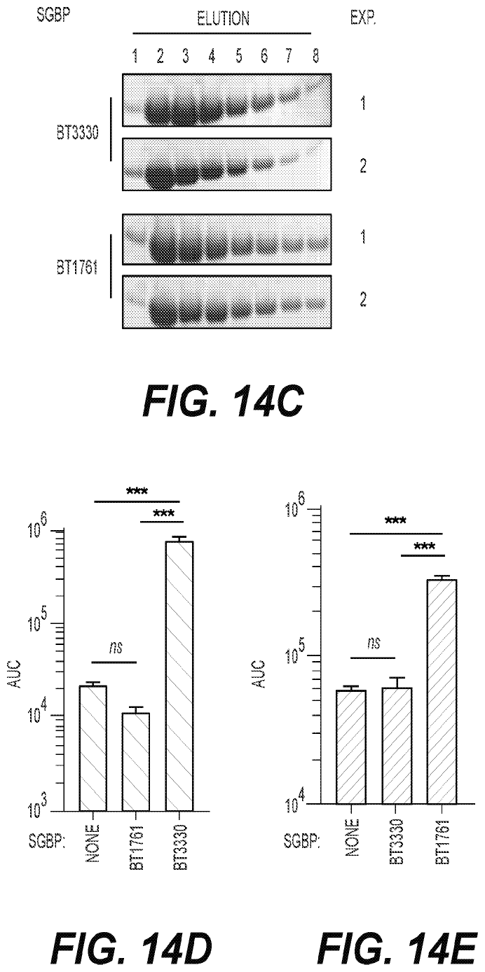

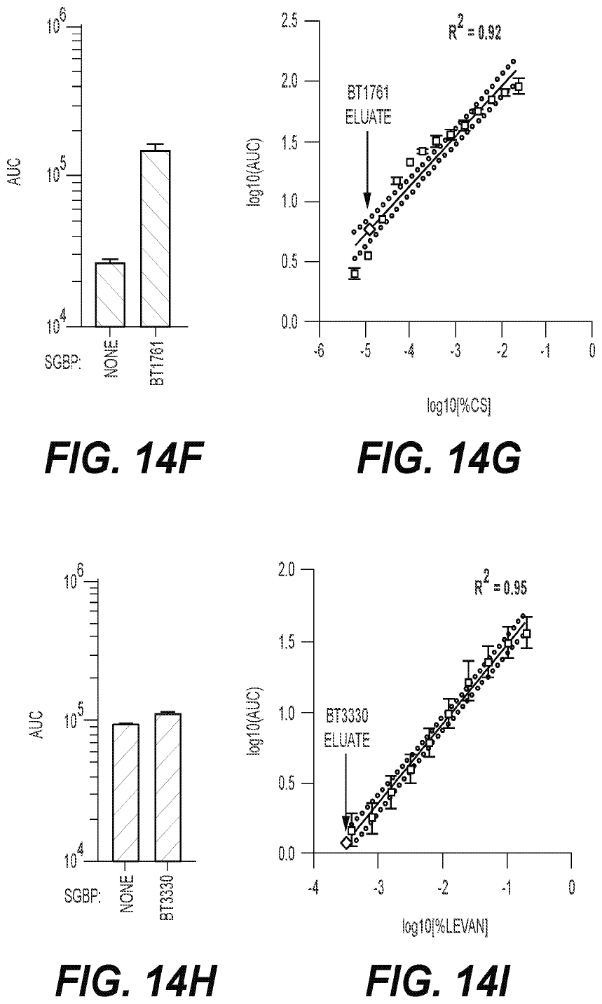

PUL-reporters facilitate target glycan isolation using SGBPs. PULs often encode SGBPs that facilitate glycan sequestration along the outer membrane (FIG. 1A). SGBPs specifically bind their cognate glycan ligands and can discriminate between compositionally similar, structurally distinct glycans. For example, BT1761, an SGBP encoded in the Bt levan utilization PUL, binds to levan but not inulin and BT3330, an SGBP encoded in the Bt CS utilization PUL differentially binds CS polymers greater than 20 disaccharide units in size. Consistent with these findings, recombinant BT3330 protein exhibited a reduced migration following affinity-PAGE in the presence of CS (FIG. 7A) but not in its absence (FIG. 7B). Conversely, BT1761 protein exhibited a decreased relative migration following affinity-PAGE in the presence of levan (FIG. 7C) but not in its absence (FIG. 7B). Furthermore, the altered migration of both proteins was specific to the presence of target glycans because BT3330 migration was similar in the presence of levan (FIG. 7C) relative to gels lacking glycan (FIG. 7B) and BT1761 migration in CS-containing gels (FIG. 7A) was similar in the absence of glycan (FIG. 7B). These results indicate that each PUL-encoded SGBP can distinguish between target and non-target glycans in vitro and putatively facilitate target glycan isolation from a heterogenous mixture. PUL-reporter strains were thought to exhibit sensitive (FIGS. 6B and 6C) and specific (FIG. 6D) detection of target glycans even in the presence of non-target molecules (FIGS. 13D and 13E), they could serve as glycan detection reagents to identify fractions enriched for glycans of interest using immobilized, recombinant SGBPs. Thus, cell lysates were combined and prepared from E. coli strains engineered to over-express either N-terminally his-tagged BT1761 or BT3330 with an equal mixture of levan and CS. After incubation, SGBPs were captured using Ni-agarose, washed extensively, and subsequently eluted with free histidine so that each fraction could be supplied directly to Bt cultures harboring either P-BT3332 or P-BT1763. Elutions 2 through 8 from columns containing BT3330 elicited significantly increased CS-responsive reporter activity compared to control reactions incubated with lysates prepared from E. coli strains harboring an empty vector (FIGS. 7D and 14A). Conversely, identical fractions supplied to the fructan-responsive reporter strain resulted in activity that was indistinguishable from those eluted from vector control containing lysates (FIGS. 7D and 14B) indicating that BT3330-containing fractions (FIG. 14C) were enriched for CS over levan. Conversely, fractions co-eluting with BT1761 (FIG. 14C) elicited increased activity from the levan-(FIGS. 7E and 14B) but not the CS-responsive reporter strain relative to elutions from a vector control (FIGS. 7E and 14A), indicating BT1761 enriched levan over CS.

To measure SGBP-mediated target glycan enrichment, pooled fractions co-eluting with either BT3330 or BT1761 that elicited significantly increased reporter activity compared to control fractions were concentrated (FIGS. 7D and 7E). The concentrated material was supplied to Bt strains harboring CS- or levan-reporter plasmids and compared to a standard curve of 2-fold dilutions of either CS- or levan (FIGS. 7F and 7G, respectively). Glycans co-eluting with BT3330 elicited increased activity from a wild-type Bt strain harboring P-BT3332 (FIGS. 7F and 14D) but not P-BT1763 (FIG. 14E). Conversely, glycans co-eluting with BT1761 stimulated levan-(FIGS. 7G and 14E) but not CS-responsive reporter activity (FIG. 14D). The BT3330 eluate contained 205.6 μg/mL CS and the BT1761 eluate contained 244.4 μg/mL levan using a simple linear regression of log 10 reduced AUC values from corresponding standard curves (FIGS. 7F and 7G, respectively). To verify these results with reporter-independent methods, total fructan or glycosaminoglycan content was measured from identical samples using colorimetric assays (Biovision and Megazyme, respectively), which indicated that concentrated eluates co-purifying with BT3330 containing 168.2 μg/mL CS (FIG. 7H) but no detectable levan and material co-purifying with BT1761 contained 229.7 μg/mL levan (FIG. 7I) but no detectable CS. To estimate the concentration of non-target glycans co-purifying with each SGBP, the concentrated eluate was supplied to hyper-sensitive strains defective for sensor deactivation, which exhibit extended target glycan detection ranges (FIGS. 13B and 13C). The concentrated eluates from BT1761 increased CS-responsive reporter activity in the gh-deficient strain harboring P-BT3332 (FIG. 14F) within the linear range of detection (FIG. 14G) indicating that 0.0000086% (86.7 ng/ml) CS co-purified with BT1761, representing a 939.7-fold enrichment for levan over CS. However, an estimate of the SGBP-mediated enrichment of CS over levan was not obtained because the concentrated BT3330 eluate stimulated 1.2-fold increased levan-responsive reporter activity in the fructose importer-deficient strain harboring P-BT1763 (FIG. 14H) but this was outside of the linear range of quantification (FIG. 14I). Collectively, these data demonstrate that PUL reporters are powerful detection tools that can facilitate target glycan isolation from heterogenous mixtures and measurement beyond the limits of commercially available solutions.

### Discussion

This disclosure provides a new reporter system that kinetically monitors transcription during anaerobic growth in prominent human gut commensal bacteria. Here, introduction of pBolux plasmids containing PUL promoters generates glycan-sensitive bacterial strains that require PUL-encoded transport, depolymerization, and detection activities (FIGS. 3-5). It is demonstrated that Bacteroides PUL sensors elicit concentration-dependent transcriptional responses (FIGS. 1G and 8C) that are recapitulated by corresponding reporter strains (FIGS. 6 and 13) and can indicate the presence of corresponding target molecules at concentrations below the limits of detection of commercially available kits (FIGS. 7F-7I). Moreover, it is demonstrated that reporter sensitivity can be genetically tuned to respond by glycan degree of polymerization by disabling glycan depolymerization machinery (FIGS. 3-4) or achieve lower limits of detection disabling PUL-encoded genes necessary for PUL deactivation (FIGS. 6F, 13B, and 13C-13F). It is also demonstrated that reporter responses are readily quantifiable and can estimate unknown target glycan concentrations (FIGS. 7F and 7G) similar to reporter-independent methods (FIGS. 7H and 7I). Thus, this example establishes a new glycomics toolset that employs Bacteroides PULs to facilitate simple, rapid, and high-throughput detection and quantification of gut microbial substrates in heterogeneous mixtures.

Examining bacterial glycan utilization has previously relied on detectable growth on commercially purified glycans or steady-state transcriptional changes following introduction of undefined glycan mixtures. It is demonstrated that PUL reporters can indicate bacterial transcriptional responses to target glycans in dramatically smaller volumes (as low as 30 μL) (FIGS. 6 and 7) and require substantially less material than similar measurements using ex vivo approaches such as qPCR, transcriptomics, or NanoLuc. Furthermore, pBolux-derived reporter strains overcome the need for kinetic sampling of bacterial cultures because changes in gene expression can be measured over time into late stationary phase (FIGS. 1I-1J) and can indicate target glycans independently of their utilization (FIGS. 1C, 1E, 3D, and 4B). Finally, because these strains can be cultured ad infinitum and do not require harvesting mRNA or cDNA synthesis, they offer a high-throughput, cost-effective tool to measure changes in gene transcription during growth. These advantages render pBolux a powerful, efficient, and relatively inexpensive tool to examine Bacteroides gene transcription in various conditions including in response to glycans.

To demonstrate the advantages of pBolux-derived plasmids, two PUL-reporters were leveraged to devise an affinity isolation approach whereby recombinant PUL-encoded SGBPs isolate target glycans from a mixture (FIG. 7). Because the corresponding reporter strains can specifically and sensitively detect target glycans even in the presence of non-targets (FIGS. 6D, 6E, 13D, and 13E), fractions containing two distinct SGBPs copurifying with miniscule amounts of their corresponding glycan ligands were identified (FIGS. 7A and 7B). Furthermore, using a strain with extended limits of target glycan detection, contaminating non-target glycan abundance demonstrated that this approach achieved 940-fold enrichment of target from non-target molecules (FIGS. 14F and 14G). It is contemplated that similar approaches could be implemented to isolate and characterize unknown PUL-targets such as those derived from the mammalian mucosa, which can exert prebiotic effects when administered in aggregate, for downstream structural characterization. In total, the dedicated glycan utilization machinery possessed by gut microbes has been harnessed to develop tools that enable simple, efficient, and inexpensive glycan detection, isolation and quantification tools that are readily scalable and distributable.

It is demonstrated how glycan-responsive reporters can harness species-specific glycan preferences to detect structurally distinct yet compositionally identical glycans (FIG. 4-5). With some Bacteroides species encoding over one hundred different PULs, many with unknown glycan targets, the accessible glycome of intestinal microbes is vast and largely uncharacterized. Moreover, some annotated PULs putatively facilitate utilization of unknown glycan substrates that confer species-specific gut colonization advantages, which represent attractive therapeutic targets and be identified by implementing analogous approaches to those described here. Because PUL reporters can indicate the presence of target glycans at extremely low abundances (FIGS. 6B&C,S6B&C FIGS. 6B, 6C, 13B, and 13C), without serving as growth substrates (FIGS. 3D,4B&C,5E&F FIGS. 3D, 4B, 4C, 5E, and 5F), and even in the presence of other glycans (FIGS. 6D&E,S6D&E FIGS. 6D, 6E, 13D, and 13E), PUL reporters used to efficiently screen various mixtures for the presence of target glycans. Thus, constructing pBolux plasmids containing promoters from PULs with unknown targets could be used to examine differential glycan abundances across low-yield biological samples in high-throughput applications enabling new glycan surveillance approaches.

### Methods

Bacterial culture. All E. coli strains were cultured on LB agar (BD) aerobically at 37° C. and inoculated from single colonies into LB media (BD) with agitation at 275 rpm. All Bacteroides strains were cultured on brain-heart infusion agar (BHI, Sigma) containing 5% horse blood (Hardy) anaerobically and inoculated from single colonies into TYG incubated under identical conditions. Bacteroides strains were sub-cultured at the indicated dilutions from stationary phase growth in TYG into minimal media containing the indicated carbon sources described in the corresponding figure legends. All strains were cultured in the presence of antibiotic selection where appropriate at the following concentrations: 100 μg/mL ampicillin, 2 μg/mL tetracycline, 25 μg/mL erythromycin.

Growth Measurements. Bacteroides strains were cultured anaerobically to stationary phase in TYG and diluted 1:200 into pre-reduced minimal media containing the indicated carbon sources [glucose, galactose, fructose arabinose, xylose, and chondroitin sulfate (Sigma); levan and inulin (Megazyme)] in pre-reduced 96-well or 384-well clear microplates (Corning). To remove free fructose from inulin, a 1% inulin solution was passed over a ZebaSpin de-salting column (ThermoFisher) prior to the addition of cultures. Kinetic absorbance measurements were taken at 600 nm every 15 minutes for 96 hours using a Tecan Infinite M-plex maintained at 37° C. in a Coy anaerobic chamber with a 2.5% hydrogen atmosphere.

Strain construction. A plasmid encoding the Psuedorhabdus luminescens lux cassette under control of the B. thetaiotaomicon rpoD promoter (BT1311) was generated by amplifying products from pmini-Tn5 luxCDABE Tc using primers W3115 and W3124 and PBT1311-rpiL* from strain NS340 with primers W2952 and W2905. The resulting amplicons were assembled with pNBU2-tetQ digested with BamHI-HF and SalI-HF (NEB) using the NEBuilder Assembly Master Mix (NEB). The resulting plasmid (pNBU2-lux-Pl) was verified by Sanger sequencing before its introduction into B. thetaiotaomicron by di-parental mating to generate strain GT962, and the insert was sub-cloned into pLYL01 using BamHI-HF and SalI-HF to generate plux-Pl.

To generate a B. thetaiotaomicron optimized lux cassette, luxC (primers W2952 and W3265), luxD (primers W3266 and W3267), luxE (primers W3268 and W3269), luxA (primers W3270 and W3271) and luxB (primers W3272 and W3273) were amplified from GT962 genomic DNA and assembled with pNBU2-tetQb digested with BamHI-HF and SalI-HF (NEB) using the NEBuilder Assembly Master Mix (NEB). The resulting plasmid (pNBU2-lux-Bt) was verified by Sanger sequencing before being sub-cloned into pLYL01 using BamHI-HF and SalI-HF to generate plux-Bt. A promoter-less Bacteroides optimized lux cassette was generated by assembling the lux cassette from plux-Bt, amplified with primers 1080 and 1011, with pLYL01 digested with BamHI (NEB) and SalI-HF (NEB) using the NEBuilder Assembly Master Mix (NEB). The resulting plasmid, pBolux, contains tandem BamHI and SpeI restriction sites for cloning and was used for all experiments as a negative control. To construct chondroitin sulfate, levan, and inulin-responsive reporters, the 300 bp region upstream of BT3332 (primers 1232 and 1373), BT1763 (primers 1150 and 1304), or (primers 1943 and 1944) were amplified and combined with pBolux digested with BamHI-HF and SpeI-HF (NEB) using the NEBuilder Assembly Master Mix (NEB).

Generating loss-of-function mutations in Bt were performed using pEXCHANGE-tdk or pKNOCK-ermGb. A Bo strain lacking BACOVA_04496 was constructed using a similar allelic exchange method with pSIE1. All mutations were verified by Sanger sequencing across the chromosomal region of interest. All strains, plasmids and primers are listed in Tables 1-4, 5a, and 5b, respectively.

TABLE 1
Strains used.

| | | |
|---|---|---|
| Name | Genotype | Plasmid |

E. coli

| | | |
|---|---|---|
| S17-1 | λpir | — |
| BL21 (DE3) | E. coli B dcm ompT hsdS(rB-mB-) gal λDE3 | — |

B. thetaiotaomicron

| | | |
|---|---|---|
| GT23 | Δtdk | — |
| VR69 | Δtdk ΔBT3348 | — |
| GT150 | Δtdk ΔBT3334 | — |
| GT165 | Δtdk ΔBT1754 | — |
| GT962 | Δtdk | pNBU2-lux-Pl |
| GT1059 | Δtdk | pNBU2-lux-Bt |
| GT3137 | Δtdk | p-lux-Pl |
| GT1541 | Δtdk | p-lux-Bo |
| GT1866 | Δtdk | pLYL01 |
| GT1867 | Δtdk | pBolux |
| GT1893 | Δtdk | P-BT1763 |
| GT1934 | Δtdk | P-BT3332 |
| GT2111 | Δtdk att-1:pNBU2-ermR | — |
| GT2618 | Δtdk ΔBT3334 | P-BT3332 |
| GT2620 | Δtdk ΔBT1754 | P-BT1763 |
| GT2926 | Δtdk ΔBT3332 | — |
| GT2939 | Δtdk ΔBT3332 | P-BT3332 |
| GT3086 | Δtdk ΔBT3324 ΔBT3350 BT4410::pKNOCK-ermGb | — |
| GT3102 | Δtdk ΔBT3348 | P-BT3332 |
| GT3117 | Δtdk ΔBT3324 ΔBT3350 BT4410::pKNOCK-ermGb | P-BT3332 |
| GT3181 | Δtdk ΔBT1760 | — |
| GT3192 | Δtdk ΔBT1760 | P-BT1763 |
| GT3196 | Δtdk ΔBT1763 | — |
| GT3199 | Δtdk ΔBT1763 | P-BT1763 |
| GT3215 | Δtdk ΔBT1760 att-1::pNBU2-ermGb | — |
| GT3216 | Δtdk ΔBT1760 att-1::pNBU2-ermGb::P_{BT1763}-BT1760 | — |
| GT3226 | Δtdk ΔBT1759 | — |
| GT3246 | Δtdk ΔBT1759 | P-BT1763 |
| GT3282 | Δtdk BT1765::pKNOCK-ermGb | — |
| GT3299 | Δtdk BT1765::pKNOCK-ermGb | P-BT1763 |
| GT3303 | Δtdk ΔBT3082 | — |
| GT3308 | Δtdk ΔBT1760-59 ΔBT3082 | — |
| GT3346 | Δtdk ΔBT1760 ΔBT3082 BT1765::pKNOCK-ermGb | — |
| GT3347 | Δtdk ΔBT1759 ΔBT3082 BT1765::pKNOCK-ermGb | — |
| GT3348 | Δtdk ΔBT1760-59 ΔBT3082 BT1765::pKNOCK-ermG | — |
| GT3358 | Δtdk ΔBT1759 ΔBT3082 BT1765::pKNOCK-ermGb | P-BT1763 |
| GT3356 | Δtdk ΔBT1760 ΔBT3082 BT1765::pKNOCK-ermGb | P-BT1763 |
| GT3360 | Δtdk ΔBT1760-59 ΔBT3082 BT1765::pKNOCK-ermG | P-BT1763 |
| GT3379 | Δtdk BT1758:pKNOCK-ermGb | — |
| GT3393 | Δtdk BT1758:pKNOCK-ermGb | P-BT1763 |
| GT3534 | Δtdk ΔBT3082 | P-BT1763 |

Bacteroides ovatus (ATCC 8483)

| | | |
|---|---|---|
| ATCC 8483 | — | — |
| GT3173 | — | P-BACOVA 04505 |
| GT3179 | BACOVA_04495::pSIE1::ΔBACOVA_04496 | — |
| GT3180 | — | pBolux |
| GT3183 | ΔBACOVA_04496 | — |
| GT3189 | ΔBACOVA_04496 | P-BACOVA_04505 |
| GT3190 | ΔBACOVA 04496 | pBolux |
| GT3489 | — | P-BACOVA 04505 |
| GT3490 | — | pBolux |

TABLE 2
Primers used.

| | | | |
|---|---|---|---|
| identifier | name | sequence (5′ → 3′) | purpose |

qPCR

| | | | |
|---|---|---|---|
| 1044 | qBT16s_f | ggtagtccacacagtaaacgatgaa (SEQ ID NO: 1) | measure 16s rRNA levels using |
| 1045 | qBT16s_r | cccgtcaaattcctttgagtttc (SEQ ID NO: 2) | qPCR |
| 1060 | qBT3332_f | tggttgtcggctatcaggaagt (SEQ ID NO: 3) | measure BT3332 mRNA levels |
| 1061 | qBT3332_r | acatctgccatgttggctttc (SEQ ID NO: 4) | using qPCR |
| 1056 | qBT1763_f | agcgtaaagccgacctgaca (SEQ ID NO: 5) | measure BT1763 mRNA levels |
| 1057 | qBT1763_r | tcaccttgcttctggatttcg (SEQ ID NO: 6) | using qPCR |

lux reporter construction

| | | | |
|---|---|---|---|
| w2952 | pNBU-P- | gctctagaactagtggatcctgatctggaagaagcaatg | clone the Bt rpoD promoter |
| | BT1311_f | aaagct (SEQ ID NO: 7) | preceding the_rpiL* RBS |
| w2905 | rpiL*_r | catattcgtttaattaaataaataatttattttattttaaa | |
| | | (SEQ ID NO: 8) | |
| w3115 | rpiL *- | atttatttaattaaacgaatatgactaaaaaaatttcattcat | clone the P. luminescens lux |
| | luxC_f | tattaacgg (SEQ ID NO: 9) | cassette preceded by the rpiL* |
| w3124 | luxE-nbu_r | aagataggcaattagtcgactcaactattaaatgcttggt | RBS into pNBU2 |
| | | ttaagcttaa (SEQ ID NO: 10) | |
| w3265 | luxC_r | tttacaatttgccatgcggattacgggacaaatacaagg | clone the Bacteroides-optimized |
| | | aacttatc (SEQ ID NO: 11) | lux cassette preceded by the |
| w3266 | luxD_f | tccgcatggcaaattgtaaattgtaaatcgtaaaatagta | rpiL* RBS into pNBU2 |
| | | atatattaatggaaaataaatccaaatataaaaccatc | |
| | | (SEQ ID NO: 12) | |
| w3267 | luxD_r | attctttatcctcctccttattaagacagcgaaatcgcttga | |
| | | (SEQ ID NO: 13) | |
| w3268 | luxE_f | taaggaggaggataaagaatatgacttcatatgttgata | |
| | | aacaagagatc (SEQ ID NO: 14) | |
| w3269 | luxE_r | agtgttaccttcattcatccttcttcacccttcatttatatca | |
| | | actattaaatgcttggtttaagctt (SEQ ID NO: | |
| | | 15) | |
| w3270 | luxA_f | ggatgaatgaaggtaacactcataaactcgaaattcttc | |
| | | attcttaatttttaattaaaatatatgaaatttggaaacttttt | |
| | | gcttac (SEQ ID NO: 16) | |
| w3271 | luxA_r | ccattgtcttattcctttctttataatagcgaacgttgtttttct | |
| | | ttaag (SEQ ID NO: 17) | |
| w3272 | luxB_f | agaaaggaataagacaatggatatgaaatttggattgtt | |
| | | cttccttaac (SEQ ID NO: 18) | |
| w3273 | luxB_r | aagataggcaattagtcgacttacatgtggtactttttaat | |
| | | attatcatcaacaa (SEQ ID NO: 19) | |
| 1080 | pLYL- | gctcggtacccggggatccactagtcactcccgcatttt | clone the Bacteroides-optimized |
| | SpeI- | aaaataaaataaattatttatttaattaaacg (SEQ ID | lux cassette into pLYL01 and |
| | rpil*_f | NO: 20) | introduce a SpeI site |
| 1011 | PLYL | tgcatgcctgcaggtcgacttacatgtggtactttttaata | |
| | luxB_r | ttatcatcaacaatattg (SEQ ID NO: 21) | |
| 1081 | pBolux-P- | gctcggtacccggggatcctgatctggaagaagcaatg | clone the Bt rpoD promoter into |
| | BT1311_f | aaagct (SEQ ID NO: 22) | pBolux |
| 1082 | pBolux-P- | aaatgcgggagtgactagtcaaagttacgacaaataatt | |
| | BT1311_r | tgttaacatacatatttaggc (SEQ ID NO: 23) | |
| 2109 | pBolux-Bo- | gctcggtacccggggatccatctggaagaagtaatgaa | clone the Bo_rpoD promoter into |
| | PrpoD_f | agctgc (SEQ ID NO: 24) | pBolux |
| 2110 | pBolux-Bo- | aaatgcgggagtgactagtcaaagttacgacaaataatt | |
| | PrpoD_r | tgttaacatacaaaa (SEQ ID NO: 25) | |
| 1150 | pBolux- | gctcggtacccggggatcctatcattcagttttctgttggt | clone the BT1763 promoter into |
| | pBT1763_f | tactttgagiga (SEQ ID NO: 26) | pBolux |
| 1304 | pBolux- | aaatgcgggagtgactagttagtttaatgttattaatttaa | |
| | pBT1763_r | aagtacgaattttctcttttcgatg (SEQ ID NO: | |
| | | 27) | |
| 1232 | pBolux- | gctcggtacccggggatccaaaatggaactgggcaat | clone the BT3332 promoter into |
| | pBT3332_f | gacagg (SEQ ID NO: 28) | pBolux |
| 1373 | pBolux- | aaatgcgggagtgactagtcctttttcttgtctggttggat | |
| | pBT3332_r | agatgtttttt (SEQ ID NO: 29) | |
| 1943 | pBolux- | gctcggtacccggggatccttgtttgttggagattgttttc | clone the BACOVA 04505 |
| | pBACOVA_ | atatcgttg (SEQ ID NO: 30) | promoter into pBolux |
| | 04505_f | | |
| 1944 | pBolux- | aaatgcgggagtgactagttagtttgatgttattaaattaa | |
| | pBACOVA_ | aagtacgaattttac (SEQ ID NO: 31) | |
| | 04505_r | | |

Engineering mutations

| | | | |
|---|---|---|---|
| 1846 | pEXC- | gctctagaactagtggatccgacagcctccagctgacg | engineer a chromosomal deletion |
| | ΔBT3332_ | g (SEQ ID NO: 32) | of BT3332 |
| | 5f | | |
| 1535 | pEXC- | catcctttttcttgtctggttggat (SEQ ID NO: 33) | |
| | ΔBT3332_ | | |
| | 5r | | |
| 1847 | pEXC- | accagacaagaaaaaggatgaaagcattaaaaataac | |
| | ΔBT3332_ | aatcatagctctattggca (SEQ ID NO: 34) | |
| | 3f | | |
| 1848 | pEXC- | aagataacattcgagtcgacatagaagctggctctttcg | |
| | ΔBT3332_ | aaatagtc (SEQ ID NO: 35) | |
| | 3r | | |
| 11492 | pEXC- | cgggatccgtggactacttttgctgaaagcgga (SEQ | engineer a chromosomal deletion |
| | ΔBT1754_ | ID NO: 36) | of BT1754 |
| | 5f | | |
| 11493 | pEXC- | tccccccgggttcatagttctttctgtaatccaattaaga | |
| | ΔBT1754_ | (SEQ ID NO: 37) | |
| | 5r | | |
| 11494 | pEXC- | tccccccgggtttcattgatatcgtaaagagggat | |
| | ΔBT1754_ | (SEQ ID NO: 38) | |
| | 3f | | |
| 11495 | pEXC- | acgcgtcgactgccacacttccgtgcactt (SEQ ID | |
| | ΔBT1754_ | NO: 39) | |
| | 3r | | |
| 1880 | pEXC- | gctctagaactagtggatcccagtaatgaagagacatta | engineer a chromosomal deletion |
| | ΔBT1763_ | cgg (SEQ ID NO: 40) | of BT1763 |
| | 5f | | |
| 1819 | pEXC- | tagtttaatgttattaatttaaaagtacgaattttctcttttcg | |
| | ΔBT1763_ | atg (SEQ ID NO: 41) | |
| | 5r | | |
| 1839 | pEXC- | aaattaataacattaaactaatgaaaaagataatatatata | |
| | ΔBT1763_ | gcaacaatcggaattacc (SEQ ID NO: 42) | |
| | 3f | | |
| 1840 | pEXC- | aagataacattcgagtcgacctgtttcaggtcttcttcgtt | |
| | ΔBT1763_ | gattcc (SEQ ID NO: 43) | |
| | 3r | | |
| 1972 | pEXC- | gctctagaactagtggatccgcttcttccgtcagtcttct | engineer a chromosomal deletion |
| | ΔBT1760_ | (SEQ ID NO: 44) | of BT1760 |
| | 5f | | |
| 1973 | pEXC- | ttatttacacaagtagttgattgcattgagag (SEQ ID | |
| | ΔBT1760_ | NO: 45) | |
| | 5r | | |
| 1974 | pEXC- | tcaactacttgtgtaaataatgaaaactacaccggcaagt | |
| | ΔBT1760_ | aacatc (SEQ ID NO: 46) | |
| | 3f | | |
| 1975 | pEXC- | aagataacattcgagtcgactatcgcaacggggcggtg | |
| | ΔBT1760_ | t (SEQ ID NO: 47) | |
| | 3r | | |
| 2011 | pEXC- | gctctagaactagtggatccctggaagatttgaaagcaa | engineer a chromosomal deletion |
| | ΔBT1759_ | ctac (SEQ ID NO: 48) | of BT1759 |
| | 5f | | |
| 2012 | pEXC- | tcaataagtgcttacctgaacgtctg (SEQ ID NO: | |
| | ΔBT1759_ | 49) | |
| | 5r | | |
| 2013 | pEXC- | ttcaggtaagcacttattgaaaaacgactttcttctccctg | |
| | ΔBT1759_ | c (SEQ ID NO: 50) | |
| | 3f | | |
| 2014 | pEXC- | aagataacattcgagtcgactgctccccacatggcaatg | |
| | ΔBT1759_ | t (SEQ ID NO: 51) | |
| | 3r | | |
| 2015 | pEXC- | tcaactacttgtgtaaataaaaaacgactttcttctccctg | engineer a chromosomal deletion |
| | ΔBT1760- | c (SEQ ID NO: 52) | of BT1760-59 |
| | 59_3f | | |
| 2052 | pEXC- | gctctagaactagtggatccccctctcaattggcgaaag | engineer a chromosomal deletion |
| | ΔBT3082_ | aaaatc (SEQ ID NO: 53) | of BT3082 |
| | 5f | | |
| 2053 | pEXC- | agctattttatttattagtttgtaaaatcggagt (SEQ ID | |
| | ΔBT3082_ | NO: 54) | |
| | 5r | | |
| 2054 | pEXC- | cgattttacaaactaataaataaaatagctacggaaatca | |
| | ΔBT3082_ | aaagctatctttgttttcag (SEQ ID NO: 55) | |
| | 3f | | |
| 2055 | pEXC- | aagataacattcgagtcgacttccactggtaggctcgat | |
| | ΔBT3082_ | g (SEQ ID NO: 56) | |
| | 3r | | |
| 2058 | pKO- | gctctagaactagtggatcctgggaacatttggctcctg | engineer a chromosomal knock- |
| | BT1765_f | c (SEQ ID NO: 57) | out of BT1765 |
| 2059 | pKO- | ggccccccctcgaggtcgacatcattgtcctgtgtatag | |
| | BT1765_r | agtccc (SEQ ID NO: 58) | |
| 2077 | pKO- | cgctctagaactagtggatccagaaaaccgtgttactca | engineer a chromosomal knock- |
| | BT1758_f | gtttgatcg (SEQ ID NO: 59) | out of BT1758 |
| 2078 | pKO- | gggccccccctcgaggtcgacaaataacagagaacac | |
| | BT1758_r | attcgagttacc (SEQ ID NO: 60) | |
| 1978 | pSIE1_ | gattagcattatgaggatcctttggctatcccggcatcga | engineer a chromosomal deletion |
| | ΔBACOVA_ | (SEQ ID NO: 61) | of BACOVA 04496 |
| | 04496_5f | | |
| 1979 | ΔBACOVA_ | tgacgtgaatagttttgatttctcatttttttcgtatttctttcta | |
| | 04496_5r | tgacc (SEQ ID NO: 62) | |
| 1980 | ΔBACOVA_ | aatcaaaactattcacgtcagaatacaataaatc (SEQ | |
| | 04496_3f | ID NO: 63) | |
| 1981 | ASIE1_ | tccaccgcggtggcggccgcgcagtatatacaaatag | |
| | dBACOVA_ | ggttacgtct (SEQ ID NO: 64) | |
| | 04496_3r | | |

BT1760 complementation

| | | | |
|---|---|---|---|
| 1818 | pNBU2- | gctctagaactagtggatcctatcattcagttttctgttggt | Complement the BT1760 deletion |
| | pBT1763_f | tactttgag (SEQ ID NO: 65) | strain in trans |
| 2007 | P-BT1763- | cgtacttttaaattaataacattaaactaatgatgaaaaat | |
| | BT1760_f | atgatcttacctatagcat (SEQ ID NO: 66) | |
| 2008 | pNBU2- | aagataggcaattagtcgactcaataagtgcttacctga | |
| | BT1760_r | acgtc (SEQ ID NO: 67) | |

SGBP over-expression

| | | | |
|---|---|---|---|
| 1723 | pT7-7-H6- | agaaggagatatacatatgcatcaccatcaccatcaca | BT1761 overexpression in BL21 |
| | BT1761_f | gtgatgacttcaaatccggcc (SEQ ID NO: 68) | (DE3) |
| 1724 | pT7-7- | gcttatcatcgataagcttttatttacacaagtagttgattg | |
| | BT1761_r | cattgagag (SEQ ID NO: 69) | |
| 2087 | pT7-6H- | agaaggagatatacatatgcatcaccatcaccatcacg | BT3330 overexpression in BL21 |
| | 4G- | gaggtggaggtgacgggctggacgaagcggtaggt | (DE3) |
| | BT3330_f | (SEQ ID NO: 70) | |
| 2088 | pT7-7- | agcttatcatcgataagcttattccactacgtttaccacca | |
| | BT3330_r | t (SEQ ID NO: 71) | |

TABLE 3
Plasmids used

| Name | Description |
|---|---|
| pLYL01 | empty multi-copy vector |
| pNBU2-erm | empty single-copy vector conferring erythromycin resistance |
| pNBU2-tet | empty single-copy vector conferring tetracycline resistance |
| pNBU-lux-Pl | The Psuedorhabdus luminescens lux cassette cloned into pNBU2-tetR |
| pNBU-lux-Bt | The Bacteroides optimized lux cassette cloned into pNBU2-tetR |
| p-lux-Pl | The Psuedorhabdus luminescens lux cassette sub-cloned into pLYL01 |
| p-lux-Bt | The Bacteroides optimized lux cassette sub-cloned into pLYL01 |
| pBolux | pLYL01 harboring BamHI and SpeI sites upstream of the Bacteroides |
| | optimized lux cassette |
| P-Bt-rpoD | 277 bp upstream of BT1311 were cloned into the BamHI and SpeI sites in |
| | pBolux |
| P-Bo-rpoD | 278 bp upstream of BACOVA_00615 were cloned into the BamHI and SpeI |
| | sites in pBolux |
| P-BT3332 | 300 bp upstream of BT3332 were cloned into pBolux |
| P-BT1763 | 300 bp upstream of BT1763 were cloned into pBolux |
| pBolux::P- | 300 bp upstream of BACOVA_04496 were cloned into pBolux |
| BACOVA_04496 | |
| pEXCHANGE-tdk | empty plasmid used to generate chromosomal deletions in Bt |
| pEXCHANGE- | plasmid used to generate a chromosomal deletion of BT3332 |
| ΔBT3332 | |
| pEXCHANGE- | plasmid used to generate a chromosomal deletion of BT1763 |
| ΔBT1763 | |
| pEXCHANGE- | plasmid used to generate a chromosomal deletion of BT1760 |
| ΔBT1760 | |
| pEXCHANGE- | plasmid used to generate a chromosomal deletion of BT1759 |
| ΔBT1759 | |
| pEXCHANGE- | plasmid used to generate a chromosomal deletion of BT1760-59 |
| ΔBT1760-59 | |
| pEXCHANGE- | plasmid used to generate a chromosomal deletion of BT1754 |
| ΔBT1754 | |
| pEXCHANGE- | plasmid used to generate a chromosomal deletion of BT3082 |
| ΔBT3082 | |
| pKNOCK-ermG | plasmid to generate chromosomal insertions in Bt |
| pKNOCK-ermG- | plasmid used to inactivate BT1765 |
| BT1765KO | |
| pKNOCK-ermG- | plasmid used to inactivate BT1758 |
| BT1758KO | |
| pKNOCK-ermG- | plasmid used to inactivate BT4410 |
| BT4410KO | |
| pSIE1 | empty plasmid to generate chromosomal deletions in Bo |
| pSIE1::ΔBACOVA_{—} | plasmid to delete BACOVA_04495 in Bo |
| 04495 | |
| pT7-7 | empty vector for protein over-expression |
| pT7-7::H6-4G- | pT7-7 construct for over-expression of BT3330 [D21-E347] with an N- |
| BT3330 | terminal hexahistidine tag |
| pT7-7::H6-BT1761 | pT7-7 construct for over-expression of BT1761 [S25-K461] with an N- |
| | terminal hexahistidine tag |

Transcript quantification. mRNA was prepared from 1.0 mL of pelleted Bt cell culture treated with RNA protect (Qiagen) using the RNeasy kit (Qiagen) according to the manufacturer's directions. cDNA was subsequently synthesized from 1.0 μg of isolated RNA using Superscript VILO IV master mix (ThermoFisher) according to the manufacturer's directions. Transcript levels were measured by qPCR using Fast SYBR Green PCR Master Mix (Applied Biosystems) and primers 1060 and 1061 (BT3332) or 1056 and 1057 (BT1763) and monitored using a QuantStudio 12K Flex instrument (Applied Biosystems). All mRNA transcripts were normalized to 16s IRNA measured from 1,000-fold diluted cDNA using primers 1044 and 1045.

Measuring bioluminescence during growth in various monosaccharides. Bt and Bo strains were cultured in TYG containing 2 μg/mL tetracycline overnight before being diluted 200-fold into minimal media containing 0.5% carbon source in a sterile 96-well white, clear-bottomed microplate (Corning 3610). Absorbance at 600 nm and luminescence were measured every 15 minutes for 48 hours using a Tecan Infinite M-plex. RLU were calculated as luminescence values divided by absorbance at 600 nm.

Measuring bioluminescence in response to glycans. Bt and Bo strains were cultured in TYG containing 2 μg/mL tetracycline overnight before being diluted 50-fold into minimal media containing 0.5% galactose anaerobically grown to mid-exponential phase at 37° C. (approximately 4 hours). Cultured cells were pelleted by centrifugation before the supernatant was removed, and cells were resuspended in 2× minimal media lacking a carbon source. Equal volumes of each cell suspension were transferred to a pre-reduced, white, clear bottom 384-well microplate (Corning 3765) containing equal volume of the indicated carbon source. Absorbance at 600 nm and luminescence were measured every 15 minutes for 18 hours using a Tecan Infinite M-Plex instrument anaerobically at 37° C. temperature. RLU were calculated as luminescence values divided by absorbance at 600 nm and normalized to identical measurements from each strain supplied only galactose.

Protein overexpression and purification. E. coli BL21 (DE3) cells were transformed with pT7-7 plasmids encoding SGBPs. Fresh transformants were cultured overnight in LB supplemented with 100 μg/mL ampicillin at 37° C. with agitation. The following day, cultures were diluted 50-fold in LB supplemented with 100 μg/mL ampicillin and incubated for approximately 2 hours at 37° C. with agitation to mid-exponential phase (OD₆₀₀˜0.45-0.6). Cultures were cooled on ice before isopropyl β-thiogalactopyranoside (IPTG) was added to a final concentration of 50 μM and incubated at 30° C. with agitation for 4 hours. Cells were pelleted by centrifugation at 7197×g for 10 min at 4° C. before the supernatant was decanted and cell pellets were frozen on dry ice and stored at −80° C. Pellets were thawed on ice, resuspended in 20 mM Tris/HCl buffer, pH 8.0, containing 100 mM NaCl and lysed in 2.0 mL tubes containing 250 μL volumes of 0.1 mm silica beads placed in a pre-chilled aluminum block using a Bead beater-96 (BioSpec) for 5 cycles of 40 second beating at 2400 rpm and 5 minutes resting at 4° C. Lysates were centrifuged at 20,000×g for 10 minutes at 4° C. and the supernatant was combined with 0.2 mL pre-equilibrated Ni-NTA sepharose and 10 mL of 20 mM Tris/HCl buffer, pH 8.0, containing 100 mM NaCl for 1 hour at room temperature with rocking. The slurry was packed into a 2 mL purification column (Pierce) and the liquid phase removed by gravity flow before washing with 3 mL of wash buffer (20 mM Tris/HCl buffer, pH 8.0, containing 100 mM NaCl). SGBPs were eluted from the column with 3 mL of elution buffer (20 mM Tris/HCl buffer, pH 8.0, containing 100 mM NaCl and 25 mM histidine), concentrated and buffer exchanged using centrifugal concentrators (Millipore) and 6 successive additions of storage buffer (10 mM Tris/HCl buffer containing 10% glycerol). Protein concentrations were estimated by BCA assay (Pierce) and stored at −80° C.

Affinity PAGE. 100 pmol of protein was combined with native PAGE loading buffer and electrophoresed in a 10% polyacrylamide matrix containing 10% (w/v) acrylamide in Tris, glycine buffer, pH 8.8. at 20 mA for 3.5 hours at room temperature. Gels contained 0.1% CS or levan and BSA was used as a negative control. Proteins were visualized by Coomassie Blue staining.

SGBP target glycan isolation. 500 μL of clarified protein lysate was pre-incubated with 1.0 mL of a mixture containing 0.1% levan and 0.1% CS overnight at 4° C. with rocking before combination with 200 μL pre-equilibrated Ni-NTA agarose (Sigma) in 9 mL of 20 mM Tris/HCl buffer, pH 8.0, containing 100 mM NaCl, for 1 hour at RT with rocking. The slurry was packed into a 2 mL purification column (Pierce) and the liquid phase removed by gravity flow before washing 4 times with 1 mL of 20 mM Tris/HCl buffer, pH 8.0, containing 100 mM NaCl. SGBPs were eluted from the column in a total of 4 mL elution buffer (20 mM Tris/HCl buffer, pH 8.0, 100 mM NaCl and 25 mM histidine). Glycan content was measured as described above whereby mid-exponential cells were pelleted by centrifugation and re-suspended into 2× minimal media containing 0.4% galactose. Each cell suspension was transferred to a pre-reduced, white, clear bottom 384-well microplate (Corning 3765) containing equal volumes of column fractions. Absorbance and luminescence were measured kinetically under anaerobic conditions as described above. Target glycan containing elution fractions were combined and treated with 0.1 mg/mL Proteinase K in 10 mM Tris, pH 7.5 containing 20 mM calcium chloride for 2 hours at 65° C. Total glycans were combined with 3 volumes of 200 proof ethanol, incubated overnight with rocking at 4° C., pelleted by centrifugation, and resuspended in one tenth the original volume of water.

Reporter-independent glycan measurements. Total levan content was assayed using the Fructan Assay Kit (K-Fruc, Megazyme), modified to include the hydrolysis and absorbance measurement at 410 nm against a standard curve of 0.2-1 mg levan. CS abundance was estimated using the Total Glycosaminoglycan Assay Kit (K2085, BioVision), according to the manufacturer's directions against a standard curve of 0.2-10 μg CS.

Statistical analysis. Repeated measurements were analyzed by paired, two-tailed Student's t-test, 1-way, or 2-way ANOVA using Graphpad Prism where appropriate as indicated in each figure legend.

### Example 2: Applications for Harnessing Gut Microbes

Glycans facilitate critical biological functions and control the mammalian gut microbiota composition by supplying differentially accessible nutrients to distinct microbial subsets. Therefore, identifying unique glycan substrates that support defined microbial populations will inform new therapeutic avenues to treat diseases via modulation of the gut microbiota composition and metabolism. However, examining heterogenous glycan mixtures for individual microbial substrates is hindered by glycan structural complexity and diversity, which presents substantial challenges to glycomics approaches. Fortuitously, gut microbes encode specialized sensor proteins that recognize unique glycan structures and in-turn activate predictable, specific, and dynamic transcriptional responses. Herein, the microbial machinery is harnessed to indicate the presence and abundance of compositionally similar, yet structurally distinct glycans, using a developed transcriptional reporter. It demonstrated how these tools can be implemented to examine glycan mixtures, isolate target molecules for downstream characterization, and quantify the recovered products. Thus, a toolkit will dramatically enhance the understanding of the mammalian intestinal environment and identify host-microbial interactions critical for human health.

### Example 3: Development of Methods for Detecting and Quantifying Glycans

Modern glycomics approaches face technical and biophysical limitations that hinder comprehensive characterization of heterogeneous glycan mixtures typically derived from biological samples. For example, glycomic analyses by mass spectrometry requires sample derivatization and ionization that bias detection due to the non-uniform labeling of individual monosaccharides, covalent decorations, and branched structures exhibited by many glycans. Conversely, nuclear magnetic resonance (NMR) precisely determines composition, sequence, and structural arrangements but requires isolation of individual glycan species to achieve optimal resolution. Isolation of individual target glycans from a complex mixture requires subsequent rounds of fractionation using sequential high-performance liquid chromatographic (HPLC) approaches to sort compositionally similar but structurally distinct molecules. Glycan binding aptamers and proteins, such as lectins, can directly indicate the presence of distinct targets but their narrow specificity paired with high-cost limits their accessibility as high-throughput glycan surveillance tools. Consequently, glycomics strategies often require the application of multiple, tandem methodologies to comprehensively examine the complex mixtures typically extracted from eukaryotic, prokaryotic, and plant materials. Therefore, new, accessible, and inexpensive technologies are imperative to facilitate simple, rapid surveillance of crude glycan preparations that indicate the presence and abundance of distinct structures.

Collectively, the challenges facing their characterization have limited the understanding of the glycans present in various ecological niches, referred to as the glycome, and has lagged behind the understanding of the abundance and dynamics of the organisms occupying those niches. This is exemplified by the mammalian intestinal environment, where the gut microbiome and its transcriptome have been extensively examined by NGS methodologies that take advantage of the linear, uniform compositions of nucleic acids. Thus, the microbial populations and their transcriptional fluctuations have been well-defined, revealing incredible inter-individual compositional variation and uncovering their contributions to human health and disease. This work has revealed how the intestinal glycome influences gut microbial fitness and metabolism, thereby driving the microbiome composition and the synthesis of clinically important metabolites. Thus, the intestinal glycome is an untapped reservoir of molecules that could be employed to modulate gut microbial abundance and metabolism for clinically beneficial outcomes. New glycomic approaches have been developed to interface with existing technologies to detect glycans that serve as gut microbial signals and growth substrates, and subsequently isolate relevant molecules from biologically-derived mixtures for downstream structural characterization using conventional approaches. Bacterial taxa that dominate in the intestinal environment have evolved specialized glycan detection and utilization machinery to identify available substrates that can serve as nutrient sources and respond by producing enzymes that facilitate substrate sequestration, importation, and consumption. Many of these bacteria employ a panoply of different glycan sensor proteins that recognize structurally distinct glycans and facilitate transcription activation of the appropriate enzymatic repertoire to efficiently utilize target glycans. The glycan detection machinery possessed by gut bacteria has been harnessed to indicate the presence and abundance of distinct glycans in vitro using a new transcriptional reporter system. Furthermore, we implemented this reporter in a high-throughput capacity to define the glycome of a biologically derived heterogenous glycan mixture through the prism of gut microbes. Moreover, this reporter-mediated detection is utilized to facilitate isolation of target glycans of interest using affinity purification strategies for downstream structural and functional characterization. Finally, it is demonstrated that these microbial responses can be readily quantified to estimate the relative abundance of recovered glycans similarly to commercially available products. Therefore, a new suite of glycomics tools have been established that can overcome many of the current challenges facing glycomics and have developed an inexpensive, readily distributable glycan surveillance platform to examine crude glycan preparations in a high-throughput manner and affinity purification tools to isolate target molecules for downstream characterization.

Moreover, because the glycans found in the intestinal environment are derived from a myriad of environmental sources including plant and animal tissue, the tools that result from this example extend far beyond the gut microbiota to enhance existing glycomics workflows. Finally, genetic tuning can modulate the responsiveness of these systems to narrow glycan structural specificity and extend the limits of detection indicating their modularity.

### Results

Bacteroides PUL sensors elicit dose-dependent transcriptional responses to cognate glycan ligands. Species of the bacterial genus, Bacteroides, are predominantly found in the mammalian intestinal tract and encode genetically linked, coordinately regulated gene clusters called polysaccharide utilization loci (PULs). Most Bacteroides species encode many different PULs, each conferring the ability to consume one or a small subset of structurally distinct glycans. PULs encode genes that facilitate the sequestration, importation, depolymerization, and detection of a target glycan (FIG. 1A). PUL transcripts are typically expressed constitutively at low levels until the target glycan is encountered in the environment, at which time PUL transcripts are rapidly and dramatically increased (FIG. 1B). These increases in PUL transcription are mediated by unique sensor proteins that bind mono-, di-, or oligo-saccharide signatures derived from the target glycan in the periplasm directly facilitating transcription of PUL genes in the cytoplasm, an activity necessary for efficient utilization of the target glycan (FIG. 1C). For example, introduction of the glycosaminoglycan, chondroitin sulfate (CS), results in a 92-fold increase in the transcript level of the Bacteroides thetaiotaomicron (Bt) CS-specific PUL, which requires the CS sensor gene, BT3334, because a mutant deficiency for the CS-sensor is unable to increase CS-PUL expression (FIG. 1D) and is unable to grow on CS as a sole carbon source (FIG. 1C). Interestingly, CS-dependent changes in PUL gene transcription are dose-dependent, such that adding increasing amounts of CS to the media results in corresponding increases in PUL-encoded susC (BT3332) transcript levels (FIG. 1E). Similarly, introduction of the common dietary fiber, levan, increases transcription of the corresponding susC gene, BT1763, 530-fold, which requires the levan sensor, BT1754 (FIG. 1F) and is necessary for levan utilization (FIG. 1G) (35). Increasing amounts of levan elicit concentration-dependent increases in the corresponding susC transcript levels (FIG. 1H), However, these responses are kinetic, and require sampling at discreet times following glycan addition (FIGS. 1H and 1I).

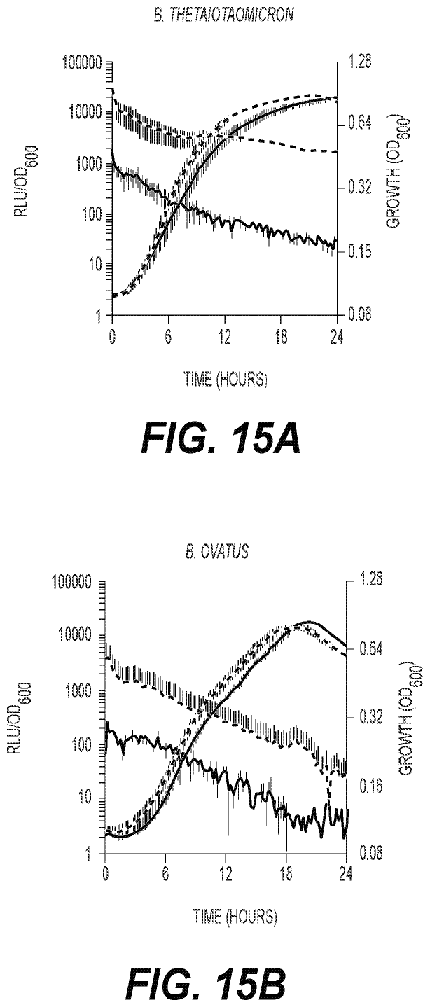

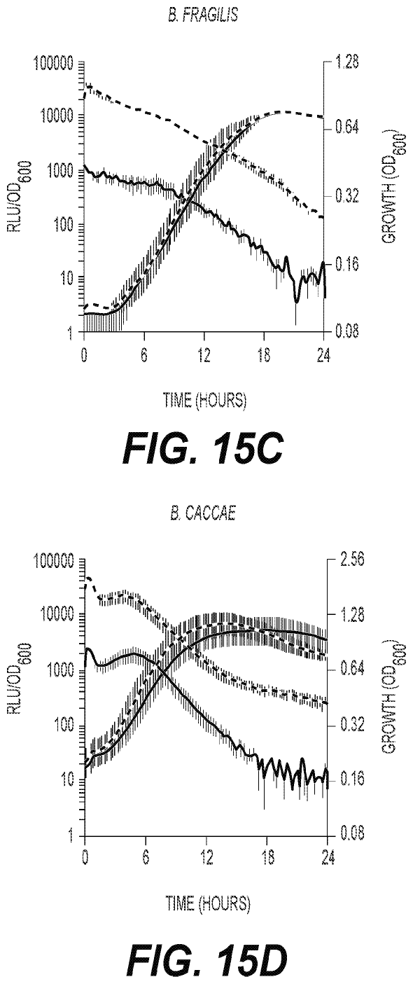

Bacteroides PUL reporters indicate the presence of target glycans. The data show that the rapid, dramatic, and predictable increases in PUL transcript levels can indicate the relative abundance of PUL-specific glycans and therefore be harnessed as glycan biosensors. However, measuring PUL transcription by qPCR or high-throughput sequencing is cumbersome, expensive, and requires kinetic sampling (FIGS. 1E, 1H, and 1I). Thus, the first transcriptional reporter plasmid (named pBolux, for Bacteroides-optimized lux) was developed that can quantify gene transcription during anaerobic growth across Bacteroides species. Because this reporter encodes a complete bacterial luciferase cassette, measuring activity is achieved kinetically, without the addition of exogenous reagents, and exhibits extremely low background as Bacteroides species do not natively produce bioluminescence. Reporter plasmids harboring the region upstream of the constitutively active rpoD gene, exhibited dramatically increased reporter activity compared to the promoter-less pBolux plasmid in Bt (FIG. 15A), Bacteroides ovatus (Bo; FIG. 15B), Bacteroides fragilis (Bf; FIG. 15C), or Bacteroides caccae (Bc, FIG. 15D) without an obvious fitness cost as strains harboring these constructs grew identically to those harboring a promoter-less control plasmid (FIG. 15A-15D, respectively).

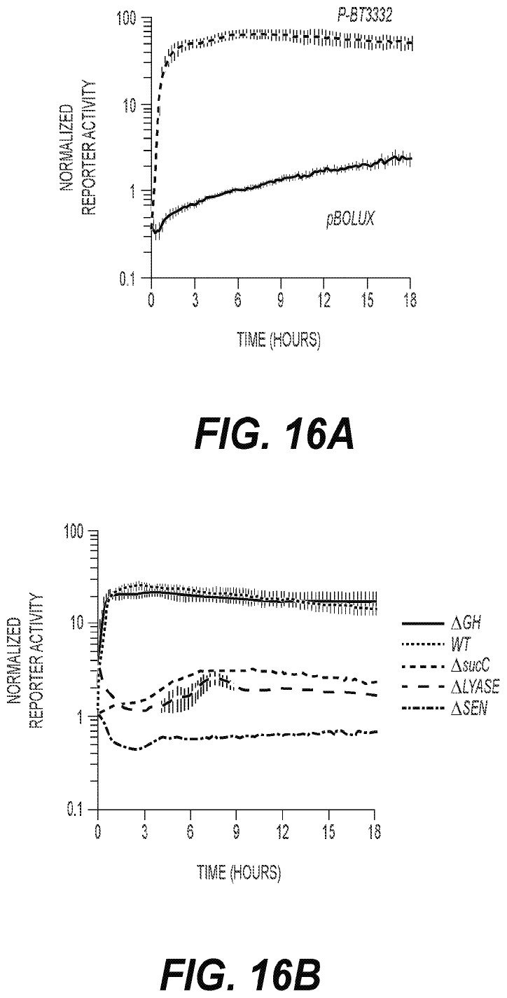

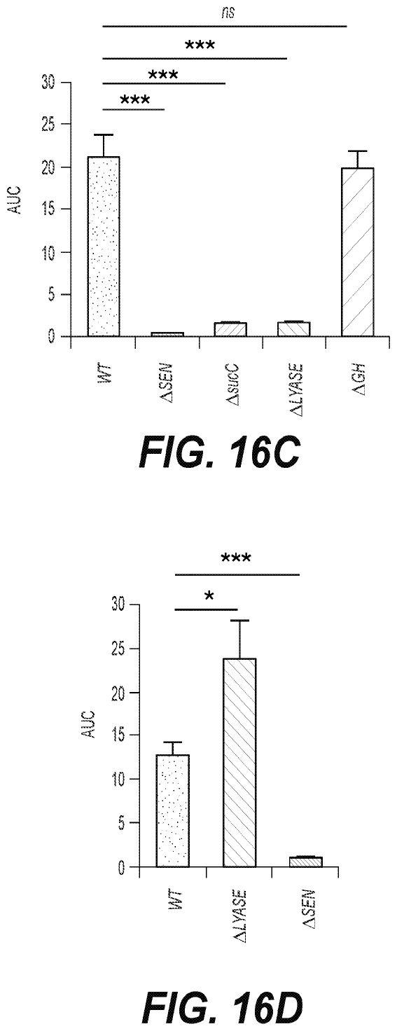

Bt PUL-promoters were cloned preceding the plasmid-encoded lux cassette and measured reporter activity in the presence or absence of known target glycans. For example, a strain harboring a reporter plasmid that included the CS-PUL promoter (i.e., region upstream of the corresponding susC gene, BT3332) resulted in a dramatically increased reporter activity following the introduction of CS compared to a control strain harboring the promoter-less plasmid (FIG. 16A The PUL-encoded activities (depicted in FIG. 1A) were responsible for these CS-induced reporter activity increases by culturing the mutants unable to grow in CS (FIG. 1C) in galactose prior to the introduction of a mixture of CS and galactose. The reporter activity was subsequently measured over 18 hours (FIG. 16B) and the area under each curve (AUC) was calculated for simple statistical comparisons (FIG. 16C). For example, CS-induced reporter activity requires the PUL-encoded outer membrane transporter SusC, because a mutant lacking the corresponding gene, BT3332, was unable to transport CS into the bacterial periplasm and exhibits dramatically reduced activity compared to wild-type Bt in the presence of CS (FIGS. 16B and 16C). Reporter activation requires the depolymerization of CS into disaccharides that bind the sensor in the bacterial periplasm (FIG. 1A) because a mutant deficient in 3 PUL-encoded CS-specific lyases (Alyase) is unable to increase reporter activity in response to CS (FIGS. 16B and 16C) but this can be overcome by the addition of pre-digested chondroitin disaccharides (FIG. 16D). In all cases, increased reporter activity requires the corresponding PUL-sensor as a mutant lacking the CS-sensor gene, BT3334, exhibited no increase in the presence of either chondroitin sulfate (FIGS. 16B and 16C) or chondroitin disaccharide (FIG. 16D). The reduced reporter activity observed in all of these mutants was due to the inability to activate the CS-sensor rather than a consequence of impairing CS utilization because a mutant lacking the CS-specific glucuronyl hydrolase, BT3348, which is necessary to hydrolyze the CS-sensor ligand and grow in CS (FIG. 1C), elicits reporter activation similar to wild-type Bt (FIGS. 16B and 16C). Thus, these engineered strains can specifically indicate the presence of target glycans in the environment by defined mechanisms possessed by PUL-encoded gene products.

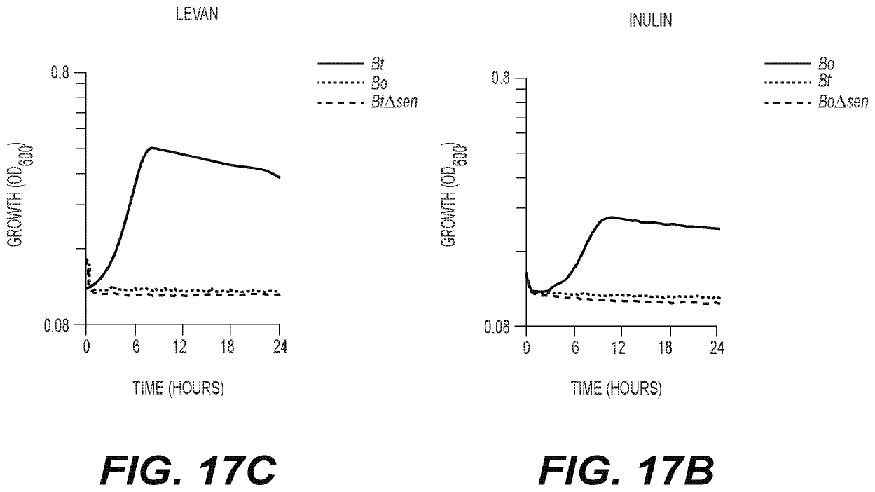

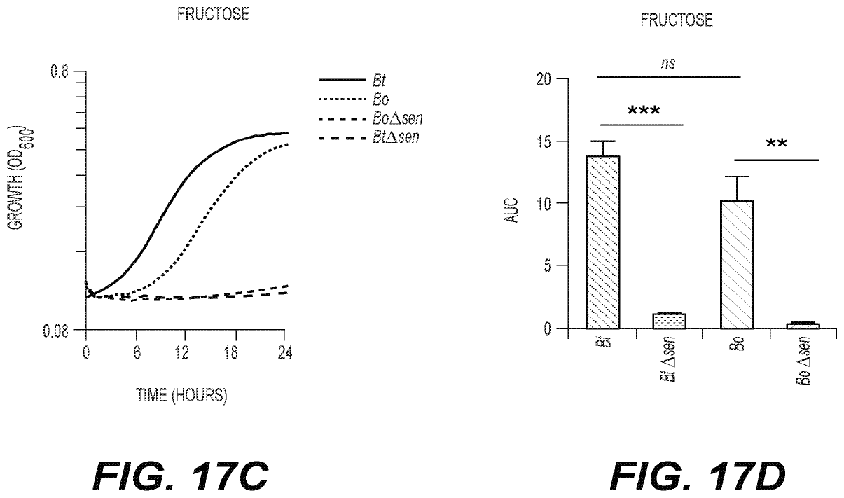

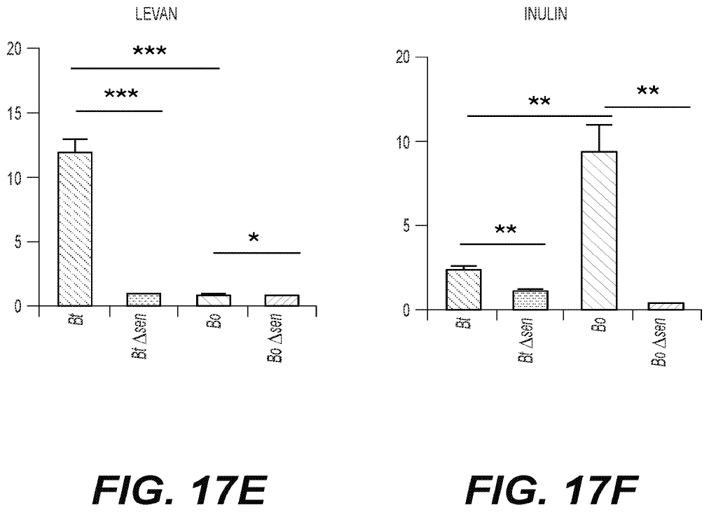

Species-specific responses distinguish between compositionally identical, structurally distinct glycans. Differentiating between compositionally identical glycans requires specialized LC/MS protocols due to the generation of molecules with identical masses following derivatization and ionization. However, different Bacteroides species encode similar PULs that confer distinct substrate specificities for compositionally identical yet structurally distinct glycans. For example, the Bt fructan PUL facilitates consumption of the β2,6-linked polyfructan, levan (FIG. 17A), but not the β2,1-linked polyfructan, inulin (FIG. 17B). Conversely, Bo encodes a highly similar PUL to the one in Bt but facilitates utilization of inulin (FIG. 17B) and is unable to mediate the consumption of levan (FIG. 17A). Bt and Bo exhibit disparate fructan specificities despite both PULs encoding nearly identical sensor proteins that bind the monosaccharide fructose, which is the sole degradation product of both inulin and levan, in-turn activating PUL transcription in the cytoplasm and facilitating growth (FIG. 17C). PUL-reporter plasmids were generated in Bt and Bo containing regions upstream of their respective fructan PUL susC genes and determined that both Bt and Bo reporter strains exhibit similar sensor-dependent reporter activity increases when supplied fructose (FIG. 17D). As expected, the Bt fructan PUL-reporter exhibited 12-fold increased activity in levan (FIG. 17E) but only 2-fold increased activity in response to inulin (FIG. 17F). Conversely, the Bo PUL-reporter exhibited a 9.3-fold increase in reporter activity following the introduction of inulin (FIG. 17F) but no increase in levan (FIG. 17E). These results demonstrate that PULs exhibit substrate specificity even when the PUL-sensor responds to identical ligands, indicating that growth substrate specificity can be mediated by additional-PUL encoded activities. Finally, these results highlight that these reporters can detect very low levels of substrates because statistically significant differences between wild-type and sensor-deficient Bt strains were detected (FIG. 17F), indicating that Bt can liberate monomeric fructose from inulin at comparatively low rates insufficient for growth (FIG. 17B). Thus, by uncoupling PUL signaling from growth, microbial responses to glycans can be examined independently of substrate utilization, facilitating detection of structurally distinct but compositionally identical glycans.

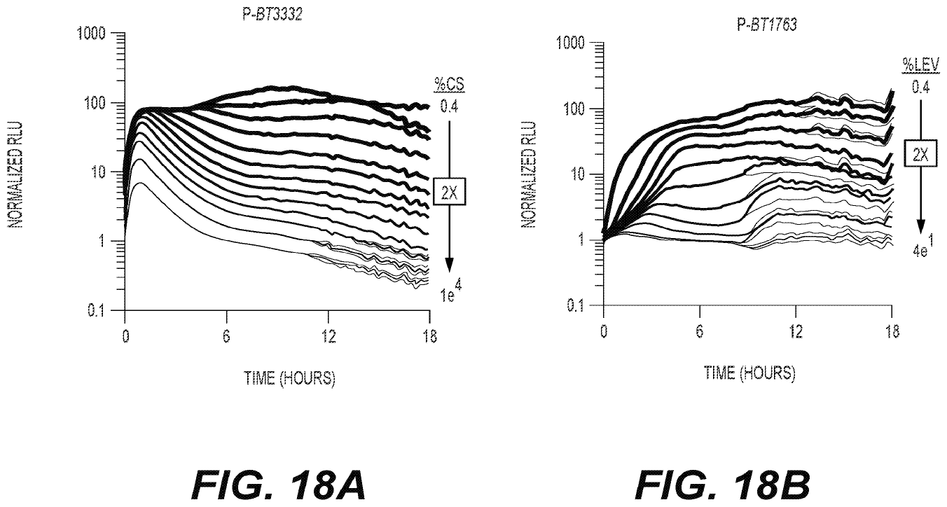

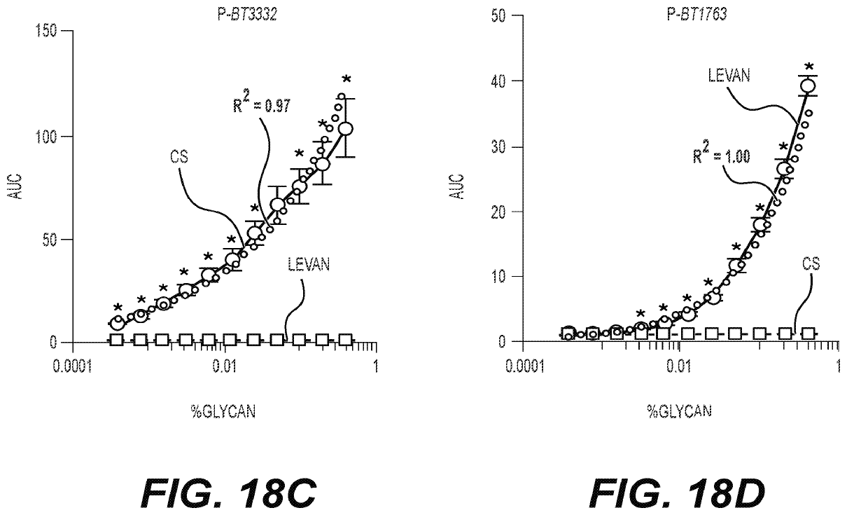

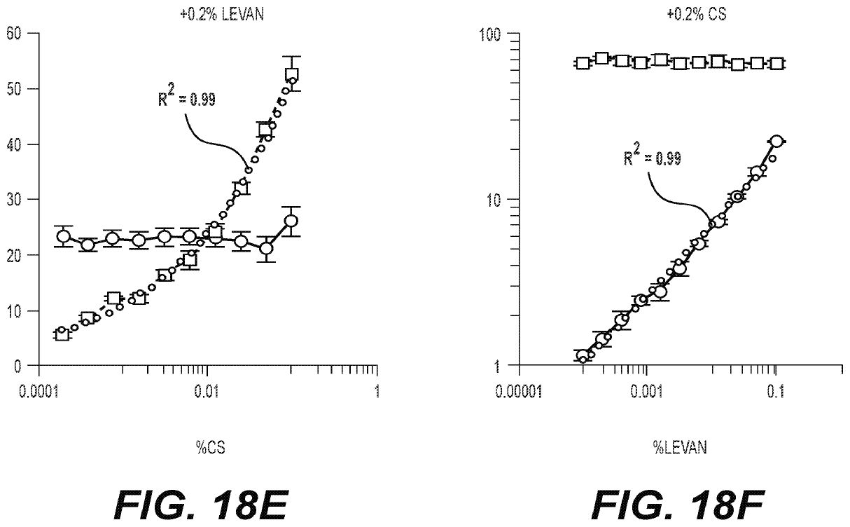

Bacteroides PUL reporters elicit linear dose-dependent responses to target glycans. PUL-sensors elicit dose-dependent increases in PUL transcript levels (FIGS. 1E and 1G) indicating that these responses could be used to quantify glycan abundance relative to a standard curve. Accordingly, PUL-reporters also exhibit dose-dependent responses to target glycans over a linear range greater than three orders of magnitude (FIGS. 18A and 18B). Quantifying the AUC following 18 hours after the introduction of two-fold serial dilutions of either CS or levan revealed a linear response from a wild-type Bt strain harboring the CS-reporter supplied between 0.4 to 0.0004% (4.0 to 0.004 mg/mL) CS but no response from any amount of levan (FIG. 18C). Conversely, strains harboring the levan-reporter plasmid exhibited similar linear dose-dependent responses between two-fold serial dilutions of levan but not CS (FIG. 18D). Furthermore, both the CS and levan-reporters retained linear responsiveness even when 0.2% of the other glycan was present (FIGS. 18E and 18F). These data indicate that the dose-dependent responses of PUL-reporters could be used as standard curves to quantify target glycan abundances present in complex heterogenous mixtures.

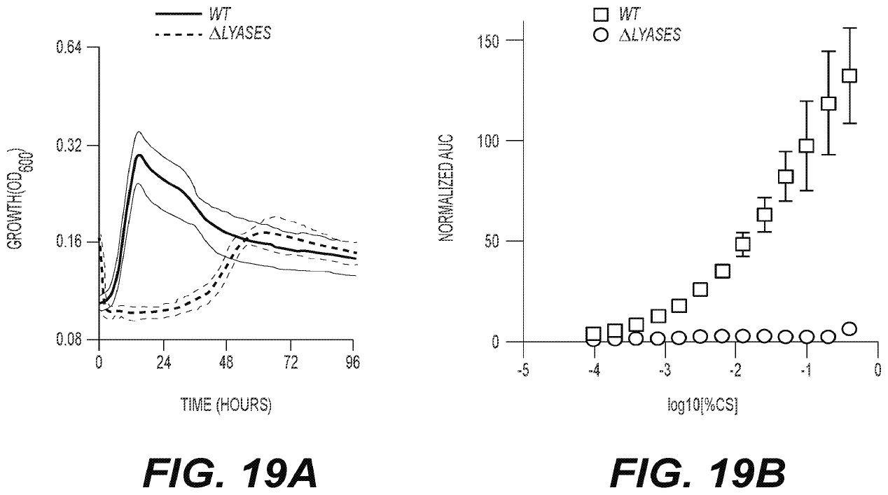

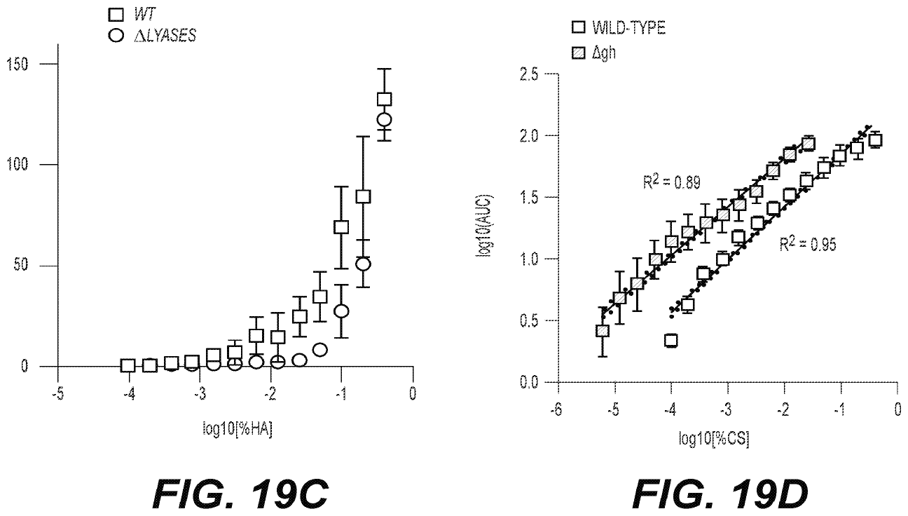

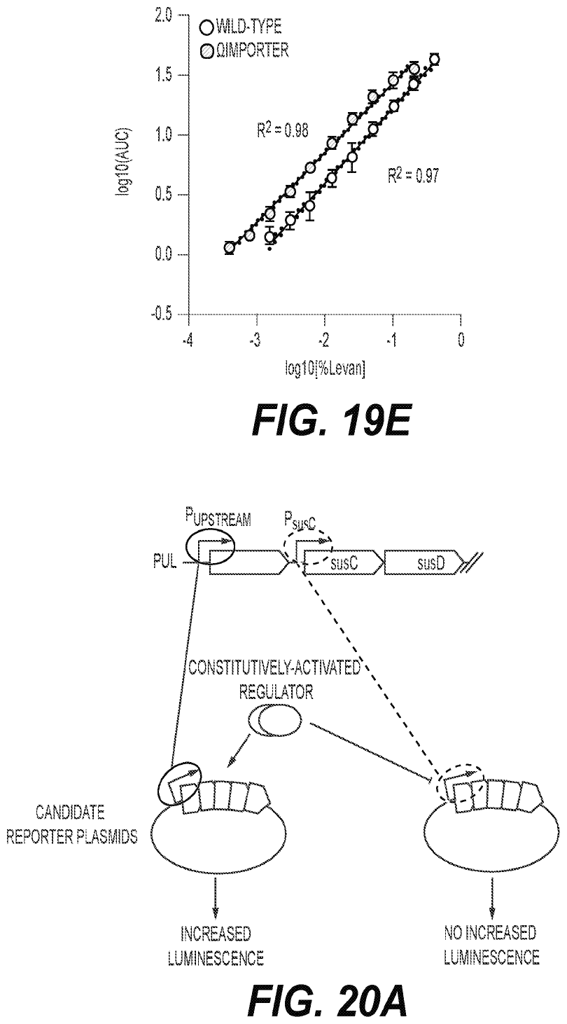

Mutations can narrow glycan specificity and enhance reporter sensitivity. PUL-sensor mediated transcription activation requires enzymatic conversion of target glycans into mono-, di-, or oligosaccharide signatures mediated by PUL-encoded polysaccharide lyases or glycosyl hydrolases that cleave the glycosidic bonds tethering distinct repeating disaccharides. The CS-PUL encodes 3-discreet CS-specific lyases that are collectively required for growth on CS (FIG. 1C) and increased PUL-reporter activity in response to CS (FIGS. 16B and 16C). These lyases also depolymerize a similar glycosaminoglycan, hyaluronan (HA), into disaccharides detected by the same PUL-sensor. However, HA depolymerization is also mediated by an additional unknown enzymatic activity that is unable to act on CS and therefore, a lyase-deficient mutant can grow on HA (FIG. 19A) but not CS (FIG. 1C). Consequently, a lyase-deficient mutant is no longer able to elicit dose-dependent responses in CS compared to wild-type Bt (FIG. 19B) but retains this ability in HA (FIG. 19C). These data demonstrate how directed mutations can further enhance glycan detection specificity by differentiating between two structurally similar, yet compositionally distinct glycans, which cannot be achieved using commercially available kits.

Following activation by binding glycan-derived ligands, PUL-sensors are deactivated and returned to steady state by the processive removal of their cognate glycan-derived ligands (FIG. 1A). Deactivation of the CS-reporter requires hydrolysis of glycan-derived disaccharides into their monosaccharide components by a glucuronyl hydrolase (gh), encoded by BT3348 in Bt. Thus, a gh-deficient mutant accumulates CS-derived disaccharides, prevents PUL-sensor deactivation, which promotes increased PUL transcription relative to wild-type Bt. Consequently, it was determined that the gh-deficient strain exhibited significant CS-reporter activity increases when supplied CS amounts as low as 0.000006% (60 ng/mL), which were undetectable by wild-type Bt harboring the same CS-reporter construct (FIG. 19D). Therefore, by successfully uncoupling PUL transcription from bacterial growth, mutations that prevent PUL-sensor deactivation can be employed, extending the lower limits of detection and quantification well below those of commercially available solutions.

### Conclusions

PUL transcription activation is directly facilitated by PUL sensor proteins that detect mono-, di-, or oligosaccharide signatures derived from their corresponding target glycan. These glycan signatures are generated by progressive and compartmentalized enzymatic activities that facilitate glycan sequestration outside of the cell, followed by target glycan importation and subsequent depolymerization (FIG. 1A). PUL reporters can capture these events and elicit dose-dependent luminescence increases following the introduction of target glycans into cultured Bacteroides reporter strains. These properties are harnessed to reveal the presence and abundance of previously undetectable glycans and dramatically enhance understanding of the interactions between gut microbes and their mammalian hosts.

### A High-Throughput Glycan Detection Platform.

A bioluminescent reporter is demonstrated to be coupled to PUL promoters from different Bacteroides species (FIG. 15) to distinguish between compositionally identical, but structurally distinct glycans (FIG. 17). It has been contemplated to develop libraries of reporter strains representing every predicted PUL across 4 different species. It is further contemplated to develop a high-throughput detection platform that harnesses arrayed reporter strains to simply, accurately, and inexpensively detect the abundance of all glycans that are recognized by these PULs. Thus, leading to the building of a comprehensive resource that can aid in identifying the presence of distinct glycans from a myriad of biological sources including plant, animal, and microbial products.

Reporter plasmids are generated representing every annotated PUL present in B. thetaiotaomicron (strain VPI-5482; 96 predicted PULs), B. ovatus (ATCC 8483; 107 predicted PULs), B. fragilis (NCTC 9343, 55 predicted PULs), and B. caccae (ATCC 43185; 60 predicted PULs). These species were chosen because they encode PUL subsets with distinct glycan specificities and biological niches. PULs are identified using computational methods that examine the genomic sequence for susCD gene pairs, which are the minimum PUL components, and various arrangements of genes whose products encode glycan binding, hydrolysis, and lyase activities. PULs are often polycistronic transcripts initiated from one or a small set of promoters upstream of the corresponding susC gene. Publicly available RNAseq datasets from each organism are used to identify putative transcription start sites to guide putative PUL promoter selection prior to cloning. Plasmids are introduced into their corresponding Bacteroides species, and the resulting strains validated by glycan-dependent and -independent approaches described below. Validated reporter strains are cultured in rich media, combined with glycerol to a final concentration of 10%, and all strains will be dispensed into 50×96-well plates for storage at −80° C. using a Tecan Evo Freedom 200 instrument located at the Penn State Cancer Institute.

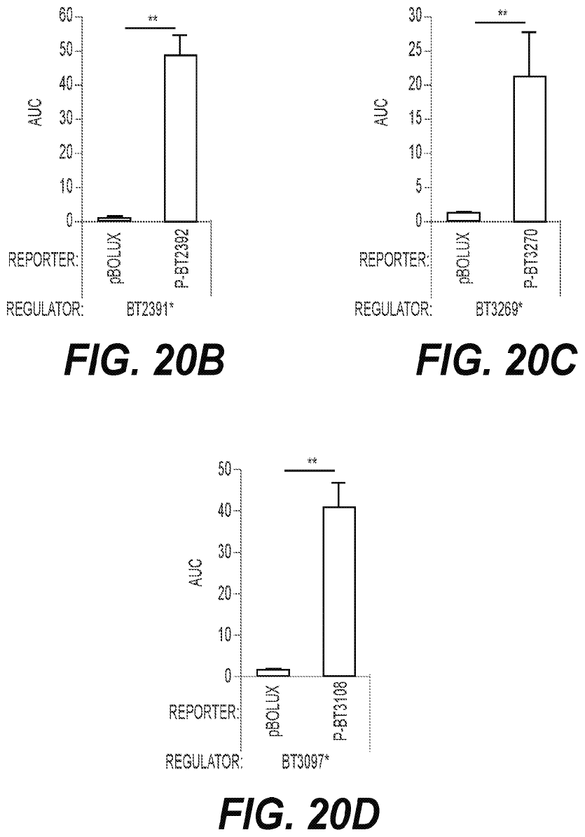

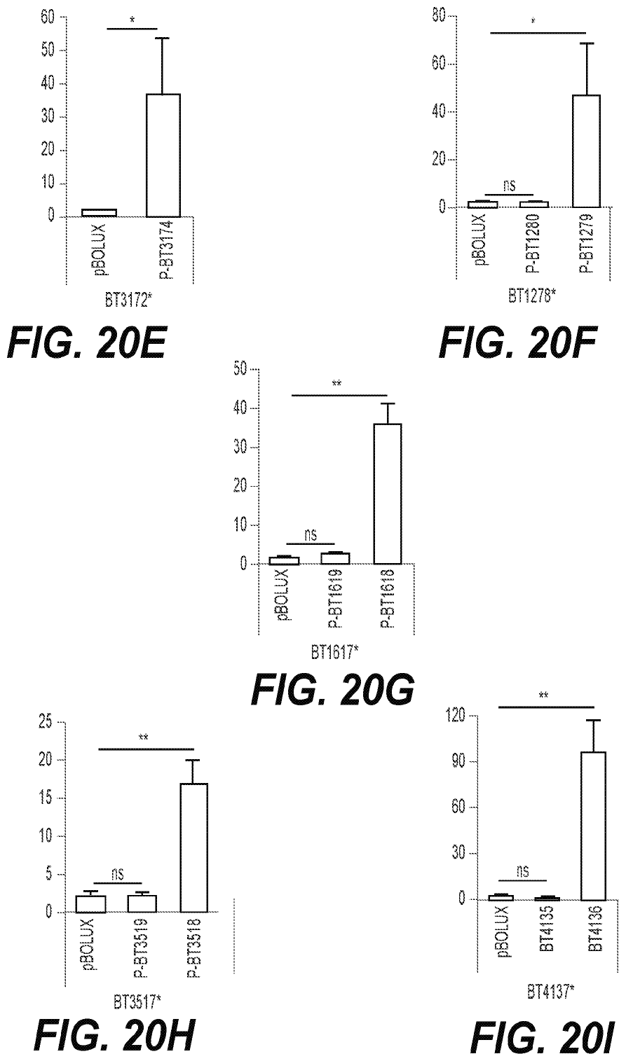

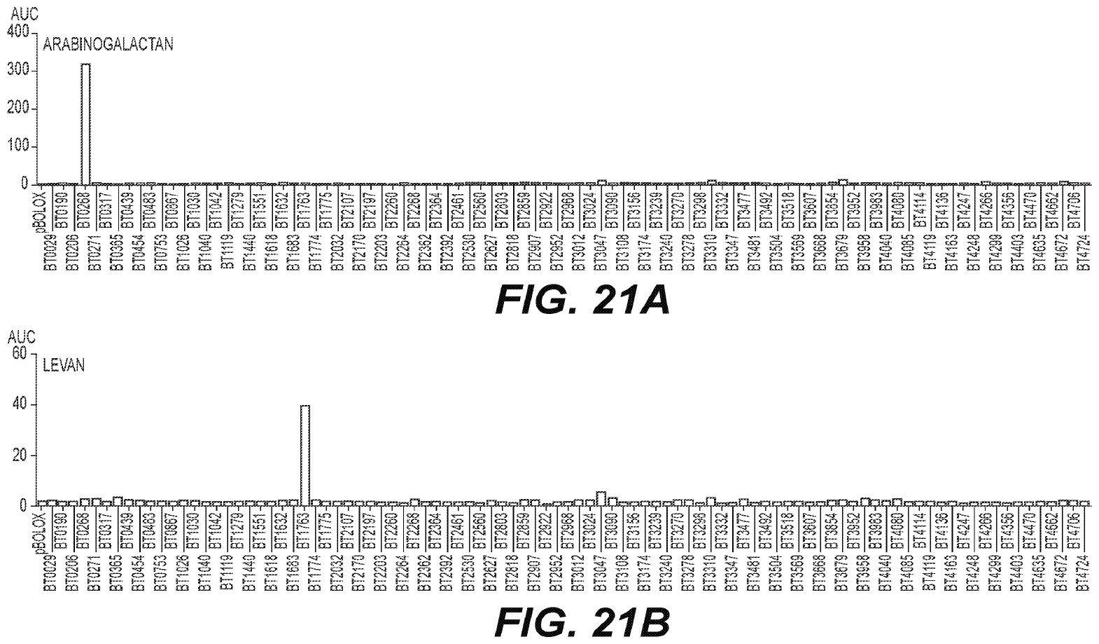

Glycan-dependent validation is achieved when significant increases in bioluminescence are detected following the addition of glycan mixtures to strains harboring putative reporter plasmids that are greater than identical cultures supplied galactose alone. Additionally, glycan-responsive reporters are introduced into mutant strains lacking corresponding PUL-sensors when available and examined for reduced PUL-reporter activation in response to identical glycan mixtures (as demonstrated for CS in FIG. 16). Glycan-independent validation are achieved using strains engineered to express constitutively active forms of the putative PUL regulator that can increase corresponding PUL transcription in the absence of target glycan, an approach we previously developed. Candidate reporter plasmids containing putative PUL promoters are introduced into strains engineered to express the corresponding constitutively active PUL regulator (cartooned in FIG. 20A). To demonstrate the feasibility of this approach, a draft array was generated including reporter constructs representing 95 predicted and experimentally established Bt PULs and validated all possible reporters using glycan-dependent or -independent approaches. Over half of the initially constructed reporters were experimentally validated (FIGS. 20B-20E). When PUL-reporters exhibited no glycan-responsive increases in engineered strains, alternative plasmids were constructed using promoter regions from upstream genes, which generated PUL-regulator-dependent bioluminescence increases (FIGS. 20E-20I). Furthermore, to determine whether constitutively active PUL-regulators elicited specific activation of target PULs, RNAseq analysis was performed comparing genome-wide transcription in strains harboring an empty vector or over-expressing BT3172*, which increased luminescence from P-BT3174 (FIG. 20E) and is a constitutively-active form of a PUL regulator important for Bt intestinal colonization and putatively senses an unknown host-derived glycan. A strain expressing BT3172* exhibited between 8 and 120-fold increases in the corresponding PUL genes, BT3171-BT3180, but no other PUL transcripts were differentially expressed, indicating specific regulation of target genes by these engineered regulatory proteins. Therefore, this strategy can be used to specifically validate each reporter in lieu of known sources containing target glycans and additional PUL reporter constructs in Bo, Bf, and Bc can be validated in this manner or by measuring reporter activity increases in conditions known to activate the corresponding PUL or using glycan-dependent approaches described above.

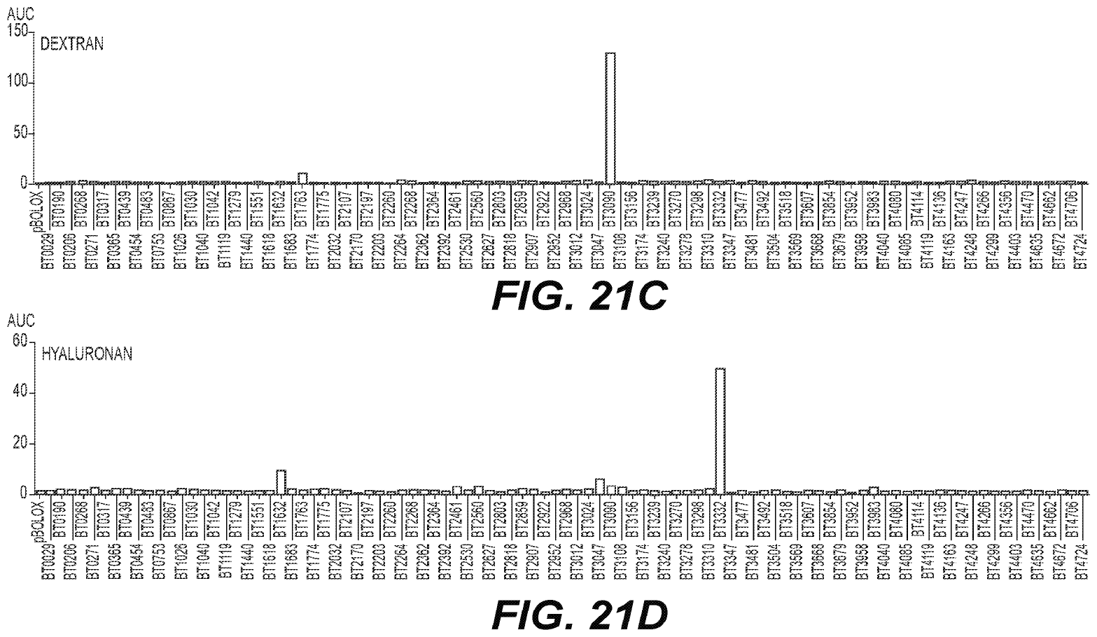

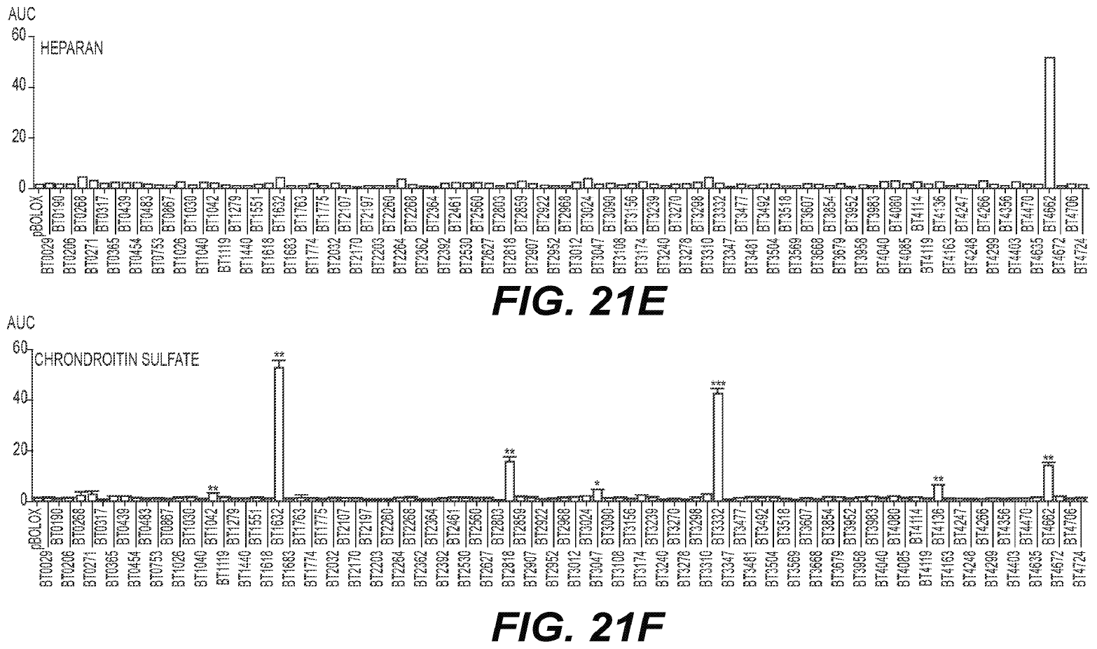

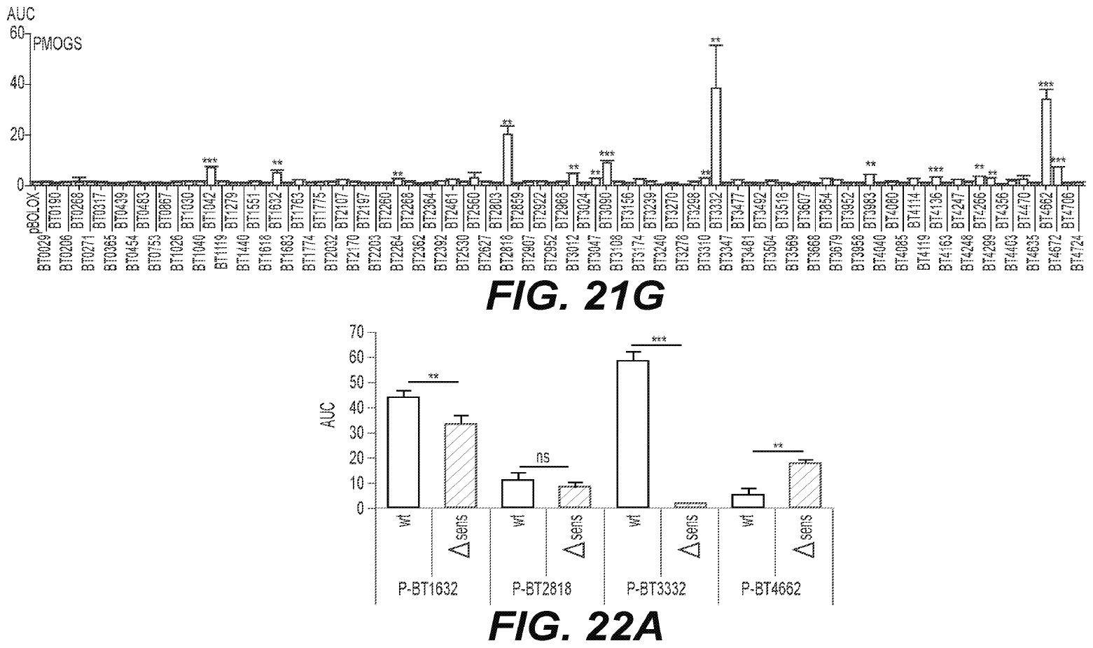

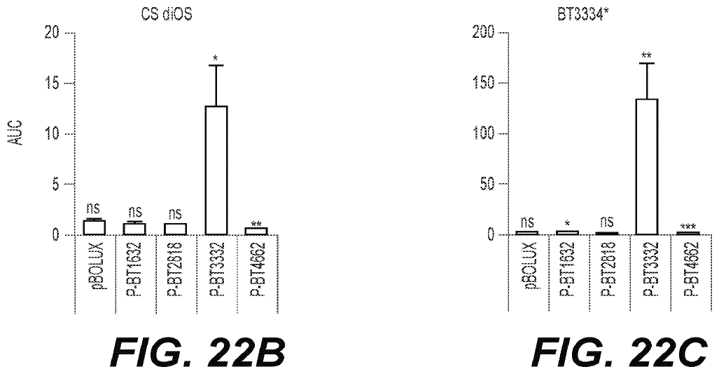

To examine the ability of individual reporters to elicit target-glycan dependent increases in reporter activity, the activity of every Bt reporter is measured following the introduction of commercially purified glycans derived from plants: arabinogalactan and levan; microbes: dextran and HA, or animal tissue: HS and CS. Because genome wide Bt transcriptional responses to these glycans have been previously characterized, bioluminescence from the corresponding reporter strain is observed to be increased. Accordingly, the addition of each glycan to the array elicited the PUL-reporter activity increases. For example, arabinogalactan increased activity from P-BT0268 315-fold (FIG. 21A), levan increased activity from P-BT1763 39-fold FIG. 21B), dextran increased P-BT3090 activity 130-fold (FIG. 21C), HA increased P-BT3332 activity 50-fold (FIG. 21D), HS increased P-BT4662 activity 50-fold (FIG. 21E) and CS increased P-BT3332 activity 42-fold (FIG. 21F). While the addition of arabinogalactan, levan, dextran, HA, and HS elicited a dominant response from the corresponding PUL reporters, CS elicited 6 additional significant responses including 3 that increased greater than 10-fold compared to the galactose treated control, P-BT1632, P-BT2818, and P-BT4662 (FIG. 21F). It was determined that the activation of these reporters was due to the presence of contaminating glycans present in the CS mixture (which is estimated at ˜70% purity by Sigma) rather than the results of promiscuous, non-specific activation by CS or the CS-sensor in the following ways: 1.) A mutant lacking the CS-sensor reduced the activation of P-BT3332 45-fold but only reduced P-BT1632 reporter 1.3-fold (FIG. 22A), 2.) The addition of purified CS disaccharide, which serves as the ligand for the CS-sensor, only activates P-BT3332 (FIG. 22B), and 3.) The overexpression of a constitutively active form of BT3334 (BT3334*) increased luminescence from P-BT3332 greater than 100-fold while the other examined reporters were less that 2-fold different (FIG. 22C). Thus, the activation of additional reporters by CS is due to the presence of additional glycans targeted by distinct Bt PULs indicating that the array can effectively detect distinct molecules present in biologically derived glycan mixtures. Consistent with this notion, 15 PUL-reporters were significantly increased following addition of a biologically-derived glycan mixture, porcine mucosal O-glycans, (PMOGs), consistent with the characterized Bt transcriptional responses. Thus, this approach can detect multiple PUL-specific ligands in biological mixtures.

Validated, arrayed reporter libraries are generated representing a total of 318 predicted PULs across 4 distinct Bacteroides species. Including all PUL-reporters in a single array requires increased array capacity such that all strains can be cultured in 384-well plates prior to the introduction of samples, which is done in 1536-well plates to accommodate 3 distinct samples per reporter strain and a negative control condition (galactose). Increasing capacity using 1536-well plates enables glycan screening to be achieved with reporters from all species simultaneously, thereby increasing the likelihood of detecting molecules of interest with smaller volumes of material than those required for screening in 384-well plates. Arrays containing all strains are manufactured by a Tecan Freedom Evo 200 that can repeatedly dispense into 384-well plates prior to long-term cryogenic storage. Cultured strains are anaerobically transferred using a semi-automated, repeating pipetting instrument. Reporter activity is measured in a Tecan Spark instrument housed in a Coy flexible anaerobic chamber over 18 hours.

An arrayed library of PUL reporters are generated that can detect small quantities of distinct glycan ligands present in crude biological extracts. Based on results with microbial and mammalian glycans, each strain exhibits accurate and sensitive reporter activity increases in response to specific glycan ligands. This offers users a rapid and inexpensive survey of known and unknown glycans as observed though the lens of the gut microbiota. Finally, tailored arrays, containing PUL-reporter subsets of interest in wild-type or mutant backgrounds that exhibit altered target glycan specificities or sensitivities (FIG. 19) are generated in 96- or 384-well microplates.

### A Glycan Isolation Platform.

Determining which PULs are activated via increased reporter activity indicates the presence of a distinct target glycan in samples with unknown glycan content. However, the identity of these molecule(s) may be unknown as only a small fraction of PULs have experimentally determined glycan targets. This is in part due to the challenges facing modern glycomics, which limit structural characterizations of heterogenous glycans mixtures. To overcome these challenges, PUL-reporters are employed as detection agents following glycan fractionation to isolate individual structures from heterogenous mixtures for downstream structural characterization. Moreover, affinity purification approaches are developed using PUL encoded surface glycan binding proteins (SGBPs) to facilitate simple, low-cost isolation of target molecules. Thus, glycans of interest can be identified in a biological sample using the arrays described previously and subsequently isolate individual glycans from these mixtures for downstream compositional and structural analysis. This stepwise approach helps circumvent challenges facing current glycomics strategies which are hindered by a paucity of simple and effective glycan separation strategies.

Bacteroides PULs frequently encode SGBPs that localize the bacterial outer membrane to aid in substrate sequestration prior to importation via the corresponding SusC transporter (FIG. 1A). SGBPs display specificity for target glycan structures and are capable of distinguishing between compositionally identical but structurally distinct glycans. Herein, SGBPs are encoded by PULs with unknown target specificities to isolate the corresponding glycan of interest from heterogenous mixtures (FIGS. 21F and 21G) that can activate distinct reporters (FIG. 7A). SGBPs fused to hexa-histidine tags are expressed in E. coli strain BL21 (DE3) for purification with nickel-conjugated agarose reagents as previously described. Immobilized recombinant protein are incubated with mixtures containing the putative glycan ligands, extensively washed to remove unbound material, and finally eluted with any bound glycans using free histidine. Histidine is used rather than imidazole because it is non-toxic to all Bacteroides cultures tested to date, allowing elution fractions to be added directly to cultured PUL-reporter strains. Corresponding PUL-reporter activity is measured following the addition of the resulting wash and elution fractions against all reporter strains that were activated by the initial glycan mixture. If elutions elicit increased reporter activity corresponding to target glycans but not non-target glycans, then these fractions are considered enriched for target glycans and analyzed by NMR.

To develop this approach, PULs with experimentally defined target specificities are chosen that encode conserved SGBPs. For example, the CS-PUL encodes BT3330 and the levan-PUL encodes BT1761, each exhibiting previously determined in vitro binding affinities for CS and levan, respectively. Accordingly. purified, recombinant over-expressed BT3330 and BT1761 specifically bind to CS and levan, respectively, using affinity-PAGE (FIGS. 7B-7D). Cell lysates prepared from E. coli strains over-expressing either BT3330 or BT1761 were individual combined with an equal mixture of CS and levan, incubated, packed into a column, washed extensively, and eluted with histidine. Eluates from both reactions contained the over-expressed SGBP (FIGS. 7E and 7F) and were supplied to either the CS- or levan reporter strains. Eluates containing BT3330 elicited significant luminescence increases in P-BT3332 but not P-BT1763 containing strains (FIG. 7E), and eluates containing BT1761 increased reporter activity from strains harboring P-BT1763 but not P-BT3332 (FIG. 7F). The elutions were pooled, concentrated, and measured using standard curves from each reporter, which indicated that material recovered from BT3330 contained 205.6 μg/mL CS (FIG. 7G) but no detectable levan. Conversely, BT1761 eluates contained 244.4 μg/mL of levan (FIG. 7H) with no detectable CS. These measurements were validated using a total glycosaminoglycans assay kit (Biovision) or fructan assay kit (Megazyme), which indicated we recovered 228.5 μg/mL CS but no levan from BT3330 and 271.5 μg/mL levan but no CS from BT1761 (FIGS. 7I and 7J, respectively). Finally, reporters that were differentially activated in response to CS (FIG. 21F) were no longer significantly increased following the addition of material co-purifying with BT3330 (FIG. 7K). Collectively, the viability of this approach is demonstrated built upon an incredibly sensitive and specific glycan-detection platform with the ability to rapidly expand the identity of PUL-target glycans.

This method is optimized by identifying stepwise changes in pH, salt concentrations, incubations times, and flow rates that improve target glycan retention. Finally, common parameters that can facilitate target glycan enrichment from SGBPs from the CS, levan, or heparan PULs are applied to PULs with unknown target glycans present in PMOGs, a more complex, commercially available biologically-derived glycan mixture (FIG. 21G) that can be used as a source of PUL-target glycans as demonstrated with BT3330 (FIG. 7L). First, a glycan isolation assay is implemented with the BT4135-PUL reporter using the validated BT4136-promoted PUL-reporter plasmid (FIG. 20I). This PUL contains the putative SGBP encoded by BT4133, which will be over-expressed, purified and incubated with a 0.1% PMOG solution. The unbound material (flow-through), wash, and elution fractions are examined using all PUL-reporters significantly activated by PMOGs (FIG. 21G). Elution fractions that elicit BT4135 PUL-reporter activation, but not non-target reporters are considered enriched for the P-BT4136 activating glycan and are structurally characterized using compositional and NMR analyses of recovered material.

Concomitant with the development of affinity approaches, sequential UHPLC protocols are implemented to fractionate PMOGs by anion exchange chromatography using DEAE over a step-wise sodium chloride gradient as previously described and subsequently fractionated using reverse phase C-18 as previously described. The resulting material is dialyzed and dried by rotary evaporator, resuspended in water, and supplied to cultured strains harboring selected PUL-reporters that exhibited increased activation upon screening with a complete array.

Affinity purification protocols are developed to simply isolate glycans that activate distinct PUL reporters using SGBPs cloned, over-expressed, and purified from the corresponding PUL. Successful implementation of this approach is demonstrated using SGBPs with known binding affinities (FIG. 7) and have contemplated implementing similar approaches with unknown glycans present in PMOGs. Therefore, a universal strategy is developed to isolate target glycans of interest from a heterogenous mixture that increases activity from a corresponding PUL-reporter.

### A Tunable Glycan Measurement System Using PUL Reporters.

Measuring the abundance of individual glycans requires expensive, non-renewable reagents that can fail to distinguish between compositionally similar molecules. It has been demonstrated that PUL-sensors elicit dose-dependent increases in target gene transcription in response to their cognate glycan ligands (FIGS. 1E and 1G), and this is recapitulated with the corresponding PUL-reporters (FIGS. 18A-18D) even in the presence of additional non-target glycans (FIG. 18E) across wide linear ranges. Therefore, glycan measurement methods are developed using standard curves derived from the AUC of PUL reporter dose-dependent responses. Additionally, mutant strains are identified and characterized that enhance PUL-reporter sensitivity and narrow target glycan specificity by taking advantage of these system's abilities to distinguish between structurally distinct glycans (FIG. 19). This example generates a simple and inexpensive approach to measuring unknown target glycan quantities present in heterogenous mixtures.

Dose-dependent reporter responses to known glycan ligands are examined to define the limits of detection and quantification for each system. A standard set of measurement conditions are optimized that generate consistent results across Bt PUL-reporters specific for 1.) CS/HA, 2.) levan, 3.) arabinan, 4.) α-mannan and 5.) heparan sulfate by measuring the corresponding reporter activity against a standard curve of purified ligand. The supporting monosaccharide identity and abundance, supplement bacterial cultures with additional macronutrients, and determine time for computing AUC is varied to identify conditions that generate the optimal linear range across all 5 reporters. Subsequently, PUL-reporter measurements are compared against lab-generated or biologically derived glycan mixtures using each of the 5 reporters and validate these measurements using commercially available kits designed to measure the corresponding glycans (CS/HA and heparan sulfate: Biovision; levan and arabinan: Megazyme; mannan: Bio-rad). In many cases, measurements may be lower than the limits of detection or quantification of commercial kits.

It has been demonstrated that a mutation preventing CS-specific PUL-sensor deactivation generates detectable PUL-reporter responses at CS levels below the limits of detection in wild-type Bt (FIG. 19D). This indicates that blocking the consumption of the PUL-ligand leads to PUL-sensor hyperactivation and increasing the sensitivity of PUL-reporters to their target glycan. The linear range for the CS PUL-reporter had dramatically shifted to linear responsiveness to CS in a Bt mutant defective for PUL-deactivation, reaching as low as 0.000006% CS compared to 0.0001% observed in wild-type Bt (FIG. 19A). Additional mutants are generated that block PUL-reporters with known glycan specificities and determine 1.) whether the corresponding PUL-reporters exhibit expanded limits of detection, 2.) the increased sensitivity elicits dose-dependent responses, and 3.) these responses can accurately measure unknown target glycan concentrations present in biological mixtures. This is determined using PULs with known target glycans that have commercially available kits to validate measurements: CS using the BT3348-deficient mutant, levan using a mutant defective for the inner membrane fructose transporter encoded by BT1758, and arabinan using a mutant lacking the inner membrane oligo-arabinose transporter, araP.

PUL-reporters produce glycan measurements resembling those generated using commercially available kits or MS/MS. Furthermore, genetically disrupting PUL-sensor deactivation increases the sensitivity of additional PUL-reporters, reducing their limits of detection and quantification without reducing specificity. Finally, disabling specific enzymatic activities “blinds” Bacteroides PUL-sensors to glycan subsets enabling narrower detection specificities relative to wild-type strains while retaining wide linear ranges of quantification.

### Outlook

The fruits of this example are the development and production of a high-throughput tool, that can rapidly surveille samples for a diverse collection of glycans derived from plant, animal, or microbial sources. The detection of an activated PUL-reporter indicates the presence of a known or unknown glycan ligand corresponding to a microbial signal or growth substrate that corresponds to a locus containing glycan binding protein(s). Therefore, activation of PUL-reporters that respond to unknown glycan structures reveals gene products that can aid in glycan isolation using the methods described herein, for downstream functional and structural analyses using existing methodologies. Finally, these PUL-reporters can perform as robust glycan measurement tools that circumvent limitations associated with measuring some glycans using mass spec, NMR, or lectins and that by harnessing defined PUL mutations, further enhancing target sensitivity and refining specificity. The products herein represent an end-to-end pipeline from crudely extracted cellular material to isolating molecules of interest that differentially activated microbial gene expression.

TABLE 4
GT1893, GT1934, and GT3173 Primers and Promoters

| | | | | | | | regu- | |
|---|---|---|---|---|---|---|---|---|
| | | | primer | | | regu- | lator | |
| strain | plasmid | susC | ID | primer sequence | promoter sequence | lator | class | glycan |
| GT1893 | P- | BT1763 | 1150 | gctcggtacccggggat | tatcattcagttttctgttggtt | BT1754 | HTCS | levan |
| | BT1763 | | | cctatcattcagttttctgt | actttgagtgattatatgcttg | | | |
| | | | | tggttactttgagtga | ttatatgaataatatcccttttt | | | |
| | | | | (SEQ ID NO: 26) | acaaggacttaaattactata | | | |
| | | | 1304 | AAATGCGGGA | caaaggtagtattttctatga | | | |
| | | | | GTGACTAGTTA | ttgagagagaatataatccc | | | |
| | | | | GTTTAATGTTA | gactttcttatgatggagaat | | | |
| | | | | TTAATTTAAAA | tattcttataaaaccctataaa | | | |
| | | | | GTACGAATTTT | atagcgttctatgcaacatat | | | |
| | | | | CTCTTTTCGAT | cttacagcatttgaaacatttt | | | |
| | | | | G (SEQ ID NO: | tttcagtcggttgacttaaat | | | |
| | | | | 27) | caaccgtaatttgcatcatc | | | |
| | | | | | gaaaagagaaaattcgtact | | | |
| | | | | | tttaaattaataacattaaact | | | |
| | | | | | a (SEQ ID NO: 74) | | | |
| GT1934 | P- | BT3332 | 1232 | GCTCGGTACCC | aaaatggaactgggcaatg | BT3334 | HTCS | chondroitin |
| | BT3332 | | | GGGGATCCAA | acaggcatccccagctgta | | | sulfate / |
| | | | | AATGGAACTG | cgatttatccgccgatccat | | | hyaluronan |
| | | | | GGCAATGACA | cggagaaaaacaatgtagc | | | |
| | | | | GG (SEQ ID NO: | gaaacagcatccggaagtt | | | |
| | | | | 28) | gtcagggagctgtccgaac | | | |
| | | | 1373 | AAATGCGGGA | tgcttgaatcggtaaagaca | | | |
| | | | | GTGACTAGTCC | agataaccaatgagaatag | | | |
| | | | | TTTTTCTTGTC | cagatttgtattattttgtatca | | | |
| | | | | TGGTTGGATA | atcttgaataattctatccata | | | |
| | | | | GATGTTTTTT | tgagaacaatcaatgcgtat | | | |
| | | | | (SEQ ID NO: 29) | atctttgccatcgtaaattca | | | |
| | | | | | acaaaatcacttaatttttaat | | | |
| | | | | | atatgaaaaaacatctatcc | | | |
| | | | | | aaccagacaagaaaaagg | | | |
| | | | | | (SEQ ID NO: 75) | | | |
| GT3173 | P- | BACOVA_ | 1943 | GCTCGGTACCC | ttgtttgttggagattgttttca | BACOVA_ | HTCS | inulin |
| | BACOVA_ | 04505 | | GGGGATCCttgttt | tatcgttgattttttcagttgac | 04496 | | |
| | 04505 | | | gttggagattgttttcata | tacaaccagtagttaactcta | | | |
| | | | | tcgttg (SEQ ID | tttttaagttcttatcacctgc | | | |
| | | | | NO: 72) | aaaggtaatattttccatactt | | | |
| | | | 1944 | AAATGCGGGA | actggagaatagatattctat | | | |
| | | | | GTGACTAGTtag | aattcttctatttcgaagaata | | | |
| | | | | tttgatgttattaaattaaa | atgtataaaaccctataaaat | | | |
| | | | | agtacgaattttac | agcgttctatgcaacaaatg | | | |
| | | | | (SEQ ID NO: 73) | ttatagcctttgaaacatttttt | | | |
| | | | | | tcagtcgtttgatataaatca | | | |
| | | | | | accgtaatttgcatcatcgat | | | |
| | | | | | atagtaaaattcgtacttttaa | | | |
| | | | | | tttaataacatcaaacta | | | |
| | | | | | (SEQ ID NO: 76) | | | |

TABLE 5A
Bt Reporter Array_Primers

| | Bt | | Primer | | |
|---|---|---|---|---|---|
| Construct | strain | SusC | number | Primer name | Sequence |
| 1 | GT1867 | — | | | empty vector |
| 2 | GT4079 | BT0029 | 2445 | pBolux-P- | AGCTCGGTACCCGGGGATCCaggacgaaaatgtaa |
| | | | | BT0028-500- | ctttgcca (SEQ ID NO: 77) |
| | | | | f | |
| | | | 2446 | pBolux-P- | ATGCGGGAGTGACTAGTacttactattttttctcgctgca |
| | | | | BT0028-500- | aaattcca (SEQ ID NO: 78) |
| | | | | r | |
| 3 | GT4080 | BT0140 | 1106 | pLYL- | GCTCGGTACCCGGGGATCCCTTCCAATGAT |
| | | | | PBT0140_fwd | ACTGAAGAGAAAATCATTGCTG (SEQ ID |
| | | | | | NO: 79) |
| | | | 2447 | pBolux-P- | ATGCGGGAGTGACTAGTaacattaaatttgaggtttaaa |
| | | | | BT0140-363- | aatagtattaaattc (SEQ ID NO: 80) |
| | | | | r | |
| 4 | GT3787 | BT0190 | 1108 | pLYL- | GCTCGGTACCCGGGGATCCTCTGGAGTCCG |
| | | | | PBT0190_fwd | TACTTAAATCCATCGA (SEQ ID NO: 81) |
| | | | 1311 | rpiL- | AAATGCGGGAGTGACTAGTAAATTTGTGC |
| | | | | PBT0190_rev_ | GTTTTAGTAATTGACTAAAGTTATTAAAAA |
| | | | | new | CAAG(SEQ ID NO: 82) |
| 5 | GT4081 | BT0206 | 2448 | pBolux-P- | AGCTCGGTACCCGGGGATCCcgtgtggcacggctg |
| | | | | BT0206-700- | cagct (SEQ ID NO: 83) |
| | | | | f | |
| | | | 1312 | rpiL- | AAATGCGGGAGTGACTAGTCTTTGATACTT |
| | | | | PBT0206_rev_ | GAATTTAAATTAATGAATTGATTTATTTTT |
| | | | | new | ACAGATTTATCTAC(SEQ ID NO: 84) |
| 6 | GT1873 | BT0268 | 1112 | pLYL- | GCTCGGTACCCGGGGATCCGTCTAAGTCTT |
| | | | | PBT0268_fwd | CTCTTTATAGGAAAATGAAATTAGC (SEQ |
| | | | | | ID NO: 85) |
| | | | 1313 | rpiL- | AAATGCGGGAGTGACTAGTACTATTAGATT |
| | | | | PBT0268_rev_ | TATAAAGTTATTTAGACCAAATAAGTATTA |
| | | | | new | GTTGTGC (SEQ ID NO: 86) |
| 7 | GT1875 | BT0317 | 1116 | pLYL- | GCTCGGTACCCGGGGATCCTTCAATTTCAA |
| | | | | PBT0317_fwd | TAATAACTTTTGTGTGTTAGAGTCCCT (SEQ |
| | | | | | ID NO: 87) |
| | | | 1315 | rpiL- | AAATGCGGGAGTGACTAGTAATACTGCTCT |
| | | | | PBT0317_rev_ | TTTAAGGTTAATAAAAATTGTCTGTGTG |
| | | | | new | (SEQ ID NO: 88) |
| 8 | GT1876 | BT0364 | 1012 | pLYL- | GCTCGGTACCCGGGGATCCTCAATGTGACA |
| | | | | PBT0365_fwd | CCAAGCGCACTG (SEQ ID NO: 89) |
| | | | 1084 | PBT0365- | AAATGCGGGAGTGACTAGTAAGTAGTAAC |
| | | | | ATGr-SpeI | GGCCATTTCCTTGTCTTC (SEQ ID NO: 90) |
| 9 | GT4117 | BT0439 | 2449 | pBolux-P- | AGCTCGGTACCCGGGGATCCaagtggattgacgatg |
| | | | | BT0439-500- | cacg (SEQ ID NO: 91) |
| | | | | f | |
| | | | 2253 | rpiL-BT0439- | ATGCGGGAGTGACTAGTgtttactagtgataataaaca |
| | | | | 381-r | gaatgtcgt (SEQ ID NO: 92) |
| 10 | GT1878 | BT0452 | 1120 | pLYL- | GCTCGGTACCCGGGGATCCAAAATAGGAA |
| | | | | PBT0454_fwd | TTTGCGTCTGATGTACAAAAACG (SEQ ID |
| | | | | | NO: 93) |
| | | | 1317 | rpiL- | AAATGCGGGAGTGACTAGTCCCCGAAACT |
| | | | | PBT0454_rev_ | CCGTAATAGACCA (SEQ ID NO: 94) |
| | | | | new | |
| 11 | GT1879 | BT0483 | 1122 | pLYL- | GCTCGGTACCCGGGGATCCGCTATGGCTAC |
| | | | | PBT0483_fwd | GGATACGGTTACG (SEQ ID NO: 95) |
| | | | 1318 | rpiL- | AAATGCGGGAGTGACTAGTAATTATCATTT |
| | | | | PBT0483_rev_ | TTTAAGGGATAATACAAATATAACAAAAA |
| | | | | new | AAACGGG (SEQ ID NO: 96) |
| 12 | GT3779 | BT0754 | 1124 | pLYL- | GCTCGGTACCCGGGGATCCTATTGCCCATC |
| | | | | PBT0754_fwd | GTCTGGAACGT (SEQ ID NO: 97) |
| | | | 1319 | rpiL- | AAATGCGGGAGTGACTAGTTTTTATCACTA |
| | | | | PBT0754_rev_ | TTAAAGGTTAACATATAGTTATTTATCGTA |
| | | | | new | GCTGC (SEQ ID NO: 98) |
| 13 | GT4083 | BT0867 | 2450 | pBolux-P- | AGCTCGGTACCCGGGGATCCgaatattgcaaaggta |
| | | | | BT0867-700- | atttttaaaattgtttttccc (SEQ ID NO: 99) |
| | | | | f | |
| | | | 1320 | rpiL- | AAATGCGGGAGTGACTAGTAATTTTGATTT |
| | | | | PBT0867_rev_ | TAAAATAATAACTAGTTTACCGGTTTTGTT |
| | | | | new | TGT (SEQ ID NO: 100) |
| 14 | GT3972 | BT1029 | 2394 | pLYL- | GCTCGGTACCCGGGGATCCGGTGGATTAC |
| | | | | PBT1030_fwd | AATGATCTTATCCATTCGTCC (SEQ ID NO: |
| | | | | | 101) |
| | | | 1322 | rpiL- | AAATGCGGGAGTGACTAGTAGATTAAATT |
| | | | | PBT1030_rev_ | TTAATTTATATAAAATGATATATTACATCA |
| | | | | new | TCAGACATCTATTCACAT (SEQ ID NO: 102) |
| 15 | GT1884 | BT1040 | 1132 | pLYL- | GCTCGGTACCCGGGGATCCTGGTGTTTCCT |
| | | | | PBT1040_fwd | TTAAAACCAAAATGCC (SEQ ID NO: 103) |
| | | | 1323 | rpiL- | AAATGCGGGAGTGACTAGTTGTGACTTTTA |
| | | | | PBT1040_rev_ | GGGGTTGGGC (SEQ ID NO: 104) |
| | | | | new | |
| 16 | GT1885 | BT1042 | 1134 | pLYL- | GCTCGGTACCCGGGGATCCTAGTTTTTCAG |
| | | | | PBT1042_fwd | AACTACTTAATGCTCTATTTATCAAATGAT |
| | | | | | TATG (SEQ ID NO: 105) |
| | | | 1324 | rpiL- | AAATGCGGGAGTGACTAGTCTTATTCTTTT |
| | | | | PBT1042_rev_ | TAATTAGAAATAGTTTTTAGGTAGTTAACA |
| | | | | new | AACATGTG (SEQ ID NO: 106) |
| 17 | GT4131 | BT1119 | 2455 | pBolux-P- | AGCTCGGTACCCGGGGATCCattaagggggggcta |
| | | | | BT1119-700f | tgctg (SEQ ID NO: 107) |
| | | | 1325 | rpiL- | AAATGCGGGAGTGACTAGTCTTGATTCGTT |
| | | | | PBT1119_rev_ | AAATCTCTGTAGATTCCCCAG (SEQ ID NO: |
| | | | | new | 108) |
| 18 | GT3780 | BT1280 | 1138 | pLYL- | GCTCGGTACCCGGGGATCCAGACCAGGCA |
| | | | | PBT1280_fwd | CCACTATCTGAG (SEQ ID NO: 109) |
| | | | 1326 | rpiL- | AAATGCGGGAGTGACTAGTTTTCTTAGTAT |
| | | | | PBT1280_rev_ | TAGGTTATTAACTAAATTATTTCACGGTAA |
| | | | | new | TTGAATAG (SEQ ID NO: 110) |
| 19 | GT1888 | BT1440 | 1140 | pLYL- | GCTCGGTACCCGGGGATCCTCTTAAAAGCG |
| | | | | PBT1440_fwd | CTTGGTTTGGCC (SEQ ID NO: 111) |
| | | | 1327 | rpiL- | AAATGCGGGAGTGACTAGTAATTTTGTGGC |
| | | | | PBT1440_rev_ | TTAGGATTTATTTGCTTGTTCTAAC (SEQ ID |
| | | | | new | NO: 112) |
| 20 | GT1889 | BT1552 | 1142 | pLYL- | GCTCGGTACCCGGGGATCCAAGGGGAAAG |
| | | | | PBT1551_fwd | TCGAAAGGTGG (SEQ ID NO: 113) |
| | | | 1328 | rpiL- | AAATGCGGGAGTGACTAGTTAAGTCCTTTT |
| | | | | PBT1551_rev_ | ACTTTTAATTATTATTAAAAACAGCCGCA |
| | | | | new | (114) |
| 21 | GT3781 | BT1619 | 1144 | pLYL- | GCTCGGTACCCGGGGATCCCATACACATCA |
| | | | | PBT1619_fwd | TCGTCCCTCCGC (SEQ ID NO: 115) |
| | | | 1329 | rpiL- | AAATGCGGGAGTGACTAGTACTTTTGTGAG |
| | | | | PBT1619_rev_ | TTAATCATTAATACTAATTTAAGTGTACAC |
| | | | | new | AAC (SEQ ID NO: 116) |
| 22 | GT1891 | BT1631 | 1146 | pLYL- | GCTCGGTACCCGGGGATCCGGGCGGAAGA |
| | | | | PBT1632_fwd | GTTTTTAAGAGAGAATATAG (SEQ ID NO: |
| | | | | | 117) |
| | | | 1330 | rpiL- | AAATGCGGGAGTGACTAGTAAGTTTATTA |
| | | | | PBT1632_rev_ | ATTAAGATTACCTTTTATATAAGTAGCGAA |
| | | | | _ncw | GTCTTTCC (SEQ ID NO: 118) |
| 23 | GT4118 | BT1683 | 2456 | pBolux-P- | AGCTCGGTACCCGGGGATCCgtactatgcagagagt |
| | | | | BT1683-700f | tacg (SEQ ID NO: 119) |
| | | | 1331 | rpiL- | AAATGCGGGAGTGACTAGTTCGTTTTAATT |
| | | | | PBT1683_rev_ | TAAAGTTAATATTAATTTTCCTTTGTTTAGT |
| | | | | new | AAAATACAATT (SEQ ID NO: 120) |
| 24 | GT1893 | BT1763 | 1150 | pLYL- | Gctcggtacccggggatcctatcattcagttttctgttggttactttgagt |
| | | | | PBT1763_fwd | ga (SEQ ID NO: 26) |
| | | | 1304 | rpiL- | AAATGCGGGAGTGACTAGTTAGTTTAATGT |
| | | | | PBT1763_rev_ | TATTAATTTAAAAGTACGAATTTTCTCTTTT |
| | | | | new | CGATG (SEQ ID NO: 27) |
| 25 | GT4119 | BT1774 | 2457 | pBolux-P- | AGCTCGGTACCCGGGGATCCgcttaattttcgcttaat |
| | | | | BT1775-700f | tgattattaa (SEQ ID NO: 121) |
| | | | 1334 | rpiL- | AAATGCGGGAGTGACTAGTCGTGATTTTCA |
| | | | | PBT1775_rev_ | AATAATTTTTCACGGTCTCTTTTTTATATTA |
| | | | | new | TAC (SEQ ID NO: 122) |
| 26 | GT3908 | BT1875 | 2346 | pBolux- | AGCTCGGTACCCGGGGATCCtggaacgtgaacttga |
| | | | | BT1876-300- | aaac (SEQ ID NO: 123) |
| | | | | f | |
| | | | 2347 | rpiL-BT1876- | ATGCGGGAGTGACTAGTatttatgctttctataaaggag |
| | | | | 300-r | ac (SEQ ID NO: 124) |
| 27 | GT1898 | BT2107 | 1160 | pLYL- | GCTCGGTACCCGGGGATCCTGTAATCTATC |
| | | | | PBT2107_fwd | TAATTTTATGTTCGTTTCATAAATTTAAAA |
| | | | | | CCATACA (SEQ ID NO: 125) |
| | | | 1337 | rpiL- | AAATGCGGGAGTGACTAGTACTCTGTGATT |
| | | | | PBT2107_rev_ | TTAAAAAGTAAGTCATAAGGGTTTTATATT |
| | | | | new | AGA (SEQ ID NO: 126) |
| 28 | GT3783 | BT2172 | 2262 | pBolux- | AGCTCGGTACCCGGGGATCCcgaccggcttccgga |
| | | | | BT2170-300- | acgtt (SEQ ID NO: 127) |
| | | | | f | |
| | | | 2263 | rpiL-BT2170- | ATGCGGGAGTGACTAGTcattcaatcttttttagtgattaa |
| | | | | 300-r | tcggt (SEQ ID NO: 128) |
| 29 | GT3782 | BT2196 | 2260 | pBolux- | AGCTCGGTACCCGGGGATCCttgcttataaatctattc |
| | | | | BT2197-300- | gtctgacag (SEQ ID NO: 129) |
| | | | | f | |
| | | | 2261 | rpiL-BT2197- | ATGCGGGAGTGACTAGTattgcgcgcttttaataagaa |
| | | | | 300-r | gacac (SEQ ID NO: 130) |
| 30 | GT3878 | BT2202 | 2266 | pBolux- | AGCTCGGTACCCGGGGATCCgctggcactggcctat |
| | | | | BT2203-M- | ggta (SEQ ID NO: 131) |
| | | | | 37-f | |
| | | | 2267 | rpiL-BT2203- | ATGCGGGAGTGACTAGTacglattaggitittaaataaa |
| | | | | M-37-r | acaataagtaaataac (SEQ ID NO: 132) |
| 31 | GT1901 | BT2260 | 1166 | pLYL- | GCTCGGTACCCGGGGATCCAATAAAAGCG |
| | | | | PBT2260_fwd | CCTCTTATAGCATGATACTATTTTTTGT |
| | | | | | (SEQ ID NO: 133) |
| | | | 1340 | rpiL- | AAATGCGGGAGTGACTAGTACCAATATAC |
| | | | | PBT2260_rev_ | GAAATCTGCAAAGTCTTTGC (SEQ ID NO: |
| | | | | new | 134) |
| 32 | GT1902 | BT2264 | 1168 | pLYL- | GCTCGGTACCCGGGGATCCGGTGGTGAGA |
| | | | | PBT2264_fwd | AATATTCGGGAAATATAAAAATAGTATCC |
| | | | | | (SEQ ID NO: 135) |
| | | | 1341 | rpiL- | AAATGCGGGAGTGACTAGTACCGATGTAC |
| | | | | PBT2264_rev_ | GAAATTTGCAAAGTCTTTG (SEQ ID NO: |
| | | | | new | 136) |
| 33 | GT1904 | BT2362 | 1172 | pLYL- | GCTCGGTACCCGGGGATCCAAAGATAGTA |
| | | | | PBT2362_fwd | AAAATCAACCCATTCCGATTCAAAG (SEQ |
| | | | | | ID NO: 137) |
| | | | 1343 | rpiL- | AAATGCGGGAGTGACTAGTGTTTTTTGTTT |
| | | | | PBT2362_rev_ | AATTGTATGTTCACTAATTGGTTTTTGAAT |
| | | | | new | AGT (SEQ ID NO: 138) |
| 34 | GT1905 | BT2364 | 1174 | pLYL- | GCTCGGTACCCGGGGATCCCTACGACCTTT |
| | | | | PBT2364_fwd | ACCGCTACCATTATCAAC (SEQ ID NO: 139) |
| | | | 1344 | rpiL- | AAATGCGGGAGTGACTAGTACTTTTATATT |
| | | | | PBT2364_rev_ | TAAAAATACATTTGCTATAAAGTCGATACA |
| | | | | new | AAGGA (SEQ ID NO: 140) |
| 35 | GT1906 | BT2393 | 1176 | pLYL- | GCTCGGTACCCGGGGATCCGCAAATGATA |
| | | | | PBT2392_fwd | AAAAAAGCAATTTTGAATGAAAAGATATA |
| | | | | | CATG (SEQ ID NO: 141) |
| | | | 1345 | rpiL- | AAATGCGGGAGTGACTAGTGTTTTTCTATA |
| | | | | PBT2392_rev_ | AGATTTTATTAGTTTGAAAAAAGGTCTTTT |
| | | | | new | AGGC (SEQ ID NO: 142) |
| 36 | GT3911 | BT2461 | 2348 | pBolux- | AGCTCGGTACCCGGGGATCCttgcaatcattacctga |
| | | | | BT2462-300- | acaaacct (SEQ ID NO: 143) |
| | | | | f | |
| | | | 2349 | rpiL-BT2462- | ATGCGGGAGTGACTAGTctttttatcgttttcatttatataa |
| | | | | 300-r | cgattc (SEQ ID NO: 144) |
| 37 | GT4137 | BT2531 | 2458 | pBolux-P- | AGCTCGGTACCCGGGGATCCgaaacgccagacga |
| | | | | BT2529-700f | atcagg (SEQ ID NO: 145) |
| | | | 2351 | rpiL-BT2529- | ATGCGGGAGTGACTAGTcaatacttttttaattttcatttct |
| | | | | 300-r | tttgc (SEQ ID NO: 146) |
| 38 | GT2372 | BT2560 | 1638 | pLYL- | GCTCGGTACCCGGGGATCCAAGTGCAGGA |
| | | | | PBT2561_fwd | GCATGAGCT (SEQ ID NO: 147) |
| | | | 1639 | rpiL- | AAATGCGGGAGTGACTAGTAGTATTAAAA |
| | | | | PBT2561_rev_ | ATTAAGACTATGCAAATGAAAGCAA (SEQ |
| | | | | | ID NO: 148) |
| 39 | GT1910 | BT2626 | 1184 | pLYL- | GCTCGGTACCCGGGGATCCAGATAGATATT |
| | | | | PBT2627_fwd | GGGAGTGCATATGTTTGTTTTTAC (SEQ ID |
| | | | | | NO: 149) |
| | | | 1349 | rpiL- | AAATGCGGGAGTGACTAGTTTCTATATTCT |
| | | | | PBT2627_rev_ | TTATATTTGATTAGTTTCTATTATATGATTA |
| | | | | new | AACAGCGGATATC (SEQ ID NO: 150) |
| 40 | GT2375 | BT2805 | 1642 | pLYL- | GCTCGGTACCCGGGGATCCTATGAAAGTC |
| | | | | PBT2803_fwd | AGCTTTTACGCCTCTTTG (SEQ ID NO: 151) |
| | | | 1643 | rpiL- | AAATGCGGGAGTGACTAGTGCATAGTTTTC |
| | | | | PBT2803_rev_ | TACATTAATAATTGGTTTATATAATCTCAA |
| | | | | new | AAATGATATCTTATA (SEQ ID NO: 152) |
| 41 | GT1912 | BT2818, | 1188 | pLYL- | GCTCGGTACCCGGGGATCCTGTTCCTTACA |
| | | BT2820 | | PBT2818_fwd | AAGCCTCCTTTTCCA (SEQ ID NO: 153) |
| | | | 1351 | rpiL- | AAATGCGGGAGTGACTAGTACTGTTTAATT |
| | | | | PBT2818_rev_ | TGATAGGTTAATAATATATTTAGTTTATAG |
| | | | | new | TTAGGTCAAC (SEQ ID NO: 154) |
| 42 | GT4138 | BT2859 | 2459 | pBolux-P- | AGCTCGGTACCCGGGGATCCactacctcaaaacgc |
| | | | | BT2859-700f | agatc (SEQ ID NO: 155) |
| | | | 1352 | rpiL- | AAATGCGGGAGTGACTAGTGTATCAGAAT |
| | | | | PBT2859_rev_ | TTTAAATTAGTATTAGAGTTCACATTTAAG |
| | | | | new | GATAAATCA (SEQ ID NO: 156) |
| 43 | GT4122 | BT2894 | 2460 | pBolux-P- | AGCTCGGTACCCGGGGATCCgttctaacttttaccgc |
| | | | | BT2896-700f | aaac (SEQ ID NO: 157) |
| | | | 1353 | rpiL- | AAATGCGGGAGTGACTAGTTTTCTTTATTA |
| | | | | PBT2896_rev_ | TTTTGGTTAAACATTTATATCATGATCCTTA |
| | | | | new | TAAAAGG (SEQ ID NO: 158) |
| 44 | GT1915 | BT2905 | 1194 | pLYL- | GCTCGGTACCCGGGGATCCTGTGGACATTG |
| | | | | PBT2907_fwd | GTACGGACG (SEQ ID NO: 159) |
| | | | 1354 | rpiL- | AAATGCGGGAGTGACTAGTAATATTATAG |
| | | | | PBT2907_rev_ | TATTTAGTTATCACTTAAAAATTTAGTTATT |
| | | | | new | ATCTTATTGTTGATTATTC (SEQ ID NO: 160) |
| 45 | BT4123 | BT2906 | 2461 | pBolux-P- | AGCTCGGTACCCGGGGATCCccgggcgaatggca |
| | | | | BT2909-700f | caccta (SEQ ID NO: 161) |
| | | | 2462 | pBolux-P- | ATGCGGGAGTGACTAGTagcattattcattttttaattatg |
| | | | | BT2909r | atac (SEQ ID NO: 162) |
| 46 | GT1917 | BT2920 | 1411 | pLYL- | GCTCGGTACCCGGGGATCCGTACATGCAG |
| | | | | PBT2922_fwd_ | GATCTATATCCCCG (SEQ ID NO: 163) |
| | | | | new | |
| | | | 1356 | rpiL- | AAATGCGGGAGTGACTAGTATATAATTAG |
| | | | | PBT2922_rev_ | ATTATTGTTTCGACTGATTGACGCAGA |
| | | | | new | (SEQ ID NO: 164) |
| 47 | GT1918 | BT2952 | 1200 | pLYL- | GCTCGGTACCCGGGGATCCCTTCGGAATTG |
| | | | | PBT2952_fwd | CTAACACCTACG (SEQ ID NO: 165) |
| | | | 1357 | rpiL- | AAATGCGGGAGTGACTAGTAAGTTAGACT |
| | | | | PBT2952_rev_ | TTTTGAATAACATAATAATTAAGTCAATAT |
| | | | | new | AAATAGTTAAGTATAGT (SEQ ID NO: 166) |
| 48 | GT3975 | BT2952 | 2399 | pBolux- | AGCTCGGTACCCGGGGATCCaatgtaaggacaagt |
| | | | | BT2956-300- | cctgagaagag (SEQ ID NO: 167) |
| | | | | f | |
| | | | 2400 | rpiL-BT2956- | ATGCGGGAGTGACTAGTgatatgattcttttagtattagc |
| | | | | 300-r | tgatggg (SEQ ID NO: 168) |
| 49 | GT1919 | BT2968 | 1202 | pLYL- | GCTCGGTACCCGGGGATCCGATGTGCACTA |
| | | | | PBT2968_fwd | TGTCTTGGCTGATG (SEQ ID NO: 169) |
| | | | 1358 | rpiL- | AAATGCGGGAGTGACTAGTAAATTATTAG |
| | | | | PBT2968_rev_ | ATTAGTTAATGATAAATACTTGTTTTCCAT |
| | | | | new | TTAAAAGAGATTC (SEQ ID NO: 170) |
| 50 | GT2365 | BT3012 | 1629 | pLYL- | GCTCGGTACCCGGGGATCCACAATCGGCT |
| | | | | PBT3011_fwd | AAAGTCATAAACCTGAC (SEQ ID NO: 171) |
| | | | 1630 | rpiL- | AAATGCGGGAGTGACTAGTCTTTTTTATAA |
| | | | | PBT3011_rev_ | TATTAATGAGTTCTATTGAATTCACGTC |
| | | | | | (SEQ ID NO: 172) |
| 51 | GT4124 | BT3024 | 2463 | pBolux-P- | AGCTCGGTACCCGGGGATCCaaaaacaatgcaaga |
| | | | | BT3024-700f | aatggaaac (SEQ ID NO: 173) |
| | | | 1360 | rpiL- | AAATGCGGGAGTGACTAGTAATTTATTGTA |
| | | | | PBT3024_rev_ | TTAGATTATAAACTCTATTCAATTGTTGAT |
| | | | | new | TGATCCA (SEQ ID NO: 174) |
| 52 | GT1922 | BT3046 | 1208 | pLYL- | GCTCGGTACCCGGGGATCCAGTTGTGTTTC |
| | | | | PBT3047_fwd | TTGCAGCCAAC (SEQ ID NO: 175) |
| | | | 1361 | rpiL. | AAATGCGGGAGTGACTAGTAACGTTTTTCT |
| | | | | PBT3047_rev_ | TTTTACATATTAAATTATTGGTTCACTAAA |
| | | | | new | AATTC (SEQ ID NO: 176) |
| 53 | GT1923 | BT3090 | 1210 | pLYL- | GCTCGGTACCCGGGGATCCGCTACTCTGTG |
| | | | | PBT3090_fwd | ACCACTATTTATAATTACCGG (SEQ ID NO: |
| | | | | | 177) |
| | | | 1362 | rpiL- | AAATGCGGGAGTGACTAGTGTACATCAAT |
| | | | | PBT3090_rev_ | TTAAAGTTAATATTAGGATTACTTTTTGTTT |
| | | | | new | TCACTG (SEQ ID NO: 178) |
| 54 | GT1924 | BT3103 | 1212 | pLYL- | GCTCGGTACCCGGGGATCCTAATGTAGCTG |
| | | | | PBT3108_fwd | GCAGACATCCG (SEQ ID NO: 179) |
| | | | 1363 | rpiL- | AAATGCGGGAGTGACTAGTATGATTCTAA |
| | | | | PBT3108_rev_ | AAAGTTAAACGTTATTTATGTATGATTGTG |
| | | | | new | ATGC (SEQ ID NO: 180) |
| 55 | GT1925 | BT3156 | 1214 | pLYL- | GCTCGGTACCCGGGGATCCACAAAAAAAC |
| | | | | PBT3156_fwd | ATTCATTCCTAAAATAAAGGATGATGGAC |
| | | | | | (SEQ ID NO: 181) |
| | | | 1364 | rpiL | AAATGCGGGAGTGACTAGTGGATAATTTA |
| | | | | PBT3156_rev_ | AAATTAATATTAGGTTAATACATTTCAGGC |
| | | | | new | AACTAGATC (SEQ ID NO: 182) |
| 56 | GT1926 | BT3174 | 1216 | pLYL- | GCTCGGTACCCGGGGATCCCTGCAAAACG |
| | | | | PBT3174_fwd | TCCTGTTTCTAAAAAATG (SEQ ID NO: 183) |
| | | | 1365 | rpiL- | AAATGCGGGAGTGACTAGTCTGTTTCCTTT |
| | | | | PBT3174_rev_ | TTTTCATAATACTACATTTAATAATAAAGA |
| | | | | new | TTCATACT (SEQ ID NO: 184) |
| 57 | GT1927 | BT3239 | 1412 | pLYL- | GCTCGGTACCCGGGGATCCAAGGAAGTGT |
| | | | | PBT3239_fwd_ | TTAGATGACATAATGATTATTTGAACAG |
| | | | | new | (SEQ ID NO: 185) |
| | | | 1413 | pLYL- | AAATGCGGGAGTGACTAGTCGTTGTACCTT |
| | | | | PBT3239_rev_ | TCACTAATACGGATGC (SEQ ID NO: 186) |
| | | | | new | |
| 58 | GT1928 | BT3240 | 1220 | pLYL- | GCTCGGTACCCGGGGATCCCCTGTCACCTT |
| | | | | PBT3240_fwd | TTTAGGTGTTTGG (SEQ ID NO: 187) |
| | | | 1414 | pLYL- | AAATGCGGGAGTGACTAGTATTCTCCTATA |
| | | | | PBT3240_rev_ | ACCTATTTTCATACTAATTATTTATATCTAA |
| | | | | new | TAT (SEQ ID NO: 188) |
| 59 | GT1929 | BT3271 | 1222 | pLYL- | GCTCGGTACCCGGGGATCCTCCTTGCTTTT |
| | | | | PBT3270_fwd | GTGGGGGTG (SEQ ID NO: 189) |
| | | | 1368 | rpiL- | AAATGCGGGAGTGACTAGTAATTTTATCTA |
| | | | | PBT3270_rev_ | TTTCTAATGAGGATTTTATCTTTGCTTTACT |
| | | | | new | ATAATTATAC (SEQ ID NO: 190) |
| 60 | GT1930 | BT3279 | 1224 | pLYL- | GCTCGGTACCCGGGGATCCTCTCTTGAAAC |
| | | | | PBT3278_fwd | TGTGAAGACTCAAAAGAAG (SEQ ID NO: |
| | | | | | 191) |
| | | | 1369 | rpiL- | AAATGCGGGAGTGACTAGTATTATTTCAGG |
| | | | | PBT3278_rev_ | TTATTTATAGCAAAGACGACTAAGAAG |
| | | | | new | (SEQ ID NO: 192) |
| 61 | GT3977 | BT3297 | 2403 | pBolux- | AGCTCGGTACCCGGGGATCCatattcctgaaagccg |
| | | | | BT3299-300- | gagaatcc (SEQ ID NO: 193) |
| | | | | f | |
| | | | 2404 | rpiL-BT3299- | ATGCGGGAGTGACTAGTgacgctatgtgtaatttttacct |
| | | | | 300-r | tgtgtg (SEQ ID NO: 194) |
| 62 | GT1933 | BT3310 | 1230 | pLYL- | GCTCGGTACCCGGGGATCCGAATACAATTT |
| | | | | PBT3310_fwd | ATAATTATCGGGCGAAAGTAAAAAACAAA |
| | | | | | GC (SEQ ID NO: 195) |
| | | | 1372 | rpiL- | AAATGCGGGAGTGACTAGTGCGTATTAATT |
| | | | | PBT3310_rev_ | TTAAAGTTTATAATTAAGGTATGTGCTTGA |
| | | | | new | CAG (SEQ ID NO: 196) |
| 63 | GT1934 | BT3332 | 1232 | pLYL- | GCTCGGTACCCGGGGATCCAAAATGGAAC |
| | | | | PBT3332_fwd | TGGGCAATGACAGG (SEQ ID NO: 28) |
| | | | 1373 | rpiL- | AAATGCGGGAGTGACTAGTCCTTTTTCTTG |
| | | | | PBT3332_rev_ | TCTGGTTGGATAGATGTTTTTT (SEQ ID NO: |
| | | | | new | 29) |
| 64 | GT1935 | BT3346 | 1234 | pLYL- | GCTCGGTACCCGGGGATCCACATTTCTCCC |
| | | | | PBT3347_fwd | TTGAAGGGC (SEQ ID NO: 197) |
| | | | 1374 | rpiL- | AAATGCGGGAGTGACTAGTAAATATAAAT |
| | | | | PBT3347_rev_ | AATAAAATGGTTAAAGTGCATCCGAACAA |
| | | | | new | TAAATATTATTGC (SEQ ID NO: 198) |
| 65 | GT1936 | BT3475 | 1236 | pLYL- | GCTCGGTACCCGGGGATCCAAAGTTAGAA |
| | | | | PBT3477_fwd | GTCATCAATTAATAGACCTTCATTTTGG |
| | | | | | (SEQ ID NO: 199) |
| | | | 1377 | rpiL- | AAATGCGGGAGTGACTAGTGATTTTGTTTT |
| | | | | PBT3492_rev_ | TTTTCATACGTAAAAAATTAATGATTAATA |
| | | | | new | AAATATTATATTGTTGG (SEQ ID NO: 201) |
| 66 | GT1938 | BT3494 | 1240 | pLYL- | GCTCGGTACCCGGGGATCCGTTTGTAGATC |
| | | | | PBT3492_fwd | CTCTTTATAAATACATCAATGAAACT (SEQ |
| | | | | | ID NO: 200) |
| | | | 1377 | rpiL- | AAATGCGGGAGTGACTAGTGATTTTGTTTT |
| | | | | PBT3492_rev_ | TTTTCATACGTAAAAAATTAATGATTAATA |
| | | | | new | AAATATTATATTGTTGG (SEQ ID NO: 201) |
| 67 | GT1939 | BT3505 | 1242 | pLYL- | GCTCGGTACCCGGGGATCCCCATTGGACTC |
| | | | | PBT3504_fwd | CCGCAAAGA (SEQ ID NO: 202) |
| | | | 1378 | rpiL- | AAATGCGGGAGTGACTAGTTTTACATTTCA |
| | | | | PBT3504_rev_ | TGTTTTTCATTCTAGTTAATAATAAATGTAT |
| | | | | new | AGTTAATTAAAC (SEQ ID NO: 203) |
| 68 | GT3784 | BT3519 | 1254 | pLYL- | GCTCGGTACCCGGGGATCCGAATAAATGT |
| | | | | PBT3519_fwd | CGAATTTGACTCAGCGCTAAG (SEQ ID NO: |
| | | | | | 204) |
| | | | 1384 | rpiL | AAATGCGGGAGTGACTAGTAATATTATTAT |
| | | | | PBT3519_rev_ | TTTATAACTTAATACTTACAGGCATATGAG |
| | | | | new | CCCC (SEQ ID NO: 205) |
| 69 | GT1940 | BT3569 | 1244 | pLYL- | GCTCGGTACCCGGGGATCCGTCTGCCTGAT |
| | | | | PBT3569_fwd | GTAAAAGAGTAGTTGCA (SEQ ID NO: 206) |
| | | | 1379 | rpiL- | AAATGCGGGAGTGACTAGTTCGTTACTATT |
| | | | | PBT3569_rev_ | TAGGTTGAAGTTTATTCTGCCG (SEQ ID NO: |
| | | | | new | 207) |
| 70 | GT1941 | BT3604 | 1246 | pLYL- | GCTCGGTACCCGGGGATCCAATGTCGCGAT |
| | | | | PBT3607_fwd | GAGCAACAG (SEQ ID NO: 208) |
| | | | 1380 | rpiL- | AAATGCGGGAGTGACTAGTAGTTTACTTTT |
| | | | | PBT3607_rev_ | TTTAGTTTCTACTCTTTTTAGTATATATTTA |
| | | | | new | ACCTATTG (SEQ ID NO: 209) |
| 71 | GT1946 | BT3680 | 1256 | pLYL- | GCTCGGTACCCGGGGATCCCGTAAAGGGA |
| | | | | PBT3679_fwd | ACTATAGTGCATCTTGC (SEQ ID NO: 210) |
| | | | 1385 | rpiL- | AAATGCGGGAGTGACTAGTAGCAAAAATA |
| | | | | PBT3679_rev_ | TTTAAGATATTAGTAAATAAAAAATTAACC |
| | | | | new | GTTCATTAATTGA (SEQ ID NO: 211) |
| 72 | GT1942 | BT3670 | 1248 | pLYL- | GCTCGGTACCCGGGGATCCATTTTTCCAGT |
| | | | | PBT3668_fwd | TCCAATCGGCATTATG (SEQ ID NO: 212) |
| | | | 1381 | rpiL- | AAATGCGGGAGTGACTAGTAAATATTCCT |
| | | | | PBT3668_rev_ | ATTAGATTATATACCGCAAATGTAACAAC |
| | | | | new | (SEQ ID NO: 213) |
| 73 | GT4084 | BT3702 | 2451 | pBolux-P- | AGCTCGGTACCCGGGGATCCtacttcctgcctcatct |
| | | | | BT3703-300- | gctttc (SEQ ID NO: 214 |
| | | | | f | |
| | | | 2452 | pBolux-P- | ATGCGGGAGTGACTAGTtctatttatggtattaaattataa |
| | | | | BT3703-300- | gctaac (SEQ ID NO: 215) |
| | | | | r | |
| 74 | GT3970 | BT3750 | 2392 | pBolux- | AGCTCGGTACCCGGGGATCCatcgtctggatggaaa |
| | | | | BT3749-300- | atctcataaag (SEQ ID NO: 216) |
| | | | | f | |
| | | | 2387 | rpiL-BT3749- | ATGCGGGAGTGACTAGTattataagttgagttactttata |
| | | | | 300-r | agtaataaacag (SEQ ID NO: 217) |
| 75 | GT1944 | BT3854 | 1252 | pLYL- | GCTCGGTACCCGGGGATCCATCGGTGGCG |
| | | | | PBT3854_fwd | GAGCCTGTCC (SEQ ID NO: 218) |
| | | | 1383 | rpiL- | AAATGCGGGAGTGACTAGTAACAGAATCC |
| | | | | PBT3854_rev_ | AAAGAAAGGATGCTCCC (SEQ ID NO: 219) |
| | | | | new | |
| 76 | GT4140 | BT3952 | 2467 | pBolux-P- | AGCTCGGTACCCGGGGATCCttatatggaagacctg |
| | | | | BT3952-700f | gaagg (SEQ ID NO: 220) |
| | | | 1386 | rpiL- | AAATGCGGGAGTGACTAGTACTACTTGTTT |
| | | | | PBT3952_rev_ | TTAATTAATAATAATAAGGTTACCAAATAC |
| | | | | new | GTTTAATACG (SEQ ID NO: 221) |
| 77 | GT3971 | BT3958 | 2393 | pBolux- | GCTCGGTACCCGGGGATCCagaggaactatagtgctt |
| | | | | BT3958-500- | cttgcg (SEQ ID NO: 222) |
| | | | | f | |
| | | | 1387 | rpiL- | AAATGCGGGAGTGACTAGTGTTACTTTGTT |
| | | | | PBT3958_rev_ | TTTTTTAATTAATACTAAGGTTATCAATCG |
| | | | | new | CCT (SEQ ID NO: 223) |
| 78 | GT1949 | BT3983 | 1262 | pLYL- | GCTCGGTACCCGGGGATCCATATTTGACTC |
| | | | | PBT3983_fwd | CGGATTATTTTCGCTGGT (SEQ ID NO: 224) |
| | | | 1388 | rpiL- | AAATGCGGGAGTGACTAGTTAGTTTTTATA |
| | | | | PBT3983_rev_ | TAAATATTAGAATCGATATTCCATTTATTA |
| | | | | new | TTTTCGATTCACA (SEQ ID NO: 225) |
| 79 | GT4128 | BT4039 | 2468 | pBolux-P- | AGCTCGGTACCCGGGGATCCagaatataacaaccttt |
| | | | | BT4040-700f | atcacg (SEQ ID NO: 226) |
| | | | 1389 | rpiL- | AAATGCGGGAGTGACTAGTAAATTAATAT |
| | | | | PBT4040_rev_ | TAGTTTTATTAAAGGTAAAAAGGGTAAAA |
| | | | | new | ACAAGTTGAAG (SEQ ID NO: 227) |
| 80 | GT4141 | BT4081 | 2469 | pBolux-P- | AGCTCGGTACCCGGGGATCCctattatcagaagalg |
| | | | | BT4080-700f | gagtga (SEQ ID NO: 228) |
| | | | 1390 | rpiL- | AAATGCGGGAGTGACTAGTAGAAAATACC |
| | | | | PBT4080_rev_ | GTTTTTAAGAATTTATATAATAATAATGAT |
| | | | | new | CATAATTTTACTTTCG (SEQ ID NO: 229) |
| 81 | GT1952 | BT4088 | 1268 | pLYL- | GCTCGGTACCCGGGGATCCATTTTCCGGGT |
| | | & 90 | | PBT4085_fwd | AGTGCTGACTTT (SEQ ID NO: 230) |
| | | | 1391 | rpiL- | AAATGCGGGAGTGACTAGTTATTATTAGTT |
| | | | | PBT4085_rev_ | CTTTTTAATATGTGTACTATTTTATTGTTAT |
| | | | | new | TTATCTAATGACGA (SEQ ID NO: 231) |
| 82 | GT1953 | BT4114 | 1270 | pLYL- | GCTCGGTACCCGGGGATCCTTGAGTAAAG |
| | | | | PBT4114_fwd | AATACACCAATCTGGAGGTATATCT (SEQ |
| | | | | | ID NO: 232) |
| | | | 1392 | rpiL- | AAATGCGGGAGTGACTAGTCTTTTGTTCTT |
| | | | | PBT4114_rev_ | TTATATTAGATTATTAATATTCGAGTGTGT |
| | | | | new | CCAC (SEQ ID NO: 233) |
| 83 | GT1954 | BT4121 | 1272 | pLYL- | GCTCGGTACCCGGGGATCCAATTGAGTGTC |
| | | | | PBT4119_fwd | CATCAGGCG (SEQ ID NO: 234) |
| | | | 1393 | rpiL- | AAATGCGGGAGTGACTAGTCATTTCAATTT |
| | | | | PBT4119_rev_ | AAAGTTAATACATATTACTATCTAATCAAT |
| | | | | new | ACTTATGTTACATGC (SEQ ID NO: 235) |
| 84 | GT2134 | BT4135 | 1274 | pLYL- | GCTCGGTACCCGGGGATCCAGTACCTACCA |
| | | | | PBT4135_fwd | ATGGAGAATTCAGC (SEQ ID NO: 236) |
| | | | 1394 | rpiL- | AAATGCGGGAGTGACTAGTACCATACAAA |
| | | | | PBT4135_rev_ | CAAAACAGGATTAATATTTGTAGTATTCTT |
| | | | | new | C (SEQ ID NO: 237) |
| 85 | GT1956 | BT4164 | 1276 | pLYL- | GCTCGGTACCCGGGGATCCACAATTATATT |
| | | | | PBT4163_fwd | TGAAAAGTTTGCCATATTGCTTTTATATAT |
| | | | | | GG (SEQ ID NO: 238) |
| | | | 1395 | rpiL- | AAATGCGGGAGTGACTAGTCTTGTCCTCAC |
| | | | | PBT4163_rev_ | AAGAGAGAAAAAAAGG (SEQ ID NO: 239) |
| | | | | new | |
| 86 | GT1957 | BT4247 | 1278 | pLYL- | GCTCGGTACCCGGGGATCCGAATGTTATGG |
| | | | | PBT4247_fwd | ATATCAATTGGAAGGACGGA (SEQ ID NO: |
| | | | | | 240) |
| | | | 1396 | rpiL- | AAATGCGGGAGTGACTAGTGATTTAACTTG |
| | | | | PBT4247_rev_ | TTGTGAGGTTAAATATTGGGTTAATTATTA |
| | | | | new | GA (SEQ ID NO: 241) |
| 87 | GT1959 | BT4267 | 1282 | pLYL- | GCTCGGTACCCGGGGATCCCTGGAATTTTT |
| | | | | PBT4266_fwd | TTTATGTAATATACCCTTTACTTCTTATTAC |
| | | | | | GG (SEQ ID NO: 242) |
| | | | 1398 | rpiL- | AAATGCGGGAGTGACTAGTTGTTTTTCTGT |
| | | | | PBT4266_rev_ | GATTAAAGGTTAATAATTAGTTGGGTTAAT |
| | | | | new | ATG (SEQ ID NO: 243) |
| 88 | GT1960 | BT4298 | 1284 | pLYL- | GCTCGGTACCCGGGGATCCTGGAGAGCAA |
| | | | | PBT4299_fwd | TAGAGACCTTATGC (SEQ ID NO: 244) |
| | | | 1399 | rpiL- | AAATGCGGGAGTGACTAGTTGTTTTTCTGT |
| | | | | PBT4299_rev_ | GATAAAAGGTTAATAATTAGATTGGTTAA |
| | | | | new | AAAAAAGTG (SEQ ID NO: 245) |
| 89 | GT3785 | BT4357 | 2274 | pBolux- | AGCTCGGTACCCGGGGATCCagaagggagtcttgc |
| | | | | BT4356-300- | actatttc (SEQ ID NO: 246) |
| | | | | f | |
| | | | 2275 | rpiL-BT4356- | ATGCGGGAGTGACTAGTcccttattccacctttttattata |
| | | | | 300-r | tgacaag (SEQ ID NO: 247) |
| 90 | GT2850 | BT4404 | 1751 | pLYL- | GCTCGGTACCCGGGGATCCGGAAGTTGAT |
| | | | | PBT4403_f500 | GCTGAAGATGTT (SEQ ID NO: 248) |
| | | | 1752 | pLYL- | AAATGCGGGAGTGACTAGTCATTAATTTCT |
| | | | | PBT4403_r | TCCTTTATACACTAAATAC (SEQ ID NO: 249) |
| 91 | GT1963 | BT4470 | 1290 | pLYL- | GCTCGGTACCCGGGGATCCTCTTTGTCTCC |
| | | | | PBT4470_fwd | GAAAAACAAGAGTCCG (SEQ ID NO: 250) |
| | | | 1402 | rpiL- | AAATGCGGGAGTGACTAGTTTCAATAAGTT |
| | | | | PBT4470_rev_ | GTTTTTCACCATCTCCGTGC (SEQ ID NO: |
| | | | | new | 251) |
| 92 | GT2367 | BT4634 | 1631 | pLYL- | GCTCGGTACCCGGGGATCCTAAAGCCAAA |
| | | | | PBT4635_fwd | AAGAAACTTACTGTACCTC (SEQ ID NO: |
| | | | | | 252) |
| | | | 1632 | rpiL- | AAATGCGGGAGTGACTAGTTTTCAATGTTG |
| | | | | PBT4635_rev_ | ATTTATCTTAGGACAATCTCAA (SEQ ID |
| | | | | | NO: 253) |
| 93 | GT1965 | BT4660 | 1294 | pLYL- | GCTCGGTACCCGGGGATCCTTTTTTTCTTCT |
| | | | | PBT4662_fwd | TTTATTGAATGATGACGGATTTTAAAATCA |
| | | | | | TC (SEQ ID NO: 254) |
| | | | 1404 | rpiL- | AAATGCGGGAGTGACTAGTACTTTATTAGG |
| | | | | PBT4662_rev_ | TTTTATAATTATTAAAAGTGTTATTTTACTG |
| | | | | new | TTGC (SEQ ID NO: 255) |
| 94 | GT1966 | BT4671 | 1296 | pLYL- | GCTCGGTACCCGGGGATCCTGTCTCCCGTG |
| | | | | PBT4672_fwd | GAATATATCAAGTCC (SEQ ID NO: 256) |
| | | | 1405 | rpiL- | AAATGCGGGAGTGACTAGTGCTATCAAAT |
| | | | | PBT4672_rev_ | TTAGTGGTTTGTTGATAGCG (SEQ ID NO: |
| | | | | new | 257) |
| 95 | GT3786 | BT4707 | 1298 | pLYL- | GCTCGGTACCCGGGGATCCCAGGCAGATG |
| | | | | PBT4707_fwd | ATACATTCACAAAGAAAAAGAT (SEQ ID |
| | | | | | NO: 258) |
| | | | 1406 | rpiL- | AAATGCGGGAGTGACTAGTAACTAAAGGT |
| | | | | PBT4707_rev_ | AGTTTTAGATTTAAAGCATATTTAGGATAT |
| | | | | new | AATGCA (SEQ ID NO: 259) |
| 96 | GT3879 | BT4724 | 1300 | pLYL- | GCTCGGTACCCGGGGATCCACTTCCTAAAG |
| | | | | PBT4724_fwd | AATACTTTACGGGAACGATTC (SEQ ID NO: |
| | | | | | 260) |
| | | | 1407 | rpiL- | AAATGCGGGAGTGACTAGTATATCAAAAC |
| | | | | PBT4724_rev_ | TTTTAAGTTGTTTGACAGGTATACCGA |
| | | | | new | (SEQ ID NO: 261) |

TABLE 5B
Bt Reporter Array_Promoters

| | | | Linked |
|---|---|---|---|
| Construct | Promoter | Promoter sequence | Regulator |
| 1 | — | — | |
| 2 | BT0028 | Aggacgaaaatgtaactttgccataacaaaaaactttatgtctaa | no linked |
| | | agatatagaaactttggataataacaaagcccgggcttgcgaagt | regulator |
| | | ctgggctttgcgcataaaaaagggtgtgccaacgttgaagtgcac | |
| | | ctcaaaagttagataaaaaacttttggggtcatttcacatttgac | |
| | | acaatctctttcttttcaactttcttatacttttatgaccaaaga | |
| | | gaaagaaaaagattatgtgcgtttgaatatgttaaaatatgaccc | |
| | | gatttgtcacgtttaggtggtatttcttttttgtcgaactaggaa | |
| | | ttttgtttttgcgacctacagattttccatttcctgttttcagac | |
| | | tgcaaatttacgaataatccatatttttatttctatatatgtaat | |
| | | aataacaaattaaagaatttaataattccttaatattcccgaaag | |
| | | aaatgtgaaataaaatatatggaattttgcagcgagaaaaaatag | |
| | | taagt | |
| | | (SEQ ID NO: 280) | |
| 3 | BT0140 | Cttccaatgatactgaagagaaaatcattgctgatgatttgatgg | BT0139 |
| | | cgatggaacggacacggttggcagccatgcccgaacagcgacggc | |
| | | tgatctatacgttaaatcgttttgaggataaatcttctccggaga | |
| | | tagcctccgaattagaattgagttgccgtacagtggaaaaccatc | |
| | | tttttctcggacgccgtgatatgcgtgacttcttccgaaactgta | |
| | | tataaaattattcatataataaacaacctttggtgcttatggaaa | |
| | | aggaatcaatactgagtgagaggtatctttatgcctcaacaaatt | |
| | | ttagagaacaagaatttaatactatttttaaacctcaaatttaat | |
| | | gtt | |
| | | (SEQ ID NO: 281) | |
| 4 | BT0189 | Tctggagtccgtacttaaatccatcgaatttatcagtgatatcaa | BT0188_89 |
| | (300) | ttataagaaagaaggcaggaatatacttctttataaaaaatgaga | |
| | | attaaaaaatagatcaatgtttaacccgttaataaggacagaatg | |
| | | aaagcatcaccctcatagaaactccctaaaaagatagaagcactc | |
| | | ctaaggaatgcttctatccgcaacccggacggatattcattcgct | |
| | | tcgacccaaatgaataacgtatcgcacgttgcttgtttttaataa | |
| | | ctttagtcaattactaaaacgcacaaattt | |
| | | (SEQ ID NO: 282) | |
| 5 | BT0206 | Cgtgtggcacggctgcagctacttttacgaatccgtagttcatct | no linked |
| | | tgtacctttttatatatagtgggtacaaaaataggaaatagtttt | regulator |
| | | ggatttttgcccttttgactctgaatcctcaaaatgaggtgtctt | |
| | | atgtcgttttaatgactattgcataagcaactaataagaatgtca | |
| | | aatggaaagtaatatggtcttatttttgacggtttatatgggata | |
| | | tatctatttgtttattaatattttgttaattatgcaataaaaaga | |
| | | catttgaggaaatttggtataaaaaatgagactttttgttttttc | |
| | | tatttaactattataattatatttgtagccaaattaaagaatttt | |
| | | ataggcaatttacacctaaacaaacgataagcacttattaatatc | |
| | | tataattttagagagatggacatacactttattagtattgcagcg | |
| | | acacaagacatttatttagacatatggcgtaatattaaaaatcag | |
| | | ccaagctaatttccctattctatatattgtatagactatttgata | |
| | | gtctgtcctggaacgatgataaaagtatacttttcaaagtcgctc | |
| | | caaggggagaagtatattactgtttcatgcgtgaagcgtaatatg | |
| | | ctaagttttcgttttgtagataaatctgtaaaaataaatcaattc | |
| | | attaatttaaattcaagtatcaaag | |
| | | (SEQ ID NO: 283) | |
| 6 | BT0268 | Gtctaagtcttctctttataggaaaatgaaattagcaacaggact | BT_0267 |
| | | gtcacccattgagtttattcgaaatataagattaaaacatggaag | |
| | | tcaattattgaaggataaatcaatatccgtagcagaggtagctta | |
| | | tgaatgcggattttctaatcctaaatattttgcaacctgttttaa | |
| | | agaagaatttggcgtaactccaaaggaatatcaaaaatcttgtta | |
| | | ggataaaaataatatgaacttaaaatacacaagtacgcgtcttaa | |
| | | tggaagatgcgtacttttccatatatcaatgatctatcccatttg | |
| | | aatgattcctgaacttatattgaacgatttttagacctgttatag | |
| | | ttaatagcgattatggtccaattttggaagtttttgaatgattag | |
| | | agaacttctttctactggataactcgcacttttgtgacgcatttg | |
| | | atgcacaactaatacttatttggtctaaataactttataaatcta | |
| | | atagt | |
| | | (SEQ ID NO: 284) | |
| 7 | BT0317 | Ttcaatttcaataataacttttgtgtgttagagtccctggaatgc | BT0326? |
| | | tgagtgtctacagcagttccagggatttctttattatcttttcat | BT4338 |
| | | ttaatgccaaagaaggttcattctgttttgaatattgtaattttg | binds |
| | | ttagccgatataattattctttttgtttaaagaaaaaaaactact | upstream |
| | | tattttcttgttatttaaaataaattcatatttttgtaatcgtag | of BT0317 |
| | | tcagaaccaaactatgaaatattgacgtcctctctacacacagac | in No C |
| | | aatttttattaaccttaaaagagcagtatt | |
| | | (SEQ ID NO: 285) | |
| 8 | BT0365 | Tcaatgtgacaccaagcgcactgctgaataacgaagaataactga | BT0366 |
| | | tataagggttgctattatgaaacaatgatagcagcccttcgtctt | |
| | | aaaatgagctcattttaagagacatccgtccaagattatactcaa | |
| | | agtgtaaaaaagacacttatataaccgtttatccacctcgaaaat | |
| | | tcatttgtccacctttaatatactcatcggttgtatttttgcgac | |
| | | actacaacgagtagaattattgttgaaccattaatatcaatatct | |
| | | atgaagacaaggaaatggccgttactactt | |
| | | (SEQ ID NO: 286) | |
| 9 | BT0439 | Ttcattatccggccagaagtttgatgaaaaacaattttatcaatc | BT0433? |
| | | aattactgacttcgaatatgagtggacattatccaaagagcacca | |
| | | tcctataatttctggagagaacccgatattgttggccaagacatt | |
| | | atctgagaaatatatgcaatatttttattaagtacatacctcaaa | |
| | | ataattataaattataaaacaaaacagcctatgaatataataccg | |
| | | aacttataattaaggaaattatcaaatctaaagaagaaaaaaata | |
| | | cagaattacttaactttaaattattaatgt | |
| | | (SEQ ID NO: 287) | |
| 10 | BT0454 | Aaaataggaatttgcgtctgatgtacaaaaacggtgtctgaagct | no linked |
| | | ttcacaaagatagagcaccctaacaagagaataagggtaaataaa | regulator |
| | | taatgatttcttttcataaatgggggtattagttaatttaacgaa | |
| | | ggcaaagataagattttctctgcatatatttaacagtcaaagcga | |
| | | caaacttgcgttttttaccaagttctcgtaggactagtcaacact | |
| | | ctgaccaccgttcttgctcttgtgatcattgacagggtcggccgc | |
| | | aagcaactggtctattacggagtttcgggg | |
| | | (SEQ ID NO: 288) | |
| 11 | BT0483 | Gctatggctacggatacggttacggacaggaaaaaggtgctaaat | no linked |
| | | cgtaatccggttctaagaataaacctgagaataaacgatagcccg | regulator |
| | | tttcaatctgattgaccacagtatatcccccaagatatagtcagt | |
| | | cagatatagccgaccgcttgattttccccaatcaagcggtttgtt | |
| | | gtattcatctacgttgacaatataatttttatatcccatatcaaa | |
| | | gaaaagcgggtatccggacaaccagatgcccgttttttttgttat | |
| | | atttgtattatcccttaaaaaatgataatt | |
| | | (SEQ ID NO: 289) | |
| 12 | BT0753 | Tattgcccatcgtctggaacgtaaattccaggtgaagatcagcat | BT0752/ |
| | | agaaagcgaacgtctgaaggccgaacacttctccggttctttcga | 53 |
| | | cagcaatcagaatatttatgatatattacacgaaatcaacgttga | |
| | | gaaacaatacacgtggaaagtcagtggtgacaccatctttattac | |
| | | cgataaaagaaagggggtaagataaaaggaaggagaaagaaaatg | |
| | | gacacagccgatagatataccggcagtccgcagctacgataaata | |
| | | actatatgttaacctttaatagtgataaaa | |
| | | (SEQ ID NO: 290) | |
| 13 | BT0867 | Ttgatgctttttttaaaagttatttcttcgtttaccctatctttt | no linked |
| | | ccttttataattctgataaagtaatatttcaggaggtatttgaaa | regulator |
| | | ttaatatgtactcataatactcatcccaccaagttctccatgaac | |
| | | ttataccgccttagagtttactaaattctatatttcacaacctaa | |
| | | tttttaatcaaaaatcaacacacaatcaatgcctatggaaatatt | |
| | | acagtattatgatttaccatccctatcgtttacaaacaaaaccgg | |
| | | taaactagttattattttaaaatcaaaatt | |
| | | (SEQ ID NO: 291) | |
| 14 | BT1030 | Ggtggattacaatgatcttatccattcgtcctttatatgaatcgg | no linked |
| | (700) | ttaaagtggaagttaaaacgatgtgaagatgtggagaagaacgat | regulator |
| | | gattggaatataaaatatttaaaagaaaattttcaacaataatgg | |
| | | ggaggggaattatgatatagtttaataaaactaagagaaaaaact | |
| | | tcagattctattttttattctttttaagagattacgattctttta | |
| | | gttgtatctttttatttatgtgaatagatgtctgatgatgtaata | |
| | | tatcattttatataaattaaaatttaatct | |
| | | (SEQ ID NO: 292) | |
| 15 | BT1040 | Tggtgtttcctttaaaaccaaaatgccgttccttttaaaccaacg | no linked |
| | | actgctccgttcgtatattttaaagctctttcatagaattgaaga | regulator |
| | | tggcatttttatgcactaagcaattgttttatgcaaacacatcgc | |
| | | ataatcttgcaataaattatacatttttttgctatttattagatt | |
| | | tcatagactttgactacctggctattcttaaaaatagaacataaa | |
| | | gaagtatgaaaacagctataaattcattactcccaatcaatcacg | |
| | | cttgccaacgcccaacccctaaaagtcaca | |
| | | (SEQ ID NO: 293) | |
| 16 | BT1042 | Tagtttttcagaactacttaatgctctatttatcaaatgattatg | no linked |
| | | tatttatttttcaaaaaaatgcttagtctaaccattttttacgaa | regulator |
| | | aaaaaatcttattttcgtttagtatggttgcagtatcaaatgtat | |
| | | tactgatgaataagcgcttatgaaaaatcatttttagtgtaacct | |
| | | ttaaatttataaatatgagaaacagagatacatagatcctttgta | |
| | | tgagtggcggttatatttattacccgcacatgtttgttaactacc | |
| | | taaaaactatttctaattaaaaagaataag | |
| | | (SEQ ID NO: 294) | |
| 17 | BT1119 | Acaaaaggagataaatgaccagatatataacagtagtattacatt | no linked |
| | | aaggtaaagaacaaccatggtatagtattattgcaatttaactat | regulator |
| | | ttaaagtttgtttataatcaaatcatttaaagatttttgtccttc | |
| | | tatgattttttgtgtttgataaatgtcgtaattaaataaaaaata | |
| | | tcacgcataatgactttttcttttttctaaatttcttcaaatgac | |
| | | aaattattgttacctttgtaatgtaatgtagttaatggttgcgct | |
| | | tataaagtccttggaaaggatttatgtataaaagaggtagatgtt | |
| | | tactctgttcttgtttggattatcagcatcactgatatgaaataa | |
| | | caaaaaaaatatacaatggctaagcggacggacaggcagggaaat | |
| | | agtacagattcccaatccccctctttataatacattgcgtataag | |
| | | atttattaactgtctggtctggggaatctacagagatttaacgaa | |
| | | tcaag | |
| | | (SEQ ID NO: 295) | |
| 18 | BT1279 | Agaccaggcaccactatctgaggtaatggatgtgatagtaaatgt | BT1278/ |
| | | agtaggtcaaatgaactataagataaaaggagaccgctgctactt | 79 |
| | | aatcccaaagtaatgggtatcatcccaaaagaaaaacagtaaaaa | |
| | | aacaacaatataataacctttttaaccactaaagattcatgcaaa | |
| | | aaagtatccggaacggagccctaactctatcccggatatgaaatc | |
| | | aagattctcaatattagagttttactctattcaattaccgtgaaa | |
| | | taatttagttaataacctaatactaagaaa | |
| | | (SEQ ID NO: 296) | |
| 19 | BT1440 | Tcttaaaagcgcttggtttggccggtatcacagaatcagtttgca | no linked |
| | | gactatcgctcataagggtatctctcctttcagcctgggcctgta | regulator |
| | | aggagagtgacagaaacagaaaaagagatagaacaatttgcttca | |
| | | tcctgtgatttttcgttgttatgattgatgctgttcgtttcacga | |
| | | atgtactacctttcgttgatttatactaataatcgtctaaaaaaa | |
| | | tgtttcgtttcctaaacaaatgagatatgtattctgttagttaga | |
| | | acaagcaaataaatcctaagccacaaaatt | |
| | | (SEQ ID NO: 297) | |
| 20 | BT1551 | Aaggggaaagtcgaaaggtggcacaaaggcagcctgctttttttc | no linked |
| | | ctgtgcatacccctccacgcaacaggccaataataaagcagcaat | regulator |
| | | atatcttttcattatcttcttcacttttttactcctgcaaatgta | |
| | | cattattccaactagattacgaaatatccgggaagaatgatagca | |
| | | gatgaacctagttaataattccttttattattatattcgcaacta | |
| | | aactgtaatcattatattattaatattattacttttgcggctgtt | |
| | | tttaataataattaaaagtaaaaggactta | |
| | | (SEQ ID NO: 298) | |
| 21 | BT1618 | Catacacatcatcgtccctccgcatctggctaccaccgatgtata | BT1617/ |
| | | taccatcaagttcaaacaaaaagcccctatcaaggagatcatgaa | 18 |
| | | tatcgtgaccaaaaccataggaaacatagactataaagtcgaaga | |
| | | cgagaacattctcttaatttattcacccctaaataagaaaggagg | |
| | | ccgatagacagaaaaaagggagtcgatatttcgcggataccaact | |
| | | cccccaaatcaaaattacttcatttaaattagttgtgtacactta | |
| | | aattagtattaatgattaactcacaaaagt | |
| | | (SEQ ID NO: 299) | |
| 22 | BT1632 | Gggcggaagagtttttaagagagaatatagtatcatgagtatatt | BT1635 |
| | | cgggtaattagagttaatgaaaggcggagatattcggaaacggat | |
| | | attgccgcctttctttttttgtctcgaaagttgtctgataatgtc | |
| | | tgttgacctcgaaaaatgcgataaaatggactattttagacattt | |
| | | ttggaagaatcatagccaaaatcgaacatcttaaatcttagatat | |
| | | aacctaaatttgtggcagtgaatgtcaggaaagacttcgctactt | |
| | | atataaaaggtaatcttaattaataaactt | |
| | | (SEQ ID NO: 300) | |
| 23 | BT1683 | Gtactatgcagagagttacggacacgcagccagttgaccggaaaa | no linked |
| | | aagtcgttctttcggaagaaacggtaaccgaacatggaattatcc | regulator |
| | | agattgcggaattcaataagaatgcaggtgcgtgatgcttcttgt | |
| | | gtgagatgctccagcatttcaccgaaaatttcttcctccttctct | |
| | | ttacttgccggtagagaagtgtccagaaattcacctgcaccatat | |
| | | accacacattgtttgacaagagaggagggaagccactttttggtc | |
| | | ttgcgtatggcggcaagcaggcgtgctacaggtctgccttcttct | |
| | | gttgccactatcagtagcggggtatatcccggagtcgattcatag | |
| | | atctgaaacaactccttggaatgaaaagtattggttcccggtaaa | |
| | | tcaggaatgtcttttccgcgataatatgttgttagtttaagtggc | |
| | | atccaaatacaaatttacactttttattcttcattcaacaataaa | |
| | | aaagagaatttgttggcttagcctaaaatttgtctttttatcctt | |
| | | tgcatatatttgaggaaaaaaaggcttggaaaagcgaatttttgt | |
| | | attattgtcgtcctttgtgagtacagaagtaaaatggaaacgtct | |
| | | atataaaagattatgctaattgtattttactaaacaaaggaaaat | |
| | | taatattaactttaaattaaaacga | |
| | | (SEQ ID NO: 301) | |
| 24 | BT1763 | Tatcattcagttttctgttggttactttgagtgattatatgcttg | BT1754 |
| | | ttatatgaataatatccctttttacaaggacttaaattactatac | |
| | | aaaggtagtattttctatgattgagagagaatataatcccgactt | |
| | | tcttatgatggagaattattcttataaaaccctataaaatagcgt | |
| | | tctatgcaacatatcttacagcatttgaaacatttttttcagtcg | |
| | | gttgacttaaatcaaccgtaatttgcatcatcgaaaagagaaaat | |
| | | tcgtacttttaaattaataacattaaacta | |
| | | (SEQ ID NO: 74) | |
| 25 | BT1775 | Gcttaattttcgcttaattgattattaaatatcccttaaatgtag | no linked |
| | | aatttatgcaaataagtatgctgaagaggggataaaagttagctg | regulator |
| | | tagttataaatattcctgttattccttattatataggaaacttat | |
| | | attatatgagaaatagggaatagtacaacttaaagcttatatcat | |
| | | ggggagcgctttgaaaatctatcaggaataacgccattgtaatac | |
| | | taaaaatccacatcttatataatcggataagtcactaattggata | |
| | | tctcctaatttgtgttatgtttgtatagtatttgccttatttacc | |
| | | tttttagcctttctacttgaaatattcttctttttgcaggaagtg | |
| | | aatactcatttagataaagaaatagggtgtggacagcatactaca | |
| | | ggatataaaacttcccccttttcctgaaattaatacctaagattg | |
| | | cgaaaaattataagaagatagtattagttttaaaattaattagaa | |
| | | attaatcagagtttaatcgtattttataaaaaatcttaagcaggc | |
| | | tcttgttactttgcatcagaactcaaaaataaccttaaatattaa | |
| | | gtttgttcagtcgacgctttcttaaataatagataaaaagggtgt | |
| | | tttcgaggtgtaacaaatagcaaatgtataatataaaaaagagac | |
| | | cgtgaaaaattatttgaaaatcacg | |
| | | (SEQ ID NO: 302) | |
| 26 | BT1876 | Tggaacgtgaacttgaaaacgaaatatataaggctatcgataaat | BT1877/ |
| | | tgccggacgaatgccgtcgggtttttgctaaaagtcgctttgaag | 76 |
| | | gaaaatcatacgaagaaatttccggtgaactgggaatatctatta | |
| | | atacagttaagtatcatataaagaatgctttagcttctttacacg | |
| | | ctcatttgagtaagtacctgatttctctattactattttttttca | |
| | | ggtaaagcgaaaaaaaatcattttctgactacccttaattaaaaa | |
| | | acaactgtctcctttatagaaagcataaat | |
| | | (SEQ ID NO: 303) | |
| 27 | BT2107 | Tgtaatctatctaattttatgttcgtttcataaatttaaaaccat | BT2103 or |
| | | acacacaaagccgcattaacatttattctcaaaacaaccattcat | BT2104 |
| | | atataaatttatattaaataaataaatctttctagttttcctgct | |
| | | ttaaatatatatatttgcaatatgtatatacatatagacattata | |
| | | taatacatatatcataagcaattaatactatgtagattaaaatat | |
| | | acagcctttatatacaagaaatcacattaaatctaatataaaacc | |
| | | cttatgacttactttttaaaatcacagagt | |
| | | (SEQ ID NO: 304) | |
| 28 | BT2170 | Cgaccggcttccggaacgttgccgggaaatattcatccgtatccg | BT2169/ |
| | | tgaagaaaaacaaagttatgcccaagtagcagaagaattggatat | 71 |
| | | cagcataaagacagttgatgcccagctacagaaagctgtctcccg | |
| | | actgaaagagatgatttcgtccagcggataaccggagatcacccg | |
| | | aaccgttcagcacttcttttttatattaacttttatttgcacaaa | |
| | | aggcgtagggagtttctgtcaatctccggtctttatcagcaaata | |
| | | aaccgattaatcactaaaaaagattgaatg | |
| | | (SEQ ID NO: 305) | |
| 29 | BT2197 | Ttgcttataaatctattcgtctgacagttgttgaactttcgtctg | BT2198/ |
| | | acagttgttgaactttcgtctaacaactgtcagacgaaagttcaa | 2197 |
| | | caactgtcagacgattaaatttaagtataatgaatactaaatctg | |
| | | tgtacatcaagaattaaatcttagagtaaatccgaacagattagt | |
| | | tcgtttcaaaacagttcttagcttaaacttattggattttacttc | |
| | | ccttccttgattttttttcattttcctttggtggtaaatgtaagt | |
| | | tgctgtgtcttcttattaaaagcgcgcaat | |
| | | (SEQ ID NO: 306) | |
| 30 | BT2203 | Gctggcactggcctatggtatgcatgcccttaaccatacaggttc | BT2204 |
| | | gtcacaagaagcaattctgcaatattccctgtttgtgcgtgagta | |
| | | caagcaaatgatgaatgaagaatattctacttcttatgcagagct | |
| | | gatgagcaaaaatcctccttttcatcgataaaaacatatcggtta | |
| | | atgatatttattcgcttattaatccgttattaacccatcactaat | |
| | | aagttgtttaaccttgctacattatgtttgcagcgatgttattta | |
| | | cttattgttttatttaaaaacctaatacgt | |
| | | (SEQ ID NO: 307) | |
| 31 | BT2260 | Aataaaagcgcctcttatagcatgatactattttttgtttataat | no linked |
| | | agctccttgttttatagatataatattgattataagataaatagt | regulator |
| | | tttcatttcggtagagaagtggtattacataaaagagagtaaaac | |
| | | attagctgaattagggtaattaactgtcaaatagatatataagct | |
| | | atttctgcttgataaaatatcgactggtgttactgatttatcccc | |
| | | tatatacctaactgattatcagttatatataaatccattagttct | |
| | | gcaaagactttgcagatttcgtatattggt | |
| | | (SEQ ID NO: 308) | |
| 32 | BT2264 | Ggtggtgagaaatattcgggaaatataaaaatagtatccaataat | no linked |
| | | aatacctcatataaataaaataaagacataactttcttataatga | regulator |
| | | atgaactaaatgattgaaatggatagaaacagggtatcattcgtt | |
| | | aaagaaaagggtagttataatgctgataaataattgattatcacg | |
| | | tttatatatggatacaataacatatatgacgattaggtttatatt | |
| | | tctcattattataataatcaacactttatataaagaattagttct | |
| | | gcaaagactttgcaaatttcgtacatcggt | |
| | | (SEQ ID NO: 309) | |
| 33 | BT2362 | Aaagatagtaaaaatcaacccattccgattcaaagtcaactttat | unknown |
| | | tttttttcaatccacaacgctctatgcctatttttgtccaatcag | |
| | | tctaaataaactgatattgatagtggcgccacatcaaatacatac | |
| | | actcagagattgaccgtcaattcacggtacaatcgcagttttaac | |
| | | caataaaatctaatagcctatggagtaaatcaaatcggtataatc | |
| | | ggaacatagatttaaatcattcagagatttaactattcaaaaacc | |
| | | aattagtgaacatacaattaaacaaaaaac | |
| | | (SEQ ID NO: 310) | |
| 34 | BT2364 | Ctacgacctttaccgctaccattatcaacggcagtgagtatcaga | unknown |
| | | atccgggatattaaattgtaagacctatatacccaatcattatcg | |
| | | gtacgataatccatatagccacgctatttatcgtaccaatgattg | |
| | | gggatattttccacacctttccacatttttccacacctccccaat | |
| | | aatatcgtttttttcaactctgctgttaatctgtatcttatcctt | |
| | | tcagcatttctttatatcctattggtatatcctttgtatcgactt | |
| | | tatagcaaatgtatttttaaatataaaagt | |
| | | (SEQ ID NO: 311) | |
| 35 | BT2392 | Gcaaatgataaaaaaagcaattttgaatgaaaagatatacatgga | BT2391 |
| | | tgaataaaagcatatgctcctgattccttgaaataccctaagttt | |
| | | gtatcctgaaaaccgaacggaaagtgtgcttcacaattatatata | |
| | | gccatttacacctttgaatctataatatttaaaacaattaatcac | |
| | | atgagagaaaaagccaaaagcccttaagtcaaattgaaatcaatt | |
| | | agaatctattaaaccttatttaaatagaaagcctaaaagaccttt | |
| | | tttcaaactaataaaatcttatagaaaaac | |
| | | (SEQ ID NO: 312) | |
| 36 | BT2462 | Ttgcaatcattacctgaacaaacctcccgtatatttactctcagc | BT2463/ |
| | | cgatttggaaataaaacaaatcgggaaattgcctgcgaattaaat | 2462 |
| | | atttcaatcaaagatgtggaatatcatatatccaaatcacttaag | |
| | | gctcttagaaaaacgctaaaagattacctgcctttattttatttc | |
| | | tttttttaccatatgtaacaccgattactacgcacgttcctttat | |
| | | cttttgaaaaaaatattctttttcgtttagggtaaaccgaagatg | |
| | | aatcgttatataaatgaaaacgataaaaag | |
| | | (SEQ ID NO: 313) | |
| 37 | BT2529 | Gaaacgccagacgaatcaggactttaccgaaaagcaatacgagca | no linked |
| | | catccacttcatgatgaagctgaccgatgatgccctcgcacaaat | regulator |
| | | gattgtggtggtagagaaaccggaacaccaaagcattgatatcaa | |
| | | caagtctttcaacatcgaaaacgaaatcaataactaccgtaacca | |
| | | actgaaaaatcagaatattctggatgtcaacaacaaggaatatga | |
| | | ttatcagatgggagtctattatatggatattatcgccgaatgtga | |
| | | gaaactgggcgactatgtagtgaatgtggtagaagccagcagtga | |
| | | tgtaaaagagaagaaagcctcttaaataaagaggaactcaatcaa | |
| | | agaaatggatagccaatttatgcgttgcagcataaaattgactat | |
| | | ccatttcttttttatttccacacagacaaaacaactcatgtccgt | |
| | | ttacaaggacactcacactttacaaagacgttacattttttacag | |
| | | acgttacgattacatttcttttccgtgaagttcccgttaagcttt | |
| | | tactgcgcctattgcaggtcttttttttgcatatacatttgcgcc | |
| | | aaaatacacaggggcctatgtttagtaccattttccaatgaccgg | |
| | | acaaaggcattccaaaagaaaaccaaatctctaatttaaagcaaa | |
| | | agaaatgaaaattaaaaaagtattg | |
| | | (SEQ ID NO: 314) | |
| 38 | BT2561 | Aagtgcaggagcatgagctaatccagtatgaatcccgttccatca | BT2561/ |
| | | cccgaagtctatcttcgcccaaagagcagacttttaaaacagaac | 62 |
| | | gtatcattttatggtttctgaataaaagaaatattcccatcctga | |
| | | aaaaagacagagaagcattttgggaaaagttgtctcctgagattc | |
| | | atgaattatataataataaatatgaaagtcagcttttacgcctct | |
| | | ttgactttacggcgtggatggaatccaaaattcggaaagaaaaac | |
| | | tgtctgaagtcctcagagcacgtgctagcgcaaaagaatgttaaa | |
| | | gagtaaggtgtttgattcgtatttcagacgataaaaacactcttt | |
| | | tcaaatctccattttctctttgtaattatataaattcgcttcaca | |
| | | ttacctctatattctctcctctatacaaggaaaatccaatataag | |
| | | atatcatttttgagattatataaaccaattattaatgtagaaaac | |
| | | tatgcatgattattgctttcatttgcatagtcttaatttttaata | |
| | | ct | |
| | | (SEQ ID NO: 315) | |
| 39 | BT2627 | Agatagatattgggagtgcatatgtttgtttttacaagatgataa | BT2628/ |
| | | agtgtattatagcgacaatatagtaaatatttgtgccaagagaaa | BT3786 |
| | | gagactgattaaatattctgagggaatggaagtttgaaaacttac | |
| | | atttgcatataattgacttgtgatttttattattagaatagttat | |
| | | tatttgttatacttttaaattaaaatgcttatgaatattatagaa | |
| | | agacatccaccatgattaatgatatccgctgtttaatcatataat | |
| | | agaaactaatcaaatataaagaatatagaa | |
| | | (SEQ ID NO: 316) | |
| 40 | BT2803 | Tatgaaagtcagcttttacgcctctttgactttacggcgtggatg | BT2802 |
| | | gaatccaaaattcggaaagaaaaactgtctgaagtcctcagagca | (rusR) |
| | | cgtgctagcgcaaaagaatgttaaagagtaaggtgtttgattcgt | |
| | | atttcagacgataaaaacactcttttcaaatctccattttctctt | |
| | | tgtaattatataaattcgcttcacattacctctatattctctcct | |
| | | ctatacaaggaaaatccaatataagatatcatttttgagattata | |
| | | taaaccaattattaatgtagaaaactatgc | |
| | | (SEQ ID NO: 317) | |
| 41 | BT2818 | Tgttccttacaaagcctccttttccattccgtctataattttgta | BT2826 |
| | | tcaaggtctacccatttgttacagatagtgtttctagctatttgt | |
| | | gcacctaatttggtgaatattgcacgtccatttttcacatcaccc | |
| | | ctaattttgcatcatcattaaaagataaatgaagttaaccttatt | |
| | | gcacataagcaaaggatatatttagaaactgaagagagaataaag | |
| | | agaataaaagtcacccccaacccggttgacctaactataaactaa | |
| | | atatattattaacctatcaaattaaacagt | |
| | | (SEQ ID NO: 318) | |
| 42 | BT2859 | Actacctcaaaacgcagatcgtatccttgctgtataaccgtcaga | BT2860 |
| | | aagaacgcgaagcattctccaagcgtccgttcttcccgacgaata | |
| | | agatgcagatgaataaggtggatgaagagtttatgaataaagtga | |
| | | ttcgtaccatcgaggaaaatatcatagataccaatctgaacgtag | |
| | | agcatcttgccgagattctgggcatgagtcgttccagtctgctgc | |
| | | ggaaaataaagatgctttccaatctgtctcctgggacttcatccg | |
| | | tttaatccgattgaggaaggctgccgaactgattcatgaaggtaa | |
| | | atacctgattggagatatctgctttatggtaggtatcaactcacc | |
| | | gtcctatttcagcaagcttttcctgaaacagtttggcatgacacc | |
| | | taaagatttcgagaaacaaagtcaggcagacaaagaaaagataga | |
| | | catgccttcgtgaccgtccgggtgcactataacaaatgatgcaca | |
| | | aaaatgaaacatctgcggaaaatatgcttcagatgtagtctggtg | |
| | | ctgcaaaaatggtattttttgcagtgatttttattataccgccta | |
| | | tatatatactcctacttttgccatcgttccggataaggaaagata | |
| | | acccctttcggtgaacggattgatttatccttaaatgtgaactct | |
| | | aatactaatttaaaattctgatac | |
| | | (SEQ ID NO: 319) | |
| 43 | BT2896 | Gttctaacttttaccgcaaactgaaaggagtattggatatgagtc | BT2897 |
| | | cgaatgagtatcttcgtttgatacgtttaaagaaagctgcccagc | |
| | | ttctgaaagatggaagatatgggattgtagaaatttcttatatgg | |
| | | taggattcaattcaccttcttatttctcaaactgttttaagaaac | |
| | | aatttggagtgttaccaaaagatttcatgcaatgaacaaattctt | |
| | | ataaaattgctctaaaactgattctgactatctctgcactgtttt | |
| | | ttggatagaatttaataatatcaggttaatttcttatatcctttt | |
| | | caggtaggttccgataactttgcgaatgtctcgaaggtaggaact | |
| | | tattttctgaccagtctatttggttgggaaaaaactttcttctag | |
| | | atcaattaatatagaagtaacggataaataccagattagaaactg | |
| | | gctttataagctattaattattattaatcaatatctaatttttcg | |
| | | cgcgaatatgattttgtatgtattgtttggtagatagggagaaga | |
| | | gttgatacataaataaggaattggcaggtattagtttttttataa | |
| | | aactcttcttccacttttcttcgctgaagtatataatccttaaat | |
| | | aatacgatttatgaacaaattccttttataaggatcatgatataa | |
| | | atgtttaaccaaaataataaagaaa | |
| | | (SEQ ID NO: 320) | |
| 44 | BT2907 | Tgtggacattggtacggacgagcgttatccagttatggcttactt | BT2910 |
| | | caaggattgacaggagtccgatatgatgctgtagataaaacactg | |
| | | tatattaattcaaaaataggagactttattagctttatcagtacg | |
| | | gagtctggatttggtaatatagaacttcgttccggaaaacctttt | |
| | | gtaaaagtagtatccggtcatatagaggttgacagatttgtcgta | |
| | | tcggggaaagtagttgaataatcaacaataagataataactaaat | |
| | | ttttaagtgataactaaatactataatatt | |
| | | (SEQ ID NO: 321) | |
| 45 | BT2909 | Ccgggcgaatggcacacctattatcctaattatcaaacaggttgg | BT2910 |
| | | agtcattattggatcggcttttgcggtccggaagttgatagctgg | |
| | | atgacgaatgagtattgttcgaaagaaagtcctgtatttaaggtc | |
| | | ggtatcaatgatgagatcgtttctttattcagaaaagctattgat | |
| | | gttgctaacgaagaacctactttatatcaacgcgtattgagtgga | |
| | | ttggtgacctatctggtagcactaatgtgtagtatcgataagaac | |
| | | cttcaggtagagaatgatgatttctcttctaaaatagattatgct | |
| | | tgtgtgctcatgaaggaattgatagatcaaccagtttctatgcaa | |
| | | gagattgccaaaaaagcaggtatgggatattctctttttcgaaag | |
| | | ttgtttaaggaacagaagaattatgcacctgtgcaatatttccag | |
| | | aatctgaaaatacagaaagccatagagttgttgactactactact | |
| | | attccggtaaaagagatagcatataggctcgattttgaatccccg | |
| | | gcttatttctctgcccggttcaagaaacaaacaggaaaatcacct | |
| | | atagaatatcgagaagaatttcgtatcaaataatgatagtgttct | |
| | | ggcaattatcgaaagtctttctcttttaagatgactaagtttgta | |
| | | tcataattaaaaaatgaataatgct | |
| | | (SEQ ID NO: 322) | |
| 46 | BT2922 | Gtacatgcaggatctatatccccgttgcattgcgcatatatacac | BT2923 |
| | | aatgtacagggcatatagatcctgcatattgcatatatatagctt | |
| | | atctgcctcttttttattgcaatattttatgtgcttttatgtggt | |
| | | atatgaaacaattatctcattcctgtttcatttggtatagatttg | |
| | | ctattctttcttctatttgagacattattgcgatttattgggata | |
| | | tttatattacccaaaaactatctcatccgctaattttgtctgcgt | |
| | | caatcagtcgaaacaataatctaattatat | |
| | | (SEQ ID NO: 323) | |
| 47 | BT2952 | Cttcggaattgctaacacctacgggactaaagttacttacaggta | no linked |
| | | cagtaattattcttgccggaatcattttctttgcgatagcagggc | regulator |
| | | acaacagagatagcaaaagccgccagacgcactctgaaacaccta | (BT2957 |
| | | ttaagaaaaatttcaggaaagggttaattatctgtttgcttgcag | maybe?) |
| | | gaatattcggacctatgattaactttgctttcgtttatggggcac | |
| | | ccttacaagaaaaggctgtagcaacgggagcatcttctctttatg | |
| | | cagccaacgtgatatggagtatcgctttgagtgccggatttatta | |
| | | ttaatctccttgaatgtatccgtttgttcggcaagaatcaatctt | |
| | | ggaaaacttaccgacatcgtacaaccggaggcctgataatggctt | |
| | | ctctggcaggggtattatggtatctgagcattatgttctatggta | |
| | | tgggaggcagttttatgggagttttaggagcttctgtgggctggg | |
| | | ctaccatgcaatcgacagctattattgcaggtaatgtggccggac | |
| | | ttgcttccggcgagtggaaaggagctacccgctatgccatccgga | |
| | | tgatggtgataggattggtatgtctcatcggaggagtggtggtga | |
| | | ttgccctctaacgactatacttaactatttatattgacttaatta | |
| | | ttatgttattcaaaaagtctaactt | |
| | | (SEQ ID NO: 324) | |
| 48 | BT2956 | Aatgtaaggacaagtcctgagaagaggttatattcactcttgcaa | no linked |
| | | tatgaaacttttcatttgcactcagaaaataggatagcttaaaaa | regulator |
| | | cggaatctctcatattcttttttgatttgattgcaatattaagag | (BT2957 |
| | | ttagccttcacaaacacaacccgtgaagactcttttttatatgtc | maybe?) |
| | | aaagcataaaaagtcggaatagcgtaaaaaaactaccgctatgaa | |
| | | taatttctatcggaaaaacgtatatggacaacctctacctttgtc | |
| | | cccatcagctaatactaaaagaatcatatc | |
| | | (SEQ ID NO: 325) | |
| 49 | BT2968 | Gatgtgcactatgtcttggctgatgtaaccttggaacaaggtaag | BT2971 |
| | | aatacaattaaaaccgttgcaacatataatggaaaagaatacact | |
| | | gacgagattgaatggaactataccggcgaaaagaagcgaagtgcc | |
| | | gattggagtgaaaacaaagaagagcacgcaggttggtaatcggag | |
| | | acgtttctgtatgtaatctgaaaaatgttgaatctaaaatataaa | |
| | | agcctatgaatgaaatgagaaagaatctcttttaaatggaaaaca | |
| | | agtatttatcattaactaatctaataattt | |
| | | (SEQ ID NO: 326) | |
| 50 | BT3011 | Atcaacgagtatgactggcgctcaagtaaatatcgctttaccaac | BT3012/ |
| | | aagccattacaggaattcatcgattttatcaaccgttcataccat | 11 |
| | | gtaaacatcataattaaagaagaaaagctcaaagagctgaagttt | |
| | | aacggaactatacgtaaagacgagccgcttaccaatatcatcgaa | |
| | | aagatatgtatcagcctcgatttaaaagaaaaacaagaaaataac | |
| | | aacatcatattatattaatacataagaatcaattatcaatatatt | |
| | | cactattaaccttttagaaacatgaaaaac | |
| | | (SEQ ID NO: 327) | |
| 51 | BT3024 | Aaaaacaatgcaagaaatggaaactgctttcatcatatccggcaa | no linked |
| | | gaatattaagaaaggactccggtttgacgatgtaagcatgatgca | regulator |
| | | atacgatgtggcgtccactatcgcccgcatctttcatttagaaca | |
| | | accacaagtttggataggtagacccatggagattgttttcaaata | |
| | | atcattcaggatgattaataggatgggaaaaggagaagctaaaat | |
| | | aactggcttctccttttttatttatctatccacgctcataagata | |
| | | ggagattataaatactatttcttataagtaacaaaatgagcagtc | |
| | | ataatagttttatttcctataagtaatagaaatcacacctccaca | |
| | | gctgttaattaccgaataatacttgcatcaacatacatatcctta | |
| | | tacctttgtcccccggtaacacaacataatataacttcaatcata | |
| | | aaatgtactcttttgacattcttaataagcaatccaactgattaa | |
| | | agcagcattcaaacattctgataggacatcttatcttttgtttat | |
| | | tcaaattaaaatacaatacgatagaattgtattagtaaatgataa | |
| | | aatactacttgtctgacggagcattatcgcctacttttgcatcat | |
| | | cagttattggttgaagtttccatggatcaatcaacaattgaatag | |
| | | agtttataatctaatacaataaatt | |
| | | (SEQ ID NO: 328) | |
| 52 | BT3047 | Agttgtgtttcttgcagccaacgtgtgtaaatcgtttatgaatga | maybe |
| | | ttctctacgttttttgaatgatttttataatgtaaaatctacact | BT3048? |
| | | cattcgtatctttgctgtagataacaacgaaataaactaatatta | |
| | | tgaaaatgaaaactaatataatctttaaaaaactaaagtcattcg | |
| | | tcaatcgatgtctttttcctgattatatgtgctgttttatcttcc | |
| | | tgcgtttataacgctgatatgccaataaagaatttttagtgaacc | |
| | | aataatttaatatgtaaaaagaaaaacgtt | |
| | | (SEQ ID NO: 329) | |
| 53 | BT3090 | Gctactctgtgaccactatttataattaccggacacgtgtccgca | BT3091 |
| | | acaaggcactcggcgaacgggatgagtttgaggctaaagtaatga | |
| | | aaatagggaaggtggaagagtaagaaatccactacttttttagca | |
| | | cggtcgctattctataatattcttattattagtgttttatgctgt | |
| | | tttgtactgtataaaccactatatttttgctatctggcaaaagaa | |
| | | gcgcatttaattgtggatacttttgtttcagtgaaaacaaaaagt | |
| | | aatcctaatattaactttaaattgatgtac | |
| | | (SEQ ID NO: 330) | |
| 54 | BT3108 | Taatgtagctggcagacatccggaactgcttgagcggttaaagca | BT3097? |
| | | ggaattttttgtgcaaacagatggattttatcgtagtgaggtgga | |
| | | agaagaaccattaaaataagcccgataaccccttctggaaagcat | |
| | | ttgattgattaggtattagattgttttgcaggaattggtgtagaa | |
| | | ggatagtttctgaacatgtgtgctcataataaaacatattttcta | |
| | | tcgtctacaccttttttcttttacttttgctgcatcacaatcata | |
| | | cataaataacgtttaactttttagaatcat | |
| | | (SEQ ID NO: 331) | |
| 55 | BT3156 | Acaaaaaaacattcattcctaaaataaaggatgatggactaattt | BT3154 |
| | | atatatccatgaataaaggagataatcttaccataatcatccctt | |
| | | aataacaattttgcagtaaatgcgttattttttgctgcgtttata | |
| | | cactattaaaataacacatttacctaattttgactcgttgaattt | |
| | | agaaaattatcacattaaatctaataatatatgaaacgaattcga | |
| | | gatttccccaccactgacaagacaacgatctagttgcctgaaatg | |
| | | tattaacctaatattaattttaaattatcc | |
| | | (SEQ ID NO: 332) | |
| 56 | BT3174 | Ctgcaaaacgtcctgtttctaaaaaatgcgggtttttgcctaaaa | BT3172 |
| | | aacagtttgtttgtcactgcacgggaagtgaatcaccatctattg | |
| | | gcatttgtttttgatcacttctctatcaataatgatcatttagcg | |
| | | gattgcacagggttcttttgatcattattgaattttaattgatga | |
| | | tttttgaatgtttccttttatgctgtggttgtatttttgtcgaag | |
| | | attgaaacttattgttagctagtgaaagtatgaatctttattatt | |
| | | aaatgtagtattatgaaaaaaaggaaacag | |
| | | (SEQ ID NO: 333) | |
| 57 | BT3239 | AAGGAAGTGTTTAGATGACATAATGATTATTTGAACAGGGCTAAA | no linked |
| | | TTTGCACATACTGACATAAATGTGACATTATGATAGTTTGATGAT | regulator |
| | | ATATATTTATCTTCATAACTATCATAATGTATTTACAATAGTTTT | |
| | | CAAATTTATTTTGAATAATTGTTTTGTATTTATAAATATGTGCTT | |
| | | ATATTTGCAAAATAGCACTAAGCGAAAATAAGCGACTTACTAATT | |
| | | CTTATAATAATAAAGAGTGTTTATGAAAAGAAAATTAATGCTGTT | |
| | | ATTGGCCTGCTTGCTGGCAAGCATAGGTCTAGTGATTGCCCAAAC | |
| | | ACCTAAAAAGGTGACAGGTGTCGTCATTTCTGAGGAAGATGATCA | |
| | | GCCTGTTGTGGGAGCATCCGTATTAGTGAAAGGTACAACG | |
| | | (SEQ | |
| | | ID NO: 334) | |
| 58 | BT3240 | CCTGTCACCTTTTTAGGTGTTTGGGCAATCACTAGACCTATGCTT | no linked |
| | | GCCAGCAAGCAGGCCAATAACAGCATTAATTTTCTTTTCATAAAC | regulator |
| | | ACTCTTTATTATTATAAGAATTAGTAAGTCGCTTATTTTCGCTTA | |
| | | GTGCTATTTTGCAAATATAAGCACATATTTATAAATACAAAACAA | |
| | | TTATTCAAAATAAATTTGAAAACTATTGTAAATACATTATGATAG | |
| | | TTATGAAGATAAATATATATCATCAAACTATCATAATGTCACATT | |
| | | TATGTCAGTATGTGCAAATTTAGCCCTGTTCAAATAATCATTATG | |
| | | TCATCTAAACACTTCCTTTTTATCTTTAACTAAGCCTTGAATAAT | |
| | | ATAAAAATTATATTAGATATAAATAATTAGTATGAAAATAGGTTA | |
| | | TAGGAGAAT | |
| | | (SEQ ID NO: 335) | |
| 59 | BT3270 | Tccttgcttttgtgggggtgaagtgactgtgatgtggcttattta | BT3269/ |
| | | agtggatgagccgttatgtttaacaggtaaacaaagcttggttag | 3270 |
| | | catttttagtaagctatatgtgttcgttccatgtctgtgtcttat | |
| | | tgcattgtatatcaattagctaagtccgaaagtgacggatgatga | |
| | | taaaatatgagtttcaaactccttagtaaaagctaaaaaatacat | |
| | | cattttaatagcggcaacgaaaaaaaacaaatattttttaagatg | |
| | | cctaattgtcatttattgatggctgaattttgattctgaaaaggg | |
| | | aatgatttgttaaatatgtcatttttgttgctgaataatgtcata | |
| | | atgatagtttttcattcttttccgttttcgtacggagagtatctc | |
| | | ataaaaaataatcatctttttgtcacccatttatggacgttatgt | |
| | | ataattatagtaaagcaaagataaaatcctcattagaaatagata | |
| | | aaatt | |
| | | (SEQ ID NO: 336) | |
| 60 | BT3278 | Tctcttgaaactgtgaagactcaaaagaagagagcaatgtccttc | BT3277/ |
| | | cttagaaaaaaattaggctcctatcattttctattattacaaatg | 3278 |
| | | ttacttccctaaaaagaagcataacactcaatccgctttacttgt | |
| | | taacacctgtttgcaatgaacaaaatgttcgttgtttttcttgat | |
| | | tattgtacaaaaagaacggtcttctaaataaataaagtatctttt | |
| | | gaataaaaaaacttcagaatcctgtccctcattttttcttcttag | |
| | | tcgtctttgctataaataacctgaaataat | |
| | | (SEQ ID NO: 337) | |
| 61 | BT3299 | Atattcctgaaagccggagaatccacaactgtcaagatgacatta | BT3302 |
| | | cctaaagaatcatttatgtattttgatgtcaacagaactcagttc | |
| | | gtcactgatccgggtatttacaatataatgcttggcttctcctct | |
| | | agagatattaaagcacaaaaaaatattcaatatacactatagatt | |
| | | gcataatcaataagtaacatgaaatatatttgtatcaaaactgta | |
| | | gcaatttacttgctcgcaaccttacctttattggcgaacgcctca | |
| | | acacacaaggtaaaaattacacatagcgtc | |
| | | (SEQ ID NO: 338) | |
| 62 | BT3310 | Gaatacaatttataattatcgggcgaaagtaaaaaacaaagctcg | BT3309 |
| | | tggttccagagaagatttcgaagatttagttcggaaaatacgttg | |
| | | atcgatgttgtaacctatccactttttaggcctctttggttgata | |
| | | ttaacttgctgtttattagtggtttgtatgtttgtataatccact | |
| | | tattataccctcattgtcaaatcacttcagtttctttatactttt | |
| | | gacctgtgcttctgaaatagtggcgaaagtcctgtcaagcacata | |
| | | ccttaattataaactttaaaattaatacgc | |
| | | (SEQ ID NO: 339) | |
| 63 | BT3332 | Aaaatggaactgggcaatgacaggcatccccagctgtacgattta | BT3334 |
| | | tccgccgatccatcggagaaaaacaatgtagcgaaacagcatccg | |
| | | gaagttgtcagggagctgtccgaactgcttgaatcggtaaagaca | |
| | | agataaccaatgagaatagcagatttgtattattttgtatcaatc | |
| | | ttgaataattctatccatatgagaacaatcaatgcgtatatcttt | |
| | | gccatcgtaaattcaacaaaatcacttaatttttaatatatgaaa | |
| | | aaacatctatccaaccagacaagaaaaagg | |
| | | (SEQ ID NO: 75) | |
| 64 | BT3347 | Acatttctcccttgaagggcatatttgcacagatattccccaaaa | no linked |
| | | gcctgcttaattatttaatgtaattgaaaatgtaattataataat | regulator |
| | | ctgactaatatataatatattctggtcatttcaaaaaaaaggcat | |
| | | tctaactcttctacatttttattatatttatttgtaattaaccga | |
| | | ttgattgttaattataattatcatcatcacacctacataaagtta | |
| | | tgcaaactaaataagaaattacttatttagttagctttgccatgt | |
| | | cttttctcagtcaaactcttcgttgatgagaatcagacatatata | |
| | | gatatttgtacttaaatctacaacatattatgagcacaacatgcc | |
| | | aacatcaacaaggtgaaggatacctaaaactcctaaagatttaga | |
| | | tacatattatgcttataataagaaagataggcaaacgttgtaagc | |
| | | aataatatttattgttcggatgcactttaaccattttattattta | |
| | | tattt | |
| | | (SEQ ID NO: 340) | |
| 65 | BT3477 | Aaagttagaagtcatcaattaatagaccttcattttgggttggta | BT3465?? |
| | | aggataaaatctgctcaaaaaaggataaatactgctcaaacaacg | |
| | | tcagagcccctattccgtatgttaaaagaacgtttgaaatccaaa | |
| | | aaagaatagattttgtcctttcttgaacacagattttccttgttt | |
| | | cttttattaattccattattttgtgtcatcaagatgagatactat | |
| | | ggaaaataaagagtttgttaaagctcgatatttgctagaaataca | |
| | | cgatttgttagccgcttctccataacatttcatcctaaatatgac | |
| | | agtactgatattatatattaaaggtataggaaaacgggtataaag | |
| | | attgcaattattcaaaggtgagatagtattaaggtaaaaagtgga | |
| | | tttaaaagaatacttagggcttgtttttttatgaggtaaataaga | |
| | | ttgatggatggataaccttggatttgaagattaatttaaagcaaa | |
| | | tattt | |
| | | (SEQ ID NO: 341) | |
| 66 | BT3492 | Gtttgtagatcctctttataaatacatcaatgaaactacttcacg | no linked |
| | | tgtgcctatcagtgactggcatgataccaaaactggcagaatgac | regulator |
| | | cggatttaaggcacgttccgtaattggaggatattggatacaagt | |
| | | gttaatggataagatgaatcattaaaaatatggacaataagttga | |
| | | aagaatactttttcgcatggatgaatcatttcagcataatacaac | |
| | | aagagataagtaatgaaccaacaatataatattttattaatcatt | |
| | | aattttttacgtatgaaaaaaaacaaaatc | |
| | | (SEQ ID NO: 342) | |
| 67 | BT3504 | Ccattggactcccgcaaagagtatacaaagtctgactggattatg | no linked |
| | | tggacagctgctatgtcttcagatttagaaactttcaagaaattc | regulator |
| | | atagatccactctataaatatatcaatgaaactacttcgcgggta | |
| | | cctatcagtgattggcatcacacagacagtggtgaatgggttggg | |
| | | tttaaagcgcgttcggttattggaggatactggatgcaagtgtta | |
| | | atggacaaaactcgatgaaatgtttaattaactatacatttatta | |
| | | ttaactagaatgaaaaacatgaaatgtaaa | |
| | | (SEQ ID NO: 343) | |
| 68 | BT3518 | Gaataaatgtcgaatttgactcagcgctaagtaagataaaatgtt | BT3517/ |
| | | ccggaaaaattgatttgaaagacaactttgagacagtaattaatg | 3518 |
| | | gattaacctttgtagcaccaatatcttatgcctatgatggacagt | |
| | | ataaaacttatcgggtcgtgaaaaaatagacacagaaaaatatag | |
| | | taattaataaaataaatctcaagaatttatggcgatttttataaa | |
| | | gagtggataatactgctgtattatccagatggggctcatatgcct | |
| | | gtaagtattaagttataaaataataatatt | |
| | | (SEQ ID NO: 344) | |
| 69 | BT3569 | Gtctgcctgatgtaaaagagtagttgcattacctccatcatgaca | no linked |
| | | agcagagagagacaaaatcaggaacaaccctataatataagaaag | regulator |
| | | aagtagcttcatagttcgtttcgttttacaaatgtaaatagaata | |
| | | aaacaaacaagcaacaaaaagacaaataattttttcacacatcat | |
| | | aattacaatgttaaaattgccctttctctctctttccctaatctt | |
| | | ttttgcctttcatttgttcactttgttactttcgccggaagacgg | |
| | | cagaataaacttcaacctaaatagtaacga | |
| | | (SEQ ID NO: 345) | |
| 70 | BT3607 | Aatgtcgcgatgagcaacagagtcaatcatgacccaacccctgaa | BT3609? |
| | | tcaaacgaaattatatatgatttttttaaatggtacttgcattca | |
| | | gcaaataaatctaccaaagagtagaaagatgcgttttttgaatac | |
| | | actttttcgtttttttgcgcaaaaaatcaactctggatgcctaac | |
| | | tttgttctatcaattcatccaagtaacctaaattacttgactaaa | |
| | | atgattagcctatgaaaaatatacactagaaaacataaaatgccc | |
| | | catctgcgaatggggcaatattaatgccatattaataaaactaat | |
| | | cataaattatcgcccaattaataaataacacaataataaattcat | |
| | | aattaataaattaaaagtgctagctcaaaggaattaataaagcta | |
| | | gcagatgattccgagttagtaataaatattaaactattaaaaatt | |
| | | caataggttaaatatatactaaaaagagtagaaactaaaaaaagt | |
| | | aaact | |
| | | (SEQ ID NO: 346) | |
| 71 | BT3679 | Cgtaaagggaactatagtgcatcttgcgtaccactatagtggcac | BT3678 |
| | | gaactaatatgactaacaaaaaacttaattgttcgtagctaagga | |
| | | cttaagaatgtgagcgataacctgcaaggagtgaactgatttcat | |
| | | taacggtttaccatataacaaattattgttttattgatgttatgt | |
| | | tatatgtggtttttattttacagatcagattatccttattccttg | |
| | | gtcgaattatagtttaaaaaggaggaatatctataattcgacctc | |
| | | agataactcatattggacctcatctggagtgacttaacttacatt | |
| | | tgcacttgttaattggtcgttgaaatcataaatcaattaatgaac | |
| | | ggttaattttttatttactaatatcttaaatatttttgct | |
| | | (SEQ ID NO: 347) | |
| 72 | BT3668 | Atttttccagttccaatcggcattatgaatagccaaacctatgtt | BT3667? |
| | | caaaacaagcgggtgcaatttatcaacaaatgaagccatagcata | |
| | | tagggtttattaatgtgaagataaagatatataaaaaataaatat | |
| | | aactcttttgatgtcgataattttaaataatatatcgatattgtt | |
| | | ttataataaatcctgctatagtcgttcatgtcgatattgtattac | |
| | | tctaaatcgtattagtcctacaatgtggactgccgagttgttaca | |
| | | tttgcggtatataatctaataggaatattt | |
| | | (SEQ ID NO: 348) | |
| 73 | BT3703 | Tacttcctgcctcatctgctttcgatttattgacagagaaaaagg | BT3705 |
| | | ttgaattaagaaatgaattaacacttccaagtcgtgaaatatacc | |
| | | ttttatccttttaataagttcacttcaacttatatttatcagaat | |
| | | acagaaaagaaataaactactgtaaaacaaaacattaacattaat | |
| | | acaaacttgtatttttattctagacaacccaaagtctcaagatgg | |
| | | ttgctatatttgcaatatccggaacagagcggagtaaaaatatta | |
| | | gttagcttataatttaataccataaataga | |
| | | (SEQ ID NO: 349) | |
| 74 | BT3749 | Atcgtctggatggaaaatctcataaagaaatagctgaagaattgg | BT3748/ |
| | | gtatcagtgtcaaaggagtggagtatcatataagcaaagctgtga | BT3749 |
| | | agctgctgcgtgataatctgaaagagtatgctcctttcctgatat | |
| | | tttttatttgatgatcttgaaaatatctttttctcctcttttttt | |
| | | aataacagcaaaaaaagttgtatttctcactagggattattttta | |
| | | gtgagtggtattctatatgaaagggggagcaaatatactcctgtt | |
| | | tattacttataaagtaactcaacttataat | |
| | | (SEQ ID NO: 350) | |
| 75 | BT3854 | Atcggtggcggagcctgtcctcatcggtgccggaaataggtctca | BT3853 |
| | | tcggtacccgaaataaatccttgcggagtaaggcactcattatac | |
| | | catcaaataataattgtttttttttatcatgaaaacattattgat | |
| | | aactagatacttatgcgaaaagaatgaaattctttatcgctatta | |
| | | atctaaaattaatatggcattaaggactggaattctatatttgct | |
| | | tcagaaacggacgggatatcctccactatgtttctgatgacttga | |
| | | gcggatcgaactataaccttataaaatgatggaaatatgaaaaac | |
| | | gacacttcttaactactgcttggacttacttgtcagagaaagttt | |
| | | tcttgagatgaaaacggtttgaatcaattaatgttaatctataaa | |
| | | ttatgaaaacaggaaagagcaaagatcatttgcaaagagtttttt | |
| | | taaggttcctttatttgataatggggagcatcctttctttggatt | |
| | | ctgtt | |
| | | (SEQ ID NO: 351) | |
| 76 | BT3952 | Ttatatggaagacctggaaggctgggcagattctattgtttgtac | BT3951 |
| | | cactacggaacaggcggattttgtcaggaagttcttgcaggtcgt | |
| | | ggagcagaatctggatcgtgaagatttgggctccacgtttgtggc | |
| | | agaacaaatgttgatgagttcccgtcagttctaccggaggtttaa | |
| | | ggagatttcgggtatgtcacccagcgatttgattaaagattaccg | |
| | | gatggagaaggccgcccgtttgttgcagaacgaggaactgtctat | |
| | | tcaggatgtaatttccgatgtaggtatatccagtcgtgcttactt | |
| | | ctacaaagagtttacccgtaagtttggcgtaactcctaaggtata | |
| | | tagagagaagcttttagggaatgacactcataaaaatcaggagtg | |
| | | aacagccaggaagcggtctggtattagttgccaagaaataaattc | |
| | | ccatgctgatctgtgctgctttttaaggttggtctcctttgtact | |
| | | ctcttttgttttccattatttattattctgatatatagggtatta | |
| | | tccagtactctattttaccatctgtactttaaacagacatttttt | |
| | | ctcttctctgttttttggcgcacaggagtatctcatttctttggc | |
| | | tattcctttaatttgcgtcaacgtattaaacgtatttggtaacct | |
| | | tattattattaattaaaaacaagtagt | |
| | | (SEQ ID NO: 352) | |
| 77 | BT3958 | Agaggaactatagtgcttcttgcgtccaactatagtggaacgaaa | BT3957 |
| | | gggaaactacagtagaacgaatgaatttggactgataagaaatta | |
| | | gtttttcaaagtctttcggttaatgattcgggattccttcattcc | |
| | | ttctgttctttggtcgtttggggtgacagatatatttgaattcat | |
| | | gtctgtatagattttatttattactgcttacgaataaacttttta | |
| | | ataaaagtggcgaaaaggcggctattataacttgatacaatgtgc | |
| | | gcaaacttgtgctctgtttgtacgtgaatgaatgctatgtgttga | |
| | | tatatagatgtttataatgtgctttatttctcctttctgttctat | |
| | | aaatgagcattttcattttgacataattttgtcacttctgcggaa | |
| | | tattcttaatattaaaagtttttaatttgcttccgcttataagaa | |
| | | ttatataggcgattgataaccttagtattaattaaaaaaaacaaa | |
| | | gtaac | |
| | | (SEQ ID NO: 353) | |
| 78 | BT3983 | Atatttgactccggattattttcgctggttgtatacggcatttac | unknown |
| | | tcgcgccacgaagacattgtatctggtgaattatcccaaagaaca | (possibly |
| | | aattttataaaagatttcttttttttcttcgtaaataattgattc | BT3994/93 |
| | | ccagtctagatgaatggggagatactcgttgtgaaaaaataaata | |
| | | tgagattatatttattttttttcaaaaacctctaagtgtttttca | |
| | | aggtatgggttgttatataaatgtgaatcgaaaataataaatgga | |
| | | atatcgattctaatatttatataaaaacta | |
| | | (SEQ ID NO: 354) | |
| 79 | BT4040 | Agaatataacaacctttatcacgctgtgtactatccaaatagcaa | no linked |
| | | ttaaatagtttctcccttacacccgtaattaaacgactgccaaaa | regulator |
| | | tgctttatatttaagaagtgagtttttcgcccggtctgaccgaaa | |
| | | caatcaaccaattaaaaaggaatagaggaataattaaactaagtt | |
| | | cttgtagaacttagcacaaaaaacagtctgttaatccatttgtat | |
| | | ctaaactaacgcatctaactaagcaatagatttaccatctttttt | |
| | | gctaaaaatcaacgattcgtgcgcaaatatactaattagtttgaa | |
| | | aaagtcatactatttttcaataatatttcatatttaaattacaaa | |
| | | taagtttttcaattattctcatttaatcaatcaacgggtagaaac | |
| | | agagtgagtatttcactattacacgtttacagcccccaatacttc | |
| | | ccattgattatcaacaacatgcattctattccatcttatcatcta | |
| | | ctatattatagttctacaagaactaagacatagaacctattattt | |
| | | ttctgactttaaatcagcatagttcagcacacaaataaaagtttt | |
| | | aacttctaattttaatgcctatgagaaagaaataagtattccaaa | |
| | | tccaagagtacccacaagtacttcaacttgtttttacccttttta | |
| | | cctttaataaaactaatattaattt | |
| | | (SEQ ID NO: 262) | |
| 80 | BT4080 | Ctattatcagaagatggagtgataaaagggcaatatagtactatg | BT4069? |
| | | ggagatacttatgttaattggacacgtgatgacaccataacggga | BT4070? |
| | | gatgtatatcaaaagagttattttgatcgtatgattgaaagaaat | |
| | | caagtaaatgcttatctgaaagaaaaggaagaagctgttcgtatt | |
| | | tggaaggaaatagaactgatatctcaaaaactacattttccctca | |
| | | gaagaattgaatcattttattcgtatttcctgttcgtatggacgc | |
| | | attaaatatgagctttttgctgtttcgtggcagattatgctatgc | |
| | | ggttatgtggcagatacaacaaaaaagtcttttaaccggatagag | |
| | | atggataaatatattactgcatttgatgatttgtggaaagaatgg | |
| | | aacgacttatctctagaaaacgataattgcccttcaatgtataag | |
| | | atttcttctaacttttttggttttcctgtaggtatacaagagacc | |
| | | attgataaatacaggaaatagttttcattttttcttcttatatgt | |
| | | tatagaatataaaaaaataaatgaatattacttagtggttagtat | |
| | | tagtctagtcgtcttactattaatagtagcaaaaagtatgagaaa | |
| | | gaaacataaaataacgaaagtaaaattatgatcattattattata | |
| | | taaattcttaaaaacggtattttct | |
| | | (SEQ ID NO: 263) | |
| 81 | BT4085 | Attttccgggtagtgctgacttttgtcgtccttatttttatcgta | ? |
| | | ttttcaattgggaaagcatatatgatgaagatgattgatagctac | |
| | | tgacggattcatcttggtaaattgatttagaaagtcatttcttca | |
| | | taaaagggagaaatgactttcttttttaatatgttctgagctttc | |
| | | ctgcatacaagtaaatttattaataaaaaacatgtaattgtttta | |
| | | gtggtatatcatttttaaatcgtcattagataaataacaataaaa | |
| | | tagtacacatattaaaaagaactaataata | |
| | | (SEQ ID NO: 264) | |
| 82 | BT4114 | Ttgagtaaagaatacaccaatctggaggtatatctgaatgagcta | BT4111 |
| | | gtgaatcatctgtatcctgcggattctaaaaaaaaaactaaaagc | |
| | | ccgaataaacagttataaaagattaagacgagagtacatttcccc | |
| | | ataaatcctcgtctttattcattttatgccggaaaaacaaaaaat | |
| | | actatcgtaaaaacaaatattacacatcccacaagccccaaaaac | |
| | | ataattttgcaccagtgagaactctaagagtggacacactcgaat | |
| | | attaataatctaatataaaagaacaaaag | |
| | | (SEQ ID NO: 265) | |
| 83 | BT4119 | Aattgagtgtccatcaggcggctagcatttagagtatgatgaatg | BT4124 |
| | | ctagccgcctgatggacacttttattctaatccaagcaaatactg | |
| | | aaaatgactagtagaaattactatgtaaagtaaaatatggaattt | |
| | | ttggtattttaatcccaaaatctcgtatctgatattttagtgata | |
| | | aaatagtgcacctaaacgctcaaaaagtcccctctctttatgttt | |
| | | tatataacttcttttgcacatatgaaacaaaaaataagaactata | |
| | | aatcaggaatattaatgaataaacatgagatgattcctgtttcaa | |
| | | ttttccccatgttttcaacgattgatttgcatgtaacataagtat | |
| | | tgattagatagtaatatgtattaactttaaattgaaatga | |
| | | (SEQ ID NO: 266) | |
| 84 | BT4136 | Agtacctaccaatggagaattcagcaacattatgcacaacccatt | BT4137 |
| | | gcctcaaacggtctatttcaatcaaaaagttcaggcacgctttat | |
| | | caaactggaagccaccactcccactgatacagccgcaaaagttga | |
| | | accaaatgaaatcggagtaacaatagccccttgattttacattca | |
| | | tcacaagtactaactaaaagcaaaaatcagaatagaaaagacgcc | |
| | | atctattttgatttttacactatataaatcttcttattatcaaat | |
| | | taactccaatgaaggcaaagaaagagaaattagtcttaatgaaga | |
| | | taacagatctttaatatataaggactgtctatccaaacttatcga | |
| | | aaaatttaattaaccacaagattcatgcttattgaaaacacaaga | |
| | | atgaattgttaacacttttaatttctatgattaatgaaaaacaaa | |
| | | atagtggtggaagaatactacaaatattaatcctgttttgtttgt | |
| | | atggt | |
| | | (SEQ ID NO: 267) | |
| 85 | BT4163 | Acaattatatttgaaaagtttgccatattgcttttatatatggtt | BT4178? |
| | | attcttgtttcaagaaaaaccggaataaattgtattatctacact | |
| | | ctttcttatttaaaaaacgcgttcagagcatgtattttcgttcaa | |
| | | aaggcggtattttccatgtttatgtacgattcttcattgctattt | |
| | | cttatataagtcgttaagtttgcaacagtttttcacaggtattat | |
| | | gataaagtgcccgaagaaaaacagaatctaacttttgtttaatta | |
| | | aaaaatagataaaatagatttatattgttacttaacagattttaa | |
| | | tattatgagaaatgtgatgaatcgtaagcggcattggctgctatt | |
| | | attgctcctctcacctttttttctctcttgtgaggacaag | |
| | | (SEQ ID NO: 268) | |
| 86 | BT4247 | Gaatgttatggatatcaattggaaggacggaagatacaacttcaa | BT4250/ |
| | | agaaaccccattatcccagctcatttcgattataaatcaaatata | BT4248 |
| | | ccaatcaaatataattctagaaggaaagtttactaaacagccttc | |
| | | attcacaggcagcatccgttatgatgaaacattggatgacgtgat | |
| | | agataaactctgtttcagccttaaccttacatacaaaaaacagaa | |
| | | tagaaaaatagtaatatataattaaccatatttctaataattaac | |
| | | ccaatatttaacctcacaacaagttaaatc | |
| | | (SEQ ID NO: 269) | |
| 87 | BT4266 | Ctggaattttttttatgtaatataccctttacttcttattacggc | |
| | | ctattcgccagagattgttggtgaaggagaaaatattattatata | |
| | | tggtagaaattaaggtgtgcattttatttcttatgcatttattct | |
| | | tttggggatctactaccttttttatttagtatatttgaaaataaa | |
| | | tagttgtgcttggctgttatgattcatttttatatttaacagatt | |
| | | gggataagttgctctagaacttggtagttttataccaataagtgt | |
| | | attttttcctgttttcataggctattcaaaggttattaagtttgt | |
| | | gtgctacttaagacggtgtaaagttattaataaaattggtgaacg | |
| | | caatgttttttttattttaattatcggcaaacgatgtttttttta | |
| | | atttttcattctactactcagatcgtaataagatagatataatct | |
| | | tttaattattgtatattgttgattattagtattttatcgctttgg | |
| | | ataccaagttctagagcgacttattccaatcggtcaattacgact | |
| | | atatttcttaataattatttatgataatgcgaaattataggccgc | |
| | | ctccgggctgtgattaaatttttattatcgtttcttcagcataga | |
| | | tcagaacagtgctgaagagagattgaattcattaaaaatttcaat | |
| | | acccccaagaaaaacaaatgaatgataacagtcaaacacaactat | |
| | | atatattccaaaacttactaaatattaagataaagcatacacacc | |
| | | caaaaacaaacgtattagagtaaaaatgcacatcttatttctacc | |
| | | acattatcataatgctagatatagaatagtaagttttctacttat | |
| | | ccatattaacccaactaattattaacctttaatcacagaaaaaca | |
| | | (SEQ ID NO: 270) | |
| 88 | BT4299 | Tggagagcaatagagaccttatgccaaattgaataattttgaaga | BT4300? |
| | | agtcgtaataggacttggtatgtatgtgtataagtgtcttatata | |
| | | taattgattaattggttgatggatgcttgggatgacaatttatga | |
| | | actatatagtagaattgagattctaaaaaaatggtgtttgttgat | |
| | | aattaaaataaaaaagatattgtatttttcgattttattaataac | |
| | | tttacaccgtcttagaatagtacatgcacttaataacctttgaat | |
| | | agcctatggaaacagtagaaaatatactttggggtataaaaatac | |
| | | caagtcctattacgacttagccccaattaatcaatatgattataa | |
| | | ccttaaattaacatttatgaaaactcttagcaacagccctccgcc | |
| | | tttcaaaatcggagaattcgatctcatttttttacttttatttca | |
| | | gcacagatcagaataggtatgatatgtattctgagttcattatca | |
| | | acttcaatacccccaatttaagaagatgaataatagcagtcaaac | |
| | | acacccatatattttcaaacttactcatacatacaaaaacaaacg | |
| | | tataccccttgtatgcataccctagttctacccacatagtagaca | |
| | | cacaagagtaggtttccctatcacttttttttaaccaatctaatt | |
| | | attaaccttttatcacagaaaaaca | |
| | | (SEQ ID NO: 271) | |
| 89 | BT4356 | Cgttcacgggaacctttacaaatcaacgtttggaaaagattctgg | BT4355/ |
| | | aatattttaaagtatcctccaaaatacgctggaaacatatcaatg | 56 |
| | | atgacaaggatggaagtgacagaaaaaaagagataatagagattt | |
| | | attgattaacctttaacaatttagtagtatgaaagcaaaacccac | |
| | | ttagaaaaaagtatgggaagattttggcagatctccccatactaa | |
| | | aatgaatcaattgaaaatgcttgtttggcagacaagcatagttaa | |
| | | ctcattttcaacttaaatcaatacaaaatt | |
| | | (SEQ ID NO: 272) | |
| 90 | BT4403 | Ggaagttgatgctgaagatgttgctcaagatattttctgtaagct | BT4402/ |
| | | ttggttacaaccggaaatatggcttgacaatgaaaatgaattaga | 03 |
| | | taactacctctttataatgaccagaaacattattctgaatatctt | |
| | | caagcatcagcaaatcgaacgggaatatcaggaaggataccttga | |
| | | gaaaacagtacttcatgaattgatagaaggggaagatgcccttaa | |
| | | taatatatattatgaagaaatgttgctggttgttcgcctgacttt | |
| | | ggaaaaaatgcctgaacggcgaaggttaatattcgagctgagtcg | |
| | | ttttaagggattaagttataaagaaatcgcagaaaagcttaatgt | |
| | | ttccatccgtacggtagagcaccaggtgtatttagcactgataga | |
| | | actaaaaaaagtgcttctatttttattttttctattttcctttaa | |
| | | gtagtgtttttcttttagttgtatttagtgtataaaggaagaaat | |
| | | ta | |
| | | (SEQ ID NO: 273) | |
| 91 | BT4470 | Tctttgtctccgaaaaacaagagtccgacaatgcgccacatatca | ? |
| | | ttgttcaggttcggataataaaaatccatcgaccaccgttttctt | |
| | | tgtggcggaaaacttcccagcatcagcaactttgcttttggtggg | |
| | | agaaagggttttaaaggatgtttttcgatttcaatctccatattg | |
| | | atgtaatttcctacaaaaataacgaatctattcttttccttcaaa | |
| | | atagttgcggaaaaaaacaaatttaaaaatatttggctatttttg | |
| | | cacggagatggtgaaaaacaacttattgaa | |
| | | (SEQ ID NO: 274) | |
| 92 | BT4635 | Ctcccaataacttattaagttataatgaacaggaaggcatccata | BT4636/ |
| | | tccaatcggtagacgtacaaaaatatatcgcctggaaagatggtt | 35 |
| | | tttacctcttccaaacagaaaaactaaaagacattgctacaaaac | |
| | | tcagcgattactatggcaaaaagatcatgatagacagtccattga | |
| | | agaccattacctgcagtggtaaactagatttgaaagaagatcttg | |
| | | acgaagttcttcagacactaatacggactgtcccggcaagaatag | |
| | | aaaaatcagatggaattatccatatttatgtaaaacattaattaa | |
| | | aatcgaaagcctatgaataaacattatccttaaaaaaataaggcc | |
| | | gaataatacccttaatatcatccggcctttgattaaattacttac | |
| | | acgttaatattaattaatctaatcattacaaatgtatgaaaaagc | |
| | | accgattatttagtcaccaaaggactaaagataaacaactattac | |
| | | taatc | |
| | | (SEQ ID NO: 275) | |
| 93 | BT4662 | Tttttttcttcttttattgaatgatgacggattttaaaatcatcc | BT4663 |
| | | atccaaacaacctaaattgtacagaacctattccatatcatatta | |
| | | caatgtcataacattatgacacataaaaaactaggatacaggaga | |
| | | taaacagcttttttaaacaaaaacaacatatgtctcaacaaattt | |
| | | gatatcaaaaaaatcatttttgaacaaagtatgaacaacctaaat | |
| | | taaagaatctaattaatgaggttacatttgcaacagtaaaataac | |
| | | acttttaataattataaaacctaataaagt | |
| | | (SEQ ID NO: 276) | |
| 94 | BT4672 | Tgtctcccgtggaatatatcaagtccatccgcatgaagaaagctg | BT4673 |
| | | ccatgttattgcaacaaaagaaattcaccgttgccgaagtgatgt | |
| | | atatggtaggattttccaatcattcttatttctccaaatgctttc | |
| | | aggcagagtttggaaaaacaccgcgccaatacttgaatgacgggc | |
| | | tgtagaggcattttagggcttttcgtcctatttttatccttatat | |
| | | gtccaatctgtgcttttatgaaaatgccaggcagtctatctttgc | |
| | | cgctatcaacaaaccactaaatttgatagc | |
| | | (SEQ ID NO: 277) | |
| 95 | BT4706 | caggcagatgatacattcacaaagaaaaagataatagtagattta | BT4705/ |
| | | tattagtaaccataaaaatgagaatagcctatgagataacacaaa | 06 |
| | | gatcgtaaaaaaaggaagaaagctgctaccaacagctgtcttccc | |
| | | ccaatttttcagattcgcggtaaggagtcttgcaaacgctaaccg | |
| | | gaatcaaaccacagttcattatagtaataaactttagttaaccgc | |
| | | aaatgtatgaaaaaaaatcattcatttattgcattatatcctaaa | |
| | | tatgctttaaatctaaaactacctttagtt | |
| | | (SEQ ID NO: 278) | |
| 96 | BT4723 | Acttcctaaagaatactttacgggaacgattcctagtggcggcat | BT4722/ |
| | | acagaacgccttgaatattctcatgcttacttcgcctttttatta | 23 |
| | | tgaaatggatggctcggtcattgtcttgaaagagaagtagataag | |
| | | catctttatcatgaaattagatatagccattcctgagttttcccc | |
| | | cggaatggctttttaatttatgaagaaccgttagttaacaagagt | |
| | | taaagatagatagccgattggacgaaaggggagagcagtcggtat | |
| | | acctgtcaaacaacttaaaagttttgatat | |
| | | (SEQ ID NO: 279) | |

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A glycan-sensing system comprising an engineered bacteria harboring a reporter plasmid, wherein the reporter plasmid comprises a luciferase reporter cassette, a first polysaccharide utilization locus (PUL), and a first glycan-specific promoter, wherein the glycan-sensing system detects and quantifies a glycan molecule.

2. The system of claim 1, wherein the glycan molecule comprises chondroitin sulfate, arabinan, heparan sulfate, hyaluronan, fructan, levan, mannan, or derivatives thereof.

3. (canceled)

4. The system of claim 1, wherein the first PUL encodes any combination of proteins comprising a surface glycan binding protein (SGBP), an outer membrane channel, a digestive enzyme, a glycan importer, or a glycan sensor.

5. The system of claim 1, wherein the first PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof.

6. The system of claim 1, wherein the first glycan specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof.

7. The system of claim 1, further comprising a second PUL.

8. The system of any one of claim 7, wherein the second PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof.

9. The system of claim 7, wherein the first PUL is interchangeable with the second PUL.

10. The system of claim 1, further comprising a second glycan-specific promoter.

11. The system of claim 10, wherein the first glycan-specific promoter is interchangeable with the second glycan-specific promoter.

12. The system of claim 10, wherein the second glycan-specific promoter comprises a CS-specific promoter, a levan-specific promoter, an inulin-specific promoter, an arabinogalactan-specific promoter, a dextran-specific promoter, a HA-specific promoter, a HS-specific promoter, an O-glycan(OG)-specific promoter, or variants thereof.

13. The system of claim 1, wherein the first or second glycan-specific promoter comprises SEQ ID NO: 74, SEQ ID NO: 75, SEQ ID NO: 76, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, or SEQ ID NO: 354.

14. The system of claim 1, wherein the reporter plasmid comprises a Bacteroides-optimized lux (Bolux) plasmid.

15. (canceled)

16. The system of claim 1, wherein the engineered bacteria originates from a Bacteroides bacterium.

17-29. (canceled)

30. A method of detecting a glycan, the method comprising:
isolating and preserving a tissue sample from a subject,
preparing and culturing the glycan-sensing system of claim 1 in a microplate in an anaerobic environment,
adding the tissue sample into the microplate, and
detecting and quantifying a light signal emitted from the system.

31-34. (canceled)

35. A method of treating or preventing a gastrointestinal disease or disorder in a subject in need thereof, the method comprising:
isolating and preserving a tissue sample from a subject,
preparing and culturing the glycan-sensing system of claim 1 in a microplate in an anaerobic environment,
adding the tissue sample into the microplate,
detecting and quantifying a light signal emitted from the system, and
performing or administering a therapeutic procedure to the subject comprising pathological amounts of a glycan relative to a control tissue comprising normal amounts of the glycan.

36-44. (canceled)

45. A kit for detecting or purifying a glycan molecule, the kit comprising:
an apparatus comprising a binding medium and a surface glycan binding protein (SGBP), wherein the binding medium comprises a matrix of biomolecules, the SGBP comprises a peptide label, and the SGBP is attached to the matrix,
a container comprising an equilibration buffer,
a container comprising a releasing buffer, and
a container comprising a washing buffer.

46-52. (canceled)

53. A reporter plasmid comprising a luciferase reporter cassette, a first polysaccharide utilization locus (PUL), and a first glycan-specific promoter.

54-56. (canceled)

57. The reporter plasmid of claim 53, wherein the first PUL encodes any combination of proteins comprising a surface glycan binding protein (SGBP), an outer membrane channel, a digestive enzyme, a glycan importer, or a glycan sensor.

58. The reporter plasmid of claim 53, wherein the first PUL comprises a chondroitin sulfate (CS)-specific PUL, a levan-specific PUL, an inulin-specific PUL, an arabinogalactan-specific PUL, a dextran-specific PUL, a hyaluronan (HA)-specific PUL, a heparan sulfate (HS)-specific PUL, an O-glycan(OG)-specific PUL, or variants thereof.

59-66. (canceled)

67. An engineered bacteria comprising the reporter plasmid of claim 53.

68-92. (canceled)
